SEQUENCE LISTING (2146 sequences)

SEQ ID NO: 1 - DNA, 20 bp, artificial
  human FXR antisense
aggcatcctctgtttgttat

SEQ ID NO: 2 - DNA, 20 bp, artificial
  human FXR antisense
cctgaggcatcctctgtttg

SEQ ID NO: 3 - DNA, 20 bp, artificial
  human FXR antisense
cgcgcccatgcggggcttct

SEQ ID NO: 4 - DNA, 20 bp, artificial
  human FXR antisense
gacgcgcccatgcggggctt

SEQ ID NO: 5 - DNA, 20 bp, artificial
  human FXR antisense
ggcatcctctgtttgttata

SEQ ID NO: 6 - DNA, 20 bp, artificial
  human FXR antisense
cgacactcttgacactttct

SEQ ID NO: 7 - DNA, 20 bp, artificial
  human FXR antisense
tgacgcgcccatgcggggct

SEQ ID NO: 8 - DNA, 20 bp, artificial
  human FXR antisense
gcgcccatgcggggcttctt

SEQ ID NO: 9 - DNA, 20 bp, artificial
  human FXR antisense
acgcgcccatgcggggcttc

SEQ ID NO: 10 - DNA, 20 bp, artificial
  human FXR antisense
ctgacgcgcccatgcggggc

SEQ ID NO: 11 - DNA, 20 bp, artificial
  human FXR antisense
ctgaggcatcctctgtttgt

SEQ ID NO: 12 - DNA, 20 bp, artificial
  human FXR antisense
cccatgcggggcttctttgt

SEQ ID NO: 13 - DNA, 20 bp, artificial
  human FXR antisense
ccatcacacagttgcccccg

SEQ ID NO: 14 - DNA, 20 bp, artificial
  human FXR antisense
tcgacactcttgacactttc

SEQ ID NO: 15 - DNA, 20 bp, artificial
  human FXR antisense
tcacacagttgcccccgttt

SEQ ID NO: 16 - DNA, 20 bp, artificial
  human FXR antisense
gaggcatcctctgtttgtta

SEQ ID NO: 17 - DNA, 20 bp, artificial
  human FXR antisense
gacactcttgacactttctt

SEQ ID NO: 18 - DNA, 20 bp, artificial
  human FXR antisense
gtcgacactcttgacacttt

SEQ ID NO: 19 - DNA, 20 bp, artificial
  human FXR antisense
cacacagttgcccccgtttt

SEQ ID NO: 20 - DNA, 20 bp, artificial
  human FXR antisense
gcatcctctgtttgttatat

SEQ ID NO: 21 - DNA, 20 bp, artificial
  human FXR antisense
ttcctgaggcatcctctgtt

SEQ ID NO: 22 - DNA, 20 bp, artificial
  human FXR antisense
gcagtgttcactttgagcta

SEQ ID NO: 23 - DNA, 20 bp, artificial
  human FXR antisense
tgaggcatcctctgtttgtt

SEQ ID NO: 24 - DNA, 20 bp, artificial
  human FXR antisense
agcagtgttcactttgagct

SEQ ID NO: 25 - DNA, 20 bp, artificial
  human FXR antisense
gttatttcctgaggcatcct

SEQ ID NO: 26 - DNA, 20 bp, artificial
  human FXR antisense
ccatgcggggcttctttgtt

SEQ ID NO: 27 - DNA, 20 bp, artificial
  human FXR antisense
catcacacagttgcccccgt

SEQ ID NO: 28 - DNA, 20 bp, artificial
  human FXR antisense
agtcgacactcttgacactt

SEQ ID NO: 29 - DNA, 20 bp, artificial
  human FXR antisense
tgttatttcctgaggcatcc

SEQ ID NO: 30 - DNA, 20 bp, artificial
  human FXR antisense
tgcactttctttatggtggt

SEQ ID NO: 31 - DNA, 20 bp, artificial
  human FXR antisense
atcacacagttgcccccgtt

SEQ ID NO: 32 - DNA, 20 bp, artificial
  human FXR antisense
cccatctctttgcatttcct

SEQ ID NO: 33 - DNA, 20 bp, artificial
  human FXR antisense
tttcctgaggcatcctctgt

SEQ ID NO: 34 - DNA, 20 bp, artificial
  human FXR antisense
gtaattcagtcaggcgaccc

SEQ ID NO: 35 - DNA, 20 bp, artificial
  human FXR antisense
tagtcgacactcttgacact

SEQ ID NO: 36 - DNA, 20 bp, artificial
  human FXR antisense
gcactttctttatggtggtc

SEQ ID NO: 37 - DNA, 20 bp, artificial
  human FXR antisense
cgcccatgcggggcttcttt

SEQ ID NO: 38 - DNA, 20 bp, artificial
  human FXR antisense
tcccatctctttgcatttcc

SEQ ID NO: 39 - DNA, 20 bp, artificial
  human FXR antisense
gcccatgcggggcttctttg

SEQ ID NO: 40 - DNA, 20 bp, artificial
  human FXR antisense
tttttttttctgttgccatt

SEQ ID NO: 41 - DNA, 20 bp, artificial
  human FXR antisense
aagcagtgttcactttgagc

SEQ ID NO: 42 - DNA, 20 bp, artificial
  human FXR antisense
gccaattagaatgcaggatt

SEQ ID NO: 43 - DNA, 20 bp, artificial
  human FXR antisense
tttttctgttgccattatgt

SEQ ID NO: 44 - DNA, 20 bp, artificial
  human FXR antisense
gctgacgcgcccatgcgggg

SEQ ID NO: 45 - DNA, 20 bp, artificial
  human FXR antisense
ttgatcctccctgctgacgc

SEQ ID NO: 46 - DNA, 20 bp, artificial
  human FXR antisense
acacagttgcccccgttttt

SEQ ID NO: 47 - DNA, 20 bp, artificial
  human FXR antisense
cagccaacattcccatctct

SEQ ID NO: 48 - DNA, 20 bp, artificial
  human FXR antisense
cactttctttatggtggtct

SEQ ID NO: 49 - DNA, 20 bp, artificial
  human FXR antisense
ttagtcgacactcttgacac

SEQ ID NO: 50 - DNA, 20 bp, artificial
  human FXR antisense
tctgcatgctgcttcacatt

SEQ ID NO: 51 - DNA, 20 bp, artificial
  human FXR antisense
ttttttttctgttgccatta

SEQ ID NO: 52 - DNA, 20 bp, artificial
  human FXR antisense
gtgttcactttgagctatgt

SEQ ID NO: 53 - DNA, 20 bp, artificial
  human FXR antisense
catcctctgtttgttatatg

SEQ ID NO: 54 - DNA, 20 bp, artificial
  human FXR antisense
cttgccaattagaatgcagg

SEQ ID NO: 55 - DNA, 20 bp, artificial
  human FXR antisense
tttttttctgttgccattat

SEQ ID NO: 56 - DNA, 20 bp, artificial
  human FXR antisense
gcatacgcctgagttcatat

SEQ ID NO: 57 - DNA, 20 bp, artificial
  human FXR antisense
tccatcacacagttgccccc

SEQ ID NO: 58 - DNA, 20 bp, artificial
  human FXR antisense
ccttagtcgacactcttgac

SEQ ID NO: 59 - DNA, 20 bp, artificial
  human FXR antisense
tccttagtcgacactcttga

SEQ ID NO: 60 - DNA, 20 bp, artificial
  human FXR antisense
atcctctgtttgttatatga

SEQ ID NO: 61 - DNA, 20 bp, artificial
  human FXR antisense
atttcctgaggcatcctctg

SEQ ID NO: 62 - DNA, 20 bp, artificial
  human FXR antisense
ttttttctgttgccattatg

SEQ ID NO: 63 - DNA, 20 bp, artificial
  human FXR antisense
ccctgctgacgcgcccatgc

SEQ ID NO: 64 - DNA, 20 bp, artificial
  human FXR antisense
tcagccaacattcccatctc

SEQ ID NO: 65 - DNA, 20 bp, artificial
  human FXR antisense
ctgcatgctgcttcacattt

SEQ ID NO: 66 - DNA, 20 bp, artificial
  human FXR antisense
tgtttgttatatgaatccat

SEQ ID NO: 67 - DNA, 20 bp, artificial
  human FXR antisense
agccaacattcccatctctt

SEQ ID NO: 68 - DNA, 20 bp, artificial
  human FXR antisense
gcatgctgcttcacattttt

SEQ ID NO: 69 - DNA, 20 bp, artificial
  human FXR antisense
tcctgaggcatcctctgttt

SEQ ID NO: 70 - DNA, 20 bp, artificial
  human FXR antisense
ttcagtcaggcgacccagga

SEQ ID NO: 71 - DNA, 20 bp, artificial
  human FXR antisense
tgccaattagaatgcaggat

SEQ ID NO: 72 - DNA, 20 bp, artificial
  human FXR antisense
ttgccaattagaatgcagga

SEQ ID NO: 73 - DNA, 20 bp, artificial
  human FXR antisense
catacgcctgagttcatata

SEQ ID NO: 74 - DNA, 20 bp, artificial
  human FXR antisense
tatttcctgaggcatcctct

SEQ ID NO: 75 - DNA, 20 bp, artificial
  human FXR antisense
ttatttcctgaggcatcctc

SEQ ID NO: 76 - DNA, 20 bp, artificial
  human FXR antisense
cagtgttcactttgagctat

SEQ ID NO: 77 - DNA, 20 bp, artificial
  human FXR antisense
tttttggtaatgcttctcct

SEQ ID NO: 78 - DNA, 20 bp, artificial
  human FXR antisense
cacagttgcccccgttttta

SEQ ID NO: 79 - DNA, 20 bp, artificial
  human FXR antisense
ttcagccaacattcccatct

SEQ ID NO: 80 - DNA, 20 bp, artificial
  human FXR antisense
taattcagtcaggcgaccca

SEQ ID NO: 81 - DNA, 20 bp, artificial
  human FXR antisense
ttttctgttgccattatgtt

SEQ ID NO: 82 - DNA, 20 bp, artificial
  human FXR antisense
atccatcacacagttgcccc

SEQ ID NO: 83 - DNA, 20 bp, artificial
  human FXR antisense
atgagagagaaaaaggagct

SEQ ID NO: 84 - DNA, 20 bp, artificial
  human FXR antisense
acactcttgacactttcttc

SEQ ID NO: 85 - DNA, 20 bp, artificial
  human FXR antisense
cacaatgtagagaaagttgt

SEQ ID NO: 86 - DNA, 20 bp, artificial
  human FXR antisense
gaagcagtgttcactttgag

SEQ ID NO: 87 - DNA, 20 bp, artificial
  human FXR antisense
atgcactttctttatggtgg

SEQ ID NO: 88 - DNA, 20 bp, artificial
  human FXR antisense
actcttgacactttcttcgc

SEQ ID NO: 89 - DNA, 20 bp, artificial
  human FXR antisense
ccatctctttgcatttcctt

SEQ ID NO: 90 - DNA, 20 bp, artificial
  human FXR antisense
catgctgcttcacatttttt

SEQ ID NO: 91 - DNA, 20 bp, artificial
  human FXR antisense
tcctctgtttgttatatgaa

SEQ ID NO: 92 - DNA, 20 bp, artificial
  human FXR antisense
cctttcagcaaagcaatctg

SEQ ID NO: 93 - DNA, 20 bp, artificial
  human FXR antisense
ggggtaaacttgtggtcgtt

SEQ ID NO: 94 - DNA, 20 bp, artificial
  human FXR antisense
tggggtaaacttgtggtcgt

SEQ ID NO: 95 - DNA, 20 bp, artificial
  human FXR antisense
gtggggtaaacttgtggtcg

SEQ ID NO: 96 - DNA, 20 bp, artificial
  human FXR antisense
tacgcctgagttcatatatt

SEQ ID NO: 97 - DNA, 20 bp, artificial
  human FXR antisense
tccctgctgacgcgcccatg

SEQ ID NO: 98 - DNA, 20 bp, artificial
  human FXR antisense
acagttgcccccgtttttac

SEQ ID NO: 99 - DNA, 20 bp, artificial
  human FXR antisense
gccaacattcccatctcttt

SEQ ID NO: 100 - DNA, 20 bp, artificial
  human FXR antisense
tgcatgctgcttcacatttt

SEQ ID NO: 101 - DNA, 20 bp, artificial
  human FXR antisense
tgttcactttgagctatgtt

SEQ ID NO: 102 - DNA, 20 bp, artificial
  human FXR antisense
atacgcctgagttcatatat

SEQ ID NO: 103 - DNA, 20 bp, artificial
  human FXR antisense
atgctgcttcacattttttc

SEQ ID NO: 104 - DNA, 20 bp, artificial
  human FXR antisense
agtgttcactttgagctatg

SEQ ID NO: 105 - DNA, 20 bp, artificial
  human FXR antisense
actttctttatggtggtctt

SEQ ID NO: 106 - DNA, 20 bp, artificial
  human FXR antisense
gttgcccccgtttttacact

SEQ ID NO: 107 - DNA, 20 bp, artificial
  human FXR antisense
gttcagttttctccctgcat

SEQ ID NO: 108 - DNA, 20 bp, artificial
  human FXR antisense
agttcagttttctccctgca

SEQ ID NO: 109 - DNA, 20 bp, artificial
  human FXR antisense
cctctgtttgttatatgaat

SEQ ID NO: 110 - DNA, 20 bp, artificial
  human FXR antisense
gcttgccaattagaatgcag

SEQ ID NO: 111 - DNA, 20 bp, artificial
  human FXR antisense
tctttgttacaggcatctct

SEQ ID NO: 112 - DNA, 20 bp, artificial
  human FXR antisense
gcatttccttagtcgacact

SEQ ID NO: 113 - DNA, 20 bp, artificial
  human FXR antisense
ctttgcatttccttagtcga

SEQ ID NO: 114 - DNA, 20 bp, artificial
  human FXR antisense
acaatgtagagaaagttgtt

SEQ ID NO: 115 - DNA, 20 bp, artificial
  human FXR antisense
gaatccaatttcgcattagg

SEQ ID NO: 116 - DNA, 20 bp, artificial
  human FXR antisense
accactcttcaggctgctgg

SEQ ID NO: 117 - DNA, 20 bp, artificial
  human FXR antisense
gtttttggtaatgcttctcc

SEQ ID NO: 118 - DNA, 20 bp, artificial
  human FXR antisense
attcagccaacattcccatc

SEQ ID NO: 119 - DNA, 20 bp, artificial
  human FXR antisense
atctgcatgctgcttcacat

SEQ ID NO: 120 - DNA, 20 bp, artificial
  human FXR antisense
tttctgttgccattatgttt

SEQ ID NO: 121 - DNA, 20 bp, artificial
  human FXR antisense
gttcactttgagctatgttt

SEQ ID NO: 122 - DNA, 20 bp, artificial
  human FXR antisense
tgatcctccctgctgacgcg

SEQ ID NO: 123 - DNA, 20 bp, artificial
  human FXR antisense
ttccttagtcgacactcttg

SEQ ID NO: 124 - DNA, 20 bp, artificial
  human FXR antisense
tctttgcatttccttagtcg

SEQ ID NO: 125 - DNA, 20 bp, artificial
  human FXR antisense
tgctgcttcacattttttct

SEQ ID NO: 126 - DNA, 20 bp, artificial
  human FXR antisense
ttgttatttcctgaggcatc

SEQ ID NO: 127 - DNA, 20 bp, artificial
  human FXR antisense
acacaatgtagagaaagttg

SEQ ID NO: 128 - DNA, 20 bp, artificial
  human FXR antisense
gcatgactttgttgtcgagg

SEQ ID NO: 129 - DNA, 20 bp, artificial
  human FXR antisense
agtggggtaaacttgtggtc

SEQ ID NO: 130 - DNA, 20 bp, artificial
  human FXR antisense
tccaatttcgcattaggata

SEQ ID NO: 131 - DNA, 20 bp, artificial
  human FXR antisense
ctcttcaggctgctgggggt

SEQ ID NO: 132 - DNA, 20 bp, artificial
  human FXR antisense
catgcggggcttctttgtta

SEQ ID NO: 133 - DNA, 20 bp, artificial
  human FXR antisense
ctcttgacactttcttcgca

SEQ ID NO: 134 - DNA, 20 bp, artificial
  human FXR antisense
cgtaattcagtcaggcgacc

SEQ ID NO: 135 - DNA, 20 bp, artificial
  human FXR antisense
tatgagagagaaaaaggagc

SEQ ID NO: 136 - DNA, 20 bp, artificial
  human FXR antisense
agaagcagtgttcactttga

SEQ ID NO: 137 - DNA, 20 bp, artificial
  human FXR antisense
aattcagtcaggcgacccag

SEQ ID NO: 138 - DNA, 20 bp, artificial
  human FXR antisense
tgagagagaaaaaggagcta

SEQ ID NO: 139 - DNA, 20 bp, artificial
  human FXR antisense
tcagaatccaatttcgcatt

SEQ ID NO: 140 - DNA, 20 bp, artificial
  human FXR antisense
cccctttgatcctccctgct

SEQ ID NO: 141 - DNA, 20 bp, artificial
  human FXR antisense
ccaacattcccatctctttg

SEQ ID NO: 142 - DNA, 20 bp, artificial
  human FXR antisense
ctgcatgactttgttgtcga

SEQ ID NO: 143 - DNA, 20 bp, artificial
  human FXR antisense
ataggtcagaatgcccagac

SEQ ID NO: 144 - DNA, 20 bp, artificial
  human FXR antisense
gagctagacccctcccctgt

SEQ ID NO: 145 - DNA, 20 bp, artificial
  human FXR antisense
ccaatttcgcattaggataa

SEQ ID NO: 146 - DNA, 20 bp, artificial
  human FXR antisense
atccaatttcgcattaggat

SEQ ID NO: 147 - DNA, 20 bp, artificial
  human FXR antisense
ggacctgccacttgttctgt

SEQ ID NO: 148 - DNA, 20 bp, artificial
  human FXR antisense
acgcctgagttcatatattc

SEQ ID NO: 149 - DNA, 20 bp, artificial
  human FXR antisense
ttctttgttacaggcatctc

SEQ ID NO: 150 - DNA, 20 bp, artificial
  human FXR antisense
tcctccctgctgacgcgccc

SEQ ID NO: 151 - DNA, 20 bp, artificial
  human FXR antisense
agttgcccccgtttttacac

SEQ ID NO: 152 - DNA, 20 bp, artificial
  human FXR antisense
tcattcacggtctgatctgc

SEQ ID NO: 153 - DNA, 20 bp, artificial
  human FXR antisense
gagttcagttttctccctgc

SEQ ID NO: 154 - DNA, 20 bp, artificial
  human FXR antisense
ttgttacaggcatctctgct

SEQ ID NO: 155 - DNA, 20 bp, artificial
  human FXR antisense
tgcatttccttagtcgacac

SEQ ID NO: 156 - DNA, 20 bp, artificial
  human FXR antisense
tttcgcattaggataagtcg

SEQ ID NO: 157 - DNA, 20 bp, artificial
  human FXR antisense
tttgttacaggcatctctgc

SEQ ID NO: 158 - DNA, 20 bp, artificial
  human FXR antisense
ccctttgatcctccctgctg

SEQ ID NO: 159 - DNA, 20 bp, artificial
  human FXR antisense
ttgcatttccttagtcgaca

SEQ ID NO: 160 - DNA, 20 bp, artificial
  human FXR antisense
catgactttgttgtcgaggt

SEQ ID NO: 161 - DNA, 20 bp, artificial
  human FXR antisense
tgcatgactttgttgtcgag

SEQ ID NO: 162 - DNA, 20 bp, artificial
  human FXR antisense
aatccaatttcgcattagga

SEQ ID NO: 163 - DNA, 20 bp, artificial
  human FXR antisense
ctttgttacaggcatctctg

SEQ ID NO: 164 - DNA, 20 bp, artificial
  human FXR antisense
tttgatcctccctgctgacg

SEQ ID NO: 165 - DNA, 20 bp, artificial
  human FXR antisense
ttttggtaatgcttctcctg

SEQ ID NO: 166 - DNA, 20 bp, artificial
  human FXR antisense
cctgcatgactttgttgtcg

SEQ ID NO: 167 - DNA, 20 bp, artificial
  human FXR antisense
gtttgttatatgaatccata

SEQ ID NO: 168 - DNA, 20 bp, artificial
  human FXR antisense
ctgtttgttatatgaatcca

SEQ ID NO: 169 - DNA, 20 bp, artificial
  human FXR antisense
agcatctcagcgtggtgatg

SEQ ID NO: 170 - DNA, 20 bp, artificial
  human FXR antisense
gggtaaacttgtggtcgttt

SEQ ID NO: 171 - DNA, 20 bp, artificial
  human FXR antisense
aacacaatgtagagaaagtt

SEQ ID NO: 172 - DNA, 20 bp, artificial
  human FXR antisense
atttcgcattaggataagtc

SEQ ID NO: 173 - DNA, 20 bp, artificial
  human FXR antisense
taccactcttcaggctgctg

SEQ ID NO: 174 - DNA, 20 bp, artificial
  human FXR antisense
tttgcatttccttagtcgac

SEQ ID NO: 175 - DNA, 20 bp, artificial
  human FXR antisense
ccctttcagcaaagcaatct

SEQ ID NO: 176 - DNA, 20 bp, artificial
  human FXR antisense
tcagtcaggcgacccaggag

SEQ ID NO: 177 - DNA, 20 bp, artificial
  human FXR antisense
cagaatccaatttcgcatta

SEQ ID NO: 178 - DNA, 20 bp, artificial
  human FXR antisense
cttagtcgacactcttgaca

SEQ ID NO: 179 - DNA, 20 bp, artificial
  human FXR antisense
tttccttagtcgacactctt

SEQ ID NO: 180 - DNA, 20 bp, artificial
  human FXR antisense
atgactttgttgtcgaggtc

SEQ ID NO: 181 - DNA, 20 bp, artificial
  human FXR antisense
ggtgatgattgaatgtccgt

SEQ ID NO: 182 - DNA, 20 bp, artificial
  human FXR antisense
atgagattttccctagttca

SEQ ID NO: 183 - DNA, 20 bp, artificial
  human FXR antisense
ttcgcattaggataagtcgg

SEQ ID NO: 184 - DNA, 20 bp, artificial
  human FXR antisense
gacctgccacttgttctgtt

SEQ ID NO: 185 - DNA, 20 bp, artificial
  human FXR antisense
cctgctgacgcgcccatgcg

SEQ ID NO: 186 - DNA, 20 bp, artificial
  human FXR antisense
ttgttgtcgaggtcacttgt

SEQ ID NO: 187 - DNA, 20 bp, artificial
  human FXR antisense
gttgttctatctagcccaat

SEQ ID NO: 188 - DNA, 20 bp, artificial
  human FXR antisense
tcactttgagctatgtttct

SEQ ID NO: 189 - DNA, 20 bp, artificial
  human FXR antisense
taggtcagaatgcccagacg

SEQ ID NO: 190 - DNA, 20 bp, artificial
  human FXR antisense
ttgcccccgtttttacactt

SEQ ID NO: 191 - DNA, 20 bp, artificial
  human FXR antisense
tatccatcacacagttgccc

SEQ ID NO: 192 - DNA, 20 bp, artificial
  human FXR antisense
cttcattcacggtctgatct

SEQ ID NO: 193 - DNA, 20 bp, artificial
  human FXR antisense
gcagaccctttcagcaaagc

SEQ ID NO: 194 - DNA, 20 bp, artificial
  human FXR antisense
aataggtcagaatgcccaga

SEQ ID NO: 195 - DNA, 20 bp, artificial
  human FXR antisense
agctagacccctcccctgta

SEQ ID NO: 196 - DNA, 20 bp, artificial
  human FXR antisense
aatgcactttctttatggtg

SEQ ID NO: 197 - DNA, 20 bp, artificial
  human FXR antisense
gtaccactcttcaggctgct

SEQ ID NO: 198 - DNA, 20 bp, artificial
  human FXR antisense
cagttgcccccgtttttaca

SEQ ID NO: 199 - DNA, 20 bp, artificial
  human FXR antisense
catctctttgcatttcctta

SEQ ID NO: 200 - DNA, 20 bp, artificial
  human FXR antisense
tgttgtcgaggtcacttgtc

SEQ ID NO: 201 - DNA, 20 bp, artificial
  human FXR antisense
tttgttatttcctgaggcat

SEQ ID NO: 202 - DNA, 20 bp, artificial
  human FXR antisense
ctcagaatccaatttcgcat

SEQ ID NO: 203 - DNA, 20 bp, artificial
  human FXR antisense
gatcctccctgctgacgcgc

SEQ ID NO: 204 - DNA, 20 bp, artificial
  human FXR antisense
gtctgatctgcatgctgctt

SEQ ID NO: 205 - DNA, 20 bp, artificial
  human FXR antisense
aaacacaatgtagagaaagt

SEQ ID NO: 206 - DNA, 20 bp, artificial
  human FXR antisense
agaatccaatttcgcattag

SEQ ID NO: 207 - DNA, 20 bp, artificial
  human FXR antisense
actcttcaggctgctggggg

SEQ ID NO: 208 - DNA, 20 bp, artificial
  human FXR antisense
tttggtaatgcttctcctga

SEQ ID NO: 209 - DNA, 20 bp, artificial
  human FXR antisense
gatctgcatgctgcttcaca

SEQ ID NO: 210 - DNA, 20 bp, artificial
  human FXR antisense
tgactttgttgtcgaggtca

SEQ ID NO: 211 - DNA, 20 bp, artificial
  human FXR antisense
ggagctagacccctcccctg

SEQ ID NO: 212 - DNA, 20 bp, artificial
  human FXR antisense
tgagattttccctagttcaa

SEQ ID NO: 213 - DNA, 20 bp, artificial
  human FXR antisense
cttctttgttacaggcatct

SEQ ID NO: 214 - DNA, 20 bp, artificial
  human FXR antisense
attcagtcaggcgacccagg

SEQ ID NO: 215 - DNA, 20 bp, artificial
  human FXR antisense
gtggtgatgattgaatgtcc

SEQ ID NO: 216 - DNA, 20 bp, artificial
  human FXR antisense
cactttgagctatgtttcta

SEQ ID NO: 217 - DNA, 20 bp, artificial
  human FXR antisense
cactcttgacactttcttcg

SEQ ID NO: 218 - DNA, 20 bp, artificial
  human FXR antisense
ggaagttacacatgtaatta

SEQ ID NO: 219 - DNA, 20 bp, artificial
  human FXR antisense
aatttcgcattaggataagt

SEQ ID NO: 220 - DNA, 20 bp, artificial
  human FXR antisense
aagtggggtaaacttgtggt

SEQ ID NO: 221 - DNA, 20 bp, artificial
  human FXR antisense
gctagacccctcccctgtaa

SEQ ID NO: 222 - DNA, 20 bp, artificial
  human FXR antisense
atatgagagagaaaaaggag

SEQ ID NO: 223 - DNA, 20 bp, artificial
  human FXR antisense
agttgttctatctagcccaa

SEQ ID NO: 224 - DNA, 20 bp, artificial
  human FXR antisense
aagttgttctatctagccca

SEQ ID NO: 225 - DNA, 20 bp, artificial
  human FXR antisense
agagaagcagtgttcacttt

SEQ ID NO: 226 - DNA, 20 bp, artificial
  human FXR antisense
tgctgacgcgcccatgcggg

SEQ ID NO: 227 - DNA, 20 bp, artificial
  human FXR antisense
ctctttgcatttccttagtc

SEQ ID NO: 228 - DNA, 20 bp, artificial
  human FXR antisense
ctgatctgcatgctgcttca

SEQ ID NO: 229 - DNA, 20 bp, artificial
  human FXR antisense
caataggtcagaatgcccag

SEQ ID NO: 230 - DNA, 20 bp, artificial
  human FXR antisense
ggcttgccaattagaatgca

SEQ ID NO: 231 - DNA, 20 bp, artificial
  human FXR antisense
ttcattcacggtctgatctg

SEQ ID NO: 232 - DNA, 20 bp, artificial
  human FXR antisense
tttgttgtcgaggtcacttg

SEQ ID NO: 233 - DNA, 20 bp, artificial
  human FXR antisense
aattttatttgttatttcct

SEQ ID NO: 234 - DNA, 20 bp, artificial
  human FXR antisense
tggtgatgattgaatgtccg

SEQ ID NO: 235 - DNA, 20 bp, artificial
  human FXR antisense
tggaagttacacatgtaatt

SEQ ID NO: 236 - DNA, 20 bp, artificial
  human FXR antisense
caatgtagagaaagttgttc

SEQ ID NO: 237 - DNA, 20 bp, artificial
  human FXR antisense
tttaaaacacaatgtagaga

SEQ ID NO: 238 - DNA, 20 bp, artificial
  human FXR antisense
ccaattagaatgcaggattc

SEQ ID NO: 239 - DNA, 20 bp, artificial
  human FXR antisense
aaatgcactttctttatggt

SEQ ID NO: 240 - DNA, 20 bp, artificial
  human FXR antisense
ctttgatcctccctgctgac

SEQ ID NO: 241 - DNA, 20 bp, artificial
  human FXR antisense
tctgatctgcatgctgcttc

SEQ ID NO: 242 - DNA, 20 bp, artificial
  human FXR antisense
cagaccctttcagcaaagca

SEQ ID NO: 243 - DNA, 20 bp, artificial
  human FXR antisense
cacaacttttgtagcacatc

SEQ ID NO: 244 - DNA, 20 bp, artificial
  human FXR antisense
cagtcaggcgacccaggaga

SEQ ID NO: 245 - DNA, 20 bp, artificial
  human FXR antisense
aggagctagacccctcccct

SEQ ID NO: 246 - DNA, 20 bp, artificial
  human FXR antisense
caatttcgcattaggataag

SEQ ID NO: 247 - DNA, 20 bp, artificial
  human FXR antisense
ctttctttatggtggtcttc

SEQ ID NO: 248 - DNA, 20 bp, artificial
  human FXR antisense
tgttacaggcatctctgcta

SEQ ID NO: 249 - DNA, 20 bp, artificial
  human FXR antisense
ctccctgctgacgcgcccat

SEQ ID NO: 250 - DNA, 20 bp, artificial
  human FXR antisense
tcttgacactttcttcgcat

SEQ ID NO: 251 - DNA, 20 bp, artificial
  human FXR antisense
atttccttagtcgacactct

SEQ ID NO: 252 - DNA, 20 bp, artificial
  human FXR antisense
ggtctgatctgcatgctgct

SEQ ID NO: 253 - DNA, 20 bp, artificial
  human FXR antisense
tctgtttgttatatgaatcc

SEQ ID NO: 254 - DNA, 20 bp, artificial
  human FXR antisense
gtgatgattgaatgtccgta

SEQ ID NO: 255 - DNA, 20 bp, artificial
  human FXR antisense
cgcctgagttcatatattcc

SEQ ID NO: 256 - DNA, 20 bp, artificial
  human FXR antisense
agaggctctgtctccacaaa

SEQ ID NO: 257 - DNA, 20 bp, artificial
  human FXR antisense
ggtagcttttttgtgaattc

SEQ ID NO: 258 - DNA, 20 bp, artificial
  human FXR antisense
acacaacttttgtagcacat

SEQ ID NO: 259 - DNA, 20 bp, artificial
  human FXR antisense
gctgcttcacattttttctc

SEQ ID NO: 260 - DNA, 20 bp, artificial
  human FXR antisense
agaacctgtacatgattggt

SEQ ID NO: 261 - DNA, 20 bp, artificial
  human FXR antisense
aatatgagagagaaaaagga

SEQ ID NO: 262 - DNA, 20 bp, artificial
  human FXR antisense
aggtgttatatattcatcag

SEQ ID NO: 263 - DNA, 20 bp, artificial
  human FXR antisense
cagcatctcagcgtggtgat

SEQ ID NO: 264 - DNA, 20 bp, artificial
  human FXR antisense
ggtaaacttgtggtcgttta

SEQ ID NO: 265 - DNA, 20 bp, artificial
  human FXR antisense
ttaaaacacaatgtagagaa

SEQ ID NO: 266 - DNA, 20 bp, artificial
  human FXR antisense
cattatgtttgctttattgc

SEQ ID NO: 267 - DNA, 20 bp, artificial
  human FXR antisense
aagagaagcagtgttcactt

SEQ ID NO: 268 - DNA, 20 bp, artificial
  human FXR antisense
tttctcagtcgcttagattt

SEQ ID NO: 269 - DNA, 20 bp, artificial
  human FXR antisense
ttttctcagtcgcttagatt

SEQ ID NO: 270 - DNA, 20 bp, artificial
  human FXR antisense
tttttctcagtcgcttagat

SEQ ID NO: 271 - DNA, 20 bp, artificial
  human FXR antisense
atttgttatttcctgaggca

SEQ ID NO: 272 - DNA, 20 bp, artificial
  human FXR antisense
gtacatgattggttgccatt

SEQ ID NO: 273 - DNA, 20 bp, artificial
  human FXR antisense
tgtacatgattggttgccat

SEQ ID NO: 274 - DNA, 20 bp, artificial
  human FXR antisense
cctgtacatgattggttgcc

SEQ ID NO: 275 - DNA, 20 bp, artificial
  human FXR antisense
gtagcttttttgtgaattct

SEQ ID NO: 276 - DNA, 20 bp, artificial
  human FXR antisense
agtcaggcgacccaggagac

SEQ ID NO: 277 - DNA, 20 bp, artificial
  human FXR antisense
catcagcatctcagcgtggt

SEQ ID NO: 278 - DNA, 20 bp, artificial
  human FXR antisense
gaggctctgtctccacaaac

SEQ ID NO: 279 - DNA, 20 bp, artificial
  human FXR antisense
ttttctccctgcatgacttt

SEQ ID NO: 280 - DNA, 20 bp, artificial
  human FXR antisense
tgcccagacggaagtttctt

SEQ ID NO: 281 - DNA, 20 bp, artificial
  human FXR antisense
gttgccattatgtttgcttt

SEQ ID NO: 282 - DNA, 20 bp, artificial
  human FXR antisense
gctcagaatccaatttcgca

SEQ ID NO: 283 - DNA, 20 bp, artificial
  human FXR antisense
gctggcatacgcctgagttc

SEQ ID NO: 284 - DNA, 20 bp, artificial
  human FXR antisense
ccctgcatgactttgttgtc

SEQ ID NO: 285 - DNA, 20 bp, artificial
  human FXR antisense
tttctccctgcatgactttg

SEQ ID NO: 286 - DNA, 20 bp, artificial
  human FXR antisense
agttttctccctgcatgact

SEQ ID NO: 287 - DNA, 20 bp, artificial
  human FXR antisense
ttcagttttctccctgcatg

SEQ ID NO: 288 - DNA, 20 bp, artificial
  human FXR antisense
atgcccagacggaagtttct

SEQ ID NO: 289 - DNA, 20 bp, artificial
  human FXR antisense
taggtgttatatattcatca

SEQ ID NO: 290 - DNA, 20 bp, artificial
  human FXR antisense
gtcaggcgacccaggagaca

SEQ ID NO: 291 - DNA, 20 bp, artificial
  human FXR antisense
ccattatgtttgctttattg

SEQ ID NO: 292 - DNA, 20 bp, artificial
  human FXR antisense
aggacctgccacttgttctg

SEQ ID NO: 293 - DNA, 20 bp, artificial
  human FXR antisense
ttcccatctctttgcatttc

SEQ ID NO: 294 - DNA, 20 bp, artificial
  human FXR antisense
attcccatctctttgcattt

SEQ ID NO: 295 - DNA, 20 bp, artificial
  human FXR antisense
gtagcacatcaagaagtggc

SEQ ID NO: 296 - DNA, 20 bp, artificial
  human FXR antisense
acaacttttgtagcacatca

SEQ ID NO: 297 - DNA, 20 bp, artificial
  human FXR antisense
ccgtaattcagtcaggcgac

SEQ ID NO: 298 - DNA, 20 bp, artificial
  human FXR antisense
tcgcattaggataagtcggg

SEQ ID NO: 299 - DNA, 20 bp, artificial
  human FXR antisense
ttcactttgagctatgtttc

SEQ ID NO: 300 - DNA, 20 bp, artificial
  human FXR antisense
tcccctttgatcctccctgc

SEQ ID NO: 301 - DNA, 20 bp, artificial
  human FXR antisense
gcttcacattttttctcagt

SEQ ID NO: 302 - DNA, 20 bp, artificial
  human FXR antisense
aggtcacttgtcgcaagtca

SEQ ID NO: 303 - DNA, 20 bp, artificial
  human FXR antisense
cagttttctccctgcatgac

SEQ ID NO: 304 - DNA, 20 bp, artificial
  human FXR antisense
gcccagacggaagtttctta

SEQ ID NO: 305 - DNA, 20 bp, artificial
  human FXR antisense
acataggtgttatatattca

SEQ ID NO: 306 - DNA, 20 bp, artificial
  human FXR antisense
acatcagattaatatgagag

SEQ ID NO: 307 - DNA, 20 bp, artificial
  human FXR antisense
gagaagcagtgttcactttg

SEQ ID NO: 308 - DNA, 20 bp, artificial
  human FXR antisense
ggcatacgcctgagttcata

SEQ ID NO: 309 - DNA, 20 bp, artificial
  human FXR antisense
cgtttttggtaatgcttctc

SEQ ID NO: 310 - DNA, 20 bp, artificial
  human FXR antisense
gactttgttgtcgaggtcac

SEQ ID NO: 311 - DNA, 20 bp, artificial
  human FXR antisense
gagattttccctagttcaac

SEQ ID NO: 312 - DNA, 20 bp, artificial
  human FXR antisense
gccattatgtttgctttatt

SEQ ID NO: 313 - DNA, 20 bp, artificial
  human FXR antisense
ttgccattatgtttgcttta

SEQ ID NO: 314 - DNA, 20 bp, artificial
  human FXR antisense
agctggcatacgcctgagtt

SEQ ID NO: 315 - DNA, 20 bp, artificial
  human FXR antisense
atcctccctgctgacgcgcc

SEQ ID NO: 316 - DNA, 20 bp, artificial
  human FXR antisense
agcattcagccaacattccc

SEQ ID NO: 317 - DNA, 20 bp, artificial
  human FXR antisense
tctccctgcatgactttgtt

SEQ ID NO: 318 - DNA, 20 bp, artificial
  human FXR antisense
tagcttttttgtgaattcta

SEQ ID NO: 319 - DNA, 20 bp, artificial
  human FXR antisense
accctttcagcaaagcaatc

SEQ ID NO: 320 - DNA, 20 bp, artificial
  human FXR antisense
tagcacatcaagaagtggct

SEQ ID NO: 321 - DNA, 20 bp, artificial
  human FXR antisense
aaaacacaatgtagagaaag

SEQ ID NO: 322 - DNA, 20 bp, artificial
  human FXR antisense
tgccattatgtttgctttat

SEQ ID NO: 323 - DNA, 20 bp, artificial
  human FXR antisense
ctgctgacgcgcccatgcgg

SEQ ID NO: 324 - DNA, 20 bp, artificial
  human FXR antisense
cctccctgctgacgcgccca

SEQ ID NO: 325 - DNA, 20 bp, artificial
  human FXR antisense
gttttctccctgcatgactt

SEQ ID NO: 326 - DNA, 20 bp, artificial
  human FXR antisense
gaacctgtacatgattggtt

SEQ ID NO: 327 - DNA, 20 bp, artificial
  human FXR antisense
tggtagcttttttgtgaatt

SEQ ID NO: 328 - DNA, 20 bp, artificial
  human FXR antisense
cagcgtggtgatgattgaat

SEQ ID NO: 329 - DNA, 20 bp, artificial
  human FXR antisense
taatatgagagagaaaaagg

SEQ ID NO: 330 - DNA, 20 bp, artificial
  human FXR antisense
tacatcagattaatatgaga

SEQ ID NO: 331 - DNA, 20 bp, artificial
  human FXR antisense
ttatgtttgctttattgcca

SEQ ID NO: 332 - DNA, 20 bp, artificial
  human FXR antisense
ctcatcccctttgatcctcc

SEQ ID NO: 333 - DNA, 20 bp, artificial
  human FXR antisense
caacattcccatctctttgc

SEQ ID NO: 334 - DNA, 20 bp, artificial
  human FXR antisense
tgtcgaggtcacttgtcgca

SEQ ID NO: 335 - DNA, 20 bp, artificial
  human FXR antisense
ctccctgcatgactttgttg

SEQ ID NO: 336 - DNA, 20 bp, artificial
  human FXR antisense
ttctccctgcatgactttgt

SEQ ID NO: 337 - DNA, 20 bp, artificial
  human FXR antisense
attttatttgttatttcctg

SEQ ID NO: 338 - DNA, 20 bp, artificial
  human FXR antisense
acctgtacatgattggttgc

SEQ ID NO: 339 - DNA, 20 bp, artificial
  human FXR antisense
gcttttttgtgaattctaca

SEQ ID NO: 340 - DNA, 20 bp, artificial
  human FXR antisense
gcaaagcaatctggtcttca

SEQ ID NO: 341 - DNA, 20 bp, artificial
  human FXR antisense
ctttcagcaaagcaatctgg

SEQ ID NO: 342 - DNA, 20 bp, artificial
  human FXR antisense
ggtgttatatattcatcaga

SEQ ID NO: 343 - DNA, 20 bp, artificial
  human FXR antisense
tatcctttatgtattgtcta

SEQ ID NO: 344 - DNA, 20 bp, artificial
  human FXR antisense
gctatgtttctaagtcttct

SEQ ID NO: 345 - DNA, 20 bp, artificial
  human FXR antisense
atgcggggcttctttgttac

SEQ ID NO: 346 - DNA, 20 bp, artificial
  human FXR antisense
gctcatcccctttgatcctc

SEQ ID NO: 347 - DNA, 20 bp, artificial
  human FXR antisense
cacggtctgatctgcatgct

SEQ ID NO: 348 - DNA, 20 bp, artificial
  human FXR antisense
ctttgttgtcgaggtcactt

SEQ ID NO: 349 - DNA, 20 bp, artificial
  human FXR antisense
aaattttatttgttatttcc

SEQ ID NO: 350 - DNA, 20 bp, artificial
  human FXR antisense
agaccctttcagcaaagcaa

SEQ ID NO: 351 - DNA, 20 bp, artificial
  human FXR antisense
ataggtgttatatattcatc

SEQ ID NO: 352 - DNA, 20 bp, artificial
  human FXR antisense
tacacaacttttgtagcaca

SEQ ID NO: 353 - DNA, 20 bp, artificial
  human FXR antisense
gttatacatcagattaatat

SEQ ID NO: 354 - DNA, 20 bp, artificial
  human FXR antisense
taaaacacaatgtagagaaa

SEQ ID NO: 355 - DNA, 20 bp, artificial
  human FXR antisense
ttttaaaacacaatgtagag

SEQ ID NO: 356 - DNA, 20 bp, artificial
  human FXR antisense
gaagtaacaatcaatttaat

SEQ ID NO: 357 - DNA, 20 bp, artificial
  human FXR antisense
tgaagtaacaatcaatttaa

SEQ ID NO: 358 - DNA, 20 bp, artificial
  human FXR antisense
ttgaagtaacaatcaattta

SEQ ID NO: 359 - DNA, 20 bp, artificial
  human FXR antisense
gcattaggataagtcgggga

SEQ ID NO: 360 - DNA, 20 bp, artificial
  human FXR antisense
taagagaagcagtgttcact

SEQ ID NO: 361 - DNA, 20 bp, artificial
  human FXR antisense
cctttgatcctccctgctga

SEQ ID NO: 362 - DNA, 20 bp, artificial
  human FXR antisense
atatccatcacacagttgcc

SEQ ID NO: 363 - DNA, 20 bp, artificial
  human FXR antisense
tttgttatatgaatccataa

SEQ ID NO: 364 - DNA, 20 bp, artificial
  human FXR antisense
agcttttttgtgaattctac

SEQ ID NO: 365 - DNA, 20 bp, artificial
  human FXR antisense
cagaatgcccagacggaagt

SEQ ID NO: 366 - DNA, 20 bp, artificial
  human FXR antisense
aggtcagaatgcccagacgg

SEQ ID NO: 367 - DNA, 20 bp, artificial
  human FXR antisense
ggactgagtcttcctctcca

SEQ ID NO: 368 - DNA, 20 bp, artificial
  human FXR antisense
gatggactttcaaggccctg

SEQ ID NO: 369 - DNA, 20 bp, artificial
  human FXR antisense
gttacaggcatctctgctac

SEQ ID NO: 370 - DNA, 20 bp, artificial
  human FXR antisense
tgcccccgtttttacacttg

SEQ ID NO: 371 - DNA, 20 bp, artificial
  human FXR antisense
atctctttgcatttccttag

SEQ ID NO: 372 - DNA, 20 bp, artificial
  human FXR antisense
ggtcacttgtcgcaagtcac

SEQ ID NO: 373 - DNA, 20 bp, artificial
  human FXR antisense
tccctgcatgactttgttgt

SEQ ID NO: 374 - DNA, 20 bp, artificial
  human FXR antisense
aaggagctagacccctcccc

SEQ ID NO: 375 - DNA, 20 bp, artificial
  human FXR antisense
tgtttgctttattgccaaga

SEQ ID NO: 376 - DNA, 20 bp, artificial
  human FXR antisense
gttcaatgagattcattttt

SEQ ID NO: 377 - DNA, 20 bp, artificial
  human FXR antisense
tgttcaatgagattcatttt

SEQ ID NO: 378 - DNA, 20 bp, artificial
  human FXR antisense
cctgccacttgttctgttaa

SEQ ID NO: 379 - DNA, 20 bp, artificial
  human FXR antisense
agtaccactcttcaggctgc

SEQ ID NO: 380 - DNA, 20 bp, artificial
  human FXR antisense
tcacggtctgatctgcatgc

SEQ ID NO: 381 - DNA, 20 bp, artificial
  human FXR antisense
ttgtcgaggtcacttgtcgc

SEQ ID NO: 382 - DNA, 20 bp, artificial
  human FXR antisense
aagaacctgtacatgattgg

SEQ ID NO: 383 - DNA, 20 bp, artificial
  human FXR antisense
taaacttgtggtcgtttact

SEQ ID NO: 384 - DNA, 20 bp, artificial
  human FXR antisense
gagagaaaaaggagctagac

SEQ ID NO: 385 - DNA, 20 bp, artificial
  human FXR antisense
atgtttgctttattgccaag

SEQ ID NO: 386 - DNA, 20 bp, artificial
  human FXR antisense
ttctttatggtggtcttcaa

SEQ ID NO: 387 - DNA, 20 bp, artificial
  human FXR antisense
actgaacattgctgtattgc

SEQ ID NO: 388 - DNA, 20 bp, artificial
  human FXR antisense
ccactcttcaggctgctggg

SEQ ID NO: 389 - DNA, 20 bp, artificial
  human FXR antisense
ctggcatacgcctgagttca

SEQ ID NO: 390 - DNA, 20 bp, artificial
  human FXR antisense
ggctctgtctccacaaacaa

SEQ ID NO: 391 - DNA, 20 bp, artificial
  human FXR antisense
gttgtcgaggtcacttgtcg

SEQ ID NO: 392 - DNA, 20 bp, artificial
  human FXR antisense
atacatcagattaatatgag

SEQ ID NO: 393 - DNA, 20 bp, artificial
  human FXR antisense
attgaagtaacaatcaattt

SEQ ID NO: 394 - DNA, 20 bp, artificial
  human FXR antisense
atgttcaatgagattcattt

SEQ ID NO: 395 - DNA, 20 bp, artificial
  human FXR antisense
gcttctttgttacaggcatc

SEQ ID NO: 396 - DNA, 20 bp, artificial
  human FXR antisense
atgaatccataataaaatgt

SEQ ID NO: 397 - DNA, 20 bp, artificial
  human FXR antisense
atcctttatgtattgtctat

SEQ ID NO: 398 - DNA, 20 bp, artificial
  human FXR antisense
atcagcatctcagcgtggtg

SEQ ID NO: 399 - DNA, 20 bp, artificial
  human FXR antisense
gagaaaaaggagctagaccc

SEQ ID NO: 400 - DNA, 20 bp, artificial
  human FXR antisense
gtggaagttacacatgtaat

SEQ ID NO: 401 - DNA, 20 bp, artificial
  human FXR antisense
gttgtggaagttacacatgt

SEQ ID NO: 402 - DNA, 20 bp, artificial
  human FXR antisense
aagtaacaatcaatttaatt

SEQ ID NO: 403 - DNA, 20 bp, artificial
  human FXR antisense
agattttccctagttcaaca

SEQ ID NO: 404 - DNA, 20 bp, artificial
  human FXR antisense
actgagtcttcctctccaga

SEQ ID NO: 405 - DNA, 20 bp, artificial
  human FXR antisense
tggactgagtcttcctctcc

SEQ ID NO: 406 - DNA, 20 bp, artificial
  human FXR antisense
agatggactttcaaggccct

SEQ ID NO: 407 - DNA, 20 bp, artificial
  human FXR antisense
gattgttttgggtcagagat

SEQ ID NO: 408 - DNA, 20 bp, artificial
  human FXR antisense
gcctgagttcatatattcca

SEQ ID NO: 409 - DNA, 20 bp, artificial
  human FXR antisense
tcttcattcacggtctgatc

SEQ ID NO: 410 - DNA, 20 bp, artificial
  human FXR antisense
ctggtagcttttttgtgaat

SEQ ID NO: 411 - DNA, 20 bp, artificial
  human FXR antisense
cgcagaccctttcagcaaag

SEQ ID NO: 412 - DNA, 20 bp, artificial
  human FXR antisense
tcagaatgcccagacggaag

SEQ ID NO: 413 - DNA, 20 bp, artificial
  human FXR antisense
gatgattgaatgtccgtaat

SEQ ID NO: 414 - DNA, 20 bp, artificial
  human FXR antisense
tgatgattgaatgtccgtaa

SEQ ID NO: 415 - DNA, 20 bp, artificial
  human FXR antisense
gagagagaaaaaggagctag

SEQ ID NO: 416 - DNA, 20 bp, artificial
  human FXR antisense
ggattccctggagcctttta

SEQ ID NO: 417 - DNA, 20 bp, artificial
  human FXR antisense
gcaggattccctggagcctt

SEQ ID NO: 418 - DNA, 20 bp, artificial
  human FXR antisense
tatgtttgctttattgccaa

SEQ ID NO: 419 - DNA, 20 bp, artificial
  human FXR antisense
ttctgttgccattatgtttg

SEQ ID NO: 420 - DNA, 20 bp, artificial
  human FXR antisense
acctgccacttgttctgtta

SEQ ID NO: 421 - DNA, 20 bp, artificial
  human FXR antisense
tgctgtattgcgagtatggt

SEQ ID NO: 422 - DNA, 20 bp, artificial
  human FXR antisense
ttggtaatgcttctcctgaa

SEQ ID NO: 423 - DNA, 20 bp, artificial
  human FXR antisense
tgcttcacattttttctcag

SEQ ID NO: 424 - DNA, 20 bp, artificial
  human FXR antisense
acggtctgatctgcatgctg

SEQ ID NO: 425 - DNA, 20 bp, artificial
  human FXR antisense
gaatgcccagacggaagttt

SEQ ID NO: 426 - DNA, 20 bp, artificial
  human FXR antisense
agcacatcaagaagtggctc

SEQ ID NO: 427 - DNA, 20 bp, artificial
  human FXR antisense
caacttttgtagcacatcaa

SEQ ID NO: 428 - DNA, 20 bp, artificial
  human FXR antisense
tgattgaatgtccgtaattc

SEQ ID NO: 429 - DNA, 20 bp, artificial
  human FXR antisense
tatacatcagattaatatga

SEQ ID NO: 430 - DNA, 20 bp, artificial
  human FXR antisense
cttttaaaacacaatgtaga

SEQ ID NO: 431 - DNA, 20 bp, artificial
  human FXR antisense
tgcaggattccctggagcct

SEQ ID NO: 432 - DNA, 20 bp, artificial
  human FXR antisense
tttcaaggccctgggaggat

SEQ ID NO: 433 - DNA, 20 bp, artificial
  human FXR antisense
actttgagctatgtttctaa

SEQ ID NO: 434 - DNA, 20 bp, artificial
  human FXR antisense
tttctttatggtggtcttca

SEQ ID NO: 435 - DNA, 20 bp, artificial
  human FXR antisense
ggggcttctttgttacaggc

SEQ ID NO: 436 - DNA, 20 bp, artificial
  human FXR antisense
gcgtttttggtaatgcttct

SEQ ID NO: 437 - DNA, 20 bp, artificial
  human FXR antisense
agaatgcccagacggaagtt

SEQ ID NO: 438 - DNA, 20 bp, artificial
  human FXR antisense
gcatactcctcttgagtcat

SEQ ID NO: 439 - DNA, 20 bp, artificial
  human FXR antisense
tgtagcacatcaagaagtgg

SEQ ID NO: 440 - DNA, 20 bp, artificial
  human FXR antisense
gtaaacttgtggtcgtttac

SEQ ID NO: 441 - DNA, 20 bp, artificial
  human FXR antisense
agttatacatcagattaata

SEQ ID NO: 442 - DNA, 20 bp, artificial
  human FXR antisense
tgatctgcatgctgcttcac

SEQ ID NO: 443 - DNA, 20 bp, artificial
  human FXR antisense
attcacggtctgatctgcat

SEQ ID NO: 444 - DNA, 20 bp, artificial
  human FXR antisense
attggttgccatttccgtca

SEQ ID NO: 445 - DNA, 20 bp, artificial
  human FXR antisense
aacctgtacatgattggttg

SEQ ID NO: 446 - DNA, 20 bp, artificial
  human FXR antisense
ttcatggtccaaagtctgaa

SEQ ID NO: 447 - DNA, 20 bp, artificial
  human FXR antisense
cttcatggtccaaagtctga

SEQ ID NO: 448 - DNA, 20 bp, artificial
  human FXR antisense
tcagcaaagcaatctggtct

SEQ ID NO: 449 - DNA, 20 bp, artificial
  human FXR antisense
ttcaaccgcagaccctttca

SEQ ID NO: 450 - DNA, 20 bp, artificial
  human FXR antisense
aatgcccagacggaagtttc

SEQ ID NO: 451 - DNA, 20 bp, artificial
  human FXR antisense
ctatcctttatgtattgtct

SEQ ID NO: 452 - DNA, 20 bp, artificial
  human FXR antisense
ttaatatgagagagaaaaag

SEQ ID NO: 453 - DNA, 20 bp, artificial
  human FXR antisense
tcaatgagattcatttttga

SEQ ID NO: 454 - DNA, 20 bp, artificial
  human FXR antisense
tgcggggcttctttgttaca

SEQ ID NO: 455 - DNA, 20 bp, artificial
  human FXR antisense
cattcccatctctttgcatt

SEQ ID NO: 456 - DNA, 20 bp, artificial
  human FXR antisense
tatttgttatttcctgaggc

SEQ ID NO: 457 - DNA, 20 bp, artificial
  human FXR antisense
cataggtgttatatattcat

SEQ ID NO: 458 - DNA, 20 bp, artificial
  human FXR antisense
gcttctctactgcctctcta

SEQ ID NO: 459 - DNA, 20 bp, artificial
  human FXR antisense
ttgaatgtccgtaattcagt

SEQ ID NO: 460 - DNA, 20 bp, artificial
  human FXR antisense
agccttttaaaacacaatgt

SEQ ID NO: 461 - DNA, 20 bp, artificial
  human FXR antisense
ttctacgatgtcttctacct

SEQ ID NO: 462 - DNA, 20 bp, artificial
  human FXR antisense
gcattcagccaacattccca

SEQ ID NO: 463 - DNA, 20 bp, artificial
  human FXR antisense
ctgtacatgattggttgcca

SEQ ID NO: 464 - DNA, 20 bp, artificial
  human FXR antisense
tttcagcaaagcaatctggt

SEQ ID NO: 465 - DNA, 20 bp, artificial
  human FXR antisense
ggtcagaatgcccagacgga

SEQ ID NO: 466 - DNA, 20 bp, artificial
  human FXR antisense
atgattgaatgtccgtaatt

SEQ ID NO: 467 - DNA, 20 bp, artificial
  human FXR antisense
tctggactgagtcttcctct

SEQ ID NO: 468 - DNA, 20 bp, artificial
  human FXR antisense
tctttatggtggtcttcaaa

SEQ ID NO: 469 - DNA, 20 bp, artificial
  human FXR antisense
ttttttctcagtcgcttaga

SEQ ID NO: 470 - DNA, 20 bp, artificial
  human FXR antisense
tcagttttctccctgcatga

SEQ ID NO: 471 - DNA, 20 bp, artificial
  human FXR antisense
cctggtagcttttttgtgaa

SEQ ID NO: 472 - DNA, 20 bp, artificial
  human FXR antisense
cagcaaagcaatctggtctt

SEQ ID NO: 473 - DNA, 20 bp, artificial
  human FXR antisense
ccaataggtcagaatgccca

SEQ ID NO: 474 - DNA, 20 bp, artificial
  human FXR antisense
ttatacatcagattaatatg

SEQ ID NO: 475 - DNA, 20 bp, artificial
  human FXR antisense
aatgtagagaaagttgttct

SEQ ID NO: 476 - DNA, 20 bp, artificial
  human FXR antisense
tctgttgccattatgtttgc

SEQ ID NO: 477 - DNA, 20 bp, artificial
  human FXR antisense
gagatggactttcaaggccc

SEQ ID NO: 478 - DNA, 20 bp, artificial
  human FXR antisense
aggctctgtctccacaaaca

SEQ ID NO: 479 - DNA, 20 bp, artificial
  human FXR antisense
gcccccgtttttacacttgt

SEQ ID NO: 480 - DNA, 20 bp, artificial
  human FXR antisense
cggtctgatctgcatgctgc

SEQ ID NO: 481 - DNA, 20 bp, artificial
  human FXR antisense
cgaccttcactgtcttcatt

SEQ ID NO: 482 - DNA, 20 bp, artificial
  human FXR antisense
gtggctcctgaagcttctct

SEQ ID NO: 483 - DNA, 20 bp, artificial
  human FXR antisense
tttgtagcacatcaagaagt

SEQ ID NO: 484 - DNA, 20 bp, artificial
  human FXR antisense
agagagaaaaaggagctaga

SEQ ID NO: 485 - DNA, 20 bp, artificial
  human FXR antisense
ttgttctatctagcccaata

SEQ ID NO: 486 - DNA, 20 bp, artificial
  human FXR antisense
ccagaggacctgccacttgt

SEQ ID NO: 487 - DNA, 20 bp, artificial
  human FXR antisense
tcatggtccaaagtctgaaa

SEQ ID NO: 488 - DNA, 20 bp, artificial
  human FXR antisense
ttacacaacttttgtagcac

SEQ ID NO: 489 - DNA, 20 bp, artificial
  human FXR antisense
atctcagcgtggtgatgatt

SEQ ID NO: 490 - DNA, 20 bp, artificial
  human FXR antisense
acatcagcatctcagcgtgg

SEQ ID NO: 491 - DNA, 20 bp, artificial
  human FXR antisense
tccccatcactgcacgtccc

SEQ ID NO: 492 - DNA, 20 bp, artificial
  human FXR antisense
ctagacccctcccctgtaat

SEQ ID NO: 493 - DNA, 20 bp, artificial
  human FXR antisense
gcccaatatttacagttgtg

SEQ ID NO: 494 - DNA, 20 bp, artificial
  human FXR antisense
gtttgctttattgccaagat

SEQ ID NO: 495 - DNA, 20 bp, artificial
  human FXR antisense
ctgagtcttcctctccagat

SEQ ID NO: 496 - DNA, 20 bp, artificial
  human FXR antisense
ttcaatgagattcatttttg

SEQ ID NO: 497 - DNA, 20 bp, artificial
  human FXR antisense
agcaaagcaatctggtcttc

SEQ ID NO: 498 - DNA, 20 bp, artificial
  human FXR antisense
gattgaatgtccgtaattca

SEQ ID NO: 499 - DNA, 20 bp, artificial
  human FXR antisense
tcagattaatatgagagaga

SEQ ID NO: 500 - DNA, 20 bp, artificial
  human FXR antisense
agtaacaatcaatttaatta

SEQ ID NO: 501 - DNA, 20 bp, artificial
  human FXR antisense
ttctggactgagtcttcctc

SEQ ID NO: 502 - DNA, 20 bp, artificial
  human FXR antisense
attgttttgggtcagagatg

SEQ ID NO: 503 - DNA, 20 bp, artificial
  human FXR antisense
cactcttcaggctgctgggg

SEQ ID NO: 504 - DNA, 20 bp, artificial
  human FXR antisense
cagctggcatacgcctgagt

SEQ ID NO: 505 - DNA, 20 bp, artificial
  human FXR antisense
ttgttatatgaatccataat

SEQ ID NO: 506 - DNA, 20 bp, artificial
  human FXR antisense
ttggttgccatttccgtcaa

SEQ ID NO: 507 - DNA, 20 bp, artificial
  human FXR antisense
gagccttttaaaacacaatg

SEQ ID NO: 508 - DNA, 20 bp, artificial
  human FXR antisense
attatgtttgctttattgcc

SEQ ID NO: 509 - DNA, 20 bp, artificial
  human FXR antisense
ttcaaggccctgggaggatt

SEQ ID NO: 510 - DNA, 20 bp, artificial
  human FXR antisense
ctgccacttgttctgttaaa

SEQ ID NO: 511 - DNA, 20 bp, artificial
  human FXR antisense
tggcatacgcctgagttcat

SEQ ID NO: 512 - DNA, 20 bp, artificial
  human FXR antisense
ctgcttcacattttttctca

SEQ ID NO: 513 - DNA, 20 bp, artificial
  human FXR antisense
actttgttgtcgaggtcact

SEQ ID NO: 514 - DNA, 20 bp, artificial
  human FXR antisense
tgagttcagttttctccctg

SEQ ID NO: 515 - DNA, 20 bp, artificial
  human FXR antisense
atgattggttgccatttccg

SEQ ID NO: 516 - DNA, 20 bp, artificial
  human FXR antisense
tacatgattggttgccattt

SEQ ID NO: 517 - DNA, 20 bp, artificial
  human FXR antisense
tcctggtagcttttttgtga

SEQ ID NO: 518 - DNA, 20 bp, artificial
  human FXR antisense
caaagcaatctggtcttcat

SEQ ID NO: 519 - DNA, 20 bp, artificial
  human FXR antisense
aacataggtgttatatattc

SEQ ID NO: 520 - DNA, 20 bp, artificial
  human FXR antisense
agcttctctactgcctctct

SEQ ID NO: 521 - DNA, 20 bp, artificial
  human FXR antisense
acttttgtagcacatcaaga

SEQ ID NO: 522 - DNA, 20 bp, artificial
  human FXR antisense
tcagcgtggtgatgattgaa

SEQ ID NO: 523 - DNA, 20 bp, artificial
  human FXR antisense
catctcagcgtggtgatgat

SEQ ID NO: 524 - DNA, 20 bp, artificial
  human FXR antisense
agttgtggaagttacacatg

SEQ ID NO: 525 - DNA, 20 bp, artificial
  human FXR antisense
cgattttgctacaaatgctc

SEQ ID NO: 526 - DNA, 20 bp, artificial
  human FXR antisense
ttgctgtattgcgagtatgg

SEQ ID NO: 527 - DNA, 20 bp, artificial
  human FXR antisense
cggggcttctttgttacagg

SEQ ID NO: 528 - DNA, 20 bp, artificial
  human FXR antisense
tgattggttgccatttccgt

SEQ ID NO: 529 - DNA, 20 bp, artificial
  human FXR antisense
tgcctctctatcctttatgt

SEQ ID NO: 530 - DNA, 20 bp, artificial
  human FXR antisense
tcagcatctcagcgtggtga

SEQ ID NO: 531 - DNA, 20 bp, artificial
  human FXR antisense
aacttgtggtcgtttactct

SEQ ID NO: 532 - DNA, 20 bp, artificial
  human FXR antisense
gccttttaaaacacaatgta

SEQ ID NO: 533 - DNA, 20 bp, artificial
  human FXR antisense
cattaggataagtcggggag

SEQ ID NO: 534 - DNA, 20 bp, artificial
  human FXR antisense
cgcattaggataagtcgggg

SEQ ID NO: 535 - DNA, 20 bp, artificial
  human FXR antisense
ttttgctacaaatgctcaga

SEQ ID NO: 536 - DNA, 20 bp, artificial
  human FXR antisense
gattttgctacaaatgctca

SEQ ID NO: 537 - DNA, 20 bp, artificial
  human FXR antisense
ttgttttgggtcagagatgg

SEQ ID NO: 538 - DNA, 20 bp, artificial
  human FXR antisense
tgtccgtaattcagtcaggc

SEQ ID NO: 539 - DNA, 20 bp, artificial
  human FXR antisense
aaacttgtggtcgtttactc

SEQ ID NO: 540 - DNA, 20 bp, artificial
  human FXR antisense
cctgagttcatatattccag

SEQ ID NO: 541 - DNA, 20 bp, artificial
  human FXR antisense
gggcttctttgttacaggca

SEQ ID NO: 542 - DNA, 20 bp, artificial
  human FXR antisense
cacattttttctcagtcgct

SEQ ID NO: 543 - DNA, 20 bp, artificial
  human FXR antisense
cttttgtagcacatcaagaa

SEQ ID NO: 544 - DNA, 20 bp, artificial
  human FXR antisense
tcttacacaacttttgtagc

SEQ ID NO: 545 - DNA, 20 bp, artificial
  human FXR antisense
agagaaaaaggagctagacc

SEQ ID NO: 546 - DNA, 20 bp, artificial
  human FXR antisense
aactgggtacaagtgaaata

SEQ ID NO: 547 - DNA, 20 bp, artificial
  human FXR antisense
cccaatatttacagttgtgg

SEQ ID NO: 548 - DNA, 20 bp, artificial
  human FXR antisense
ggagccttttaaaacacaat

SEQ ID NO: 549 - DNA, 20 bp, artificial
  human FXR antisense
tgttgccattatgtttgctt

SEQ ID NO: 550 - DNA, 20 bp, artificial
  human FXR antisense
attctggactgagtcttcct

SEQ ID NO: 551 - DNA, 20 bp, artificial
  human FXR antisense
ggccctgggaggattctgga

SEQ ID NO: 552 - DNA, 20 bp, artificial
  human FXR antisense
gctctgtctccacaaacaac

SEQ ID NO: 553 - DNA, 20 bp, artificial
  human FXR antisense
cttgacactttcttcgcatg

SEQ ID NO: 554 - DNA, 20 bp, artificial
  human FXR antisense
ttctcagtcgcttagattta

SEQ ID NO: 555 - DNA, 20 bp, artificial
  human FXR antisense
ctgaaatcctggtagctttt

SEQ ID NO: 556 - DNA, 20 bp, artificial
  human FXR antisense
ttgtagcacatcaagaagtg

SEQ ID NO: 557 - DNA, 20 bp, artificial
  human FXR antisense
tcaggcgacccaggagacag

SEQ ID NO: 558 - DNA, 20 bp, artificial
  human FXR antisense
tctcagcgtggtgatgattg

SEQ ID NO: 559 - DNA, 20 bp, artificial
  human FXR antisense
aagttatacatcagattaat

SEQ ID NO: 560 - DNA, 20 bp, artificial
  human FXR antisense
aggattccctggagcctttt

SEQ ID NO: 561 - DNA, 20 bp, artificial
  human FXR antisense
caattagaatgcaggattcc

SEQ ID NO: 562 - DNA, 20 bp, artificial
  human FXR antisense
ctttcaaggccctgggagga

SEQ ID NO: 563 - DNA, 20 bp, artificial
  human FXR antisense
tacgatgtcttctacctcct

SEQ ID NO: 564 - DNA, 20 bp, artificial
  human FXR antisense
tcttcaggctgctgggggta

SEQ ID NO: 565 - DNA, 20 bp, artificial
  human FXR antisense
cagcgtttttggtaatgctt

SEQ ID NO: 566 - DNA, 20 bp, artificial
  human FXR antisense
tgaatccataataaaatgta

SEQ ID NO: 567 - DNA, 20 bp, artificial
  human FXR antisense
tggttgccatttccgtcaaa

SEQ ID NO: 568 - DNA, 20 bp, artificial
  human FXR antisense
caagaacctgtacatgattg

SEQ ID NO: 569 - DNA, 20 bp, artificial
  human FXR antisense
agtctgaaatcctggtagct

SEQ ID NO: 570 - DNA, 20 bp, artificial
  human FXR antisense
tcaaccgcagaccctttcag

SEQ ID NO: 571 - DNA, 20 bp, artificial
  human FXR antisense
ttcgaattctttcttccaat

SEQ ID NO: 572 - DNA, 20 bp, artificial
  human FXR antisense
agtggctcctgaagcttctc

SEQ ID NO: 573 - DNA, 20 bp, artificial
  human FXR antisense
gaggattttcaggctggtga

SEQ ID NO: 574 - DNA, 20 bp, artificial
  human FXR antisense
attgaatgtccgtaattcag

SEQ ID NO: 575 - DNA, 20 bp, artificial
  human FXR antisense
cttgtggtcgtttactctcc

SEQ ID NO: 576 - DNA, 20 bp, artificial
  human FXR antisense
agaaaaaggagctagacccc

SEQ ID NO: 577 - DNA, 20 bp, artificial
  human FXR antisense
agaaagttgttctatctagc

SEQ ID NO: 578 - DNA, 20 bp, artificial
  human FXR antisense
ctttatggtggtcttcaaaa

SEQ ID NO: 579 - DNA, 20 bp, artificial
  human FXR antisense
gtgagttcagttttctccct

SEQ ID NO: 580 - DNA, 20 bp, artificial
  human FXR antisense
ccgcagaccctttcagcaaa

SEQ ID NO: 581 - DNA, 20 bp, artificial
  human FXR antisense
ctttcttccaataggtcaga

SEQ ID NO: 582 - DNA, 20 bp, artificial
  human FXR antisense
ttctttcttccaataggtca

SEQ ID NO: 583 - DNA, 20 bp, artificial
  human FXR antisense
tttcgaattctttcttccaa

SEQ ID NO: 584 - DNA, 20 bp, artificial
  human FXR antisense
agcatactcctcttgagtca

SEQ ID NO: 585 - DNA, 20 bp, artificial
  human FXR antisense
gaagtggggtaaacttgtgg

SEQ ID NO: 586 - DNA, 20 bp, artificial
  human FXR antisense
attaatatgagagagaaaaa

SEQ ID NO: 587 - DNA, 20 bp, artificial
  human FXR antisense
atgtagagaaagttgttcta

SEQ ID NO: 588 - DNA, 20 bp, artificial
  human FXR antisense
attaggataagtcggggaga

SEQ ID NO: 589 - DNA, 20 bp, artificial
  human FXR antisense
gattctggactgagtcttcc

SEQ ID NO: 590 - DNA, 20 bp, artificial
  human FXR antisense
ttcaggctgctgggggtaga

SEQ ID NO: 591 - DNA, 20 bp, artificial
  human FXR antisense
agcgtttttggtaatgcttc

SEQ ID NO: 592 - DNA, 20 bp, artificial
  human FXR antisense
catttccttagtcgacactc

SEQ ID NO: 593 - DNA, 20 bp, artificial
  human FXR antisense
aagcattcagccaacattcc

SEQ ID NO: 594 - DNA, 20 bp, artificial
  human FXR antisense
ggttgccatttccgtcaaaa

SEQ ID NO: 595 - DNA, 20 bp, artificial
  human FXR antisense
cttcaaccgcagaccctttc

SEQ ID NO: 596 - DNA, 20 bp, artificial
  human FXR antisense
tctttcttccaataggtcag

SEQ ID NO: 597 - DNA, 20 bp, artificial
  human FXR antisense
atttcgaattctttcttcca

SEQ ID NO: 598 - DNA, 20 bp, artificial
  human FXR antisense
ttttgtagcacatcaagaag

SEQ ID NO: 599 - DNA, 20 bp, artificial
  human FXR antisense
ctggtgaatcttacacaact

SEQ ID NO: 600 - DNA, 20 bp, artificial
  human FXR antisense
gtaatccccatcactgcacg

SEQ ID NO: 601 - DNA, 20 bp, artificial
  human FXR antisense
gggcttgccaattagaatgc

SEQ ID NO: 602 - DNA, 20 bp, artificial
  human FXR antisense
gtaagatgagcaaaatgaga

SEQ ID NO: 603 - DNA, 20 bp, artificial
  human FXR antisense
attttgctacaaatgctcag

SEQ ID NO: 604 - DNA, 20 bp, artificial
  human FXR antisense
ggactttcaaggccctggga

SEQ ID NO: 605 - DNA, 20 bp, artificial
  human FXR antisense
gcggggcttctttgttacag

SEQ ID NO: 606 - DNA, 20 bp, artificial
  human FXR antisense
tcacattttttctcagtcgc

SEQ ID NO: 607 - DNA, 20 bp, artificial
  human FXR antisense
tgttatatgaatccataata

SEQ ID NO: 608 - DNA, 20 bp, artificial
  human FXR antisense
catactcctcttgagtcatt

SEQ ID NO: 609 - DNA, 20 bp, artificial
  human FXR antisense
ctctctatcctttatgtatt

SEQ ID NO: 610 - DNA, 20 bp, artificial
  human FXR antisense
cagttgtggaagttacacat

SEQ ID NO: 611 - DNA, 20 bp, artificial
  human FXR antisense
atatttacagttgtggaagt

SEQ ID NO: 612 - DNA, 20 bp, artificial
  human FXR antisense
gattccctggagccttttaa

SEQ ID NO: 613 - DNA, 20 bp, artificial
  human FXR antisense
taagatgagcaaaatgagat

SEQ ID NO: 614 - DNA, 20 bp, artificial
  human FXR antisense
ccagaggctctgtctccaca

SEQ ID NO: 615 - DNA, 20 bp, artificial
  human FXR antisense
acattttttctcagtcgctt

SEQ ID NO: 616 - DNA, 20 bp, artificial
  human FXR antisense
cattcacggtctgatctgca

SEQ ID NO: 617 - DNA, 20 bp, artificial
  human FXR antisense
acttgtcgcaagtcacgacc

SEQ ID NO: 618 - DNA, 20 bp, artificial
  human FXR antisense
gaccctttcagcaaagcaat

SEQ ID NO: 619 - DNA, 20 bp, artificial
  human FXR antisense
cttccaataggtcagaatgc

SEQ ID NO: 620 - DNA, 20 bp, artificial
  human FXR antisense
tcctttatgtattgtctatc

SEQ ID NO: 621 - DNA, 20 bp, artificial
  human FXR antisense
cagattaatatgagagagaa

SEQ ID NO: 622 - DNA, 20 bp, artificial
  human FXR antisense
gaagttacacatgtaattac

SEQ ID NO: 623 - DNA, 20 bp, artificial
  human FXR antisense
tgttctatctagcccaatat

SEQ ID NO: 624 - DNA, 20 bp, artificial
  human FXR antisense
gattttccctagttcaacag

SEQ ID NO: 625 - DNA, 20 bp, artificial
  human FXR antisense
ctccttggattgttttgggt

SEQ ID NO: 626 - DNA, 20 bp, artificial
  human FXR antisense
tccaggaaactaagagaagc

SEQ ID NO: 627 - DNA, 20 bp, artificial
  human FXR antisense
aatgttcaatgagattcatt

SEQ ID NO: 628 - DNA, 20 bp, artificial
  human FXR antisense
tcaggctgctgggggtagaa

SEQ ID NO: 629 - DNA, 20 bp, artificial
  human FXR antisense
tctcctgaagaaacctttac

SEQ ID NO: 630 - DNA, 20 bp, artificial
  human FXR antisense
gtcttcattcacggtctgat

SEQ ID NO: 631 - DNA, 20 bp, artificial
  human FXR antisense
tatgaatccataataaaatg

SEQ ID NO: 632 - DNA, 20 bp, artificial
  human FXR antisense
tcttattgaaaatctcagct

SEQ ID NO: 633 - DNA, 20 bp, artificial
  human FXR antisense
ctcttgagtcattttcagtt

SEQ ID NO: 634 - DNA, 20 bp, artificial
  human FXR antisense
cctcttgagtcattttcagt

SEQ ID NO: 635 - DNA, 20 bp, artificial
  human FXR antisense
ccttttaaaacacaatgtag

SEQ ID NO: 636 - DNA, 20 bp, artificial
  human FXR antisense
agaatgcaggattccctgga

SEQ ID NO: 637 - DNA, 20 bp, artificial
  human FXR antisense
ctgagttcatatattccagg

SEQ ID NO: 638 - DNA, 20 bp, artificial
  human FXR antisense
gaaatcctggtagctttttt

SEQ ID NO: 639 - DNA, 20 bp, artificial
  human FXR antisense
tctgaaatcctggtagcttt

SEQ ID NO: 640 - DNA, 20 bp, artificial
  human FXR antisense
tcttcatggtccaaagtctg

SEQ ID NO: 641 - DNA, 20 bp, artificial
  human FXR antisense
tgaggattttcaggctggtg

SEQ ID NO: 642 - DNA, 20 bp, artificial
  human FXR antisense
atgtccgtaattcagtcagg

SEQ ID NO: 643 - DNA, 20 bp, artificial
  human FXR antisense
cccctcccctgtaatcccca

SEQ ID NO: 644 - DNA, 20 bp, artificial
  human FXR antisense
gagaaagttgttctatctag

SEQ ID NO: 645 - DNA, 20 bp, artificial
  human FXR antisense
agagaaagttgttctatcta

SEQ ID NO: 646 - DNA, 20 bp, artificial
  human FXR antisense
aacagggcttgccaattaga

SEQ ID NO: 647 - DNA, 20 bp, artificial
  human FXR antisense
acaatcaatttaattaggca

SEQ ID NO: 648 - DNA, 20 bp, artificial
  human FXR antisense
ctgttgccattatgtttgct

SEQ ID NO: 649 - DNA, 20 bp, artificial
  human FXR antisense
tgctcagaatccaatttcgc

SEQ ID NO: 650 - DNA, 20 bp, artificial
  human FXR antisense
atggactttcaaggccctgg

SEQ ID NO: 651 - DNA, 20 bp, artificial
  human FXR antisense
gagctatgtttctaagtctt

SEQ ID NO: 652 - DNA, 20 bp, artificial
  human FXR antisense
caatgagattcatttttgat

SEQ ID NO: 653 - DNA, 20 bp, artificial
  human FXR antisense
cccagaggacctgccacttg

SEQ ID NO: 654 - DNA, 20 bp, artificial
  human FXR antisense
gagtaccactcttcaggctg

SEQ ID NO: 655 - DNA, 20 bp, artificial
  human FXR antisense
agctcatcccctttgatcct

SEQ ID NO: 656 - DNA, 20 bp, artificial
  human FXR antisense
ttctcctgaagaaaccttta

SEQ ID NO: 657 - DNA, 20 bp, artificial
  human FXR antisense
cttcacattttttctcagtc

SEQ ID NO: 658 - DNA, 20 bp, artificial
  human FXR antisense
tgaaatcctggtagcttttt

SEQ ID NO: 659 - DNA, 20 bp, artificial
  human FXR antisense
aatctggtcttcatggtcca

SEQ ID NO: 660 - DNA, 20 bp, artificial
  human FXR antisense
cccagacggaagtttcttat

SEQ ID NO: 661 - DNA, 20 bp, artificial
  human FXR antisense
ggctcctgaagcttctctac

SEQ ID NO: 662 - DNA, 20 bp, artificial
  human FXR antisense
ctcagcgtggtgatgattga

SEQ ID NO: 663 - DNA, 20 bp, artificial
  human FXR antisense
gcatctcagcgtggtgatga

SEQ ID NO: 664 - DNA, 20 bp, artificial
  human FXR antisense
gaaaaaggagctagacccct

SEQ ID NO: 665 - DNA, 20 bp, artificial
  human FXR antisense
gcgattttgctacaaatgct

SEQ ID NO: 666 - DNA, 20 bp, artificial
  human FXR antisense
cagagatggactttcaaggc

SEQ ID NO: 667 - DNA, 20 bp, artificial
  human FXR antisense
tgagctatgtttctaagtct

SEQ ID NO: 668 - DNA, 20 bp, artificial
  human FXR antisense
attgctgtattgcgagtatg

SEQ ID NO: 669 - DNA, 20 bp, artificial
  human FXR antisense
acatgattggttgccatttc

SEQ ID NO: 670 - DNA, 20 bp, artificial
  human FXR antisense
gtctgaaatcctggtagctt

SEQ ID NO: 671 - DNA, 20 bp, artificial
  human FXR antisense
catggtccaaagtctgaaat

SEQ ID NO: 672 - DNA, 20 bp, artificial
  human FXR antisense
caaccgcagaccctttcagc

SEQ ID NO: 673 - DNA, 20 bp, artificial
  human FXR antisense
attctttcttccaataggtc

SEQ ID NO: 674 - DNA, 20 bp, artificial
  human FXR antisense
gttgaggattttcaggctgg

SEQ ID NO: 675 - DNA, 20 bp, artificial
  human FXR antisense
gtgttgaggattttcaggct

SEQ ID NO: 676 - DNA, 20 bp, artificial
  human FXR antisense
ttgtggaagttacacatgta

SEQ ID NO: 677 - DNA, 20 bp, artificial
  human FXR antisense
gccctgggaggattctggac

SEQ ID NO: 678 - DNA, 20 bp, artificial
  human FXR antisense
atgtttctaagtcttctttt

SEQ ID NO: 679 - DNA, 20 bp, artificial
  human FXR antisense
tgagttcatatattccagga

SEQ ID NO: 680 - DNA, 20 bp, artificial
  human FXR antisense
acagcgtttttggtaatgct

SEQ ID NO: 681 - DNA, 20 bp, artificial
  human FXR antisense
ttgacactttcttcgcatgt

SEQ ID NO: 682 - DNA, 20 bp, artificial
  human FXR antisense
tgtcttcattcacggtctga

SEQ ID NO: 683 - DNA, 20 bp, artificial
  human FXR antisense
tcacttgtcgcaagtcacga

SEQ ID NO: 684 - DNA, 20 bp, artificial
  human FXR antisense
atatgaatccataataaaat

SEQ ID NO: 685 - DNA, 20 bp, artificial
  human FXR antisense
ggtccaaagtctgaaatcct

SEQ ID NO: 686 - DNA, 20 bp, artificial
  human FXR antisense
cttattgaaaatctcagctg

SEQ ID NO: 687 - DNA, 20 bp, artificial
  human FXR antisense
tctatcctttatgtattgtc

SEQ ID NO: 688 - DNA, 20 bp, artificial
  human FXR antisense
actgcctctctatcctttat

SEQ ID NO: 689 - DNA, 20 bp, artificial
  human FXR antisense
ggtgaatcttacacaacttt

SEQ ID NO: 690 - DNA, 20 bp, artificial
  human FXR antisense
atcagattaatatgagagag

SEQ ID NO: 691 - DNA, 20 bp, artificial
  human FXR antisense
tgtggaagttacacatgtaa

SEQ ID NO: 692 - DNA, 20 bp, artificial
  human FXR antisense
gaaagttgttctatctagcc

SEQ ID NO: 693 - DNA, 20 bp, artificial
  human FXR antisense
aagatgagcaaaatgagatt

SEQ ID NO: 694 - DNA, 20 bp, artificial
  human FXR antisense
tttgctacaaatgctcagaa

SEQ ID NO: 695 - DNA, 20 bp, artificial
  human FXR antisense
ggattctggactgagtcttc

SEQ ID NO: 696 - DNA, 20 bp, artificial
  human FXR antisense
ggattgttttgggtcagaga

SEQ ID NO: 697 - DNA, 20 bp, artificial
  human FXR antisense
acgatgtcttctacctcctt

SEQ ID NO: 698 - DNA, 20 bp, artificial
  human FXR antisense
ctaagagaagcagtgttcac

SEQ ID NO: 699 - DNA, 20 bp, artificial
  human FXR antisense
gaaatgcactttctttatgg

SEQ ID NO: 700 - DNA, 20 bp, artificial
  human FXR antisense
ctctgtctccacaaacaaca

SEQ ID NO: 701 - DNA, 20 bp, artificial
  human FXR antisense
tctctttgcatttccttagt

SEQ ID NO: 702 - DNA, 20 bp, artificial
  human FXR antisense
ctctgtttgttatatgaatc

SEQ ID NO: 703 - DNA, 20 bp, artificial
  human FXR antisense
aaaattttatttgttatttc

SEQ ID NO: 704 - DNA, 20 bp, artificial
  human FXR antisense
atcctggtagcttttttgtg

SEQ ID NO: 705 - DNA, 20 bp, artificial
  human FXR antisense
gtcagaatgcccagacggaa

SEQ ID NO: 706 - DNA, 20 bp, artificial
  human FXR antisense
aaacataggtgttatatatt

SEQ ID NO: 707 - DNA, 20 bp, artificial
  human FXR antisense
tggtgaatcttacacaactt

SEQ ID NO: 708 - DNA, 20 bp, artificial
  human FXR antisense
tgtaatccccatcactgcac

SEQ ID NO: 709 - DNA, 20 bp, artificial
  human FXR antisense
cttctacgatgtcttctacc

SEQ ID NO: 710 - DNA, 20 bp, artificial
  human FXR antisense
cagaggacctgccacttgtt

SEQ ID NO: 711 - DNA, 20 bp, artificial
  human FXR antisense
ttacaggcatctctgctacc

SEQ ID NO: 712 - DNA, 20 bp, artificial
  human FXR antisense
acgaccttcactgtcttcat

SEQ ID NO: 713 - DNA, 20 bp, artificial
  human FXR antisense
cacttgtcgcaagtcacgac

SEQ ID NO: 714 - DNA, 20 bp, artificial
  human FXR antisense
gtagaagagtctgttgatct

SEQ ID NO: 715 - DNA, 20 bp, artificial
  human FXR antisense
gattggttgccatttccgtc

SEQ ID NO: 716 - DNA, 20 bp, artificial
  human FXR antisense
tccaataggtcagaatgccc

SEQ ID NO: 717 - DNA, 20 bp, artificial
  human FXR antisense
acagggcttgccaattagaa

SEQ ID NO: 718 - DNA, 20 bp, artificial
  human FXR antisense
agatgagcaaaatgagattt

SEQ ID NO: 719 - DNA, 20 bp, artificial
  human FXR antisense
tttgctttattgccaagatt

SEQ ID NO: 720 - DNA, 20 bp, artificial
  human FXR antisense
agagatggactttcaaggcc

SEQ ID NO: 721 - DNA, 20 bp, artificial
  human FXR antisense
gaggacctgccacttgttct

SEQ ID NO: 722 - DNA, 20 bp, artificial
  human FXR antisense
acattgctgtattgcgagta

SEQ ID NO: 723 - DNA, 20 bp, artificial
  human FXR antisense
aaatcctggtagcttttttg

SEQ ID NO: 724 - DNA, 20 bp, artificial
  human FXR antisense
gtcttcatggtccaaagtct

SEQ ID NO: 725 - DNA, 20 bp, artificial
  human FXR antisense
atctggtcttcatggtccaa

SEQ ID NO: 726 - DNA, 20 bp, artificial
  human FXR antisense
tactgcctctctatccttta

SEQ ID NO: 727 - DNA, 20 bp, artificial
  human FXR antisense
gtccgtaattcagtcaggcg

SEQ ID NO: 728 - DNA, 20 bp, artificial
  human FXR antisense
acagttgtggaagttacaca

SEQ ID NO: 729 - DNA, 20 bp, artificial
  human FXR antisense
tatttacagttgtggaagtt

SEQ ID NO: 730 - DNA, 20 bp, artificial
  human FXR antisense
gttctatctagcccaatatt

SEQ ID NO: 731 - DNA, 20 bp, artificial
  human FXR antisense
tgtagagaaagttgttctat

SEQ ID NO: 732 - DNA, 20 bp, artificial
  human FXR antisense
cttttttgtgaattctacaa

SEQ ID NO: 733 - DNA, 20 bp, artificial
  human FXR antisense
ttcttattgaaaatctcagc

SEQ ID NO: 734 - DNA, 20 bp, artificial
  human FXR antisense
cttacacaacttttgtagca

SEQ ID NO: 735 - DNA, 20 bp, artificial
  human FXR antisense
gaatcttacacaacttttgt

SEQ ID NO: 736 - DNA, 20 bp, artificial
  human FXR antisense
gtgaatcttacacaactttt

SEQ ID NO: 737 - DNA, 20 bp, artificial
  human FXR antisense
agcccaatatttacagttgt

SEQ ID NO: 738 - DNA, 20 bp, artificial
  human FXR antisense
cagggcttgccaattagaat

SEQ ID NO: 739 - DNA, 20 bp, artificial
  human FXR antisense
acctccttggattgttttgg

SEQ ID NO: 740 - DNA, 20 bp, artificial
  human FXR antisense
tctacgatgtcttctacctc

SEQ ID NO: 741 - DNA, 20 bp, artificial
  human FXR antisense
gtctgaagtttcatcttgag

SEQ ID NO: 742 - DNA, 20 bp, artificial
  human FXR antisense
tgtctgaagtttcatcttga

SEQ ID NO: 743 - DNA, 20 bp, artificial
  human FXR antisense
agagtaccactcttcaggct

SEQ ID NO: 744 - DNA, 20 bp, artificial
  human FXR antisense
acaggcatctctgctacctc

SEQ ID NO: 745 - DNA, 20 bp, artificial
  human FXR antisense
tacaggcatctctgctacct

SEQ ID NO: 746 - DNA, 20 bp, artificial
  human FXR antisense
cccccgtttttacacttgta

SEQ ID NO: 747 - DNA, 20 bp, artificial
  human FXR antisense
gttgccatttccgtcaaaat

SEQ ID NO: 748 - DNA, 20 bp, artificial
  human FXR antisense
catgattggttgccatttcc

SEQ ID NO: 749 - DNA, 20 bp, artificial
  human FXR antisense
ccaaagtctgaaatcctggt

SEQ ID NO: 750 - DNA, 20 bp, artificial
  human FXR antisense
tccaaagtctgaaatcctgg

SEQ ID NO: 751 - DNA, 20 bp, artificial
  human FXR antisense
ccagacggaagtttcttatt

SEQ ID NO: 752 - DNA, 20 bp, artificial
  human FXR antisense
tcgaattctttcttccaata

SEQ ID NO: 753 - DNA, 20 bp, artificial
  human FXR antisense
taaacataggtgttatatat

SEQ ID NO: 754 - DNA, 20 bp, artificial
  human FXR antisense
ccctcccctgtaatccccat

SEQ ID NO: 755 - DNA, 20 bp, artificial
  human FXR antisense
tcttgagtgaaactgggtac

SEQ ID NO: 756 - DNA, 20 bp, artificial
  human FXR antisense
ttcatcaagatttcttgagt

SEQ ID NO: 757 - DNA, 20 bp, artificial
  human FXR antisense
tagaatgcaggattccctgg

SEQ ID NO: 758 - DNA, 20 bp, artificial
  human FXR antisense
aattgaagtaacaatcaatt

SEQ ID NO: 759 - DNA, 20 bp, artificial
  human FXR antisense
tattgccaagattgaataca

SEQ ID NO: 760 - DNA, 20 bp, artificial
  human FXR antisense
ctcagctggcatacgcctga

SEQ ID NO: 761 - DNA, 20 bp, artificial
  human FXR antisense
cagaggctctgtctccacaa

SEQ ID NO: 762 - DNA, 20 bp, artificial
  human FXR antisense
ttatttgttatttcctgagg

SEQ ID NO: 763 - DNA, 20 bp, artificial
  human FXR antisense
ccaggagacaggcaaagtgt

SEQ ID NO: 764 - DNA, 20 bp, artificial
  human FXR antisense
actgggtacaagtgaaataa

SEQ ID NO: 765 - DNA, 20 bp, artificial
  human FXR antisense
caatcaatttaattaggcaa

SEQ ID NO: 766 - DNA, 20 bp, artificial
  human FXR antisense
ggcttctttgttacaggcat

SEQ ID NO: 767 - DNA, 20 bp, artificial
  human FXR antisense
gtcacttgtcgcaagtcacg

SEQ ID NO: 768 - DNA, 20 bp, artificial
  human FXR antisense
aagtctgaaatcctggtagc

SEQ ID NO: 769 - DNA, 20 bp, artificial
  human FXR antisense
ttattgaaaatctcagctga

SEQ ID NO: 770 - DNA, 20 bp, artificial
  human FXR antisense
atcttacacaacttttgtag

SEQ ID NO: 771 - DNA, 20 bp, artificial
  human FXR antisense
tgaatcttacacaacttttg

SEQ ID NO: 772 - DNA, 20 bp, artificial
  human FXR antisense
cttgagtgaaactgggtaca

SEQ ID NO: 773 - DNA, 20 bp, artificial
  human FXR antisense
tttcttgagtgaaactgggt

SEQ ID NO: 774 - DNA, 20 bp, artificial
  human FXR antisense
attccctggagccttttaaa

SEQ ID NO: 775 - DNA, 20 bp, artificial
  human FXR antisense
gccaagattgaatacaactc

SEQ ID NO: 776 - DNA, 20 bp, artificial
  human FXR antisense
tcctctccagatcccagcga

SEQ ID NO: 777 - DNA, 20 bp, artificial
  human FXR antisense
ggtcagagatggactttcaa

SEQ ID NO: 778 - DNA, 20 bp, artificial
  human FXR antisense
tatgtttctaagtcttcttt

SEQ ID NO: 779 - DNA, 20 bp, artificial
  human FXR antisense
cattgctgtattgcgagtat

SEQ ID NO: 780 - DNA, 20 bp, artificial
  human FXR antisense
ctgaacattgctgtattgcg

SEQ ID NO: 781 - DNA, 20 bp, artificial
  human FXR antisense
taaaattttatttgttattt

SEQ ID NO: 782 - DNA, 20 bp, artificial
  human FXR antisense
cctctctatcctttatgtat

SEQ ID NO: 783 - DNA, 20 bp, artificial
  human FXR antisense
agtgttgaggattttcaggc

SEQ ID NO: 784 - DNA, 20 bp, artificial
  human FXR antisense
acttgtggtcgtttactctc

SEQ ID NO: 785 - DNA, 20 bp, artificial
  human FXR antisense
gattaatatgagagagaaaa

SEQ ID NO: 786 - DNA, 20 bp, artificial
  human FXR antisense
ccctgggaggattctggact

SEQ ID NO: 787 - DNA, 20 bp, artificial
  human FXR antisense
catatccatcacacagttgc

SEQ ID NO: 788 - DNA, 20 bp, artificial
  human FXR antisense
cacgaccttcactgtcttca

SEQ ID NO: 789 - DNA, 20 bp, artificial
  human FXR antisense
gtcgaggtcacttgtcgcaa

SEQ ID NO: 790 - DNA, 20 bp, artificial
  human FXR antisense
ttaaaattttatttgttatt

SEQ ID NO: 791 - DNA, 20 bp, artificial
  human FXR antisense
tttaaaattttatttgttat

SEQ ID NO: 792 - DNA, 20 bp, artificial
  human FXR antisense
gtccaaagtctgaaatcctg

SEQ ID NO: 793 - DNA, 20 bp, artificial
  human FXR antisense
accgcagaccctttcagcaa

SEQ ID NO: 794 - DNA, 20 bp, artificial
  human FXR antisense
tcctcttgagtcattttcag

SEQ ID NO: 795 - DNA, 20 bp, artificial
  human FXR antisense
tctctatcctttatgtattg

SEQ ID NO: 796 - DNA, 20 bp, artificial
  human FXR antisense
cccatcactgcacgtcccag

SEQ ID NO: 797 - DNA, 20 bp, artificial
  human FXR antisense
agattaatatgagagagaaa

SEQ ID NO: 798 - DNA, 20 bp, artificial
  human FXR antisense
aattagaatgcaggattccc

SEQ ID NO: 799 - DNA, 20 bp, artificial
  human FXR antisense
gactgagtcttcctctccag

SEQ ID NO: 800 - DNA, 20 bp, artificial
  human FXR antisense
gaatgttcaatgagattcat

SEQ ID NO: 801 - DNA, 20 bp, artificial
  human FXR antisense
agtctcagctggcatacgcc

SEQ ID NO: 802 - DNA, 20 bp, artificial
  human FXR antisense
catctctgctacctcagttt

SEQ ID NO: 803 - DNA, 20 bp, artificial
  human FXR antisense
tctggtcttcatggtccaaa

SEQ ID NO: 804 - DNA, 20 bp, artificial
  human FXR antisense
aaccgcagaccctttcagca

SEQ ID NO: 805 - DNA, 20 bp, artificial
  human FXR antisense
tcttccaataggtcagaatg

SEQ ID NO: 806 - DNA, 20 bp, artificial
  human FXR antisense
ctctactgcctctctatcct

SEQ ID NO: 807 - DNA, 20 bp, artificial
  human FXR antisense
tggagccttttaaaacacaa

SEQ ID NO: 808 - DNA, 20 bp, artificial
  human FXR antisense
ccctggagccttttaaaaca

SEQ ID NO: 809 - DNA, 20 bp, artificial
  human FXR antisense
aaatgagattttccctagtt

SEQ ID NO: 810 - DNA, 20 bp, artificial
  human FXR antisense
cctccttggattgttttggg

SEQ ID NO: 811 - DNA, 20 bp, artificial
  human FXR antisense
ctccttctacgatgtcttct

SEQ ID NO: 812 - DNA, 20 bp, artificial
  human FXR antisense
tgccacttgttctgttaaaa

SEQ ID NO: 813 - DNA, 20 bp, artificial
  human FXR antisense
gctacctcagtttctccctg

SEQ ID NO: 814 - DNA, 20 bp, artificial
  human FXR antisense
tgctacctcagtttctccct

SEQ ID NO: 815 - DNA, 20 bp, artificial
  human FXR antisense
ctgctacctcagtttctccc

SEQ ID NO: 816 - DNA, 20 bp, artificial
  human FXR antisense
atccagaggctctgtctcca

SEQ ID NO: 817 - DNA, 20 bp, artificial
  human FXR antisense
cttctcctgaagaaaccttt

SEQ ID NO: 818 - DNA, 20 bp, artificial
  human FXR antisense
tggtaatgcttctcctgaag

SEQ ID NO: 819 - DNA, 20 bp, artificial
  human FXR antisense
ttcacggtctgatctgcatg

SEQ ID NO: 820 - DNA, 20 bp, artificial
  human FXR antisense
ccataataaaatgtagaaga

SEQ ID NO: 821 - DNA, 20 bp, artificial
  human FXR antisense
acaagaacctgtacatgatt

SEQ ID NO: 822 - DNA, 20 bp, artificial
  human FXR antisense
tggtccaaagtctgaaatcc

SEQ ID NO: 823 - DNA, 20 bp, artificial
  human FXR antisense
ggtcttcatggtccaaagtc

SEQ ID NO: 824 - DNA, 20 bp, artificial
  human FXR antisense
ctctatcctttatgtattgt

SEQ ID NO: 825 - DNA, 20 bp, artificial
  human FXR antisense
gctcctgaagcttctctact

SEQ ID NO: 826 - DNA, 20 bp, artificial
  human FXR antisense
tgttgaggattttcaggctg

SEQ ID NO: 827 - DNA, 20 bp, artificial
  human FXR antisense
cgtggtgatgattgaatgtc

SEQ ID NO: 828 - DNA, 20 bp, artificial
  human FXR antisense
ccccatcactgcacgtccca

SEQ ID NO: 829 - DNA, 20 bp, artificial
  human FXR antisense
taatccccatcactgcacgt

SEQ ID NO: 830 - DNA, 20 bp, artificial
  human FXR antisense
ttcttgagtgaaactgggta

SEQ ID NO: 831 - DNA, 20 bp, artificial
  human FXR antisense
ctggagccttttaaaacaca

SEQ ID NO: 832 - DNA, 20 bp, artificial
  human FXR antisense
aacaatcaatttaattaggc

SEQ ID NO: 833 - DNA, 20 bp, artificial
  human FXR antisense
cctctccagatcccagcgat

SEQ ID NO: 834 - DNA, 20 bp, artificial
  human FXR antisense
cttcctctccagatcccagc

SEQ ID NO: 835 - DNA, 20 bp, artificial
  human FXR antisense
aggattctggactgagtctt

SEQ ID NO: 836 - DNA, 20 bp, artificial
  human FXR antisense
tgttttgggtcagagatgga

SEQ ID NO: 837 - DNA, 20 bp, artificial
  human FXR antisense
agaggacctgccacttgttc

SEQ ID NO: 838 - DNA, 20 bp, artificial
  human FXR antisense
cttcaggctgctgggggtag

SEQ ID NO: 839 - DNA, 20 bp, artificial
  human FXR antisense
tccagaggctctgtctccac

SEQ ID NO: 840 - DNA, 20 bp, artificial
  human FXR antisense
cattcagccaacattcccat

SEQ ID NO: 841 - DNA, 20 bp, artificial
  human FXR antisense
atggtccaaagtctgaaatc

SEQ ID NO: 842 - DNA, 20 bp, artificial
  human FXR antisense
aaagcaatctggtcttcatg

SEQ ID NO: 843 - DNA, 20 bp, artificial
  human FXR antisense
ttcagcaaagcaatctggtc

SEQ ID NO: 844 - DNA, 20 bp, artificial
  human FXR antisense
gtgttatatattcatcagag

SEQ ID NO: 845 - DNA, 20 bp, artificial
  human FXR antisense
ctgcctctctatcctttatg

SEQ ID NO: 846 - DNA, 20 bp, artificial
  human FXR antisense
ttgaggattttcaggctggt

SEQ ID NO: 847 - DNA, 20 bp, artificial
  human FXR antisense
gcgtggtgatgattgaatgt

SEQ ID NO: 848 - DNA, 20 bp, artificial
  human FXR antisense
tgaaactgggtacaagtgaa

SEQ ID NO: 849 - DNA, 20 bp, artificial
  human FXR antisense
caagatttcttgagtgaaac

SEQ ID NO: 850 - DNA, 20 bp, artificial
  human FXR antisense
ttagaatgcaggattccctg

SEQ ID NO: 851 - DNA, 20 bp, artificial
  human FXR antisense
agattgaatacaactcttta

SEQ ID NO: 852 - DNA, 20 bp, artificial
  human FXR antisense
ttgctacaaatgctcagaat

SEQ ID NO: 853 - DNA, 20 bp, artificial
  human FXR antisense
ttcctctccagatcccagcg

SEQ ID NO: 854 - DNA, 20 bp, artificial
  human FXR antisense
tccttggattgttttgggtc

SEQ ID NO: 855 - DNA, 20 bp, artificial
  human FXR antisense
ctacgatgtcttctacctcc

SEQ ID NO: 856 - DNA, 20 bp, artificial
  human FXR antisense
ttcactccttctacgatgtc

SEQ ID NO: 857 - DNA, 20 bp, artificial
  human FXR antisense
tttcactccttctacgatgt

SEQ ID NO: 858 - DNA, 20 bp, artificial
  human FXR antisense
ttcatttttgatcccatcca

SEQ ID NO: 859 - DNA, 20 bp, artificial
  human FXR antisense
gctgtattgcgagtatggtt

SEQ ID NO: 860 - DNA, 20 bp, artificial
  human FXR antisense
gagagtaccactcttcaggc

SEQ ID NO: 861 - DNA, 20 bp, artificial
  human FXR antisense
ttcactgtcttcattcacgg

SEQ ID NO: 862 - DNA, 20 bp, artificial
  human FXR antisense
tggctcctgaagcttctcta

SEQ ID NO: 863 - DNA, 20 bp, artificial
  human FXR antisense
aggattttcaggctggtgaa

SEQ ID NO: 864 - DNA, 20 bp, artificial
  human FXR antisense
tcactgcacgtcccagattt

SEQ ID NO: 865 - DNA, 20 bp, artificial
  human FXR antisense
gttacacatgtaattacaac

SEQ ID NO: 866 - DNA, 20 bp, artificial
  human FXR antisense
tccctggagccttttaaaac

SEQ ID NO: 867 - DNA, 20 bp, artificial
  human FXR antisense
gctacaaatgctcagaatcc

SEQ ID NO: 868 - DNA, 20 bp, artificial
  human FXR antisense
tggtggtcttcaaaaaaaac

SEQ ID NO: 869 - DNA, 20 bp, artificial
  human FXR antisense
tacctcagtttctccctggt

SEQ ID NO: 870 - DNA, 20 bp, artificial
  human FXR antisense
actgtcttcattcacggtct

SEQ ID NO: 871 - DNA, 20 bp, artificial
  human FXR antisense
tcactgtcttcattcacggt

SEQ ID NO: 872 - DNA, 20 bp, artificial
  human FXR antisense
tgatctggggtgagttcagt

SEQ ID NO: 873 - DNA, 20 bp, artificial
  human FXR antisense
gcttcaaccgcagacccttt

SEQ ID NO: 874 - DNA, 20 bp, artificial
  human FXR antisense
ctactgcctctctatccttt

SEQ ID NO: 875 - DNA, 20 bp, artificial
  human FXR antisense
aatcttacacaacttttgta

SEQ ID NO: 876 - DNA, 20 bp, artificial
  human FXR antisense
gacatcagcatctcagcgtg

SEQ ID NO: 877 - DNA, 20 bp, artificial
  human FXR antisense
ttgtggtcgtttactctcca

SEQ ID NO: 878 - DNA, 20 bp, artificial
  human FXR antisense
aaagttatacatcagattaa

SEQ ID NO: 879 - DNA, 20 bp, artificial
  human FXR antisense
aaactgggtacaagtgaaat

SEQ ID NO: 880 - DNA, 20 bp, artificial
  human FXR antisense
ttccctggagccttttaaaa

SEQ ID NO: 881 - DNA, 20 bp, artificial
  human FXR antisense
aatgagattttccctagttc

SEQ ID NO: 882 - DNA, 20 bp, artificial
  human FXR antisense
tgagtcttcctctccagatc

SEQ ID NO: 883 - DNA, 20 bp, artificial
  human FXR antisense
actttcaaggccctgggagg

SEQ ID NO: 884 - DNA, 20 bp, artificial
  human FXR antisense
tcactccttctacgatgtct

SEQ ID NO: 885 - DNA, 20 bp, artificial
  human FXR antisense
gtattgcgagtatggttcca

SEQ ID NO: 886 - DNA, 20 bp, artificial
  human FXR antisense
aactgaacattgctgtattg

SEQ ID NO: 887 - DNA, 20 bp, artificial
  human FXR antisense
gttatatgaatccataataa

SEQ ID NO: 888 - DNA, 20 bp, artificial
  human FXR antisense
tctcagctgaacgaaggaac

SEQ ID NO: 889 - DNA, 20 bp, artificial
  human FXR antisense
ttatgtattgtctatctgga

SEQ ID NO: 890 - DNA, 20 bp, artificial
  human FXR antisense
cttctctactgcctctctat

SEQ ID NO: 891 - DNA, 20 bp, artificial
  human FXR antisense
aacttttgtagcacatcaag

SEQ ID NO: 892 - DNA, 20 bp, artificial
  human FXR antisense
caggcgacccaggagacagg

SEQ ID NO: 893 - DNA, 20 bp, artificial
  human FXR antisense
aatgtccgtaattcagtcag

SEQ ID NO: 894 - DNA, 20 bp, artificial
  human FXR antisense
agtgaaactgggtacaagtg

SEQ ID NO: 895 - DNA, 20 bp, artificial
  human FXR antisense
aaacagggcttgccaattag

SEQ ID NO: 896 - DNA, 20 bp, artificial
  human FXR antisense
tggtaagatgagcaaaatga

SEQ ID NO: 897 - DNA, 20 bp, artificial
  human FXR antisense
ctggactgagtcttcctctc

SEQ ID NO: 898 - DNA, 20 bp, artificial
  human FXR antisense
ccttctacgatgtcttctac

SEQ ID NO: 899 - DNA, 20 bp, artificial
  human FXR antisense
atggtggtcttcaaaaaaaa

SEQ ID NO: 900 - DNA, 20 bp, artificial
  human FXR antisense
gcatctctgctacctcagtt

SEQ ID NO: 901 - DNA, 20 bp, artificial
  human FXR antisense
tcttcgcatgtacatatcca

SEQ ID NO: 902 - DNA, 20 bp, artificial
  human FXR antisense
cttcactgtcttcattcacg

SEQ ID NO: 903 - DNA, 20 bp, artificial
  human FXR antisense
aatcctggtagcttttttgt

SEQ ID NO: 904 - DNA, 20 bp, artificial
  human FXR antisense
tgaaaatctcagctgaacga

SEQ ID NO: 905 - DNA, 20 bp, artificial
  human FXR antisense
atcactgcacgtcccagatt

SEQ ID NO: 906 - DNA, 20 bp, artificial
  human FXR antisense
caggattccctggagccttt

SEQ ID NO: 907 - DNA, 20 bp, artificial
  human FXR antisense
attagaatgcaggattccct

SEQ ID NO: 908 - DNA, 20 bp, artificial
  human FXR antisense
tcagagatggactttcaagg

SEQ ID NO: 909 - DNA, 20 bp, artificial
  human FXR antisense
gtcagagatggactttcaag

SEQ ID NO: 910 - DNA, 20 bp, artificial
  human FXR antisense
caggctgctgggggtagaaa

SEQ ID NO: 911 - DNA, 20 bp, artificial
  human FXR antisense
tcagctggcatacgcctgag

SEQ ID NO: 912 - DNA, 20 bp, artificial
  human FXR antisense
tctcagctggcatacgcctg

SEQ ID NO: 913 - DNA, 20 bp, artificial
  human FXR antisense
catcccctttgatcctccct

SEQ ID NO: 914 - DNA, 20 bp, artificial
  human FXR antisense
cagctcatcccctttgatcc

SEQ ID NO: 915 - DNA, 20 bp, artificial
  human FXR antisense
atagtggtatccagaggctc

SEQ ID NO: 916 - DNA, 20 bp, artificial
  human FXR antisense
cacagcgtttttggtaatgc

SEQ ID NO: 917 - DNA, 20 bp, artificial
  human FXR antisense
gaccttcactgtcttcattc

SEQ ID NO: 918 - DNA, 20 bp, artificial
  human FXR antisense
ttttaaaattttatttgtta

SEQ ID NO: 919 - DNA, 20 bp, artificial
  human FXR antisense
ttcttccaataggtcagaat

SEQ ID NO: 920 - DNA, 20 bp, artificial
  human FXR antisense
tttcttccaataggtcagaa

SEQ ID NO: 921 - DNA, 20 bp, artificial
  human FXR antisense
ctgtaatccccatcactgca

SEQ ID NO: 922 - DNA, 20 bp, artificial
  human FXR antisense
tagacccctcccctgtaatc

SEQ ID NO: 923 - DNA, 20 bp, artificial
  human FXR antisense
gtgaaactgggtacaagtga

SEQ ID NO: 924 - DNA, 20 bp, artificial
  human FXR antisense
aagttacacatgtaattaca

SEQ ID NO: 925 - DNA, 20 bp, artificial
  human FXR antisense
attaggcaaacagggcttgc

SEQ ID NO: 926 - DNA, 20 bp, artificial
  human FXR antisense
gtaacaatcaatttaattag

SEQ ID NO: 927 - DNA, 20 bp, artificial
  human FXR antisense
gattgaatacaactctttaa

SEQ ID NO: 928 - DNA, 20 bp, artificial
  human FXR antisense
attgccaagattgaatacaa

SEQ ID NO: 929 - DNA, 20 bp, artificial
  human FXR antisense
ccaggaaactaagagaagca

SEQ ID NO: 930 - DNA, 20 bp, artificial
  human FXR antisense
acattcccatctctttgcat

SEQ ID NO: 931 - DNA, 20 bp, artificial
  human FXR antisense
tcagttaacaagcattcagc

SEQ ID NO: 932 - DNA, 20 bp, artificial
  human FXR antisense
tctcagtcgcttagatttac

SEQ ID NO: 933 - DNA, 20 bp, artificial
  human FXR antisense
tgtagaagagtctgttgatc

SEQ ID NO: 934 - DNA, 20 bp, artificial
  human FXR antisense
attgaaaatctcagctgaac

SEQ ID NO: 935 - DNA, 20 bp, artificial
  human FXR antisense
cctgtaatccccatcactgc

SEQ ID NO: 936 - DNA, 20 bp, artificial
  human FXR antisense
atcaagatttcttgagtgaa

SEQ ID NO: 937 - DNA, 20 bp, artificial
  human FXR antisense
tacagttgtggaagttacac

SEQ ID NO: 938 - DNA, 20 bp, artificial
  human FXR antisense
agtgtctgaagtttcatctt

SEQ ID NO: 939 - DNA, 20 bp, artificial
  human FXR antisense
tcatttttgatcccatccaa

SEQ ID NO: 940 - DNA, 20 bp, artificial
  human FXR antisense
ggttctgtcccagaggacct

SEQ ID NO: 941 - DNA, 20 bp, artificial
  human FXR antisense
tgcgagtatggttccacttc

SEQ ID NO: 942 - DNA, 20 bp, artificial
  human FXR antisense
ctcctgaagaaacctttaca

SEQ ID NO: 943 - DNA, 20 bp, artificial
  human FXR antisense
aacattcccatctctttgca

SEQ ID NO: 944 - DNA, 20 bp, artificial
  human FXR antisense
ctcagtcgcttagatttaca

SEQ ID NO: 945 - DNA, 20 bp, artificial
  human FXR antisense
aagcttctctactgcctctc

SEQ ID NO: 946 - DNA, 20 bp, artificial
  human FXR antisense
caagaagtggctcctgaagc

SEQ ID NO: 947 - DNA, 20 bp, artificial
  human FXR antisense
tcaagaagtggctcctgaag

SEQ ID NO: 948 - DNA, 20 bp, artificial
  human FXR antisense
atcaagaagtggctcctgaa

SEQ ID NO: 949 - DNA, 20 bp, artificial
  human FXR antisense
ggattttcaggctggtgaat

SEQ ID NO: 950 - DNA, 20 bp, artificial
  human FXR antisense
agaagtggggtaaacttgtg

SEQ ID NO: 951 - DNA, 20 bp, artificial
  human FXR antisense
atttcttgagtgaaactggg

SEQ ID NO: 952 - DNA, 20 bp, artificial
  human FXR antisense
aatatttacagttgtggaag

SEQ ID NO: 953 - DNA, 20 bp, artificial
  human FXR antisense
aaagttgttctatctagccc

SEQ ID NO: 954 - DNA, 20 bp, artificial
  human FXR antisense
gatgagcaaaatgagatttt

SEQ ID NO: 955 - DNA, 20 bp, artificial
  human FXR antisense
tacctccttggattgttttg

SEQ ID NO: 956 - DNA, 20 bp, artificial
  human FXR antisense
cttcgcatgtacatatccat

SEQ ID NO: 957 - DNA, 20 bp, artificial
  human FXR antisense
tgacactttcttcgcatgta

SEQ ID NO: 958 - DNA, 20 bp, artificial
  human FXR antisense
ccttcactgtcttcattcac

SEQ ID NO: 959 - DNA, 20 bp, artificial
  human FXR antisense
tggtcttcatggtccaaagt

SEQ ID NO: 960 - DNA, 20 bp, artificial
  human FXR antisense
ggcgacccaggagacaggca

SEQ ID NO: 961 - DNA, 20 bp, artificial
  human FXR antisense
gagtgaaactgggtacaagt

SEQ ID NO: 962 - DNA, 20 bp, artificial
  human FXR antisense
tcaagatttcttgagtgaaa

SEQ ID NO: 963 - DNA, 20 bp, artificial
  human FXR antisense
gaatgcaggattccctggag

SEQ ID NO: 964 - DNA, 20 bp, artificial
  human FXR antisense
aatcaatttaattaggcaaa

SEQ ID NO: 965 - DNA, 20 bp, artificial
  human FXR antisense
attttccctagttcaacaga

SEQ ID NO: 966 - DNA, 20 bp, artificial
  human FXR antisense
ccaagattgaatacaactct

SEQ ID NO: 967 - DNA, 20 bp, artificial
  human FXR antisense
aaggccctgggaggattctg

SEQ ID NO: 968 - DNA, 20 bp, artificial
  human FXR antisense
caaggccctgggaggattct

SEQ ID NO: 969 - DNA, 20 bp, artificial
  human FXR antisense
ggtggtcttcaaaaaaaact

SEQ ID NO: 970 - DNA, 20 bp, artificial
  human FXR antisense
tatagtggtatccagaggct

SEQ ID NO: 971 - DNA, 20 bp, artificial
  human FXR antisense
agttaacaagcattcagcca

SEQ ID NO: 972 - DNA, 20 bp, artificial
  human FXR antisense
catcactgcacgtcccagat

SEQ ID NO: 973 - DNA, 20 bp, artificial
  human FXR antisense
atggtaagatgagcaaaatg

SEQ ID NO: 974 - DNA, 20 bp, artificial
  human FXR antisense
gagtcttcctctccagatcc

SEQ ID NO: 975 - DNA, 20 bp, artificial
  human FXR antisense
aggaaactaagagaagcagt

SEQ ID NO: 976 - DNA, 20 bp, artificial
  human FXR antisense
tttcaattgaaatgcacttt

SEQ ID NO: 977 - DNA, 20 bp, artificial
  human FXR antisense
ttgcgagtatggttccactt

SEQ ID NO: 978 - DNA, 20 bp, artificial
  human FXR antisense
tgtattgcgagtatggttcc

SEQ ID NO: 979 - DNA, 20 bp, artificial
  human FXR antisense
aacattgctgtattgcgagt

SEQ ID NO: 980 - DNA, 20 bp, artificial
  human FXR antisense
ctacctcagtttctccctgg

SEQ ID NO: 981 - DNA, 20 bp, artificial
  human FXR antisense
ggtgagttcagttttctccc

SEQ ID NO: 982 - DNA, 20 bp, artificial
  human FXR antisense
ttacagttgtggaagttaca

SEQ ID NO: 983 - DNA, 20 bp, artificial
  human FXR antisense
ttaggcaaacagggcttgcc

SEQ ID NO: 984 - DNA, 20 bp, artificial
  human FXR antisense
ttcatcttgaggaaatgtcc

SEQ ID NO: 985 - DNA, 20 bp, artificial
  human FXR antisense
tacacttgtacacagcgttt

SEQ ID NO: 986 - DNA, 20 bp, artificial
  human FXR antisense
ttacacttgtacacagcgtt

SEQ ID NO: 987 - DNA, 20 bp, artificial
  human FXR antisense
tttacacttgtacacagcgt

SEQ ID NO: 988 - DNA, 20 bp, artificial
  human FXR antisense
gaatccataataaaatgtag

SEQ ID NO: 989 - DNA, 20 bp, artificial
  human FXR antisense
ctcagctgaacgaaggaaca

SEQ ID NO: 990 - DNA, 20 bp, artificial
  human FXR antisense
gaaaatctcagctgaacgaa

SEQ ID NO: 991 - DNA, 20 bp, artificial
  human FXR antisense
tattgaaaatctcagctgaa

SEQ ID NO: 992 - DNA, 20 bp, artificial
  human FXR antisense
aggctggtgaatcttacaca

SEQ ID NO: 993 - DNA, 20 bp, artificial
  human FXR antisense
ttcaggctggtgaatcttac

SEQ ID NO: 994 - DNA, 20 bp, artificial
  human FXR antisense
agcgtggtgatgattgaatg

SEQ ID NO: 995 - DNA, 20 bp, artificial
  human FXR antisense
ttctatctagcccaatattt

SEQ ID NO: 996 - DNA, 20 bp, artificial
  human FXR antisense
gaattgaagtaacaatcaat

SEQ ID NO: 997 - DNA, 20 bp, artificial
  human FXR antisense
attgaatacaactctttaat

SEQ ID NO: 998 - DNA, 20 bp, artificial
  human FXR antisense
tgtttctaagtcttcttttc

SEQ ID NO: 999 - DNA, 20 bp, artificial
  human FXR antisense
ctatgtttctaagtcttctt

SEQ ID NO: 1000 - DNA, 20 bp, artificial
  human FXR antisense
ttgagctatgtttctaagtc

SEQ ID NO: 1001 - DNA, 20 bp, artificial
  human FXR antisense
gaaactaagagaagcagtgt

SEQ ID NO: 1002 - DNA, 20 bp, artificial
  human FXR antisense
ttttcaattgaaatgcactt

SEQ ID NO: 1003 - DNA, 20 bp, artificial
  human FXR antisense
tttttcaattgaaatgcact

SEQ ID NO: 1004 - DNA, 20 bp, artificial
  human FXR antisense
tctgtctccacaaacaacac

SEQ ID NO: 1005 - DNA, 20 bp, artificial
  human FXR antisense
tatccagaggctctgtctcc

SEQ ID NO: 1006 - DNA, 20 bp, artificial
  human FXR antisense
accttcactgtcttcattca

SEQ ID NO: 1007 - DNA, 20 bp, artificial
  human FXR antisense
agtcacgaccttcactgtct

SEQ ID NO: 1008 - DNA, 20 bp, artificial
  human FXR antisense
gtagagaaagttgttctatc

SEQ ID NO: 1009 - DNA, 20 bp, artificial
  human FXR antisense
acaactctttaataaaatat

SEQ ID NO: 1010 - DNA, 20 bp, artificial
  human FXR antisense
tttcttctttcactccttct

SEQ ID NO: 1011 - DNA, 20 bp, artificial
  human FXR antisense
gtttctaagtcttcttttct

SEQ ID NO: 1012 - DNA, 20 bp, artificial
  human FXR antisense
aaatccaggaaactaagaga

SEQ ID NO: 1013 - DNA, 20 bp, artificial
  human FXR antisense
tttatggtggtcttcaaaaa

SEQ ID NO: 1014 - DNA, 20 bp, artificial
  human FXR antisense
ttgaaatgcactttctttat

SEQ ID NO: 1015 - DNA, 20 bp, artificial
  human FXR antisense
attgaaatgcactttcttta

SEQ ID NO: 1016 - DNA, 20 bp, artificial
  human FXR antisense
tctctgctacctcagtttct

SEQ ID NO: 1017 - DNA, 20 bp, artificial
  human FXR antisense
ttcgcatgtacatatccatc

SEQ ID NO: 1018 - DNA, 20 bp, artificial
  human FXR antisense
ttccaataggtcagaatgcc

SEQ ID NO: 1019 - DNA, 20 bp, artificial
  human FXR antisense
aagtggctcctgaagcttct

SEQ ID NO: 1020 - DNA, 20 bp, artificial
  human FXR antisense
caggagacaggcaaagtgtt

SEQ ID NO: 1021 - DNA, 20 bp, artificial
  human FXR antisense
tccgtaattcagtcaggcga

SEQ ID NO: 1022 - DNA, 20 bp, artificial
  human FXR antisense
agttacacatgtaattacaa

SEQ ID NO: 1023 - DNA, 20 bp, artificial
  human FXR antisense
atcccagcgattttgctaca

SEQ ID NO: 1024 - DNA, 20 bp, artificial
  human FXR antisense
tcttcctctccagatcccag

SEQ ID NO: 1025 - DNA, 20 bp, artificial
  human FXR antisense
gtcttctacctccttggatt

SEQ ID NO: 1026 - DNA, 20 bp, artificial
  human FXR antisense
atgagattcatttttgatcc

SEQ ID NO: 1027 - DNA, 20 bp, artificial
  human FXR antisense
aatgagattcatttttgatc

SEQ ID NO: 1028 - DNA, 20 bp, artificial
  human FXR antisense
ggtaggtaaatgggaatgtt

SEQ ID NO: 1029 - DNA, 20 bp, artificial
  human FXR antisense
tcagtcgcttagatttacac

SEQ ID NO: 1030 - DNA, 20 bp, artificial
  human FXR antisense
tttcttattgaaaatctcag

SEQ ID NO: 1031 - DNA, 20 bp, artificial
  human FXR antisense
cgaattctttcttccaatag

SEQ ID NO: 1032 - DNA, 20 bp, artificial
  human FXR antisense
ctaaacataggtgttatata

SEQ ID NO: 1033 - DNA, 20 bp, artificial
  human FXR antisense
cacatcaagaagtggctcct

SEQ ID NO: 1034 - DNA, 20 bp, artificial
  human FXR antisense
tttcaggctggtgaatctta

SEQ ID NO: 1035 - DNA, 20 bp, artificial
  human FXR antisense
cccaggagacaggcaaagtg

SEQ ID NO: 1036 - DNA, 20 bp, artificial
  human FXR antisense
ctgggtacaagtgaaataaa

SEQ ID NO: 1037 - DNA, 20 bp, artificial
  human FXR antisense
aagatttcttgagtgaaact

SEQ ID NO: 1038 - DNA, 20 bp, artificial
  human FXR antisense
attcatcaagatttcttgag

SEQ ID NO: 1039 - DNA, 20 bp, artificial
  human FXR antisense
aaaatgagattttccctagt

SEQ ID NO: 1040 - DNA, 20 bp, artificial
  human FXR antisense
ggtaagatgagcaaaatgag

SEQ ID NO: 1041 - DNA, 20 bp, artificial
  human FXR antisense
agtcggggagacaatgaggt

SEQ ID NO: 1042 - DNA, 20 bp, artificial
  human FXR antisense
atgctcagaatccaatttcg

SEQ ID NO: 1043 - DNA, 20 bp, artificial
  human FXR antisense
caaatgctcagaatccaatt

SEQ ID NO: 1044 - DNA, 20 bp, artificial
  human FXR antisense
actaagagaagcagtgttca

SEQ ID NO: 1045 - DNA, 20 bp, artificial
  human FXR antisense
ctgaagtttcatcttgagga

SEQ ID NO: 1046 - DNA, 20 bp, artificial
  human FXR antisense
tggtaggtaaatgggaatgt

SEQ ID NO: 1047 - DNA, 20 bp, artificial
  human FXR antisense
tcacgaccttcactgtcttc

SEQ ID NO: 1048 - DNA, 20 bp, artificial
  human FXR antisense
ctacaagaacctgtacatga

SEQ ID NO: 1049 - DNA, 20 bp, artificial
  human FXR antisense
aattctacaagaacctgtac

SEQ ID NO: 1050 - DNA, 20 bp, artificial
  human FXR antisense
tcagctgaacgaaggaacat

SEQ ID NO: 1051 - DNA, 20 bp, artificial
  human FXR antisense
atctcagctgaacgaaggaa

SEQ ID NO: 1052 - DNA, 20 bp, artificial
  human FXR antisense
ttgaaaatctcagctgaacg

SEQ ID NO: 1053 - DNA, 20 bp, artificial
  human FXR antisense
tgtggtcgtttactctccat

SEQ ID NO: 1054 - DNA, 20 bp, artificial
  human FXR antisense
agacccctcccctgtaatcc

SEQ ID NO: 1055 - DNA, 20 bp, artificial
  human FXR antisense
caagtgaaataaaggaaagt

SEQ ID NO: 1056 - DNA, 20 bp, artificial
  human FXR antisense
agggcttgccaattagaatg

SEQ ID NO: 1057 - DNA, 20 bp, artificial
  human FXR antisense
taggcaaacagggcttgcca

SEQ ID NO: 1058 - DNA, 20 bp, artificial
  human FXR antisense
atacaactctttaataaaat

SEQ ID NO: 1059 - DNA, 20 bp, artificial
  human FXR antisense
agctatgtttctaagtcttc

SEQ ID NO: 1060 - DNA, 20 bp, artificial
  human FXR antisense
aatccaggaaactaagagaa

SEQ ID NO: 1061 - DNA, 20 bp, artificial
  human FXR antisense
tgaacattgctgtattgcga

SEQ ID NO: 1062 - DNA, 20 bp, artificial
  human FXR antisense
cttttaaaattttatttgtt

SEQ ID NO: 1063 - DNA, 20 bp, artificial
  human FXR antisense
gccatttccgtcaaaatgag

SEQ ID NO: 1064 - DNA, 20 bp, artificial
  human FXR antisense
tgccatttccgtcaaaatga

SEQ ID NO: 1065 - DNA, 20 bp, artificial
  human FXR antisense
gtgaattctacaagaacctg

SEQ ID NO: 1066 - DNA, 20 bp, artificial
  human FXR antisense
tggactttcaaggccctggg

SEQ ID NO: 1067 - DNA, 20 bp, artificial
  human FXR antisense
tggattgttttgggtcagag

SEQ ID NO: 1068 - DNA, 20 bp, artificial
  human FXR antisense
aaactaagagaagcagtgtt

SEQ ID NO: 1069 - DNA, 20 bp, artificial
  human FXR antisense
ctccaaagtgtctgaagttt

SEQ ID NO: 1070 - DNA, 20 bp, artificial
  human FXR antisense
aattgaaatgcactttcttt

SEQ ID NO: 1071 - DNA, 20 bp, artificial
  human FXR antisense
catttttgatcccatccaaa

SEQ ID NO: 1072 - DNA, 20 bp, artificial
  human FXR antisense
gttctgtcccagaggacctg

SEQ ID NO: 1073 - DNA, 20 bp, artificial
  human FXR antisense
atcccctttgatcctccctg

SEQ ID NO: 1074 - DNA, 20 bp, artificial
  human FXR antisense
cattttttctcagtcgctta

SEQ ID NO: 1075 - DNA, 20 bp, artificial
  human FXR antisense
tcttcttttaaaattttatt

SEQ ID NO: 1076 - DNA, 20 bp, artificial
  human FXR antisense
tacaagaacctgtacatgat

SEQ ID NO: 1077 - DNA, 20 bp, artificial
  human FXR antisense
tctacaagaacctgtacatg

SEQ ID NO: 1078 - DNA, 20 bp, artificial
  human FXR antisense
gctgaacgaaggaacatagc

SEQ ID NO: 1079 - DNA, 20 bp, artificial
  human FXR antisense
tgttatatattcatcagaga

SEQ ID NO: 1080 - DNA, 20 bp, artificial
  human FXR antisense
agaagtggctcctgaagctt

SEQ ID NO: 1081 - DNA, 20 bp, artificial
  human FXR antisense
gattttcaggctggtgaatc

SEQ ID NO: 1082 - DNA, 20 bp, artificial
  human FXR antisense
acccaggagacaggcaaagt

SEQ ID NO: 1083 - DNA, 20 bp, artificial
  human FXR antisense
gtgaaataaaggaaagttat

SEQ ID NO: 1084 - DNA, 20 bp, artificial
  human FXR antisense
agtgaaataaaggaaagtta

SEQ ID NO: 1085 - DNA, 20 bp, artificial
  human FXR antisense
tgagtgaaactgggtacaag

SEQ ID NO: 1086 - DNA, 20 bp, artificial
  human FXR antisense
agaattgaagtaacaatcaa

SEQ ID NO: 1087 - DNA, 20 bp, artificial
  human FXR antisense
agcaaaatgagattttccct

SEQ ID NO: 1088 - DNA, 20 bp, artificial
  human FXR antisense
tatggtaagatgagcaaaat

SEQ ID NO: 1089 - DNA, 20 bp, artificial
  human FXR antisense
ttgccaagattgaatacaac

SEQ ID NO: 1090 - DNA, 20 bp, artificial
  human FXR antisense
tgctacaaatgctcagaatc

SEQ ID NO: 1091 - DNA, 20 bp, artificial
  human FXR antisense
tcccagcgattttgctacaa

SEQ ID NO: 1092 - DNA, 20 bp, artificial
  human FXR antisense
tcaaggccctgggaggattc

SEQ ID NO: 1093 - DNA, 20 bp, artificial
  human FXR antisense
ggaatgttcaatgagattca

SEQ ID NO: 1094 - DNA, 20 bp, artificial
  human FXR antisense
ttcacattttttctcagtcg

SEQ ID NO: 1095 - DNA, 20 bp, artificial
  human FXR antisense
ttgccatttccgtcaaaatg

SEQ ID NO: 1096 - DNA, 20 bp, artificial
  human FXR antisense
aagcaatctggtcttcatgg

SEQ ID NO: 1097 - DNA, 20 bp, artificial
  human FXR antisense
aattctttcttccaataggt

SEQ ID NO: 1098 - DNA, 20 bp, artificial
  human FXR antisense
gcctctctatcctttatgta

SEQ ID NO: 1099 - DNA, 20 bp, artificial
  human FXR antisense
gaaagttatacatcagatta

SEQ ID NO: 1100 - DNA, 20 bp, artificial
  human FXR antisense
caatatttacagttgtggaa

SEQ ID NO: 1101 - DNA, 20 bp, artificial
  human FXR antisense
ctccagatcccagcgatttt

SEQ ID NO: 1102 - DNA, 20 bp, artificial
  human FXR antisense
ctctccagatcccagcgatt

SEQ ID NO: 1103 - DNA, 20 bp, artificial
  human FXR antisense
tgtcttctacctccttggat

SEQ ID NO: 1104 - DNA, 20 bp, artificial
  human FXR antisense
gatcccatccaaatttttca

SEQ ID NO: 1105 - DNA, 20 bp, artificial
  human FXR antisense
tcatcccctttgatcctccc

SEQ ID NO: 1106 - DNA, 20 bp, artificial
  human FXR antisense
tcgcatgtacatatccatca

SEQ ID NO: 1107 - DNA, 20 bp, artificial
  human FXR antisense
cataataaaatgtagaagag

SEQ ID NO: 1108 - DNA, 20 bp, artificial
  human FXR antisense
tgaattctacaagaacctgt

SEQ ID NO: 1109 - DNA, 20 bp, artificial
  human FXR antisense
tgtgaattctacaagaacct

SEQ ID NO: 1110 - DNA, 20 bp, artificial
  human FXR antisense
ctggtcttcatggtccaaag

SEQ ID NO: 1111 - DNA, 20 bp, artificial
  human FXR antisense
cagacggaagtttcttattg

SEQ ID NO: 1112 - DNA, 20 bp, artificial
  human FXR antisense
tttatgtattgtctatctgg

SEQ ID NO: 1113 - DNA, 20 bp, artificial
  human FXR antisense
gatttcacagagaagtgggg

SEQ ID NO: 1114 - DNA, 20 bp, artificial
  human FXR antisense
agatttcacagagaagtggg

SEQ ID NO: 1115 - DNA, 20 bp, artificial
  human FXR antisense
cctggagccttttaaaacac

SEQ ID NO: 1116 - DNA, 20 bp, artificial
  human FXR antisense
aggcaaacagggcttgccaa

SEQ ID NO: 1117 - DNA, 20 bp, artificial
  human FXR antisense
ttttccctagttcaacagat

SEQ ID NO: 1118 - DNA, 20 bp, artificial
  human FXR antisense
tatatgcaatatggtaagat

SEQ ID NO: 1119 - DNA, 20 bp, artificial
  human FXR antisense
atatatgcaatatggtaaga

SEQ ID NO: 1120 - DNA, 20 bp, artificial
  human FXR antisense
ctctttaataaaatatatgc

SEQ ID NO: 1121 - DNA, 20 bp, artificial
  human FXR antisense
cttgttctgttaaaacacca

SEQ ID NO: 1122 - DNA, 20 bp, artificial
  human FXR antisense
acttgttctgttaaaacacc

SEQ ID NO: 1123 - DNA, 20 bp, artificial
  human FXR antisense
gccacttgttctgttaaaac

SEQ ID NO: 1124 - DNA, 20 bp, artificial
  human FXR antisense
tggttccacttccaggttct

SEQ ID NO: 1125 - DNA, 20 bp, artificial
  human FXR antisense
gagttcatatattccaggag

SEQ ID NO: 1126 - DNA, 20 bp, artificial
  human FXR antisense
ttatagtggtatccagaggc

SEQ ID NO: 1127 - DNA, 20 bp, artificial
  human FXR antisense
taacaagcattcagccaaca

SEQ ID NO: 1128 - DNA, 20 bp, artificial
  human FXR antisense
cgaggtcacttgtcgcaagt

SEQ ID NO: 1129 - DNA, 20 bp, artificial
  human FXR antisense
tagaagagtctgttgatctg

SEQ ID NO: 1130 - DNA, 20 bp, artificial
  human FXR antisense
aatccataataaaatgtaga

SEQ ID NO: 1131 - DNA, 20 bp, artificial
  human FXR antisense
gaaactgggtacaagtgaaa

SEQ ID NO: 1132 - DNA, 20 bp, artificial
  human FXR antisense
actctttaataaaatatatg

SEQ ID NO: 1133 - DNA, 20 bp, artificial
  human FXR antisense
aaatgctcagaatccaattt

SEQ ID NO: 1134 - DNA, 20 bp, artificial
  human FXR antisense
ctacctccttggattgtttt

SEQ ID NO: 1135 - DNA, 20 bp, artificial
  human FXR antisense
actccttctacgatgtcttc

SEQ ID NO: 1136 - DNA, 20 bp, artificial
  human FXR antisense
atttttcaattgaaatgcac

SEQ ID NO: 1137 - DNA, 20 bp, artificial
  human FXR antisense
ctgtattgcgagtatggttc

SEQ ID NO: 1138 - DNA, 20 bp, artificial
  human FXR antisense
ggtaatgcttctcctgaaga

SEQ ID NO: 1139 - DNA, 20 bp, artificial
  human FXR antisense
ctgtcttcattcacggtctg

SEQ ID NO: 1140 - DNA, 20 bp, artificial
  human FXR antisense
cactgtcttcattcacggtc

SEQ ID NO: 1141 - DNA, 20 bp, artificial
  human FXR antisense
gtcacgaccttcactgtctt

SEQ ID NO: 1142 - DNA, 20 bp, artificial
  human FXR antisense
aagtcacgaccttcactgtc

SEQ ID NO: 1143 - DNA, 20 bp, artificial
  human FXR antisense
gatctggggtgagttcagtt

SEQ ID NO: 1144 - DNA, 20 bp, artificial
  human FXR antisense
atgtagaagagtctgttgat

SEQ ID NO: 1145 - DNA, 20 bp, artificial
  human FXR antisense
ttttttgtgaattctacaag

SEQ ID NO: 1146 - DNA, 20 bp, artificial
  human FXR antisense
ctcctcttgagtcattttca

SEQ ID NO: 1147 - DNA, 20 bp, artificial
  human FXR antisense
aagtgttgaggattttcagg

SEQ ID NO: 1148 - DNA, 20 bp, artificial
  human FXR antisense
gacaggcaaagtgttgagga

SEQ ID NO: 1149 - DNA, 20 bp, artificial
  human FXR antisense
aaaggagctagacccctccc

SEQ ID NO: 1150 - DNA, 20 bp, artificial
  human FXR antisense
catcagattaatatgagaga

SEQ ID NO: 1151 - DNA, 20 bp, artificial
  human FXR antisense
aagtgaaataaaggaaagtt

SEQ ID NO: 1152 - DNA, 20 bp, artificial
  human FXR antisense
ttacacatgtaattacaaca

SEQ ID NO: 1153 - DNA, 20 bp, artificial
  human FXR antisense
aggccctgggaggattctgg

SEQ ID NO: 1154 - DNA, 20 bp, artificial
  human FXR antisense
caattgaaatgcactttctt

SEQ ID NO: 1155 - DNA, 20 bp, artificial
  human FXR antisense
gtaggtaaatgggaatgttc

SEQ ID NO: 1156 - DNA, 20 bp, artificial
  human FXR antisense
ggtagagagtctcagctggc

SEQ ID NO: 1157 - DNA, 20 bp, artificial
  human FXR antisense
ttttacacttgtacacagcg

SEQ ID NO: 1158 - DNA, 20 bp, artificial
  human FXR antisense
tcgcaagtcacgaccttcac

SEQ ID NO: 1159 - DNA, 20 bp, artificial
  human FXR antisense
caaagtctgaaatcctggta

SEQ ID NO: 1160 - DNA, 20 bp, artificial
  human FXR antisense
gcaatctggtcttcatggtc

SEQ ID NO: 1161 - DNA, 20 bp, artificial
  human FXR antisense
agagcatactcctcttgagt

SEQ ID NO: 1162 - DNA, 20 bp, artificial
  human FXR antisense
acatcaagaagtggctcctg

SEQ ID NO: 1163 - DNA, 20 bp, artificial
  human FXR antisense
ggctggtgaatcttacacaa

SEQ ID NO: 1164 - DNA, 20 bp, artificial
  human FXR antisense
gcgacccaggagacaggcaa

SEQ ID NO: 1165 - DNA, 20 bp, artificial
  human FXR antisense
cgtcccagatttcacagaga

SEQ ID NO: 1166 - DNA, 20 bp, artificial
  human FXR antisense
aaaaaggagctagacccctc

SEQ ID NO: 1167 - DNA, 20 bp, artificial
  human FXR antisense
aaggaaagttatacatcaga

SEQ ID NO: 1168 - DNA, 20 bp, artificial
  human FXR antisense
aatacaactctttaataaaa

SEQ ID NO: 1169 - DNA, 20 bp, artificial
  human FXR antisense
ttattgccaagattgaatac

SEQ ID NO: 1170 - DNA, 20 bp, artificial
  human FXR antisense
acaaatgctcagaatccaat

SEQ ID NO: 1171 - DNA, 20 bp, artificial
  human FXR antisense
tccagatcccagcgattttg

SEQ ID NO: 1172 - DNA, 20 bp, artificial
  human FXR antisense
tccttctacgatgtcttcta

SEQ ID NO: 1173 - DNA, 20 bp, artificial
  human FXR antisense
cactccttctacgatgtctt

SEQ ID NO: 1174 - DNA, 20 bp, artificial
  human FXR antisense
gtctcagctggcatacgcct

SEQ ID NO: 1175 - DNA, 20 bp, artificial
  human FXR antisense
cctttacacccctcacaggt

SEQ ID NO: 1176 - DNA, 20 bp, artificial
  human FXR antisense
gaggtcacttgtcgcaagtc

SEQ ID NO: 1177 - DNA, 20 bp, artificial
  human FXR antisense
ttctacaagaacctgtacat

SEQ ID NO: 1178 - DNA, 20 bp, artificial
  human FXR antisense
gtttcttattgaaaatctca

SEQ ID NO: 1179 - DNA, 20 bp, artificial
  human FXR antisense
gaattctttcttccaatagg

SEQ ID NO: 1180 - DNA, 20 bp, artificial
  human FXR antisense
actaaacataggtgttatat

SEQ ID NO: 1181 - DNA, 20 bp, artificial
  human FXR antisense
tcttgagtcattttcagttc

SEQ ID NO: 1182 - DNA, 20 bp, artificial
  human FXR antisense
tctactgcctctctatcctt

SEQ ID NO: 1183 - DNA, 20 bp, artificial
  human FXR antisense
gcacatcaagaagtggctcc

SEQ ID NO: 1184 - DNA, 20 bp, artificial
  human FXR antisense
tgacatcagcatctcagcgt

SEQ ID NO: 1185 - DNA, 20 bp, artificial
  human FXR antisense
tgccaagattgaatacaact

SEQ ID NO: 1186 - DNA, 20 bp, artificial
  human FXR antisense
tgctttattgccaagattga

SEQ ID NO: 1187 - DNA, 20 bp, artificial
  human FXR antisense
aagtcggggagacaatgagg

SEQ ID NO: 1188 - DNA, 20 bp, artificial
  human FXR antisense
gaggattctggactgagtct

SEQ ID NO: 1189 - DNA, 20 bp, artificial
  human FXR antisense
ccttggattgttttgggtca

SEQ ID NO: 1190 - DNA, 20 bp, artificial
  human FXR antisense
tttcatcttgaggaaatgtc

SEQ ID NO: 1191 - DNA, 20 bp, artificial
  human FXR antisense
gagtctcagctggcatacgc

SEQ ID NO: 1192 - DNA, 20 bp, artificial
  human FXR antisense
acctcagtttctccctggta

SEQ ID NO: 1193 - DNA, 20 bp, artificial
  human FXR antisense
gtatccagaggctctgtctc

SEQ ID NO: 1194 - DNA, 20 bp, artificial
  human FXR antisense
gttaacaagcattcagccaa

SEQ ID NO: 1195 - DNA, 20 bp, artificial
  human FXR antisense
tcgaggtcacttgtcgcaag

SEQ ID NO: 1196 - DNA, 20 bp, artificial
  human FXR antisense
attttcaggctggtgaatct

SEQ ID NO: 1197 - DNA, 20 bp, artificial
  human FXR antisense
ggtcgtttactctccatgac

SEQ ID NO: 1198 - DNA, 20 bp, artificial
  human FXR antisense
ccaatatttacagttgtgga

SEQ ID NO: 1199 - DNA, 20 bp, artificial
  human FXR antisense
cagatagaattgaagtaaca

SEQ ID NO: 1200 - DNA, 20 bp, artificial
  human FXR antisense
gaatacaactctttaataaa

SEQ ID NO: 1201 - DNA, 20 bp, artificial
  human FXR antisense
cgatgtcttctacctccttg

SEQ ID NO: 1202 - DNA, 20 bp, artificial
  human FXR antisense
aagtgtctgaagtttcatct

SEQ ID NO: 1203 - DNA, 20 bp, artificial
  human FXR antisense
actccaaagtgtctgaagtt

SEQ ID NO: 1204 - DNA, 20 bp, artificial
  human FXR antisense
ttgttctgttaaaacaccaa

SEQ ID NO: 1205 - DNA, 20 bp, artificial
  human FXR antisense
tatggttccacttccaggtt

SEQ ID NO: 1206 - DNA, 20 bp, artificial
  human FXR antisense
ctctgctacctcagtttctc

SEQ ID NO: 1207 - DNA, 20 bp, artificial
  human FXR antisense
cacttgtacacagcgttttt

SEQ ID NO: 1208 - DNA, 20 bp, artificial
  human FXR antisense
cactttcttcgcatgtacat

SEQ ID NO: 1209 - DNA, 20 bp, artificial
  human FXR antisense
cgcaagtcacgaccttcact

SEQ ID NO: 1210 - DNA, 20 bp, artificial
  human FXR antisense
agcaatctggtcttcatggt

SEQ ID NO: 1211 - DNA, 20 bp, artificial
  human FXR antisense
atactcctcttgagtcattt

SEQ ID NO: 1212 - DNA, 20 bp, artificial
  human FXR antisense
aagcagagcatactcctctt

SEQ ID NO: 1213 - DNA, 20 bp, artificial
  human FXR antisense
tatgtattgtctatctggag

SEQ ID NO: 1214 - DNA, 20 bp, artificial
  human FXR antisense
aatccccatcactgcacgtc

SEQ ID NO: 1215 - DNA, 20 bp, artificial
  human FXR antisense
acaagtgaaataaaggaaag

SEQ ID NO: 1216 - DNA, 20 bp, artificial
  human FXR antisense
tagaattgaagtaacaatca

SEQ ID NO: 1217 - DNA, 20 bp, artificial
  human FXR antisense
gtcggggagacaatgaggtg

SEQ ID NO: 1218 - DNA, 20 bp, artificial
  human FXR antisense
ttggattgttttgggtcaga

SEQ ID NO: 1219 - DNA, 20 bp, artificial
  human FXR antisense
tgaaatgcactttctttatg

SEQ ID NO: 1220 - DNA, 20 bp, artificial
  human FXR antisense
tgatcccatccaaatttttc

SEQ ID NO: 1221 - DNA, 20 bp, artificial
  human FXR antisense
gttccacttccaggttctgt

SEQ ID NO: 1222 - DNA, 20 bp, artificial
  human FXR antisense
ggcatctctgctacctcagt

SEQ ID NO: 1223 - DNA, 20 bp, artificial
  human FXR antisense
gtcgcaagtcacgaccttca

SEQ ID NO: 1224 - DNA, 20 bp, artificial
  human FXR antisense
ctgaacgaaggaacatagct

SEQ ID NO: 1225 - DNA, 20 bp, artificial
  human FXR antisense
cagctgaacgaaggaacata

SEQ ID NO: 1226 - DNA, 20 bp, artificial
  human FXR antisense
ctatttcgaattctttcttc

SEQ ID NO: 1227 - DNA, 20 bp, artificial
  human FXR antisense
cagagcatactcctcttgag

SEQ ID NO: 1228 - DNA, 20 bp, artificial
  human FXR antisense
ctttatgtattgtctatctg

SEQ ID NO: 1229 - DNA, 20 bp, artificial
  human FXR antisense
gaatgtccgtaattcagtca

SEQ ID NO: 1230 - DNA, 20 bp, artificial
  human FXR antisense
tggtcgtttactctccatga

SEQ ID NO: 1231 - DNA, 20 bp, artificial
  human FXR antisense
ttgagtgaaactgggtacaa

SEQ ID NO: 1232 - DNA, 20 bp, artificial
  human FXR antisense
aagattgaatacaactcttt

SEQ ID NO: 1233 - DNA, 20 bp, artificial
  human FXR antisense
gatcccagcgattttgctac

SEQ ID NO: 1234 - DNA, 20 bp, artificial
  human FXR antisense
gactttcaaggccctgggag

SEQ ID NO: 1235 - DNA, 20 bp, artificial
  human FXR antisense
tttgggtcagagatggactt

SEQ ID NO: 1236 - DNA, 20 bp, artificial
  human FXR antisense
tcttctacctccttggattg

SEQ ID NO: 1237 - DNA, 20 bp, artificial
  human FXR antisense
tttgatcccatccaaatttt

SEQ ID NO: 1238 - DNA, 20 bp, artificial
  human FXR antisense
tttttgatcccatccaaatt

SEQ ID NO: 1239 - DNA, 20 bp, artificial
  human FXR antisense
gcgagtatggttccacttcc

SEQ ID NO: 1240 - DNA, 20 bp, artificial
  human FXR antisense
aaactgaacattgctgtatt

SEQ ID NO: 1241 - DNA, 20 bp, artificial
  human FXR antisense
ggctgctgggggtagaaacc

SEQ ID NO: 1242 - DNA, 20 bp, artificial
  human FXR antisense
tggtagagagtctcagctgg

SEQ ID NO: 1243 - DNA, 20 bp, artificial
  human FXR antisense
acctttacacccctcacagg

SEQ ID NO: 1244 - DNA, 20 bp, artificial
  human FXR antisense
gcttctcctgaagaaacctt

SEQ ID NO: 1245 - DNA, 20 bp, artificial
  human FXR antisense
cagttaacaagcattcagcc

SEQ ID NO: 1246 - DNA, 20 bp, artificial
  human FXR antisense
tactcctcttgagtcatttt

SEQ ID NO: 1247 - DNA, 20 bp, artificial
  human FXR antisense
gacaggataacaattgctgt

SEQ ID NO: 1248 - DNA, 20 bp, artificial
  human FXR antisense
cctttatgtattgtctatct

SEQ ID NO: 1249 - DNA, 20 bp, artificial
  human FXR antisense
catcaagaagtggctcctga

SEQ ID NO: 1250 - DNA, 20 bp, artificial
  human FXR antisense
caaacagggcttgccaatta

SEQ ID NO: 1251 - DNA, 20 bp, artificial
  human FXR antisense
tttaattaggcaaacagggc

SEQ ID NO: 1252 - DNA, 20 bp, artificial
  human FXR antisense
atcaatttaattaggcaaac

SEQ ID NO: 1253 - DNA, 20 bp, artificial
  human FXR antisense
aactctttaataaaatatat

SEQ ID NO: 1254 - DNA, 20 bp, artificial
  human FXR antisense
tttattgccaagattgaata

SEQ ID NO: 1255 - DNA, 20 bp, artificial
  human FXR antisense
agtcttcctctccagatccc

SEQ ID NO: 1256 - DNA, 20 bp, artificial
  human FXR antisense
ccacttgttctgttaaaaca

SEQ ID NO: 1257 - DNA, 20 bp, artificial
  human FXR antisense
ttgtacacagcgtttttggt

SEQ ID NO: 1258 - DNA, 20 bp, artificial
  human FXR antisense
tttcagttaacaagcattca

SEQ ID NO: 1259 - DNA, 20 bp, artificial
  human FXR antisense
ttttatttgttatttcctga

SEQ ID NO: 1260 - DNA, 20 bp, artificial
  human FXR antisense
tacaagtgaaataaaggaaa

SEQ ID NO: 1261 - DNA, 20 bp, artificial
  human FXR antisense
tttacagttgtggaagttac

SEQ ID NO: 1262 - DNA, 20 bp, artificial
  human FXR antisense
tctatctagcccaatattta

SEQ ID NO: 1263 - DNA, 20 bp, artificial
  human FXR antisense
aggctgctgggggtagaaac

SEQ ID NO: 1264 - DNA, 20 bp, artificial
  human FXR antisense
atagcttcaaccgcagaccc

SEQ ID NO: 1265 - DNA, 20 bp, artificial
  human FXR antisense
ccatcactgcacgtcccaga

SEQ ID NO: 1266 - DNA, 20 bp, artificial
  human FXR antisense
acagatagaattgaagtaac

SEQ ID NO: 1267 - DNA, 20 bp, artificial
  human FXR antisense
atatggtaagatgagcaaaa

SEQ ID NO: 1268 - DNA, 20 bp, artificial
  human FXR antisense
tacaactctttaataaaata

SEQ ID NO: 1269 - DNA, 20 bp, artificial
  human FXR antisense
tctgaagtttcatcttgagg

SEQ ID NO: 1270 - DNA, 20 bp, artificial
  human FXR antisense
ttccacttccaggttctgtc

SEQ ID NO: 1271 - DNA, 20 bp, artificial
  human FXR antisense
atctctgctacctcagtttc

SEQ ID NO: 1272 - DNA, 20 bp, artificial
  human FXR antisense
caggcatctctgctacctca

SEQ ID NO: 1273 - DNA, 20 bp, artificial
  human FXR antisense
ctgtctccacaaacaacaca

SEQ ID NO: 1274 - DNA, 20 bp, artificial
  human FXR antisense
atttcagttaacaagcattc

SEQ ID NO: 1275 - DNA, 20 bp, artificial
  human FXR antisense
attttttctcagtcgcttag

SEQ ID NO: 1276 - DNA, 20 bp, artificial
  human FXR antisense
tctgttgatctggggtgagt

SEQ ID NO: 1277 - DNA, 20 bp, artificial
  human FXR antisense
gtctgttgatctggggtgag

SEQ ID NO: 1278 - DNA, 20 bp, artificial
  human FXR antisense
ccactatttcgaattctttc

SEQ ID NO: 1279 - DNA, 20 bp, artificial
  human FXR antisense
agacaggataacaattgctg

SEQ ID NO: 1280 - DNA, 20 bp, artificial
  human FXR antisense
caaaatgagattttccctag

SEQ ID NO: 1281 - DNA, 20 bp, artificial
  human FXR antisense
atgagcaaaatgagattttc

SEQ ID NO: 1282 - DNA, 20 bp, artificial
  human FXR antisense
tatgcaatatggtaagatga

SEQ ID NO: 1283 - DNA, 20 bp, artificial
  human FXR antisense
atgtcttctacctccttgga

SEQ ID NO: 1284 - DNA, 20 bp, artificial
  human FXR antisense
ttgatcccatccaaattttt

SEQ ID NO: 1285 - DNA, 20 bp, artificial
  human FXR antisense
ttttgatcccatccaaattt

SEQ ID NO: 1286 - DNA, 20 bp, artificial
  human FXR antisense
atggttccacttccaggttc

SEQ ID NO: 1287 - DNA, 20 bp, artificial
  human FXR antisense
gaacattgctgtattgcgag

SEQ ID NO: 1288 - DNA, 20 bp, artificial
  human FXR antisense
ggaaatctgtggttgaactt

SEQ ID NO: 1289 - DNA, 20 bp, artificial
  human FXR antisense
ccctggtagagagtctcagc

SEQ ID NO: 1290 - DNA, 20 bp, artificial
  human FXR antisense
actttcttcgcatgtacata

SEQ ID NO: 1291 - DNA, 20 bp, artificial
  human FXR antisense
caagcattcagccaacattc

SEQ ID NO: 1292 - DNA, 20 bp, artificial
  human FXR antisense
ttatatgaatccataataaa

SEQ ID NO: 1293 - DNA, 20 bp, artificial
  human FXR antisense
agcttcaaccgcagaccctt

SEQ ID NO: 1294 - DNA, 20 bp, artificial
  human FXR antisense
gttatatattcatcagagat

SEQ ID NO: 1295 - DNA, 20 bp, artificial
  human FXR antisense
gcacgtcccagatttcacag

SEQ ID NO: 1296 - DNA, 20 bp, artificial
  human FXR antisense
aatgcaggattccctggagc

SEQ ID NO: 1297 - DNA, 20 bp, artificial
  human FXR antisense
caactctttaataaaatata

SEQ ID NO: 1298 - DNA, 20 bp, artificial
  human FXR antisense
gtgtctgaagtttcatcttg

SEQ ID NO: 1299 - DNA, 20 bp, artificial
  human FXR antisense
atcccatccaaatttttcaa

SEQ ID NO: 1300 - DNA, 20 bp, artificial
  human FXR antisense
tgagattcatttttgatccc

SEQ ID NO: 1301 - DNA, 20 bp, artificial
  human FXR antisense
ggttccacttccaggttctg

SEQ ID NO: 1302 - DNA, 20 bp, artificial
  human FXR antisense
tcctgaagaaacctttacac

SEQ ID NO: 1303 - DNA, 20 bp, artificial
  human FXR antisense
gaattctacaagaacctgta

SEQ ID NO: 1304 - DNA, 20 bp, artificial
  human FXR antisense
aactaaacataggtgttata

SEQ ID NO: 1305 - DNA, 20 bp, artificial
  human FXR antisense
gaagtggctcctgaagcttc

SEQ ID NO: 1306 - DNA, 20 bp, artificial
  human FXR antisense
cagatttcacagagaagtgg

SEQ ID NO: 1307 - DNA, 20 bp, artificial
  human FXR antisense
tgcacgtcccagatttcaca

SEQ ID NO: 1308 - DNA, 20 bp, artificial
  human FXR antisense
atccccatcactgcacgtcc

SEQ ID NO: 1309 - DNA, 20 bp, artificial
  human FXR antisense
tattcatcaagatttcttga

SEQ ID NO: 1310 - DNA, 20 bp, artificial
  human FXR antisense
gctttattgccaagattgaa

SEQ ID NO: 1311 - DNA, 20 bp, artificial
  human FXR antisense
aactaagagaagcagtgttc

SEQ ID NO: 1312 - DNA, 20 bp, artificial
  human FXR antisense
ttatggtggtcttcaaaaaa

SEQ ID NO: 1313 - DNA, 20 bp, artificial
  human FXR antisense
acacagctcatcccctttga

SEQ ID NO: 1314 - DNA, 20 bp, artificial
  human FXR antisense
acacttgtacacagcgtttt

SEQ ID NO: 1315 - DNA, 20 bp, artificial
  human FXR antisense
ctgttgatctggggtgagtt

SEQ ID NO: 1316 - DNA, 20 bp, artificial
  human FXR antisense
aatgtagaagagtctgttga

SEQ ID NO: 1317 - DNA, 20 bp, artificial
  human FXR antisense
ttttcaggctggtgaatctt

SEQ ID NO: 1318 - DNA, 20 bp, artificial
  human FXR antisense
gagaagtggggtaaacttgt

SEQ ID NO: 1319 - DNA, 20 bp, artificial
  human FXR antisense
ttttgggtcagagatggact

SEQ ID NO: 1320 - DNA, 20 bp, artificial
  human FXR antisense
tttgagctatgtttctaagt

SEQ ID NO: 1321 - DNA, 20 bp, artificial
  human FXR antisense
cacttgttctgttaaaacac

SEQ ID NO: 1322 - DNA, 20 bp, artificial
  human FXR antisense
ttccaggagagtaccactct

SEQ ID NO: 1323 - DNA, 20 bp, artificial
  human FXR antisense
gacactttcttcgcatgtac

SEQ ID NO: 1324 - DNA, 20 bp, artificial
  human FXR antisense
tcgcttagatttacactgaa

SEQ ID NO: 1325 - DNA, 20 bp, artificial
  human FXR antisense
ttgatctggggtgagttcag

SEQ ID NO: 1326 - DNA, 20 bp, artificial
  human FXR antisense
ataataaaatgtagaagagt

SEQ ID NO: 1327 - DNA, 20 bp, artificial
  human FXR antisense
agacggaagtttcttattga

SEQ ID NO: 1328 - DNA, 20 bp, artificial
  human FXR antisense
caggctggtgaatcttacac

SEQ ID NO: 1329 - DNA, 20 bp, artificial
  human FXR antisense
tcaggctggtgaatcttaca

SEQ ID NO: 1330 - DNA, 20 bp, artificial
  human FXR antisense
gcaaacagggcttgccaatt

SEQ ID NO: 1331 - DNA, 20 bp, artificial
  human FXR antisense
tcaatttaattaggcaaaca

SEQ ID NO: 1332 - DNA, 20 bp, artificial
  human FXR antisense
aatgctcagaatccaatttc

SEQ ID NO: 1333 - DNA, 20 bp, artificial
  human FXR antisense
tttctaagtcttcttttctt

SEQ ID NO: 1334 - DNA, 20 bp, artificial
  human FXR antisense
atccaggaaactaagagaag

SEQ ID NO: 1335 - DNA, 20 bp, artificial
  human FXR antisense
gaaaattcatctgtggtagg

SEQ ID NO: 1336 - DNA, 20 bp, artificial
  human FXR antisense
ttcatatattccaggagagt

SEQ ID NO: 1337 - DNA, 20 bp, artificial
  human FXR antisense
gttcatatattccaggagag

SEQ ID NO: 1338 - DNA, 20 bp, artificial
  human FXR antisense
ccgtttttacacttgtacac

SEQ ID NO: 1339 - DNA, 20 bp, artificial
  human FXR antisense
tagatttacactgaatttca

SEQ ID NO: 1340 - DNA, 20 bp, artificial
  human FXR antisense
tgtcgcaagtcacgaccttc

SEQ ID NO: 1341 - DNA, 20 bp, artificial
  human FXR antisense
ttgtcgcaagtcacgacctt

SEQ ID NO: 1342 - DNA, 20 bp, artificial
  human FXR antisense
atccataataaaatgtagaa

SEQ ID NO: 1343 - DNA, 20 bp, artificial
  human FXR antisense
attctacaagaacctgtaca

SEQ ID NO: 1344 - DNA, 20 bp, artificial
  human FXR antisense
aggaacatagcttcaaccgc

SEQ ID NO: 1345 - DNA, 20 bp, artificial
  human FXR antisense
actatttcgaattctttctt

SEQ ID NO: 1346 - DNA, 20 bp, artificial
  human FXR antisense
actcctcttgagtcattttc

SEQ ID NO: 1347 - DNA, 20 bp, artificial
  human FXR antisense
taagcagagcatactcctct

SEQ ID NO: 1348 - DNA, 20 bp, artificial
  human FXR antisense
aagaagtggctcctgaagct

SEQ ID NO: 1349 - DNA, 20 bp, artificial
  human FXR antisense
ttaattaggcaaacagggct

SEQ ID NO: 1350 - DNA, 20 bp, artificial
  human FXR antisense
gcaatatggtaagatgagca

SEQ ID NO: 1351 - DNA, 20 bp, artificial
  human FXR antisense
ctttattgccaagattgaat

SEQ ID NO: 1352 - DNA, 20 bp, artificial
  human FXR antisense
cctgggaggattctggactg

SEQ ID NO: 1353 - DNA, 20 bp, artificial
  human FXR antisense
ctttcactccttctacgatg

SEQ ID NO: 1354 - DNA, 20 bp, artificial
  human FXR antisense
gctgctgggggtagaaaccc

SEQ ID NO: 1355 - DNA, 20 bp, artificial
  human FXR antisense
ggagagtaccactcttcagg

SEQ ID NO: 1356 - DNA, 20 bp, artificial
  human FXR antisense
tccaggagagtaccactctt

SEQ ID NO: 1357 - DNA, 20 bp, artificial
  human FXR antisense
aggcatctctgctacctcag

SEQ ID NO: 1358 - DNA, 20 bp, artificial
  human FXR antisense
acatatccatcacacagttg

SEQ ID NO: 1359 - DNA, 20 bp, artificial
  human FXR antisense
ttcttcgcatgtacatatcc

SEQ ID NO: 1360 - DNA, 20 bp, artificial
  human FXR antisense
tttatttgttatttcctgag

SEQ ID NO: 1361 - DNA, 20 bp, artificial
  human FXR antisense
tcttttaaaattttatttgt

SEQ ID NO: 1362 - DNA, 20 bp, artificial
  human FXR antisense
aaagtctgaaatcctggtag

SEQ ID NO: 1363 - DNA, 20 bp, artificial
  human FXR antisense
gacccaggagacaggcaaag

SEQ ID NO: 1364 - DNA, 20 bp, artificial
  human FXR antisense
ggaaagttatacatcagatt

SEQ ID NO: 1365 - DNA, 20 bp, artificial
  human FXR antisense
atattcatcaagatttcttg

SEQ ID NO: 1366 - DNA, 20 bp, artificial
  human FXR antisense
tttccctagttcaacagata

SEQ ID NO: 1367 - DNA, 20 bp, artificial
  human FXR antisense
tgaatacaactctttaataa

SEQ ID NO: 1368 - DNA, 20 bp, artificial
  human FXR antisense
gtcttcctctccagatccca

SEQ ID NO: 1369 - DNA, 20 bp, artificial
  human FXR antisense
ggaaactaagagaagcagtg

SEQ ID NO: 1370 - DNA, 20 bp, artificial
  human FXR antisense
gtggtcttcaaaaaaaactc

SEQ ID NO: 1371 - DNA, 20 bp, artificial
  human FXR antisense
tcaattgaaatgcactttct

SEQ ID NO: 1372 - DNA, 20 bp, artificial
  human FXR antisense
aggttctgtcccagaggacc

SEQ ID NO: 1373 - DNA, 20 bp, artificial
  human FXR antisense
agttcatatattccaggaga

SEQ ID NO: 1374 - DNA, 20 bp, artificial
  human FXR antisense
gtagagagtctcagctggca

SEQ ID NO: 1375 - DNA, 20 bp, artificial
  human FXR antisense
tatttcgaattctttcttcc

SEQ ID NO: 1376 - DNA, 20 bp, artificial
  human FXR antisense
cactatttcgaattctttct

SEQ ID NO: 1377 - DNA, 20 bp, artificial
  human FXR antisense
gcagagcatactcctcttga

SEQ ID NO: 1378 - DNA, 20 bp, artificial
  human FXR antisense
catgacatcagcatctcagc

SEQ ID NO: 1379 - DNA, 20 bp, artificial
  human FXR antisense
tacacatgtaattacaacat

SEQ ID NO: 1380 - DNA, 20 bp, artificial
  human FXR antisense
tagagaaagttgttctatct

SEQ ID NO: 1381 - DNA, 20 bp, artificial
  human FXR antisense
aatatggtaagatgagcaaa

SEQ ID NO: 1382 - DNA, 20 bp, artificial
  human FXR antisense
atatgcaatatggtaagatg

SEQ ID NO: 1383 - DNA, 20 bp, artificial
  human FXR antisense
tttaataaaatatatgcaat

SEQ ID NO: 1384 - DNA, 20 bp, artificial
  human FXR antisense
ttgctttattgccaagattg

SEQ ID NO: 1385 - DNA, 20 bp, artificial
  human FXR antisense
tcggggagacaatgaggtga

SEQ ID NO: 1386 - DNA, 20 bp, artificial
  human FXR antisense
ttgggtcagagatggacttt

SEQ ID NO: 1387 - DNA, 20 bp, artificial
  human FXR antisense
tgaagtttcatcttgaggaa

SEQ ID NO: 1388 - DNA, 20 bp, artificial
  human FXR antisense
atttttgatcccatccaaat

SEQ ID NO: 1389 - DNA, 20 bp, artificial
  human FXR antisense
taggtaaatgggaatgttca

SEQ ID NO: 1390 - DNA, 20 bp, artificial
  human FXR antisense
cgcttagatttacactgaat

SEQ ID NO: 1391 - DNA, 20 bp, artificial
  human FXR antisense
agaagagtctgttgatctgg

SEQ ID NO: 1392 - DNA, 20 bp, artificial
  human FXR antisense
accactatttcgaattcttt

SEQ ID NO: 1393 - DNA, 20 bp, artificial
  human FXR antisense
tctaagtcttcttttcttct

SEQ ID NO: 1394 - DNA, 20 bp, artificial
  human FXR antisense
tccaaagtgtctgaagtttc

SEQ ID NO: 1395 - DNA, 20 bp, artificial
  human FXR antisense
attgcgagtatggttccact

SEQ ID NO: 1396 - DNA, 20 bp, artificial
  human FXR antisense
tctggggtgagttcagtttt

SEQ ID NO: 1397 - DNA, 20 bp, artificial
  human FXR antisense
gctggtgaatcttacacaac

SEQ ID NO: 1398 - DNA, 20 bp, artificial
  human FXR antisense
aaaaggagctagacccctcc

SEQ ID NO: 1399 - DNA, 20 bp, artificial
  human FXR antisense
tgggtacaagtgaaataaag

SEQ ID NO: 1400 - DNA, 20 bp, artificial
  human FXR antisense
taacaatcaatttaattagg

SEQ ID NO: 1401 - DNA, 20 bp, artificial
  human FXR antisense
tctccagatcccagcgattt

SEQ ID NO: 1402 - DNA, 20 bp, artificial
  human FXR antisense
tcatcttgaggaaatgtcca

SEQ ID NO: 1403 - DNA, 20 bp, artificial
  human FXR antisense
aggaaatctgtggttgaact

SEQ ID NO: 1404 - DNA, 20 bp, artificial
  human FXR antisense
tctgcactgaattcttcttt

SEQ ID NO: 1405 - DNA, 20 bp, artificial
  human FXR antisense
ttctgcactgaattcttctt

SEQ ID NO: 1406 - DNA, 20 bp, artificial
  human FXR antisense
tgaacgaaggaacatagctt

SEQ ID NO: 1407 - DNA, 20 bp, artificial
  human FXR antisense
cttgagtcattttcagttcc

SEQ ID NO: 1408 - DNA, 20 bp, artificial
  human FXR antisense
ctagcccaatatttacagtt

SEQ ID NO: 1409 - DNA, 20 bp, artificial
  human FXR antisense
aaaatatatgcaatatggta

SEQ ID NO: 1410 - DNA, 20 bp, artificial
  human FXR antisense
tctttaataaaatatatgca

SEQ ID NO: 1411 - DNA, 20 bp, artificial
  human FXR antisense
gaaatccaggaaactaagag

SEQ ID NO: 1412 - DNA, 20 bp, artificial
  human FXR antisense
tcccatccaaatttttcaat

SEQ ID NO: 1413 - DNA, 20 bp, artificial
  human FXR antisense
gtggtaggtaaatgggaatg

SEQ ID NO: 1414 - DNA, 20 bp, artificial
  human FXR antisense
gagtatggttccacttccag

SEQ ID NO: 1415 - DNA, 20 bp, artificial
  human FXR antisense
ctttcttcgcatgtacatat

SEQ ID NO: 1416 - DNA, 20 bp, artificial
  human FXR antisense
acactttcttcgcatgtaca

SEQ ID NO: 1417 - DNA, 20 bp, artificial
  human FXR antisense
agtctgttgatctggggtga

SEQ ID NO: 1418 - DNA, 20 bp, artificial
  human FXR antisense
ggaacatagcttcaaccgca

SEQ ID NO: 1419 - DNA, 20 bp, artificial
  human FXR antisense
atgtattgtctatctggaga

SEQ ID NO: 1420 - DNA, 20 bp, artificial
  human FXR antisense
ttctctactgcctctctatc

SEQ ID NO: 1421 - DNA, 20 bp, artificial
  human FXR antisense
ctgcacgtcccagatttcac

SEQ ID NO: 1422 - DNA, 20 bp, artificial
  human FXR antisense
cctagttcaacagatagaat

SEQ ID NO: 1423 - DNA, 20 bp, artificial
  human FXR antisense
ttaataaaatatatgcaata

SEQ ID NO: 1424 - DNA, 20 bp, artificial
  human FXR antisense
ttaggataagtcggggagac

SEQ ID NO: 1425 - DNA, 20 bp, artificial
  human FXR antisense
cttctacctccttggattgt

SEQ ID NO: 1426 - DNA, 20 bp, artificial
  human FXR antisense
tattgcgagtatggttccac

SEQ ID NO: 1427 - DNA, 20 bp, artificial
  human FXR antisense
cttgtcgcaagtcacgacct

SEQ ID NO: 1428 - DNA, 20 bp, artificial
  human FXR antisense
tttttgtgaattctacaaga

SEQ ID NO: 1429 - DNA, 20 bp, artificial
  human FXR antisense
catagcttcaaccgcagacc

SEQ ID NO: 1430 - DNA, 20 bp, artificial
  human FXR antisense
gacggaagtttcttattgaa

SEQ ID NO: 1431 - DNA, 20 bp, artificial
  human FXR antisense
gtcccagatttcacagagaa

SEQ ID NO: 1432 - DNA, 20 bp, artificial
  human FXR antisense
aggaaagttatacatcagat

SEQ ID NO: 1433 - DNA, 20 bp, artificial
  human FXR antisense
aatattcatcaagatttctt

SEQ ID NO: 1434 - DNA, 20 bp, artificial
  human FXR antisense
taaaatatatgcaatatggt

SEQ ID NO: 1435 - DNA, 20 bp, artificial
  human FXR antisense
aattcatctgtggtaggtaa

SEQ ID NO: 1436 - DNA, 20 bp, artificial
  human FXR antisense
ctcacaggtcagtgcattat

SEQ ID NO: 1437 - DNA, 20 bp, artificial
  human FXR antisense
tgtacacagcgtttttggta

SEQ ID NO: 1438 - DNA, 20 bp, artificial
  human FXR antisense
cgcatgtacatatccatcac

SEQ ID NO: 1439 - DNA, 20 bp, artificial
  human FXR antisense
gatttacactgaatttcagt

SEQ ID NO: 1440 - DNA, 20 bp, artificial
  human FXR antisense
ctgcactgaattcttctttt

SEQ ID NO: 1441 - DNA, 20 bp, artificial
  human FXR antisense
tcagagataccactatttcg

SEQ ID NO: 1442 - DNA, 20 bp, artificial
  human FXR antisense
gtcattttcagttccccaat

SEQ ID NO: 1443 - DNA, 20 bp, artificial
  human FXR antisense
agtcattttcagttccccaa

SEQ ID NO: 1444 - DNA, 20 bp, artificial
  human FXR antisense
aacaattgctgtaagcagag

SEQ ID NO: 1445 - DNA, 20 bp, artificial
  human FXR antisense
agatttcttgagtgaaactg

SEQ ID NO: 1446 - DNA, 20 bp, artificial
  human FXR antisense
atgcaggattccctggagcc

SEQ ID NO: 1447 - DNA, 20 bp, artificial
  human FXR antisense
ccctagttcaacagatagaa

SEQ ID NO: 1448 - DNA, 20 bp, artificial
  human FXR antisense
caagattgaatacaactctt

SEQ ID NO: 1449 - DNA, 20 bp, artificial
  human FXR antisense
taagtcggggagacaatgag

SEQ ID NO: 1450 - DNA, 20 bp, artificial
  human FXR antisense
tcttctttcactccttctac

SEQ ID NO: 1451 - DNA, 20 bp, artificial
  human FXR antisense
gggaatgttcaatgagattc

SEQ ID NO: 1452 - DNA, 20 bp, artificial
  human FXR antisense
tccacttccaggttctgtcc

SEQ ID NO: 1453 - DNA, 20 bp, artificial
  human FXR antisense
atcagagataccactatttc

SEQ ID NO: 1454 - DNA, 20 bp, artificial
  human FXR antisense
cgtttactctccatgacatc

SEQ ID NO: 1455 - DNA, 20 bp, artificial
  human FXR antisense
taattaggcaaacagggctt

SEQ ID NO: 1456 - DNA, 20 bp, artificial
  human FXR antisense
agttcaacagatagaattga

SEQ ID NO: 1457 - DNA, 20 bp, artificial
  human FXR antisense
tgcaatatggtaagatgagc

SEQ ID NO: 1458 - DNA, 20 bp, artificial
  human FXR antisense
tgggtcagagatggactttc

SEQ ID NO: 1459 - DNA, 20 bp, artificial
  human FXR antisense
tcttttcttctttcactcct

SEQ ID NO: 1460 - DNA, 20 bp, artificial
  human FXR antisense
taataggatgacgaggaaat

SEQ ID NO: 1461 - DNA, 20 bp, artificial
  human FXR antisense
ataataggatgacgaggaaa

SEQ ID NO: 1462 - DNA, 20 bp, artificial
  human FXR antisense
cctcacaggtcagtgcatta

SEQ ID NO: 1463 - DNA, 20 bp, artificial
  human FXR antisense
ccctcacaggtcagtgcatt

SEQ ID NO: 1464 - DNA, 20 bp, artificial
  human FXR antisense
cttgtacacagcgtttttgg

SEQ ID NO: 1465 - DNA, 20 bp, artificial
  human FXR antisense
tagcttcaaccgcagaccct

SEQ ID NO: 1466 - DNA, 20 bp, artificial
  human FXR antisense
caggcaaagtgttgaggatt

SEQ ID NO: 1467 - DNA, 20 bp, artificial
  human FXR antisense
agacaggcaaagtgttgagg

SEQ ID NO: 1468 - DNA, 20 bp, artificial
  human FXR antisense
gtggtcgtttactctccatg

SEQ ID NO: 1469 - DNA, 20 bp, artificial
  human FXR antisense
cactgcacgtcccagatttc

SEQ ID NO: 1470 - DNA, 20 bp, artificial
  human FXR antisense
aatatatgcaatatggtaag

SEQ ID NO: 1471 - DNA, 20 bp, artificial
  human FXR antisense
ttgaatacaactctttaata

SEQ ID NO: 1472 - DNA, 20 bp, artificial
  human FXR antisense
ggaggattctggactgagtc

SEQ ID NO: 1473 - DNA, 20 bp, artificial
  human FXR antisense
gagattcatttttgatccca

SEQ ID NO: 1474 - DNA, 20 bp, artificial
  human FXR antisense
tgttctgttaaaacaccaaa

SEQ ID NO: 1475 - DNA, 20 bp, artificial
  human FXR antisense
caggttctgtcccagaggac

SEQ ID NO: 1476 - DNA, 20 bp, artificial
  human FXR antisense
attatagtggtatccagagg

SEQ ID NO: 1477 - DNA, 20 bp, artificial
  human FXR antisense
ccccgtttttacacttgtac

SEQ ID NO: 1478 - DNA, 20 bp, artificial
  human FXR antisense
tttcttcgcatgtacatatc

SEQ ID NO: 1479 - DNA, 20 bp, artificial
  human FXR antisense
acaagcattcagccaacatt

SEQ ID NO: 1480 - DNA, 20 bp, artificial
  human FXR antisense
aaatgagaaaattttcttct

SEQ ID NO: 1481 - DNA, 20 bp, artificial
  human FXR antisense
gaacgaaggaacatagcttc

SEQ ID NO: 1482 - DNA, 20 bp, artificial
  human FXR antisense
taacaattgctgtaagcaga

SEQ ID NO: 1483 - DNA, 20 bp, artificial
  human FXR antisense
ctcctgaagcttctctactg

SEQ ID NO: 1484 - DNA, 20 bp, artificial
  human FXR antisense
agagaagtggggtaaacttg

SEQ ID NO: 1485 - DNA, 20 bp, artificial
  human FXR antisense
cccctgtaatccccatcact

SEQ ID NO: 1486 - DNA, 20 bp, artificial
  human FXR antisense
atagaattgaagtaacaatc

SEQ ID NO: 1487 - DNA, 20 bp, artificial
  human FXR antisense
ttttcttctttcactccttc

SEQ ID NO: 1488 - DNA, 20 bp, artificial
  human FXR antisense
ttcatctgtggtaggtaaat

SEQ ID NO: 1489 - DNA, 20 bp, artificial
  human FXR antisense
agaaaattcatctgtggtag

SEQ ID NO: 1490 - DNA, 20 bp, artificial
  human FXR antisense
tctgctacctcagtttctcc

SEQ ID NO: 1491 - DNA, 20 bp, artificial
  human FXR antisense
cacgtcccagatttcacaga

SEQ ID NO: 1492 - DNA, 20 bp, artificial
  human FXR antisense
cctcccctgtaatccccatc

SEQ ID NO: 1493 - DNA, 20 bp, artificial
  human FXR antisense
acacatgtaattacaacata

SEQ ID NO: 1494 - DNA, 20 bp, artificial
  human FXR antisense
tccctagttcaacagataga

SEQ ID NO: 1495 - DNA, 20 bp, artificial
  human FXR antisense
tgagcaaaatgagattttcc

SEQ ID NO: 1496 - DNA, 20 bp, artificial
  human FXR antisense
atgcaatatggtaagatgag

SEQ ID NO: 1497 - DNA, 20 bp, artificial
  human FXR antisense
atgtccagaagaaatccagg

SEQ ID NO: 1498 - DNA, 20 bp, artificial
  human FXR antisense
gtttcatcttgaggaaatgt

SEQ ID NO: 1499 - DNA, 20 bp, artificial
  human FXR antisense
attcatctgtggtaggtaaa

SEQ ID NO: 1500 - DNA, 20 bp, artificial
  human FXR antisense
aaattcatctgtggtaggta

SEQ ID NO: 1501 - DNA, 20 bp, artificial
  human FXR antisense
gaaatctgtggttgaacttg

SEQ ID NO: 1502 - DNA, 20 bp, artificial
  human FXR antisense
tcatatattccaggagagta

SEQ ID NO: 1503 - DNA, 20 bp, artificial
  human FXR antisense
caacacacagctcatcccct

SEQ ID NO: 1504 - DNA, 20 bp, artificial
  human FXR antisense
cgtttttacacttgtacaca

SEQ ID NO: 1505 - DNA, 20 bp, artificial
  human FXR antisense
tacatatccatcacacagtt

SEQ ID NO: 1506 - DNA, 20 bp, artificial
  human FXR antisense
agatttacactgaatttcag

SEQ ID NO: 1507 - DNA, 20 bp, artificial
  human FXR antisense
ttccgtcaaaatgagaaaat

SEQ ID NO: 1508 - DNA, 20 bp, artificial
  human FXR antisense
gataacaattgctgtaagca

SEQ ID NO: 1509 - DNA, 20 bp, artificial
  human FXR antisense
cgacccaggagacaggcaaa

SEQ ID NO: 1510 - DNA, 20 bp, artificial
  human FXR antisense
gagcaaaatgagattttccc

SEQ ID NO: 1511 - DNA, 20 bp, artificial
  human FXR antisense
aactccaaagtgtctgaagt

SEQ ID NO: 1512 - DNA, 20 bp, artificial
  human FXR antisense
ggaataataggatgacgagg

SEQ ID NO: 1513 - DNA, 20 bp, artificial
  human FXR antisense
tccctggtagagagtctcag

SEQ ID NO: 1514 - DNA, 20 bp, artificial
  human FXR antisense
ggtatccagaggctctgtct

SEQ ID NO: 1515 - DNA, 20 bp, artificial
  human FXR antisense
cctgaagaaacctttacacc

SEQ ID NO: 1516 - DNA, 20 bp, artificial
  human FXR antisense
agctgaacgaaggaacatag

SEQ ID NO: 1517 - DNA, 20 bp, artificial
  human FXR antisense
aggagacaggcaaagtgttg

SEQ ID NO: 1518 - DNA, 20 bp, artificial
  human FXR antisense
atgacatcagcatctcagcg

SEQ ID NO: 1519 - DNA, 20 bp, artificial
  human FXR antisense
actgcacgtcccagatttca

SEQ ID NO: 1520 - DNA, 20 bp, artificial
  human FXR antisense
tgaaataaaggaaagttata

SEQ ID NO: 1521 - DNA, 20 bp, artificial
  human FXR antisense
aacagatagaattgaagtaa

SEQ ID NO: 1522 - DNA, 20 bp, artificial
  human FXR antisense
agatcccagcgattttgcta

SEQ ID NO: 1523 - DNA, 20 bp, artificial
  human FXR antisense
tatggtggtcttcaaaaaaa

SEQ ID NO: 1524 - DNA, 20 bp, artificial
  human FXR antisense
ttcaattgaaatgcactttc

SEQ ID NO: 1525 - DNA, 20 bp, artificial
  human FXR antisense
tgcttctcctgaagaaacct

SEQ ID NO: 1526 - DNA, 20 bp, artificial
  human FXR antisense
acttgtacacagcgtttttg

SEQ ID NO: 1527 - DNA, 20 bp, artificial
  human FXR antisense
cagagaagtggggtaaactt

SEQ ID NO: 1528 - DNA, 20 bp, artificial
  human FXR antisense
catcaagatttcttgagtga

SEQ ID NO: 1529 - DNA, 20 bp, artificial
  human FXR antisense
tagcccaatatttacagttg

SEQ ID NO: 1530 - DNA, 20 bp, artificial
  human FXR antisense
gggtcagagatggactttca

SEQ ID NO: 1531 - DNA, 20 bp, artificial
  human FXR antisense
gttttgggtcagagatggac

SEQ ID NO: 1532 - DNA, 20 bp, artificial
  human FXR antisense
agaaatccaggaaactaaga

SEQ ID NO: 1533 - DNA, 20 bp, artificial
  human FXR antisense
tgtccagaagaaatccagga

SEQ ID NO: 1534 - DNA, 20 bp, artificial
  human FXR antisense
aaaattcatctgtggtaggt

SEQ ID NO: 1535 - DNA, 20 bp, artificial
  human FXR antisense
tcccagaggacctgccactt

SEQ ID NO: 1536 - DNA, 20 bp, artificial
  human FXR antisense
aacctttacacccctcacag

SEQ ID NO: 1537 - DNA, 20 bp, artificial
  human FXR antisense
atctggggtgagttcagttt

SEQ ID NO: 1538 - DNA, 20 bp, artificial
  human FXR antisense
tatatgaatccataataaaa

SEQ ID NO: 1539 - DNA, 20 bp, artificial
  human FXR antisense
catttccgtcaaaatgagaa

SEQ ID NO: 1540 - DNA, 20 bp, artificial
  human FXR antisense
acatagcttcaaccgcagac

SEQ ID NO: 1541 - DNA, 20 bp, artificial
  human FXR antisense
tctctactgcctctctatcc

SEQ ID NO: 1542 - DNA, 20 bp, artificial
  human FXR antisense
tcccctgtaatccccatcac

SEQ ID NO: 1543 - DNA, 20 bp, artificial
  human FXR antisense
aggtaaatgggaatgttcaa

SEQ ID NO: 1544 - DNA, 20 bp, artificial
  human FXR antisense
gggtgagttcagttttctcc

SEQ ID NO: 1545 - DNA, 20 bp, artificial
  human FXR antisense
agtttcttattgaaaatctc

SEQ ID NO: 1546 - DNA, 20 bp, artificial
  human FXR antisense
agcagagcatactcctcttg

SEQ ID NO: 1547 - DNA, 20 bp, artificial
  human FXR antisense
tcatcaagatttcttgagtg

SEQ ID NO: 1548 - DNA, 20 bp, artificial
  human FXR antisense
atgtaattacaacataaata

SEQ ID NO: 1549 - DNA, 20 bp, artificial
  human FXR antisense
caacagatagaattgaagta

SEQ ID NO: 1550 - DNA, 20 bp, artificial
  human FXR antisense
ctagttcaacagatagaatt

SEQ ID NO: 1551 - DNA, 20 bp, artificial
  human FXR antisense
gcaaaatgagattttcccta

SEQ ID NO: 1552 - DNA, 20 bp, artificial
  human FXR antisense
ctttgagctatgtttctaag

SEQ ID NO: 1553 - DNA, 20 bp, artificial
  human FXR antisense
acaggcaaagtgttgaggat

SEQ ID NO: 1554 - DNA, 20 bp, artificial
  human FXR antisense
tctagcccaatatttacagt

SEQ ID NO: 1555 - DNA, 20 bp, artificial
  human FXR antisense
tatctagcccaatatttaca

SEQ ID NO: 1556 - DNA, 20 bp, artificial
  human FXR antisense
ttcttctttcactccttcta

SEQ ID NO: 1557 - DNA, 20 bp, artificial
  human FXR antisense
aagaaaattcatctgtggta

SEQ ID NO: 1558 - DNA, 20 bp, artificial
  human FXR antisense
gttctgttaaaacaccaaat

SEQ ID NO: 1559 - DNA, 20 bp, artificial
  human FXR antisense
agagtctcagctggcatacg

SEQ ID NO: 1560 - DNA, 20 bp, artificial
  human FXR antisense
cctggtagagagtctcagct

SEQ ID NO: 1561 - DNA, 20 bp, artificial
  human FXR antisense
atgtacatatccatcacaca

SEQ ID NO: 1562 - DNA, 20 bp, artificial
  human FXR antisense
cttctgcactgaattcttct

SEQ ID NO: 1563 - DNA, 20 bp, artificial
  human FXR antisense
caatctggtcttcatggtcc

SEQ ID NO: 1564 - DNA, 20 bp, artificial
  human FXR antisense
aaactaaacataggtgttat

SEQ ID NO: 1565 - DNA, 20 bp, artificial
  human FXR antisense
acagagaagtggggtaaact

SEQ ID NO: 1566 - DNA, 20 bp, artificial
  human FXR antisense
atctagcccaatatttacag

SEQ ID NO: 1567 - DNA, 20 bp, artificial
  human FXR antisense
ataaaatatatgcaatatgg

SEQ ID NO: 1568 - DNA, 20 bp, artificial
  human FXR antisense
aaagtgtctgaagtttcatc

SEQ ID NO: 1569 - DNA, 20 bp, artificial
  human FXR antisense
tgtctccacaaacaacacac

SEQ ID NO: 1570 - DNA, 20 bp, artificial
  human FXR antisense
tgcactgaattcttctttta

SEQ ID NO: 1571 - DNA, 20 bp, artificial
  human FXR antisense
ccagatttcacagagaagtg

SEQ ID NO: 1572 - DNA, 20 bp, artificial
  human FXR antisense
tcccagatttcacagagaag

SEQ ID NO: 1573 - DNA, 20 bp, artificial
  human FXR antisense
acccctcccctgtaatcccc

SEQ ID NO: 1574 - DNA, 20 bp, artificial
  human FXR antisense
ataagtcggggagacaatga

SEQ ID NO: 1575 - DNA, 20 bp, artificial
  human FXR antisense
ttctttcactccttctacga

SEQ ID NO: 1576 - DNA, 20 bp, artificial
  human FXR antisense
caggaaactaagagaagcag

SEQ ID NO: 1577 - DNA, 20 bp, artificial
  human FXR antisense
ccaaatttttcaattgaaat

SEQ ID NO: 1578 - DNA, 20 bp, artificial
  human FXR antisense
tcatctgtggtaggtaaatg

SEQ ID NO: 1579 - DNA, 20 bp, artificial
  human FXR antisense
ccaggttctgtcccagagga

SEQ ID NO: 1580 - DNA, 20 bp, artificial
  human FXR antisense
ttccaggttctgtcccagag

SEQ ID NO: 1581 - DNA, 20 bp, artificial
  human FXR antisense
gaaactgaacattgctgtat

SEQ ID NO: 1582 - DNA, 20 bp, artificial
  human FXR antisense
tcacaggtcagtgcattata

SEQ ID NO: 1583 - DNA, 20 bp, artificial
  human FXR antisense
gtcgcttagatttacactga

SEQ ID NO: 1584 - DNA, 20 bp, artificial
  human FXR antisense
gtcaaaatgagaaaattttc

SEQ ID NO: 1585 - DNA, 20 bp, artificial
  human FXR antisense
ccatttccgtcaaaatgaga

SEQ ID NO: 1586 - DNA, 20 bp, artificial
  human FXR antisense
taccactatttcgaattctt

SEQ ID NO: 1587 - DNA, 20 bp, artificial
  human FXR antisense
acaggataacaattgctgta

SEQ ID NO: 1588 - DNA, 20 bp, artificial
  human FXR antisense
gatgtcttctacctccttgg

SEQ ID NO: 1589 - DNA, 20 bp, artificial
  human FXR antisense
tctttcactccttctacgat

SEQ ID NO: 1590 - DNA, 20 bp, artificial
  human FXR antisense
ctccctggtagagagtctca

SEQ ID NO: 1591 - DNA, 20 bp, artificial
  human FXR antisense
taataaaatgtagaagagtc

SEQ ID NO: 1592 - DNA, 20 bp, artificial
  human FXR antisense
gtttactctccatgacatca

SEQ ID NO: 1593 - DNA, 20 bp, artificial
  human FXR antisense
ataaatattcatcaagattt

SEQ ID NO: 1594 - DNA, 20 bp, artificial
  human FXR antisense
gtccagaagaaatccaggaa

SEQ ID NO: 1595 - DNA, 20 bp, artificial
  human FXR antisense
aaagaaaattcatctgtggt

SEQ ID NO: 1596 - DNA, 20 bp, artificial
  human FXR antisense
ctggtagagagtctcagctg

SEQ ID NO: 1597 - DNA, 20 bp, artificial
  human FXR antisense
aaaatgagaaaattttcttc

SEQ ID NO: 1598 - DNA, 20 bp, artificial
  human FXR antisense
tcattttcagttccccaata

SEQ ID NO: 1599 - DNA, 20 bp, artificial
  human FXR antisense
gtcgtttactctccatgaca

SEQ ID NO: 1600 - DNA, 20 bp, artificial
  human FXR antisense
cggggagacaatgaggtgag

SEQ ID NO: 1601 - DNA, 20 bp, artificial
  human FXR antisense
taggataagtcggggagaca

SEQ ID NO: 1602 - DNA, 20 bp, artificial
  human FXR antisense
ctacaaatgctcagaatcca

SEQ ID NO: 1603 - DNA, 20 bp, artificial
  human FXR antisense
ttcttttcttctttcactcc

SEQ ID NO: 1604 - DNA, 20 bp, artificial
  human FXR antisense
ctgtggtaggtaaatgggaa

SEQ ID NO: 1605 - DNA, 20 bp, artificial
  human FXR antisense
tctgtcccagaggacctgcc

SEQ ID NO: 1606 - DNA, 20 bp, artificial
  human FXR antisense
cgagtatggttccacttcca

SEQ ID NO: 1607 - DNA, 20 bp, artificial
  human FXR antisense
gaggaaatctgtggttgaac

SEQ ID NO: 1608 - DNA, 20 bp, artificial
  human FXR antisense
ctttacacccctcacaggtc

SEQ ID NO: 1609 - DNA, 20 bp, artificial
  human FXR antisense
aatttcagttaacaagcatt

SEQ ID NO: 1610 - DNA, 20 bp, artificial
  human FXR antisense
caagtcacgaccttcactgt

SEQ ID NO: 1611 - DNA, 20 bp, artificial
  human FXR antisense
gaacatagcttcaaccgcag

SEQ ID NO: 1612 - DNA, 20 bp, artificial
  human FXR antisense
aatctcagctgaacgaagga

SEQ ID NO: 1613 - DNA, 20 bp, artificial
  human FXR antisense
gagcatactcctcttgagtc

SEQ ID NO: 1614 - DNA, 20 bp, artificial
  human FXR antisense
caggataacaattgctgtaa

SEQ ID NO: 1615 - DNA, 20 bp, artificial
  human FXR antisense
tctccatgacatcagcatct

SEQ ID NO: 1616 - DNA, 20 bp, artificial
  human FXR antisense
aattaggcaaacagggcttg

SEQ ID NO: 1617 - DNA, 20 bp, artificial
  human FXR antisense
gtcccagaggacctgccact

SEQ ID NO: 1618 - DNA, 20 bp, artificial
  human FXR antisense
cacagctcatcccctttgat

SEQ ID NO: 1619 - DNA, 20 bp, artificial
  human FXR antisense
aacaagcattcagccaacat

SEQ ID NO: 1620 - DNA, 20 bp, artificial
  human FXR antisense
cagtcgcttagatttacact

SEQ ID NO: 1621 - DNA, 20 bp, artificial
  human FXR antisense
aatgagaaaattttcttctg

SEQ ID NO: 1622 - DNA, 20 bp, artificial
  human FXR antisense
catcagagataccactattt

SEQ ID NO: 1623 - DNA, 20 bp, artificial
  human FXR antisense
cattttcagttccccaatac

SEQ ID NO: 1624 - DNA, 20 bp, artificial
  human FXR antisense
ctcccctgtaatccccatca

SEQ ID NO: 1625 - DNA, 20 bp, artificial
  human FXR antisense
gttcaacagatagaattgaa

SEQ ID NO: 1626 - DNA, 20 bp, artificial
  human FXR antisense
aggataagtcggggagacaa

SEQ ID NO: 1627 - DNA, 20 bp, artificial
  human FXR antisense
tccagaagaaatccaggaaa

SEQ ID NO: 1628 - DNA, 20 bp, artificial
  human FXR antisense
aaatttttcaattgaaatgc

SEQ ID NO: 1629 - DNA, 20 bp, artificial
  human FXR antisense
gtaaatgggaatgttcaatg

SEQ ID NO: 1630 - DNA, 20 bp, artificial
  human FXR antisense
tggaataataggatgacgag

SEQ ID NO: 1631 - DNA, 20 bp, artificial
  human FXR antisense
tatattccaggagagtacca

SEQ ID NO: 1632 - DNA, 20 bp, artificial
  human FXR antisense
tagagagtctcagctggcat

SEQ ID NO: 1633 - DNA, 20 bp, artificial
  human FXR antisense
tgaagaaacctttacacccc

SEQ ID NO: 1634 - DNA, 20 bp, artificial
  human FXR antisense
gcttagatttacactgaatt

SEQ ID NO: 1635 - DNA, 20 bp, artificial
  human FXR antisense
tgttgatctggggtgagttc

SEQ ID NO: 1636 - DNA, 20 bp, artificial
  human FXR antisense
ttgtgaattctacaagaacc

SEQ ID NO: 1637 - DNA, 20 bp, artificial
  human FXR antisense
ttatatattcatcagagata

SEQ ID NO: 1638 - DNA, 20 bp, artificial
  human FXR antisense
ggataacaattgctgtaagc

SEQ ID NO: 1639 - DNA, 20 bp, artificial
  human FXR antisense
aggcaaagtgttgaggattt

SEQ ID NO: 1640 - DNA, 20 bp, artificial
  human FXR antisense
aatttttcaattgaaatgca

SEQ ID NO: 1641 - DNA, 20 bp, artificial
  human FXR antisense
ggtaaatgggaatgttcaat

SEQ ID NO: 1642 - DNA, 20 bp, artificial
  human FXR antisense
ttctgttaaaacaccaaata

SEQ ID NO: 1643 - DNA, 20 bp, artificial
  human FXR antisense
tccaggttctgtcccagagg

SEQ ID NO: 1644 - DNA, 20 bp, artificial
  human FXR antisense
cacacagctcatcccctttg

SEQ ID NO: 1645 - DNA, 20 bp, artificial
  human FXR antisense
ttcagttaacaagcattcag

SEQ ID NO: 1646 - DNA, 20 bp, artificial
  human FXR antisense
atttccgtcaaaatgagaaa

SEQ ID NO: 1647 - DNA, 20 bp, artificial
  human FXR antisense
aacgaaggaacatagcttca

SEQ ID NO: 1648 - DNA, 20 bp, artificial
  human FXR antisense
ttttcagttccccaatactt

SEQ ID NO: 1649 - DNA, 20 bp, artificial
  human FXR antisense
taataaaatatatgcaatat

SEQ ID NO: 1650 - DNA, 20 bp, artificial
  human FXR antisense
ctttaataaaatatatgcaa

SEQ ID NO: 1651 - DNA, 20 bp, artificial
  human FXR antisense
gggagacaatgaggtgagga

SEQ ID NO: 1652 - DNA, 20 bp, artificial
  human FXR antisense
tctgtggtaggtaaatggga

SEQ ID NO: 1653 - DNA, 20 bp, artificial
  human FXR antisense
cccgtttttacacttgtaca

SEQ ID NO: 1654 - DNA, 20 bp, artificial
  human FXR antisense
ttaacaagcattcagccaac

SEQ ID NO: 1655 - DNA, 20 bp, artificial
  human FXR antisense
ctggggtgagttcagttttc

SEQ ID NO: 1656 - DNA, 20 bp, artificial
  human FXR antisense
ttcttttaaaattttatttg

SEQ ID NO: 1657 - DNA, 20 bp, artificial
  human FXR antisense
ttgagtcattttcagttccc

SEQ ID NO: 1658 - DNA, 20 bp, artificial
  human FXR antisense
caaagtgttgaggattttca

SEQ ID NO: 1659 - DNA, 20 bp, artificial
  human FXR antisense
aaatattcatcaagatttct

SEQ ID NO: 1660 - DNA, 20 bp, artificial
  human FXR antisense
tgtggtaggtaaatgggaat

SEQ ID NO: 1661 - DNA, 20 bp, artificial
  human FXR antisense
ctgtcccagaggacctgcca

SEQ ID NO: 1662 - DNA, 20 bp, artificial
  human FXR antisense
ccaggagagtaccactcttc

SEQ ID NO: 1663 - DNA, 20 bp, artificial
  human FXR antisense
cccctcacaggtcagtgcat

SEQ ID NO: 1664 - DNA, 20 bp, artificial
  human FXR antisense
gtacacagcgtttttggtaa

SEQ ID NO: 1665 - DNA, 20 bp, artificial
  human FXR antisense
attcttcttttaaaatttta

SEQ ID NO: 1666 - DNA, 20 bp, artificial
  human FXR antisense
aacatagcttcaaccgcaga

SEQ ID NO: 1667 - DNA, 20 bp, artificial
  human FXR antisense
tgaatgtccgtaattcagtc

SEQ ID NO: 1668 - DNA, 20 bp, artificial
  human FXR antisense
gatttcttgagtgaaactgg

SEQ ID NO: 1669 - DNA, 20 bp, artificial
  human FXR antisense
cttttcttctttcactcctt

SEQ ID NO: 1670 - DNA, 20 bp, artificial
  human FXR antisense
tccaaatttttcaattgaaa

SEQ ID NO: 1671 - DNA, 20 bp, artificial
  human FXR antisense
attcatttttgatcccatcc

SEQ ID NO: 1672 - DNA, 20 bp, artificial
  human FXR antisense
agattcatttttgatcccat

SEQ ID NO: 1673 - DNA, 20 bp, artificial
  human FXR antisense
ccaggttggaataataggat

SEQ ID NO: 1674 - DNA, 20 bp, artificial
  human FXR antisense
gaagaaacctttacacccct

SEQ ID NO: 1675 - DNA, 20 bp, artificial
  human FXR antisense
gtacatatccatcacacagt

SEQ ID NO: 1676 - DNA, 20 bp, artificial
  human FXR antisense
gttgatctggggtgagttca

SEQ ID NO: 1677 - DNA, 20 bp, artificial
  human FXR antisense
gagtctgttgatctggggtg

SEQ ID NO: 1678 - DNA, 20 bp, artificial
  human FXR antisense
ttgtctatctggagacagga

SEQ ID NO: 1679 - DNA, 20 bp, artificial
  human FXR antisense
acgtcccagatttcacagag

SEQ ID NO: 1680 - DNA, 20 bp, artificial
  human FXR antisense
tgtaattacaacataaatat

SEQ ID NO: 1681 - DNA, 20 bp, artificial
  human FXR antisense
gaagtttcatcttgaggaaa

SEQ ID NO: 1682 - DNA, 20 bp, artificial
  human FXR antisense
aataaaatgtagaagagtct

SEQ ID NO: 1683 - DNA, 20 bp, artificial
  human FXR antisense
tccataataaaatgtagaag

SEQ ID NO: 1684 - DNA, 20 bp, artificial
  human FXR antisense
ttttgtgaattctacaagaa

SEQ ID NO: 1685 - DNA, 20 bp, artificial
  human FXR antisense
aaaactaaacataggtgtta

SEQ ID NO: 1686 - DNA, 20 bp, artificial
  human FXR antisense
ctgtaagcagagcatactcc

SEQ ID NO: 1687 - DNA, 20 bp, artificial
  human FXR antisense
gagacaggataacaattgct

SEQ ID NO: 1688 - DNA, 20 bp, artificial
  human FXR antisense
tgtctatctggagacaggat

SEQ ID NO: 1689 - DNA, 20 bp, artificial
  human FXR antisense
aaatatatgcaatatggtaa

SEQ ID NO: 1690 - DNA, 20 bp, artificial
  human FXR antisense
ttctaagtcttcttttcttc

SEQ ID NO: 1691 - DNA, 20 bp, artificial
  human FXR antisense
taaatgggaatgttcaatga

SEQ ID NO: 1692 - DNA, 20 bp, artificial
  human FXR antisense
catctgtggtaggtaaatgg

SEQ ID NO: 1693 - DNA, 20 bp, artificial
  human FXR antisense
tagtggtatccagaggctct

SEQ ID NO: 1694 - DNA, 20 bp, artificial
  human FXR antisense
agtcgcttagatttacactg

SEQ ID NO: 1695 - DNA, 20 bp, artificial
  human FXR antisense
aattgctgtaagcagagcat

SEQ ID NO: 1696 - DNA, 20 bp, artificial
  human FXR antisense
ccctgtaatccccatcactg

SEQ ID NO: 1697 - DNA, 20 bp, artificial
  human FXR antisense
tacaaatgctcagaatccaa

SEQ ID NO: 1698 - DNA, 20 bp, artificial
  human FXR antisense
cattatagtggtatccagag

SEQ ID NO: 1699 - DNA, 20 bp, artificial
  human FXR antisense
taatgcttctcctgaagaaa

SEQ ID NO: 1700 - DNA, 20 bp, artificial
  human FXR antisense
tcaaaatgagaaaattttct

SEQ ID NO: 1701 - DNA, 20 bp, artificial
  human FXR antisense
tttccgtcaaaatgagaaaa

SEQ ID NO: 1702 - DNA, 20 bp, artificial
  human FXR antisense
acggaagtttcttattgaaa

SEQ ID NO: 1703 - DNA, 20 bp, artificial
  human FXR antisense
cccagatttcacagagaagt

SEQ ID NO: 1704 - DNA, 20 bp, artificial
  human FXR antisense
cacatgtaattacaacataa

SEQ ID NO: 1705 - DNA, 20 bp, artificial
  human FXR antisense
atttaattaggcaaacaggg

SEQ ID NO: 1706 - DNA, 20 bp, artificial
  human FXR antisense
ccagcgattttgctacaaat

SEQ ID NO: 1707 - DNA, 20 bp, artificial
  human FXR antisense
ctgggaggattctggactga

SEQ ID NO: 1708 - DNA, 20 bp, artificial
  human FXR antisense
cccatccaaatttttcaatt

SEQ ID NO: 1709 - DNA, 20 bp, artificial
  human FXR antisense
tgggaatgttcaatgagatt

SEQ ID NO: 1710 - DNA, 20 bp, artificial
  human FXR antisense
aggagagtaccactcttcag

SEQ ID NO: 1711 - DNA, 20 bp, artificial
  human FXR antisense
atatattccaggagagtacc

SEQ ID NO: 1712 - DNA, 20 bp, artificial
  human FXR antisense
acacacagctcatccccttt

SEQ ID NO: 1713 - DNA, 20 bp, artificial
  human FXR antisense
gtctccacaaacaacacaca

SEQ ID NO: 1714 - DNA, 20 bp, artificial
  human FXR antisense
aatgcttctcctgaagaaac

SEQ ID NO: 1715 - DNA, 20 bp, artificial
  human FXR antisense
tacacagcgtttttggtaat

SEQ ID NO: 1716 - DNA, 20 bp, artificial
  human FXR antisense
cttcttttaaaattttattt

SEQ ID NO: 1717 - DNA, 20 bp, artificial
  human FXR antisense
aaagtgttgaggattttcag

SEQ ID NO: 1718 - DNA, 20 bp, artificial
  human FXR antisense
gacccctcccctgtaatccc

SEQ ID NO: 1719 - DNA, 20 bp, artificial
  human FXR antisense
atttacagttgtggaagtta

SEQ ID NO: 1720 - DNA, 20 bp, artificial
  human FXR antisense
caatatggtaagatgagcaa

SEQ ID NO: 1721 - DNA, 20 bp, artificial
  human FXR antisense
agtttcatcttgaggaaatg

SEQ ID NO: 1722 - DNA, 20 bp, artificial
  human FXR antisense
gattcatttttgatcccatc

SEQ ID NO: 1723 - DNA, 20 bp, artificial
  human FXR antisense
aataataggatgacgaggaa

SEQ ID NO: 1724 - DNA, 20 bp, artificial
  human FXR antisense
cccaggttggaataatagga

SEQ ID NO: 1725 - DNA, 20 bp, artificial
  human FXR antisense
acacagcgtttttggtaatg

SEQ ID NO: 1726 - DNA, 20 bp, artificial
  human FXR antisense
tcgtttactctccatgacat

SEQ ID NO: 1727 - DNA, 20 bp, artificial
  human FXR antisense
ataaaggaaagttatacatc

SEQ ID NO: 1728 - DNA, 20 bp, artificial
  human FXR antisense
ttcaacagatagaattgaag

SEQ ID NO: 1729 - DNA, 20 bp, artificial
  human FXR antisense
cttggattgttttgggtcag

SEQ ID NO: 1730 - DNA, 20 bp, artificial
  human FXR antisense
caaatttttcaattgaaatg

SEQ ID NO: 1731 - DNA, 20 bp, artificial
  human FXR antisense
cgaggaaatctgtggttgaa

SEQ ID NO: 1732 - DNA, 20 bp, artificial
  human FXR antisense
tggtatccagaggctctgtc

SEQ ID NO: 1733 - DNA, 20 bp, artificial
  human FXR antisense
aaatctcagctgaacgaagg

SEQ ID NO: 1734 - DNA, 20 bp, artificial
  human FXR antisense
tcatcagagataccactatt

SEQ ID NO: 1735 - DNA, 20 bp, artificial
  human FXR antisense
attgtctatctggagacagg

SEQ ID NO: 1736 - DNA, 20 bp, artificial
  human FXR antisense
cccagcgattttgctacaaa

SEQ ID NO: 1737 - DNA, 20 bp, artificial
  human FXR antisense
gggaggattctggactgagt

SEQ ID NO: 1738 - DNA, 20 bp, artificial
  human FXR antisense
gaaatgtccagaagaaatcc

SEQ ID NO: 1739 - DNA, 20 bp, artificial
  human FXR antisense
aaggaacatagcttcaaccg

SEQ ID NO: 1740 - DNA, 20 bp, artificial
  human FXR antisense
tgagtcattttcagttcccc

SEQ ID NO: 1741 - DNA, 20 bp, artificial
  human FXR antisense
gtaagcagagcatactcctc

SEQ ID NO: 1742 - DNA, 20 bp, artificial
  human FXR antisense
ggcaaagtgttgaggatttt

SEQ ID NO: 1743 - DNA, 20 bp, artificial
  human FXR antisense
attacaacataaatattcat

SEQ ID NO: 1744 - DNA, 20 bp, artificial
  human FXR antisense
cagcgattttgctacaaatg

SEQ ID NO: 1745 - DNA, 20 bp, artificial
  human FXR antisense
ttctacctccttggattgtt

SEQ ID NO: 1746 - DNA, 20 bp, artificial
  human FXR antisense
cttctttcactccttctacg

SEQ ID NO: 1747 - DNA, 20 bp, artificial
  human FXR antisense
acgaggaaatctgtggttga

SEQ ID NO: 1748 - DNA, 20 bp, artificial
  human FXR antisense
gacgaggaaatctgtggttg

SEQ ID NO: 1749 - DNA, 20 bp, artificial
  human FXR antisense
ctgctgggggtagaaaccca

SEQ ID NO: 1750 - DNA, 20 bp, artificial
  human FXR antisense
ataccactatttcgaattct

SEQ ID NO: 1751 - DNA, 20 bp, artificial
  human FXR antisense
attttcagttccccaatact

SEQ ID NO: 1752 - DNA, 20 bp, artificial
  human FXR antisense
tgtattgtctatctggagac

SEQ ID NO: 1753 - DNA, 20 bp, artificial
  human FXR antisense
tcctgaagcttctctactgc

SEQ ID NO: 1754 - DNA, 20 bp, artificial
  human FXR antisense
ctatctagcccaatatttac

SEQ ID NO: 1755 - DNA, 20 bp, artificial
  human FXR antisense
tagttcaacagatagaattg

SEQ ID NO: 1756 - DNA, 20 bp, artificial
  human FXR antisense
ccatccaaatttttcaattg

SEQ ID NO: 1757 - DNA, 20 bp, artificial
  human FXR antisense
ttctgtcccagaggacctgc

SEQ ID NO: 1758 - DNA, 20 bp, artificial
  human FXR antisense
ctgggggtagaaacccaggt

SEQ ID NO: 1759 - DNA, 20 bp, artificial
  human FXR antisense
tattccaggagagtaccact

SEQ ID NO: 1760 - DNA, 20 bp, artificial
  human FXR antisense
agagagtctcagctggcata

SEQ ID NO: 1761 - DNA, 20 bp, artificial
  human FXR antisense
gtggtatccagaggctctgt

SEQ ID NO: 1762 - DNA, 20 bp, artificial
  human FXR antisense
atgagaaaattttcttctgc

SEQ ID NO: 1763 - DNA, 20 bp, artificial
  human FXR antisense
tttgtgaattctacaagaac

SEQ ID NO: 1764 - DNA, 20 bp, artificial
  human FXR antisense
gagtcattttcagttcccca

SEQ ID NO: 1765 - DNA, 20 bp, artificial
  human FXR antisense
gatagaattgaagtaacaat

SEQ ID NO: 1766 - DNA, 20 bp, artificial
  human FXR antisense
ggataagtcggggagacaat

SEQ ID NO: 1767 - DNA, 20 bp, artificial
  human FXR antisense
catcttgaggaaatgtccag

SEQ ID NO: 1768 - DNA, 20 bp, artificial
  human FXR antisense
aaaaaactccaaagtgtctg

SEQ ID NO: 1769 - DNA, 20 bp, artificial
  human FXR antisense
aaaaaaactccaaagtgtct

SEQ ID NO: 1770 - DNA, 20 bp, artificial
  human FXR antisense
gtatggttccacttccaggt

SEQ ID NO: 1771 - DNA, 20 bp, artificial
  human FXR antisense
aaatctgtggttgaacttgg

SEQ ID NO: 1772 - DNA, 20 bp, artificial
  human FXR antisense
atgcttctcctgaagaaacc

SEQ ID NO: 1773 - DNA, 20 bp, artificial
  human FXR antisense
tggggtgagttcagttttct

SEQ ID NO: 1774 - DNA, 20 bp, artificial
  human FXR antisense
ttcttcttttaaaattttat

SEQ ID NO: 1775 - DNA, 20 bp, artificial
  human FXR antisense
aattcttcttttaaaatttt

SEQ ID NO: 1776 - DNA, 20 bp, artificial
  human FXR antisense
caattgctgtaagcagagca

SEQ ID NO: 1777 - DNA, 20 bp, artificial
  human FXR antisense
acaattgctgtaagcagagc

SEQ ID NO: 1778 - DNA, 20 bp, artificial
  human FXR antisense
aggcgacccaggagacaggc

SEQ ID NO: 1779 - DNA, 20 bp, artificial
  human FXR antisense
agatagaattgaagtaacaa

SEQ ID NO: 1780 - DNA, 20 bp, artificial
  human FXR antisense
tcaacagatagaattgaagt

SEQ ID NO: 1781 - DNA, 20 bp, artificial
  human FXR antisense
agtcttcttttcttctttca

SEQ ID NO: 1782 - DNA, 20 bp, artificial
  human FXR antisense
aagtcttcttttcttctttc

SEQ ID NO: 1783 - DNA, 20 bp, artificial
  human FXR antisense
cagaagaaatccaggaaact

SEQ ID NO: 1784 - DNA, 20 bp, artificial
  human FXR antisense
aaaagaaaattcatctgtgg

SEQ ID NO: 1785 - DNA, 20 bp, artificial
  human FXR antisense
ggaaactgaacattgctgta

SEQ ID NO: 1786 - DNA, 20 bp, artificial
  human FXR antisense
atattccaggagagtaccac

SEQ ID NO: 1787 - DNA, 20 bp, artificial
  human FXR antisense
gtttctccctggtagagagt

SEQ ID NO: 1788 - DNA, 20 bp, artificial
  human FXR antisense
acgaaggaacatagcttcaa

SEQ ID NO: 1789 - DNA, 20 bp, artificial
  human FXR antisense
aaaatctcagctgaacgaag

SEQ ID NO: 1790 - DNA, 20 bp, artificial
  human FXR antisense
ggagacaggataacaattgc

SEQ ID NO: 1791 - DNA, 20 bp, artificial
  human FXR antisense
aataaaggaaagttatacat

SEQ ID NO: 1792 - DNA, 20 bp, artificial
  human FXR antisense
aaactccaaagtgtctgaag

SEQ ID NO: 1793 - DNA, 20 bp, artificial
  human FXR antisense
aataggatgacgaggaaatc

SEQ ID NO: 1794 - DNA, 20 bp, artificial
  human FXR antisense
cagtttctccctggtagaga

SEQ ID NO: 1795 - DNA, 20 bp, artificial
  human FXR antisense
caaaatgagaaaattttctt

SEQ ID NO: 1796 - DNA, 20 bp, artificial
  human FXR antisense
gtaattacaacataaatatt

SEQ ID NO: 1797 - DNA, 20 bp, artificial
  human FXR antisense
agcgattttgctacaaatgc

SEQ ID NO: 1798 - DNA, 20 bp, artificial
  human FXR antisense
catccaaatttttcaattga

SEQ ID NO: 1799 - DNA, 20 bp, artificial
  human FXR antisense
tctccctggtagagagtctc

SEQ ID NO: 1800 - DNA, 20 bp, artificial
  human FXR antisense
ttagatttacactgaatttc

SEQ ID NO: 1801 - DNA, 20 bp, artificial
  human FXR antisense
attgctgtaagcagagcata

SEQ ID NO: 1802 - DNA, 20 bp, artificial
  human FXR antisense
gaagcttctctactgcctct

SEQ ID NO: 1803 - DNA, 20 bp, artificial
  human FXR antisense
tctacctccttggattgttt

SEQ ID NO: 1804 - DNA, 20 bp, artificial
  human FXR antisense
catatattccaggagagtac

SEQ ID NO: 1805 - DNA, 20 bp, artificial
  human FXR antisense
ctccacaaacaacacacagc

SEQ ID NO: 1806 - DNA, 20 bp, artificial
  human FXR antisense
ttcatcagagataccactat

SEQ ID NO: 1807 - DNA, 20 bp, artificial
  human FXR antisense
aaaaactaaacataggtgtt

SEQ ID NO: 1808 - DNA, 20 bp, artificial
  human FXR antisense
gcaaagtgttgaggattttc

SEQ ID NO: 1809 - DNA, 20 bp, artificial
  human FXR antisense
aattacaacataaatattca

SEQ ID NO: 1810 - DNA, 20 bp, artificial
  human FXR antisense
aatgtccagaagaaatccag

SEQ ID NO: 1811 - DNA, 20 bp, artificial
  human FXR antisense
ataggatgacgaggaaatct

SEQ ID NO: 1812 - DNA, 20 bp, artificial
  human FXR antisense
catgtacatatccatcacac

SEQ ID NO: 1813 - DNA, 20 bp, artificial
  human FXR antisense
ggggtgagttcagttttctc

SEQ ID NO: 1814 - DNA, 20 bp, artificial
  human FXR antisense
gaattcttcttttaaaattt

SEQ ID NO: 1815 - DNA, 20 bp, artificial
  human FXR antisense
gtctatctggagacaggata

SEQ ID NO: 1816 - DNA, 20 bp, artificial
  human FXR antisense
ggcaaacagggcttgccaat

SEQ ID NO: 1817 - DNA, 20 bp, artificial
  human FXR antisense
aacaacacacagctcatccc

SEQ ID NO: 1818 - DNA, 20 bp, artificial
  human FXR antisense
agtggtatccagaggctctg

SEQ ID NO: 1819 - DNA, 20 bp, artificial
  human FXR antisense
tttttacacttgtacacagc

SEQ ID NO: 1820 - DNA, 20 bp, artificial
  human FXR antisense
gaatttcagttaacaagcat

SEQ ID NO: 1821 - DNA, 20 bp, artificial
  human FXR antisense
cttagatttacactgaattt

SEQ ID NO: 1822 - DNA, 20 bp, artificial
  human FXR antisense
aggataacaattgctgtaag

SEQ ID NO: 1823 - DNA, 20 bp, artificial
  human FXR antisense
tatctggagacaggataaca

SEQ ID NO: 1824 - DNA, 20 bp, artificial
  human FXR antisense
ccagatcccagcgattttgc

SEQ ID NO: 1825 - DNA, 20 bp, artificial
  human FXR antisense
taagtcttcttttcttcttt

SEQ ID NO: 1826 - DNA, 20 bp, artificial
  human FXR antisense
atgggaatgttcaatgagat

SEQ ID NO: 1827 - DNA, 20 bp, artificial
  human FXR antisense
ttctccctggtagagagtct

SEQ ID NO: 1828 - DNA, 20 bp, artificial
  human FXR antisense
acccctcacaggtcagtgca

SEQ ID NO: 1829 - DNA, 20 bp, artificial
  human FXR antisense
ctgaagaaacctttacaccc

SEQ ID NO: 1830 - DNA, 20 bp, artificial
  human FXR antisense
ttcacagagaagtggggtaa

SEQ ID NO: 1831 - DNA, 20 bp, artificial
  human FXR antisense
aataaaatatatgcaatatg

SEQ ID NO: 1832 - DNA, 20 bp, artificial
  human FXR antisense
cagatcccagcgattttgct

SEQ ID NO: 1833 - DNA, 20 bp, artificial
  human FXR antisense
tttctccctggtagagagtc

SEQ ID NO: 1834 - DNA, 20 bp, artificial
  human FXR antisense
acaacacacagctcatcccc

SEQ ID NO: 1835 - DNA, 20 bp, artificial
  human FXR antisense
tttacacccctcacaggtca

SEQ ID NO: 1836 - DNA, 20 bp, artificial
  human FXR antisense
gtaatgcttctcctgaagaa

SEQ ID NO: 1837 - DNA, 20 bp, artificial
  human FXR antisense
gagataccactatttcgaat

SEQ ID NO: 1838 - DNA, 20 bp, artificial
  human FXR antisense
gagacaggcaaagtgttgag

SEQ ID NO: 1839 - DNA, 20 bp, artificial
  human FXR antisense
ccagaagaaatccaggaaac

SEQ ID NO: 1840 - DNA, 20 bp, artificial
  human FXR antisense
tctgttaaaacaccaaataa

SEQ ID NO: 1841 - DNA, 20 bp, artificial
  human FXR antisense
gtttttacacttgtacacag

SEQ ID NO: 1842 - DNA, 20 bp, artificial
  human FXR antisense
tttcagttccccaatacttt

SEQ ID NO: 1843 - DNA, 20 bp, artificial
  human FXR antisense
gctgtaagcagagcatactc

SEQ ID NO: 1844 - DNA, 20 bp, artificial
  human FXR antisense
tattgtctatctggagacag

SEQ ID NO: 1845 - DNA, 20 bp, artificial
  human FXR antisense
agacaatgaggtgaggagga

SEQ ID NO: 1846 - DNA, 20 bp, artificial
  human FXR antisense
tctggagacaggataacaat

SEQ ID NO: 1847 - DNA, 20 bp, artificial
  human FXR antisense
atctggagacaggataacaa

SEQ ID NO: 1848 - DNA, 20 bp, artificial
  human FXR antisense
cctgaagcttctctactgcc

SEQ ID NO: 1849 - DNA, 20 bp, artificial
  human FXR antisense
ttacaacataaatattcatc

SEQ ID NO: 1850 - DNA, 20 bp, artificial
  human FXR antisense
gataagtcggggagacaatg

SEQ ID NO: 1851 - DNA, 20 bp, artificial
  human FXR antisense
cttcttttcttctttcactc

SEQ ID NO: 1852 - DNA, 20 bp, artificial
  human FXR antisense
tgaatttcagttaacaagca

SEQ ID NO: 1853 - DNA, 20 bp, artificial
  human FXR antisense
aaaattttcttctgcactga

SEQ ID NO: 1854 - DNA, 20 bp, artificial
  human FXR antisense
cataaatattcatcaagatt

SEQ ID NO: 1855 - DNA, 20 bp, artificial
  human FXR antisense
ggggagacaatgaggtgagg

SEQ ID NO: 1856 - DNA, 20 bp, artificial
  human FXR antisense
agaagaaatccaggaaacta

SEQ ID NO: 1857 - DNA, 20 bp, artificial
  human FXR antisense
gttggaataataggatgacg

SEQ ID NO: 1858 - DNA, 20 bp, artificial
  human FXR antisense
caggttggaataataggatg

SEQ ID NO: 1859 - DNA, 20 bp, artificial
  human FXR antisense
aaaatgtagaagagtctgtt

SEQ ID NO: 1860 - DNA, 20 bp, artificial
  human FXR antisense
tgagaaaattttcttctgca

SEQ ID NO: 1861 - DNA, 20 bp, artificial
  human FXR antisense
ataacaattgctgtaagcag

SEQ ID NO: 1862 - DNA, 20 bp, artificial
  human FXR antisense
tggagacaggataacaattg

SEQ ID NO: 1863 - DNA, 20 bp, artificial
  human FXR antisense
tttcacagagaagtggggta

SEQ ID NO: 1864 - DNA, 20 bp, artificial
  human FXR antisense
cacttccaggttctgtccca

SEQ ID NO: 1865 - DNA, 20 bp, artificial
  human FXR antisense
gcattatagtggtatccaga

SEQ ID NO: 1866 - DNA, 20 bp, artificial
  human FXR antisense
cggaagtttcttattgaaaa

SEQ ID NO: 1867 - DNA, 20 bp, artificial
  human FXR antisense
agataccactatttcgaatt

SEQ ID NO: 1868 - DNA, 20 bp, artificial
  human FXR antisense
cagagataccactatttcga

SEQ ID NO: 1869 - DNA, 20 bp, artificial
  human FXR antisense
taaaaactaaacataggtgt

SEQ ID NO: 1870 - DNA, 20 bp, artificial
  human FXR antisense
tgaagcttctctactgcctc

SEQ ID NO: 1871 - DNA, 20 bp, artificial
  human FXR antisense
taaaggaaagttatacatca

SEQ ID NO: 1872 - DNA, 20 bp, artificial
  human FXR antisense
gaggaaatgtccagaagaaa

SEQ ID NO: 1873 - DNA, 20 bp, artificial
  human FXR antisense
atccaaatttttcaattgaa

SEQ ID NO: 1874 - DNA, 20 bp, artificial
  human FXR antisense
gaaaaagaaaattcatctgt

SEQ ID NO: 1875 - DNA, 20 bp, artificial
  human FXR antisense
cttccaggttctgtcccaga

SEQ ID NO: 1876 - DNA, 20 bp, artificial
  human FXR antisense
aatctgtggttgaacttggg

SEQ ID NO: 1877 - DNA, 20 bp, artificial
  human FXR antisense
gaataataggatgacgagga

SEQ ID NO: 1878 - DNA, 20 bp, artificial
  human FXR antisense
attccaggagagtaccactc

SEQ ID NO: 1879 - DNA, 20 bp, artificial
  human FXR antisense
tcagtttctccctggtagag

SEQ ID NO: 1880 - DNA, 20 bp, artificial
  human FXR antisense
ttacacccctcacaggtcag

SEQ ID NO: 1881 - DNA, 20 bp, artificial
  human FXR antisense
gcatgtacatatccatcaca

SEQ ID NO: 1882 - DNA, 20 bp, artificial
  human FXR antisense
tgtaagcagagcatactcct

SEQ ID NO: 1883 - DNA, 20 bp, artificial
  human FXR antisense
taaatattcatcaagatttc

SEQ ID NO: 1884 - DNA, 20 bp, artificial
  human FXR antisense
atctgtggtaggtaaatggg

SEQ ID NO: 1885 - DNA, 20 bp, artificial
  human FXR antisense
aacacacagctcatcccctt

SEQ ID NO: 1886 - DNA, 20 bp, artificial
  human FXR antisense
tttacactgaatttcagtta

SEQ ID NO: 1887 - DNA, 20 bp, artificial
  human FXR antisense
agagataccactatttcgaa

SEQ ID NO: 1888 - DNA, 20 bp, artificial
  human FXR antisense
cacagagaagtggggtaaac

SEQ ID NO: 1889 - DNA, 20 bp, artificial
  human FXR antisense
gggtagaaacccaggttgga

SEQ ID NO: 1890 - DNA, 20 bp, artificial
  human FXR antisense
attttcttctgcactgaatt

SEQ ID NO: 1891 - DNA, 20 bp, artificial
  human FXR antisense
ccaatacttttataaaaact

SEQ ID NO: 1892 - DNA, 20 bp, artificial
  human FXR antisense
caatttaattaggcaaacag

SEQ ID NO: 1893 - DNA, 20 bp, artificial
  human FXR antisense
ggtcttcaaaaaaaactcca

SEQ ID NO: 1894 - DNA, 20 bp, artificial
  human FXR antisense
ccacttccaggttctgtccc

SEQ ID NO: 1895 - DNA, 20 bp, artificial
  human FXR antisense
gtagaaacccaggttggaat

SEQ ID NO: 1896 - DNA, 20 bp, artificial
  human FXR antisense
tttataaaaactaaacatag

SEQ ID NO: 1897 - DNA, 20 bp, artificial
  human FXR antisense
ccatgacatcagcatctcag

SEQ ID NO: 1898 - DNA, 20 bp, artificial
  human FXR antisense
atttcacagagaagtggggt

SEQ ID NO: 1899 - DNA, 20 bp, artificial
  human FXR antisense
aaataaaggaaagttataca

SEQ ID NO: 1900 - DNA, 20 bp, artificial
  human FXR antisense
tgaggaggaggagagagtct

SEQ ID NO: 1901 - DNA, 20 bp, artificial
  human FXR antisense
tcttcttttcttctttcact

SEQ ID NO: 1902 - DNA, 20 bp, artificial
  human FXR antisense
ggaaatgtccagaagaaatc

SEQ ID NO: 1903 - DNA, 20 bp, artificial
  human FXR antisense
aaaaactccaaagtgtctga

SEQ ID NO: 1904 - DNA, 20 bp, artificial
  human FXR antisense
atttacactgaatttcagtt

SEQ ID NO: 1905 - DNA, 20 bp, artificial
  human FXR antisense
gcaagtcacgaccttcactg

SEQ ID NO: 1906 - DNA, 20 bp, artificial
  human FXR antisense
aaatgtagaagagtctgttg

SEQ ID NO: 1907 - DNA, 20 bp, artificial
  human FXR antisense
cgaaggaacatagcttcaac

SEQ ID NO: 1908 - DNA, 20 bp, artificial
  human FXR antisense
tatatattcatcagagatac

SEQ ID NO: 1909 - DNA, 20 bp, artificial
  human FXR antisense
ttcagttccccaatactttt

SEQ ID NO: 1910 - DNA, 20 bp, artificial
  human FXR antisense
acatgtaattacaacataaa

SEQ ID NO: 1911 - DNA, 20 bp, artificial
  human FXR antisense
ccaaagtgtctgaagtttca

SEQ ID NO: 1912 - DNA, 20 bp, artificial
  human FXR antisense
ttggggaaactgaacattgc

SEQ ID NO: 1913 - DNA, 20 bp, artificial
  human FXR antisense
agagtctgttgatctggggt

SEQ ID NO: 1914 - DNA, 20 bp, artificial
  human FXR antisense
aaaaagaaaattcatctgtg

SEQ ID NO: 1915 - DNA, 20 bp, artificial
  human FXR antisense
agtatggttccacttccagg

SEQ ID NO: 1916 - DNA, 20 bp, artificial
  human FXR antisense
gggaaactgaacattgctgt

SEQ ID NO: 1917 - DNA, 20 bp, artificial
  human FXR antisense
tctccacaaacaacacacag

SEQ ID NO: 1918 - DNA, 20 bp, artificial
  human FXR antisense
gataccactatttcgaattc

SEQ ID NO: 1919 - DNA, 20 bp, artificial
  human FXR antisense
acataaatattcatcaagat

SEQ ID NO: 1920 - DNA, 20 bp, artificial
  human FXR antisense
tgtcccagaggacctgccac

SEQ ID NO: 1921 - DNA, 20 bp, artificial
  human FXR antisense
tagaaacccaggttggaata

SEQ ID NO: 1922 - DNA, 20 bp, artificial
  human FXR antisense
tccacaaacaacacacagct

SEQ ID NO: 1923 - DNA, 20 bp, artificial
  human FXR antisense
tccgtcaaaatgagaaaatt

SEQ ID NO: 1924 - DNA, 20 bp, artificial
  human FXR antisense
ttttataaaaactaaacata

SEQ ID NO: 1925 - DNA, 20 bp, artificial
  human FXR antisense
gtattgtctatctggagaca

SEQ ID NO: 1926 - DNA, 20 bp, artificial
  human FXR antisense
tccatgacatcagcatctca

SEQ ID NO: 1927 - DNA, 20 bp, artificial
  human FXR antisense
accaaataaattttcagaaa

SEQ ID NO: 1928 - DNA, 20 bp, artificial
  human FXR antisense
tttactctccatgacatcag

SEQ ID NO: 1929 - DNA, 20 bp, artificial
  human FXR antisense
aatttaattaggcaaacagg

SEQ ID NO: 1930 - DNA, 20 bp, artificial
  human FXR antisense
aaatgtccagaagaaatcca

SEQ ID NO: 1931 - DNA, 20 bp, artificial
  human FXR antisense
aatgggaatgttcaatgaga

SEQ ID NO: 1932 - DNA, 20 bp, artificial
  human FXR antisense
cttggggaaactgaacattg

SEQ ID NO: 1933 - DNA, 20 bp, artificial
  human FXR antisense
cctcagtttctccctggtag

SEQ ID NO: 1934 - DNA, 20 bp, artificial
  human FXR antisense
gaagagtctgttgatctggg

SEQ ID NO: 1935 - DNA, 20 bp, artificial
  human FXR antisense
atatattcatcagagatacc

SEQ ID NO: 1936 - DNA, 20 bp, artificial
  human FXR antisense
ctctccatgacatcagcatc

SEQ ID NO: 1937 - DNA, 20 bp, artificial
  human FXR antisense
ggaggaggagagagtctcgt

SEQ ID NO: 1938 - DNA, 20 bp, artificial
  human FXR antisense
tgggaggattctggactgag

SEQ ID NO: 1939 - DNA, 20 bp, artificial
  human FXR antisense
aaaaaaaactccaaagtgtc

SEQ ID NO: 1940 - DNA, 20 bp, artificial
  human FXR antisense
tggtcttcaaaaaaaactcc

SEQ ID NO: 1941 - DNA, 20 bp, artificial
  human FXR antisense
ccaaataaattttcagaaaa

SEQ ID NO: 1942 - DNA, 20 bp, artificial
  human FXR antisense
acagctcatcccctttgatc

SEQ ID NO: 1943 - DNA, 20 bp, artificial
  human FXR antisense
ccgtcaaaatgagaaaattt

SEQ ID NO: 1944 - DNA, 20 bp, artificial
  human FXR antisense
aagtttcttattgaaaatct

SEQ ID NO: 1945 - DNA, 20 bp, artificial
  human FXR antisense
caaagtgtctgaagtttcat

SEQ ID NO: 1946 - DNA, 20 bp, artificial
  human FXR antisense
tgacgaggaaatctgtggtt

SEQ ID NO: 1947 - DNA, 20 bp, artificial
  human FXR antisense
agaaacccaggttggaataa

SEQ ID NO: 1948 - DNA, 20 bp, artificial
  human FXR antisense
tgtacatatccatcacacag

SEQ ID NO: 1949 - DNA, 20 bp, artificial
  human FXR antisense
ttttcttctgcactgaattc

SEQ ID NO: 1950 - DNA, 20 bp, artificial
  human FXR antisense
aattttcttctgcactgaat

SEQ ID NO: 1951 - DNA, 20 bp, artificial
  human FXR antisense
gtacaagtgaaataaaggaa

SEQ ID NO: 1952 - DNA, 20 bp, artificial
  human FXR antisense
tacaacataaatattcatca

SEQ ID NO: 1953 - DNA, 20 bp, artificial
  human FXR antisense
gacaatgaggtgaggaggag

SEQ ID NO: 1954 - DNA, 20 bp, artificial
  human FXR antisense
atcttgaggaaatgtccaga

SEQ ID NO: 1955 - DNA, 20 bp, artificial
  human FXR antisense
tttcagaaaaagaaaattca

SEQ ID NO: 1956 - DNA, 20 bp, artificial
  human FXR antisense
caccaaataaattttcagaa

SEQ ID NO: 1957 - DNA, 20 bp, artificial
  human FXR antisense
acaggtcagtgcattatagt

SEQ ID NO: 1958 - DNA, 20 bp, artificial
  human FXR antisense
ttgaggaaatgtccagaaga

SEQ ID NO: 1959 - DNA, 20 bp, artificial
  human FXR antisense
cacaggtcagtgcattatag

SEQ ID NO: 1960 - DNA, 20 bp, artificial
  human FXR antisense
caatacttttataaaaacta

SEQ ID NO: 1961 - DNA, 20 bp, artificial
  human FXR antisense
aaaggaaagttatacatcag

SEQ ID NO: 1962 - DNA, 20 bp, artificial
  human FXR antisense
aaaactccaaagtgtctgaa

SEQ ID NO: 1963 - DNA, 20 bp, artificial
  human FXR antisense
ctcagtttctccctggtaga

SEQ ID NO: 1964 - DNA, 20 bp, artificial
  human FXR antisense
acactgaatttcagttaaca

SEQ ID NO: 1965 - DNA, 20 bp, artificial
  human FXR antisense
gagacaatgaggtgaggagg

SEQ ID NO: 1966 - DNA, 20 bp, artificial
  human FXR antisense
ttttcagaaaaagaaaattc

SEQ ID NO: 1967 - DNA, 20 bp, artificial
  human FXR antisense
attttcagaaaaagaaaatt

SEQ ID NO: 1968 - DNA, 20 bp, artificial
  human FXR antisense
atctgtggttgaacttgggg

SEQ ID NO: 1969 - DNA, 20 bp, artificial
  human FXR antisense
ggttggaataataggatgac

SEQ ID NO: 1970 - DNA, 20 bp, artificial
  human FXR antisense
aaacaacacacagctcatcc

SEQ ID NO: 1971 - DNA, 20 bp, artificial
  human FXR antisense
aagaaacctttacacccctc

SEQ ID NO: 1972 - DNA, 20 bp, artificial
  human FXR antisense
taaaatgtagaagagtctgt

SEQ ID NO: 1973 - DNA, 20 bp, artificial
  human FXR antisense
ctgaattcttcttttaaaat

SEQ ID NO: 1974 - DNA, 20 bp, artificial
  human FXR antisense
ttcttctgcactgaattctt

SEQ ID NO: 1975 - DNA, 20 bp, artificial
  human FXR antisense
tttcttctgcactgaattct

SEQ ID NO: 1976 - DNA, 20 bp, artificial
  human FXR antisense
gaaggaacatagcttcaacc

SEQ ID NO: 1977 - DNA, 20 bp, artificial
  human FXR antisense
ataaaaactaaacataggtg

SEQ ID NO: 1978 - DNA, 20 bp, artificial
  human FXR antisense
gtcttcttttcttctttcac

SEQ ID NO: 1979 - DNA, 20 bp, artificial
  human FXR antisense
ctaagtcttcttttcttctt

SEQ ID NO: 1980 - DNA, 20 bp, artificial
  human FXR antisense
ttggaataataggatgacga

SEQ ID NO: 1981 - DNA, 20 bp, artificial
  human FXR antisense
gaaacccaggttggaataat

SEQ ID NO: 1982 - DNA, 20 bp, artificial
  human FXR antisense
ccacaaacaacacacagctc

SEQ ID NO: 1983 - DNA, 20 bp, artificial
  human FXR antisense
tacacccctcacaggtcagt

SEQ ID NO: 1984 - DNA, 20 bp, artificial
  human FXR antisense
tgaattcttcttttaaaatt

SEQ ID NO: 1985 - DNA, 20 bp, artificial
  human FXR antisense
ttgctgtaagcagagcatac

SEQ ID NO: 1986 - DNA, 20 bp, artificial
  human FXR antisense
ctccatgacatcagcatctc

SEQ ID NO: 1987 - DNA, 20 bp, artificial
  human FXR antisense
tcacagagaagtggggtaaa

SEQ ID NO: 1988 - DNA, 20 bp, artificial
  human FXR antisense
ggtacaagtgaaataaagga

SEQ ID NO: 1989 - DNA, 20 bp, artificial
  human FXR antisense
atgacgaggaaatctgtggt

SEQ ID NO: 1990 - DNA, 20 bp, artificial
  human FXR antisense
gggggtagaaacccaggttg

SEQ ID NO: 1991 - DNA, 20 bp, artificial
  human FXR antisense
aaacctttacacccctcaca

SEQ ID NO: 1992 - DNA, 20 bp, artificial
  human FXR antisense
aagagtctgttgatctgggg

SEQ ID NO: 1993 - DNA, 20 bp, artificial
  human FXR antisense
cgtcaaaatgagaaaatttt

SEQ ID NO: 1994 - DNA, 20 bp, artificial
  human FXR antisense
tatattcatcagagatacca

SEQ ID NO: 1995 - DNA, 20 bp, artificial
  human FXR antisense
catgtaattacaacataaat

SEQ ID NO: 1996 - DNA, 20 bp, artificial
  human FXR antisense
tcttgaggaaatgtccagaa

SEQ ID NO: 1997 - DNA, 20 bp, artificial
  human FXR antisense
aacccaggttggaataatag

SEQ ID NO: 1998 - DNA, 20 bp, artificial
  human FXR antisense
aaacccaggttggaataata

SEQ ID NO: 1999 - DNA, 20 bp, artificial
  human FXR antisense
caggagagtaccactcttca

SEQ ID NO: 2000 - DNA, 20 bp, artificial
  human FXR antisense
agaaacctttacacccctca

SEQ ID NO: 2001 - DNA, 20 bp, artificial
  human FXR antisense
gagaaaattttcttctgcac

SEQ ID NO: 2002 - DNA, 20 bp, artificial
  human FXR antisense
ggtgaggaggaggagagagt

SEQ ID NO: 2003 - DNA, 20 bp, artificial
  human FXR antisense
aagtttcatcttgaggaaat

SEQ ID NO: 2004 - DNA, 20 bp, artificial
  human FXR antisense
gtcttcaaaaaaaactccaa

SEQ ID NO: 2005 - DNA, 20 bp, artificial
  human FXR antisense
aggatgacgaggaaatctgt

SEQ ID NO: 2006 - DNA, 20 bp, artificial
  human FXR antisense
agtttctccctggtagagag

SEQ ID NO: 2007 - DNA, 20 bp, artificial
  human FXR antisense
atacttttataaaaactaaa

SEQ ID NO: 2008 - DNA, 20 bp, artificial
  human FXR antisense
agaaaaagaaaattcatctg

SEQ ID NO: 2009 - DNA, 20 bp, artificial
  human FXR antisense
ctgtggttgaacttggggaa

SEQ ID NO: 2010 - DNA, 20 bp, artificial
  human FXR antisense
taggatgacgaggaaatctg

SEQ ID NO: 2011 - DNA, 20 bp, artificial
  human FXR antisense
tgggggtagaaacccaggtt

SEQ ID NO: 2012 - DNA, 20 bp, artificial
  human FXR antisense
ttactctccatgacatcagc

SEQ ID NO: 2013 - DNA, 20 bp, artificial
  human FXR antisense
ctatctggagacaggataac

SEQ ID NO: 2014 - DNA, 20 bp, artificial
  human FXR antisense
actctccatgacatcagcat

SEQ ID NO: 2015 - DNA, 20 bp, artificial
  human FXR antisense
aacttggggaaactgaacat

SEQ ID NO: 2016 - DNA, 20 bp, artificial
  human FXR antisense
tgctgtaagcagagcatact

SEQ ID NO: 2017 - DNA, 20 bp, artificial
  human FXR antisense
gaacttggggaaactgaaca

SEQ ID NO: 2018 - DNA, 20 bp, artificial
  human FXR antisense
aggttggaataataggatga

SEQ ID NO: 2019 - DNA, 20 bp, artificial
  human FXR antisense
acccaggttggaataatagg

SEQ ID NO: 2020 - DNA, 20 bp, artificial
  human FXR antisense
ggggtagaaacccaggttgg

SEQ ID NO: 2021 - DNA, 20 bp, artificial
  human FXR antisense
tacactgaatttcagttaac

SEQ ID NO: 2022 - DNA, 20 bp, artificial
  human FXR antisense
gaagtttcttattgaaaatc

SEQ ID NO: 2023 - DNA, 20 bp, artificial
  human FXR antisense
tacttttataaaaactaaac

SEQ ID NO: 2024 - DNA, 20 bp, artificial
  human FXR antisense
aatacttttataaaaactaa

SEQ ID NO: 2025 - DNA, 20 bp, artificial
  human FXR antisense
gggtacaagtgaaataaagg

SEQ ID NO: 2026 - DNA, 20 bp, artificial
  human FXR antisense
gaggtgaggaggaggagaga

SEQ ID NO: 2027 - DNA, 20 bp, artificial
  human FXR antisense
acaccaaataaattttcaga

SEQ ID NO: 2028 - DNA, 20 bp, artificial
  human FXR antisense
ggtagaaacccaggttggaa

SEQ ID NO: 2029 - DNA, 20 bp, artificial
  human FXR antisense
tgctgggggtagaaacccag

SEQ ID NO: 2030 - DNA, 20 bp, artificial
  human FXR antisense
cactgaattcttcttttaaa

SEQ ID NO: 2031 - DNA, 20 bp, artificial
  human FXR antisense
acttttataaaaactaaaca

SEQ ID NO: 2032 - DNA, 20 bp, artificial
  human FXR antisense
cccaatacttttataaaaac

SEQ ID NO: 2033 - DNA, 20 bp, artificial
  human FXR antisense
gttccccaatacttttataa

SEQ ID NO: 2034 - DNA, 20 bp, artificial
  human FXR antisense
acaacataaatattcatcaa

SEQ ID NO: 2035 - DNA, 20 bp, artificial
  human FXR antisense
ggagacaatgaggtgaggag

SEQ ID NO: 2036 - DNA, 20 bp, artificial
  human FXR antisense
aattttcagaaaaagaaaat

SEQ ID NO: 2037 - DNA, 20 bp, artificial
  human FXR antisense
ttacactgaatttcagttaa

SEQ ID NO: 2038 - DNA, 20 bp, artificial
  human FXR antisense
aaattttcttctgcactgaa

SEQ ID NO: 2039 - DNA, 20 bp, artificial
  human FXR antisense
aggaggaggagagagtctcg

SEQ ID NO: 2040 - DNA, 20 bp, artificial
  human FXR antisense
acttggggaaactgaacatt

SEQ ID NO: 2041 - DNA, 20 bp, artificial
  human FXR antisense
tcttctgcactgaattcttc

SEQ ID NO: 2042 - DNA, 20 bp, artificial
  human FXR antisense
tataaaaactaaacataggt

SEQ ID NO: 2043 - DNA, 20 bp, artificial
  human FXR antisense
ctgaagcttctctactgcct

SEQ ID NO: 2044 - DNA, 20 bp, artificial
  human FXR antisense
tactctccatgacatcagca

SEQ ID NO: 2045 - DNA, 20 bp, artificial
  human FXR antisense
aacataaatattcatcaaga

SEQ ID NO: 2046 - DNA, 20 bp, artificial
  human FXR antisense
taattacaacataaatattc

SEQ ID NO: 2047 - DNA, 20 bp, artificial
  human FXR antisense
actgaattcttcttttaaaa

SEQ ID NO: 2048 - DNA, 20 bp, artificial
  human FXR antisense
gcactgaattcttcttttaa

SEQ ID NO: 2049 - DNA, 20 bp, artificial
  human FXR antisense
ttcagaaaaagaaaattcat

SEQ ID NO: 2050 - DNA, 20 bp, artificial
  human FXR antisense
gaaataaaggaaagttatac

SEQ ID NO: 2051 - DNA, 20 bp, artificial
  human FXR antisense
tgaggaaatgtccagaagaa

SEQ ID NO: 2052 - DNA, 20 bp, artificial
  human FXR antisense
caaaaaaaactccaaagtgt

SEQ ID NO: 2053 - DNA, 20 bp, artificial
  human FXR antisense
aaatgggaatgttcaatgag

SEQ ID NO: 2054 - DNA, 20 bp, artificial
  human FXR antisense
agaaaattttcttctgcact

SEQ ID NO: 2055 - DNA, 20 bp, artificial
  human FXR antisense
ggagacaggcaaagtgttga

SEQ ID NO: 2056 - DNA, 20 bp, artificial
  human FXR antisense
aggtgaggaggaggagagag

SEQ ID NO: 2057 - DNA, 20 bp, artificial
  human FXR antisense
ggggaaactgaacattgctg

SEQ ID NO: 2058 - DNA, 20 bp, artificial
  human FXR antisense
gaaacctttacacccctcac

SEQ ID NO: 2059 - DNA, 20 bp, artificial
  human FXR antisense
ctggagacaggataacaatt

SEQ ID NO: 2060 - DNA, 20 bp, artificial
  human FXR antisense
aggaaatgtccagaagaaat

SEQ ID NO: 2061 - DNA, 20 bp, artificial
  human FXR antisense
tctgtggttgaacttgggga

SEQ ID NO: 2062 - DNA, 20 bp, artificial
  human FXR antisense
acacccctcacaggtcagtg

SEQ ID NO: 2063 - DNA, 20 bp, artificial
  human FXR antisense
cagaaaaagaaaattcatct

SEQ ID NO: 2064 - DNA, 20 bp, artificial
  human FXR antisense
cttgaggaaatgtccagaag

SEQ ID NO: 2065 - DNA, 20 bp, artificial
  human FXR antisense
aaattttcagaaaaagaaaa

SEQ ID NO: 2066 - DNA, 20 bp, artificial
  human FXR antisense
taaattttcagaaaaagaaa

SEQ ID NO: 2067 - DNA, 20 bp, artificial
  human FXR antisense
caaacaacacacagctcatc

SEQ ID NO: 2068 - DNA, 20 bp, artificial
  human FXR antisense
cagttccccaatacttttat

SEQ ID NO: 2069 - DNA, 20 bp, artificial
  human FXR antisense
acaatgaggtgaggaggagg

SEQ ID NO: 2070 - DNA, 20 bp, artificial
  human FXR antisense
cttcaaaaaaaactccaaag

SEQ ID NO: 2071 - DNA, 20 bp, artificial
  human FXR antisense
acttccaggttctgtcccag

SEQ ID NO: 2072 - DNA, 20 bp, artificial
  human FXR antisense
tcagaaaaagaaaattcatc

SEQ ID NO: 2073 - DNA, 20 bp, artificial
  human FXR antisense
ctgaatttcagttaacaagc

SEQ ID NO: 2074 - DNA, 20 bp, artificial
  human FXR antisense
ggaagtttcttattgaaaat

SEQ ID NO: 2075 - DNA, 20 bp, artificial
  human FXR antisense
cttttataaaaactaaacat

SEQ ID NO: 2076 - DNA, 20 bp, artificial
  human FXR antisense
ataaattttcagaaaaagaa

SEQ ID NO: 2077 - DNA, 20 bp, artificial
  human FXR antisense
agttccccaatacttttata

SEQ ID NO: 2078 - DNA, 20 bp, artificial
  human FXR antisense
caaataaattttcagaaaaa

SEQ ID NO: 2079 - DNA, 20 bp, artificial
  human FXR antisense
aaaacaccaaataaattttc

SEQ ID NO: 2080 - DNA, 20 bp, artificial
  human FXR antisense
gagagtctcagctggcatac

SEQ ID NO: 2081 - DNA, 20 bp, artificial
  human FXR antisense
tggggaaactgaacattgct

SEQ ID NO: 2082 - DNA, 20 bp, artificial
  human FXR antisense
ttccctagttcaacagatag

SEQ ID NO: 2083 - DNA, 20 bp, artificial
  human FXR antisense
cacaaacaacacacagctca

SEQ ID NO: 2084 - DNA, 20 bp, artificial
  human FXR antisense
cactgaatttcagttaacaa

SEQ ID NO: 2085 - DNA, 20 bp, artificial
  human FXR antisense
ttccccaatacttttataaa

SEQ ID NO: 2086 - DNA, 20 bp, artificial
  human FXR antisense
tcttcaaaaaaaactccaaa

SEQ ID NO: 2087 - DNA, 20 bp, artificial
  human FXR antisense
gttaaaacaccaaataaatt

SEQ ID NO: 2088 - DNA, 20 bp, artificial
  human FXR antisense
ggtcagtgcattatagtggt

SEQ ID NO: 2089 - DNA, 20 bp, artificial
  human FXR antisense
tgaggtgaggaggaggagag

SEQ ID NO: 2090 - DNA, 20 bp, artificial
  human FXR antisense
gctgggggtagaaacccagg

SEQ ID NO: 2091 - DNA, 20 bp, artificial
  human FXR antisense
tcagttccccaatactttta

SEQ ID NO: 2092 - DNA, 20 bp, artificial
  human FXR antisense
gaagaaatccaggaaactaa

SEQ ID NO: 2093 - DNA, 20 bp, artificial
  human FXR antisense
aacaccaaataaattttcag

SEQ ID NO: 2094 - DNA, 20 bp, artificial
  human FXR antisense
gatgacgaggaaatctgtgg

SEQ ID NO: 2095 - DNA, 20 bp, artificial
  human FXR antisense
gaaaattttcttctgcactg

SEQ ID NO: 2096 - DNA, 20 bp, artificial
  human FXR antisense
caggtcagtgcattatagtg

SEQ ID NO: 2097 - DNA, 20 bp, artificial
  human FXR antisense
cacccctcacaggtcagtgc

SEQ ID NO: 2098 - DNA, 20 bp, artificial
  human FXR antisense
ttataaaaactaaacatagg

SEQ ID NO: 2099 - DNA, 20 bp, artificial
  human FXR antisense
gaggaggaggagagagtctc

SEQ ID NO: 2100 - DNA, 20 bp, artificial
  human FXR antisense
acaaacaacacacagctcat

SEQ ID NO: 2101 - DNA, 20 bp, artificial
  human FXR antisense
ggatgacgaggaaatctgtg

SEQ ID NO: 2102 - DNA, 20 bp, artificial
  human FXR antisense
gtcagtgcattatagtggta

SEQ ID NO: 2103 - DNA, 20 bp, artificial
  human FXR antisense
ttaaaacaccaaataaattt

SEQ ID NO: 2104 - DNA, 20 bp, artificial
  human FXR antisense
aataaattttcagaaaaaga

SEQ ID NO: 2105 - DNA, 20 bp, artificial
  human FXR antisense
aggtcagtgcattatagtgg

SEQ ID NO: 2106 - DNA, 20 bp, artificial
  human FXR antisense
aagaaatccaggaaactaag

SEQ ID NO: 2107 - DNA, 20 bp, artificial
  human FXR antisense
ataaaatgtagaagagtctg

SEQ ID NO: 2108 - DNA, 20 bp, artificial
  human FXR antisense
gtgaggaggaggagagagtc

SEQ ID NO: 2109 - DNA, 20 bp, artificial
  human FXR antisense
caacataaatattcatcaag

SEQ ID NO: 2110 - DNA, 20 bp, artificial
  human FXR antisense
ctgttaaaacaccaaataaa

SEQ ID NO: 2111 - DNA, 20 bp, artificial
  human FXR antisense
actgaatttcagttaacaag

SEQ ID NO: 2112 - DNA, 20 bp, artificial
  human FXR antisense
gtggttgaacttggggaaac

SEQ ID NO: 2113 - DNA, 20 bp, artificial
  human FXR antisense
atgaggtgaggaggaggaga

SEQ ID NO: 2114 - DNA, 20 bp, artificial
  human FXR antisense
tgttaaaacaccaaataaat

SEQ ID NO: 2115 - DNA, 20 bp, artificial
  human FXR antisense
tctatctggagacaggataa

SEQ ID NO: 2116 - DNA, 20 bp, artificial
  human FXR antisense
taaaacaccaaataaatttt

SEQ ID NO: 2117 - DNA, 20 bp, artificial
  human FXR antisense
aaacaccaaataaattttca

SEQ ID NO: 2118 - DNA, 20 bp, artificial
  human FXR antisense
tgaacttggggaaactgaac

SEQ ID NO: 2119 - DNA, 20 bp, artificial
  human FXR antisense
tgtggttgaacttggggaaa

SEQ ID NO: 2120 - DNA, 20 bp, artificial
  human FXR antisense
ggttgaacttggggaaactg

SEQ ID NO: 2121 - DNA, 20 bp, artificial
  human FXR antisense
aaataaattttcagaaaaag

SEQ ID NO: 2122 - DNA, 20 bp, artificial
  human FXR antisense
ttcaaaaaaaactccaaagt

SEQ ID NO: 2123 - DNA, 20 bp, artificial
  human FXR antisense
tggttgaacttggggaaact

SEQ ID NO: 2124 - DNA, 20 bp, artificial
  human FXR antisense
gtgcattatagtggtatcca

SEQ ID NO: 2125 - DNA, 20 bp, artificial
  human FXR antisense
atattcatcagagataccac

SEQ ID NO: 2126 - DNA, 20 bp, artificial
  human FXR antisense
tattcatcagagataccact

SEQ ID NO: 2127 - DNA, 20 bp, artificial
  human FXR antisense
aatgaggtgaggaggaggag

SEQ ID NO: 2128 - DNA, 20 bp, artificial
  human FXR antisense
ttgaacttggggaaactgaa

SEQ ID NO: 2129 - DNA, 20 bp, artificial
  human FXR antisense
tgcattatagtggtatccag

SEQ ID NO: 2130 - DNA, 20 bp, artificial
  human FXR antisense
attcatcagagataccacta

SEQ ID NO: 2131 - DNA, 20 bp, artificial
  human FXR antisense
caatgaggtgaggaggagga

SEQ ID NO: 2132 - DNA, 20 bp, artificial
  human FXR antisense
tcagtgcattatagtggtat

SEQ ID NO: 2133 - DNA, 20 bp, artificial
  human FXR antisense
gttgaacttggggaaactga

SEQ ID NO: 2134 - DNA, 20 bp, artificial
  human FXR antisense
tccccaatacttttataaaa

SEQ ID NO: 2135 - DNA, 20 bp, artificial
  human FXR antisense
ccccaatacttttataaaaa

SEQ ID NO: 2136 - DNA, 20 bp, artificial
  human FXR antisense
tcaaaaaaaactccaaagtg

SEQ ID NO: 2137 - DNA, 20 bp, artificial
  human FXR antisense
agtgcattatagtggtatcc

SEQ ID NO: 2138 - DNA, 20 bp, artificial
  human FXR antisense
cagtgcattatagtggtatc

SEQ ID NO: 2139 - DNA, 20 bp, Artificial
  human FXR forward primer
ctgggtcgcctgactgaatt

SEQ ID NO: 2140 - DNA, 24 bp, Artificial
  human FXR reverse primer
ggtcgtttactctccatgacatca

SEQ ID NO: 2141 - DNA, 27 bp, Artificial
  human FXR PCR primer
cggacattcaatcatcaccacgctgag

SEQ ID NO: 2142 - DNA, 20 bp, Artificial
  human cyclophilin forward primer
cccaccgtgttcttcgacat

SEQ ID NO: 2143 - DNA, 22 bp, Artificial
  human cyclophilin reverse primer
tttctgctgtctttgggacctt

SEQ ID NO: 2144 - DNA, 24 bp, Artificial
  human cyclophilin PCR primer
cgcgtctcctttgagctgtttgca

SEQ ID NO: 2145 - DNA, 2218 bp, Homo sapiens
acgagactctctcctcctcctcacctcattgtctccccgacttatcctaatgcgaaattggattctgagcatttgtagcaaaatcgctgggatctggagaggaagactcagtccagaatcctcccagggccttgaaagtccatctctgacccaaaacaatccaaggaggtagaagacatcgtagaaggagtgaaagaagaaaagaagacttagaaacatagctcaaagtgaacactgcttctcttagtttcctggatttcttctggacatttcctcaagatgaaacttcagacactttggagttttttttgaagaccaccataaagaaagtgcatttcaattgaaaaatttggatgggatcaaaaatgaatctcattgaacattcccatttacctaccacagatgaattttctttttctgaaaatttatttggtgttttaacagaacaagtggcaggtcctctgggacagaacctggaagtggaaccatactcgcaatacagcaatgttcagtttccccaagttcaaccacagatttcctcgtcatcctattattccaacctgggtttctacccccagcagcctgaagagtggtactctcctggaatatatgaactcaggcgtatgccagctgagactctctaccagggagaaactgaggtagcagagatgcctgtaacaaagaagccccgcatgggcgcgtcagcagggaggatcaaaggggatgagctgtgtgttgtttgtggagacagagcctctggataccactataatgcactgacctgtgaggggtgtaaaggtttcttcaggagaagcattaccaaaaacgctgtgtacaagtgtaaaaacgggggcaactgtgtgatggatatgtacatgcgaagaaagtgtcaagagtgtcgactaaggaaatgcaaagagatgggaatgttggctgaatgcttgttaactgaaattcagtgtaaatctaagcgactgagaaaaaatgtgaagcagcatgcagatcagaccgtgaatgaagacagtgaaggtcgtgacttgcgacaagtgacctcgacaacaaagtcatgcagggagaaaactgaactcaccccagatcaacagactcttctacattttattatggattcatataacaaacagaggatgcctcaggaaataacaaataaaattttaaaagaagaattcagtgcagaagaaaattttctcattttgacggaaatggcaaccaatcatgtacaggttcttgtagaattcacaaaaaagctaccaggatttcagactttggaccatgaagaccagattgctttgctgaaagggtctgcggttgaagctatgttccttcgttcagctgagattttcaataagaaacttccgtctgggcattctgacctattggaagaaagaattcgaaatagtggtatctctgatgaatatataacacctatgtttagtttttataaaagtattggggaactgaaaatgactcaagaggagtatgctctgcttacagcaattgttatcctgtctccagatagacaatacataaaggatagagaggcagtagagaagcttcaggagccacttcttgatgtgctacaaaagttgtgtaagattcaccagcctgaaaatcctcaacactttgcctgtctcctgggtcgcctgactgaattacggacattcaatcatcaccacgctgagatgctgatgtcatggagagtaaacgaccacaagtttaccccacttctctgtgaaatctgggacgtgcagtgatggggattacaggggaggggtctagctcctttttctctctcatattaatctgatgtataactttcctttatttcacttgtacccagtttcactcaagaaatcttgatgaatatttatgttgtaattacatgtgtaacttccacaactgtaaatattgggctagatagaacaactttctctacattgtgttttaaaaggctccagggaatcctgcattctaattggcaagccctgtttgcctaattaaattgattgttacttcaattctatctgttgaactagggaaaatctcattttgctcatcttaccatattgcatatattttattaaagagttgtattcaatcttggcaataaagcaaacataatggcaacagaaaaaaaaaaaaaaaaaaaaaaaaaaaaaaaaaaaaaaaaaaaaaaaaaaaaaaaaaaaaaaaaaaaaaa

SEQ ID NO: 2146 - protein, 472 aa, Homo sapiens
MGSKMNLIEHSHLPTTDEFSFSENLFGVLTEQVAGPLGQNLEVEPYSQYSNVQFPQVQPQISSSSYYSNLGFYPQQPEEWYSPGIYELRRMPAETLYQGETEVAEMPVTKKPRMGASAGRIKGDELCVVCGDRASGYHYNALTCEGCKGFFRRSITKNAVYKCKNGGNCVMDMYMRRKCQECRLRKCKEMGMLAECLLTEIQCKSKRLRKNVKQHADQTVNEDSEGRDLRQVTSTTKSCREKTELTPDQQTLLHFIMDSYNKQRMPQEITNKILKEEFSAEENFLILTEMATNHVQVLVEFTKKLPGFQTLDHEDQIALLKGSAVEAMFLRSAEIFNKKLPSGHSDLLEERIRNSGISDEYITPMFSFYKSIGELKMTQEEYALLTAIVILSPDRQYIKDREAVEKLQEPLLDVLQKLCKIHQPENPQHFACLLGRLTELRTFNHHHAEMLMSWRVNDHKFTPLLCEIWDVQ

(19) United States
(12) Patent Application Publication
(10) Pub. No.: US 2006/0211640 A1
(43) Pub. Date: Sep. 21, 2006

(54) Antisense modulation of farnesoid X receptor expression

(76) Inventor: Christopher D. Kane, Waterford, CT (US)

Correspondence Address: Pfizer, Inc., 5th Floor, 575 Maryville Centre Drive, St. Louis, MO, 63141, US

(21) Appl. No.: 10/670,984

(22) Filed: Sep. 25, 2003

Related U.S. Application Data
(60) Provisional application No. 60/413,588, filed on Sep. 25, 2002.

(51) Int. Cl. A61K 48/00; C07H 21/02; C07F 9/6512

(52) U.S. Cl. 514/44; 514/81; 536/23.1; 544/244

(57) ABSTRACT

Antisense compounds, compositions, and methods are provided for modulating the expression of Farnesoid X Receptor (FXR). The compositions comprise antisense compounds, particularly antisense oligonucleotides, targeted to nucleic acids encoding FXR. Methods of using these compounds for modulation of FXR expression and for treatment of diseases associated with expression of FXR are provided.

The present application claims priority under Title 35, United States Code, §119 to U.S. Provisional application Ser. No. 60/413,588, filed Sep. 25, 2002, which is incorporated by reference in its entirety as if written herein.

### FIELD OF THE INVENTION

The present invention provides compositions and methods for modulating the expression of Farnesoid X Receptor (FXR) alternatively referred to as FXR, RIP14, NR1H4, and Bile Acid Receptor (BAR). In particular, this invention relates to antisense compounds, particularly oligonucleotides, specifically hybridizable with nucleic acids encoding FXR. Such oligonucleotides have been shown to modulate the expression of FXR.

### BACKGROUND OF THE INVENTION

Cholesterol is essential for a number of cellular processes, including membrane biogenesis and steroid hormone and bile acid biosynthesis. It is the building block for each of the major classes of lipoproteins found in cells of the human body. Accordingly, cholesterol biosynthesis and catabolism are highly regulated and coordinated processes. A number of diseases and/or disorders have been linked to alterations in cholesterol metabolism or catabolism including atherosclerosis, gallstone formation, and ischemic heart disease. An understanding of the pathways involved in cholesterol homeostasis is essential to the development of useful therapeutics for treatment of these diseases and disorders.

The metabolism of cholesterol to bile acids represents a major pathway for cholesterol elimination from the body, accounting for approximately half of the daily excretion. These cholesterol metabolites are formed in the liver and secreted into the duodenum of the intestine, where they have important roles in the solubilization and absorption of dietary lipids and vitamins. Most bile acids (approximately 95%) are subsequently reabsorbed in the ileum and returned to the liver via the enterohepatic circulatory system.

Cytochrome P450 7A (CYP7A) is a liver specific enzyme that catalyzes the first and rate-limiting step in one of the two pathways for bile acid biosynthesis (Chiang, J. Y. L. 1998 Front. Biosci. 3:176-193; Russell, D. W. and K. D. Setchell. 1992 Biochemistry 31:4737-4749). The gene encoding CYP7A is regulated by a variety of endogenous, small, lipophilic molecules including steroid and thyroid hormones, cholesterol, and bile acids. Notably, CYP7A expression is stimulated by cholesterol feeding and repressed by bile acids. Thus, CYP7A expression is both positively (stimulated or induced) and negatively (inhibited or repressed) regulated.

CYP7A expression is regulated by several members of the nuclear receptor family of ligand-activated transcription factors (Chiang, J. Y. L. 1998 Front. Biosci. 3:176-193; Gustafsson, J. A. 1999 Science 284:1285-1286; Russell, D. W. 1999 Cell 97:539-542). Recently, two nuclear receptors, the liver X receptor (LXR; NR1H3; Apfel, R. et al. 1994 Mol. Cell. Biol. 14:7025-7035; Willy, P. J. et al. 1995 Genes Devel. 9:1033-1045) and the farnesoid X receptor (FXR; NR1H4; Forman, B. M. et al. 1995 Cell 81:687-693; Seol, W. et al. 1995 Mol. Endocrinol. 9:72-85) were implicated in the positive and negative regulation of CYP7A (Peet, D. J. et al. 1998 Curr. Opin. Genet. Develop. 8:571-575; Russell, D. W. 1999 Cell 97:539-542). Both LXR and FXR are abundantly expressed in the liver and bind to their cognate hormone response elements as heterodimers with the 9-cis retinoic acid receptor, RXR (Mangelsdorf, D. J. and R. M. Evans. 1995 Cell 83:841-850).

LXR is activated by the cholesterol derivative 24,25(S) epoxycholesterol and binds to a response element in the CYP7A promoter (Lehmann, J. M. et al. 1997 J. Biol. Chem. 272:3137-3140). CYP7A is not induced in response to cholesterol feeding in mice lacking LXR (Peet, D. J. et al. 1998 Cell 93:693-704). Moreover, these animals accumulate massive amounts of cholesterol in their livers when fed a high cholesterol diet. These studies establish LXR as a cholesterol sensor responsible for positive regulation of CYP7A expression.

Bile acids stimulate the expression of genes involved in bile acid transport such as the intestinal bile acid binding protein (I-BABP) and repress CYP7A as well as other genes involved in bile acid biosynthesis such as CYP8B (which converts chenodeoxycholic acid to cholic acid), and CYP27 (which catalyzes the first step in the alternative pathway for bile acid synthesis; Javitt, N. B. 1994 FASEB J. 8:1308-1311; Russell, D. W. and K. D. Setchell 1992 Biochemistry 31:4737-4749). Recently, FXR was shown to be a bile acid receptor (Makishima, M. et al. 1999 Science 284:1362-1365; Parks, D. J. et al. 1999 Science 284:1365-1368; Wang, H. 1999 Mol. Cell 3:543-553). Several different bile acids, including chenodeoxycholic acid and its glycine and taurine conjugates were demonstrated to bind to and activate FXR at physiologic concentrations. In addition, DNA response elements for the FXR/RXR heterodimer were identified in both the human and mouse I-BABP promoters, indicating that FXR mediates positive effects of bile acids on I-BABP expression (Grober, J. et al. 1999 J. Biol. Chem. 274:29749-29754; Makishima, M. et al. 1999 Science 284:1362-1365). Further, the rank order of bile acids that activate FXR correlates with that for repression of CYP7A in a hepatocyte-derived cell line (Makishima, M. et al. 1999 Science 284:1362-1365). Thus, these studies indicate that FXR also has a role in the negative effects of bile acids on gene expression.

However, the molecular mechanism of bile acid mediated repression of CYP7A, and specifically the role of FXR in this process is unclear. Since the CYP7A promoter lacks a strong FXR/RXR binding site (Chiang, J. Y. and D. Stroup. 1994 J. Biol. Chem. 269:17502-17507; Chiang, J. Y. et al. 2000 J. Biol. Chem. 275:10918-10924), it is unlikely that the effect is from the direct interaction of FXR

An additional nuclear receptor also involved in the expression of CYP7A is the liver receptor homolog-1 (LRH1, also called CPF, hB1F, and NR5A2), a monomeric orphan nuclear receptor that functions as a tissue specific transcription factor (Becker-Andre et al 1993 Biochem. Biophys. Res. Comm. 194:1371-1379; Galarneau et al 1996 Mol. Cell. Biol. 16:3853-3865; Li et al 1998 J. Biol. Chem. 273:29022-29031; Nitta et al 1999 Proc. Natl. Acad. Sci. USA 96: 6660-6665). High level expression of LRH1 has been shown in the liver, pancreas, and ovary, with less abundant expression in the colon, intestine, and the adrenal gland (Nitta et al 1999 Proc. Natl. Acad. Sci. USA 96: 6660-6665; Li et al 1998 J. Biol. Chem. 273:29022-29031; Repa and Mangelsdorf 2000 Ann Rev. Cell. Dev, Wang et al 2001 J. Mol. Endo. 27:255-258). Whereas the biological role for LRH-1 is still emerging, it is clear that LRH-1 is required for hepatic expression of CYP7A and maximizes this expression via synergizing with LXR (Nitta et al 1999 Proc. Natl. Acad. Sci. USA 96: 6660-6665; Lu et al 2000 Mol. Cell 6:507-517).

LRH1 can also induce the expression of short heterodimer partner (SHP, NR0B2), an orphan nuclear receptor that represses transcription and inhibits the function of other nuclear receptors (Seol et al 1996 Science 272:1336-1339, Johansson et al 1999 J. Biol. Chem. 274:345-353, Lee et al 1999 J. Biol. Chem. 274:20869-20873). SHP is also a direct gene target of FXR and SHP expression is upregulated via FXR agonist compounds including the bile acid CDCA and the synthetic FXR agonist GW4064 (Lu et al 2000 Mol. Cell 6:507-517, Goodwin et al 2000 Mol. Cell 6: 517-526). Therefore, FXR agonists indirectly repress CYP7a via induction of the repressor SHP, which subsequently binds to and represses the transcriptional activity of LRH1 on the CYP7A promoter (Lu et al 2000 Mol. Cell 6:507-517; Goodwin et al 2000 Mol. Cell 6: 517-526). These finding demonstrate the existence of complex regulatory cascades involving five different nuclear receptors including FXR, RXR, LXR, LRH, and SHP, that coordinately govern bile acid synthesis and cholesterol and lipid homeostasis.

Recent findings concerning human loss of function mutations in the CYP7a locus as well as pharmacological studies describing the discovery of a naturally occurring FXR antagonist point to the potential beneficial therapeutic indications of an FXR antagonist. Studies performed by Pullinger et al (2002 J. Clin Invest. 110: 109-117) show that human patients harboring a loss of function mutation in CYP7a present with a hypercholesterolemic phenotype coupled with profound resistance to HMG-CoA reductase inhibitors (also known generically as “statins”). Additionally, two independent groups have reported that a natural product termed Guggulsterone functions as an FXR antagonist. Guggulsterone represses SHP expression and SHP-dependent repression of CYP7a, resulting in lowered LDL and triglyceride in mouse models (Urizar et al 2002 Science: 1703-1706; Wu, J. et al 2002 Mol Endocrinol. 16:1590-7). Given these results, any genetic or pharmacological means of elevating CYP7a expression or activity in humans would be likely to have a beneficial therapeutic effect upon cholesterol metabolism and homeostasis. For example, the ability to inhibit FXR expression and therefore FXR-dependent upregulation of SHP should prevent bile acid mediated feedback repression of CYP7a.

Despite the variety of Farnesoid X Receptor inhibitors disclosed in the art, there still remains a need for therapeutic agents capable of effectively and specifically inhibiting the function of the Farnesoid X Receptor (FXR)

Antisense technology is emerging as an effective means for reducing the expression of specific gene products and may therefore prove to be uniquely useful in a number of therapeutic, diagnostic, and research applications for the modulation of FXR expression.

### SUMMARY OF THE INVENTION

The present invention is directed to antisense compounds, particularly oligonucleotides, which are targeted to a nucleic acid encoding Farnesoid X Receptor (FXR), and which modulate the expression of FXR. Pharmaceutical and other compositions comprising the antisense compounds of the invention are also provided. Further provided are methods of modulating the expression of FXR in cells or tissues comprising contacting said cells or tissues with one or more of the antisense compounds or compositions of the invention. Further provided are methods of treating an animal, particularly a human, suspected of having or being prone to a disease or condition associated with expression of FXR by administering a therapeutically or prophylactically effective amount of one or more of the antisense compounds or compositions of the invention.

### DETAILED DESCRIPTION OF THE INVENTION

The present invention employs oligomeric antisense compounds, particularly oligonucleotides, for use in modulating the function of nucleic acid molecules encoding FXR, ultimately modulating the amount of FXR produced. This is accomplished by providing antisense compounds, which specifically hybridize with one or more nucleic acids encoding FXR. As used herein, the terms “target nucleic acid” and “nucleic acid encoding FXR” encompass DNA encoding FXR, RNA (including pre-mRNA and mRNA) transcribed from such DNA, and also cDNA derived from such RNA. The specific hybridization of an oligomeric compound with its target nucleic acid interferes with the normal function of the nucleic acid. This modulation of function of a target nucleic acid by compounds, which specifically hybridize to it, is generally referred to as “antisense”. The functions of DNA to be interfered with include replication and transcription. The functions of RNA to be interfered with include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of FXR. In the context of the present invention, “modulation” means either an increase (stimulation) or a decrease (inhibition) in the expression of a gene. In the context of the present invention, inhibition is the preferred form of modulation, of gene expression and mRNA is a preferred target.

It is preferred to target specific nucleic acids for antisense. “Targeting” an antisense compound to a particular nucleic acid, in the context of this invention, is a multistep process. The process usually begins with the identification of a nucleic acid sequence whose function is to be modulated. This may be, for example, a cellular gene (or mRNA transcribed from the gene) whose expression is associated with a particular disorder or disease state, or a nucleic acid molecule from an infectious agent. In the present invention, the target is a nucleic acid molecule encoding FXR. The targeting process also includes determination of a site or sites within this gene for the antisense interaction to occur such that the desired effect, e.g., detection or modulation of expression of the protein, will result. Within the context of the present invention, a preferred intragenic site is the region encompassing the translation initiation or termination codon of the open reading frame (ORF) of the gene. Since, as is known in the art, the translation initiation codon is typically 5′-AUG (in transcribed mRNA molecules; 5′-ATG in the corresponding DNA molecule), the translation initiation codon is also referred to as the “AUG codon,” the “start codon” or the “AUG start codon”. A minority of genes have a translation initiation codon having the RNA sequence 5′-GUG, 5′-UUG or 5′-CUG, and 5′-AUA, 5′-ACG and 5′-CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA molecule transcribed from a gene encoding FXR, regardless of the sequence(s) of such codons.

It is also known in the art that a translation termination codon (or “stop codon”) of a gene may have one of three sequences, i.e. 5′-UAA, 5′-UAG and 5′-UGA (the corresponding DNA sequences are 5′-TAA, 5′-TAG and 5′-TGA, respectively). The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon.

The open reading frame (ORF) or “coding region,” which is known in the art to refer to the region between the translation initiation codon and the translation termination codon, is also a region which may be targeted effectively. Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA or corresponding nucleotides on the gene, and the 3′ untranslated region (3′UTR), known in the art to refer to the portion of an mRNA in the 3′ direction from the translation termination codon, and thus including nucleotides between the translation termination codon and 3′ end of an mRNA or corresponding nucleotides on the gene. The 5′ cap of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap. The 5′ cap region may also be a preferred target region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. mRNA splice sites, i.e., intron-exon junctions, may also be preferred target regions, and are particularly useful in situations where aberrant splicing is implicated in disease, or where an overproduction of a particular mRNA splice product is implicated in disease. Aberrant fusion junctions due to rearrangements or deletions are also preferred targets. It has also been found that introns can also be effective, and therefore preferred, target regions for antisense compounds targeted, for example, to DNA or pre-mRNA.

Once one or more target sites have been identified, oligonucleotides are chosen which are sufficiently complementary to the target, i.e., hybridize sufficiently well and with sufficient specificity, to give the desired effect.

In the context of this invention, “hybridization” means hydrogen bonding, which may be Watson-Crick, Hoogsteen, or reversed Hoogsteen hydrogen bonding, between complementary nucleoside or nucleotide bases. For example, adenine and thymine are complementary nucleobases, which pair through the formation of hydrogen bonds. “Complementary,” as used herein, refers to the capacity for precise pairing between two nucleotides. For example, if a nucleotide at a certain position of an oligonucleotide is capable of hydrogen bonding with a nucleotide at the same position of a DNA or RNA molecule, then the oligonucleotide and the DNA or RNA are considered to be complementary to each other at that position. The oligonucleotide and the DNA or RNA are complementary to each other when a sufficient number of corresponding positions in each molecule are occupied by nucleotides which can hydrogen bond with each other. Thus, “specifically hybridizable” and “complementary” are terms which are used to indicate a sufficient degree of complementarity or precise pairing such that stable and specific binding occurs between the oligonucleotide and the DNA or RNA target. It is understood in the art that the sequence of an antisense compound need not be 100% complementary to that of its target nucleic acid to be specifically hybridizable. An antisense compound is specifically hybridizable when binding of the compound to the target DNA or RNA molecule interferes with the normal function of the target DNA or RNA to cause a loss of utility, and there is a sufficient degree of complementarity to avoid non-specific binding of the antisense compound to non-target sequences under conditions in which specific binding is desired, i.e., under physiological conditions in the case of in vivo assays or therapeutic treatment, and in the case of in vitro assays, under conditions in which the assays are performed.

Antisense compounds are commonly used as research reagents and diagnostics. For example, antisense oligonucleotides, which are able to inhibit gene expression with exquisite specificity, are often used by those of ordinary skill to elucidate the function of particular genes. Antisense compounds are also used, for example, to distinguish between functions of various members of a biological pathway. Antisense modulation has, therefore, been harnessed for research use.

The specificity and sensitivity of antisense is also harnessed by those of skill in the art for therapeutic uses. Antisense oligonucleotides have been employed as therapeutic moieties in the treatment of disease states in animals and man. Antisense oligonucleotides have been safely and effectively administered to humans and numerous clinical trials are presently underway. It is thus established that oligonucleotides can be useful therapeutic modalities that can be configured to be useful in treatment regimes for treatment of cells, tissues and animals, especially humans. In the context of this invention, the term “oligonucleotide” refers to an oligomer or polymer of ribonucleic acid (RNA) or deoxyribonucleic acid (DNA) or mimetics thereof. This term includes oligonucleotides composed of naturally occurring nucleobases, sugars and covalent internucleoside (backbone) linkages as well as oligonucleotides having non-naturally occurring portions which function similarly. Such modified or substituted oligonucleotides are often preferred over native forms because of desirable properties such as, for example, enhanced cellular uptake, enhanced affinity for nucleic acid target and increased stability in the presence of nucleases.

While antisense oligonucleotides are a preferred form of antisense compound, the present invention comprehends other oligomeric antisense compounds, including but not limited to oligonucleotide mimetics such as are described below. The antisense compounds in accordance with this invention preferably comprise from about 8 to about 30 nucleobases (i.e. from about 8 to about 30 linked nucleo sides). Particularly preferred antisense compounds are antisense oligonucleotides, even more preferably those comprising from about 12 to about 25 nucleobases. As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′, or 5′ hydroxyl moiety of the sugar. In forming oligonucleotides, the phosphate groups covalently link adjacent nucleosides to one another to form a linear polymeric compound. In turn the respective ends of this linear polymeric structure can be further joined to form a circular structure, however, open linear structures are generally preferred. Within the oligonucleotide structure, the phosphate groups are commonly referred to as forming the internucleoside backbone of the oligonucleotide. The normal I linkage or backbone of RNA and DNA is a 3′ to 5′ phosphodiester linkage.

Specific examples of preferred antisense compounds useful in this invention include oligonucleotides containing modified backbones or non-natural internucleoside linkages. As defined in this specification, oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Preferred modified oligonucleotide backbones include, for example, phosphorothioates, chiral phosphorothioates, phosphorodithioates, phosphotriesters, aminoalkylphosphotriesters, methyl and other alkyl phosphonates including 3′alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050, each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, ach of which is herein incorporated by reference.

In other preferred oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al. (Science, 1991, 254, 1497-1500).

Most preferred embodiments of the invention are oligonucleotides with phosphorothioate backbones and oligonucleosides with heteroatom backbones, and in particular —CH₂—NH—O—CH₂—, —CH₂—N(CH₃)—O—CH₂— [known as a methylene (methylimino) or MMI backbone], —CH₂—O—N(CH₃)—CH₂—, —CH₂N(CH₃)—N(CH₃)—CH₂— and —O—N(CH₃)—CH₂—CH₂— [wherein the native phosphodiester backbone is represented as —O—P—O—CH₂—] of the above referenced U.S. Pat. No. 5,489,677, and the amide backbones of the above referenced U.S. Pat. No. 5,602,240. Also preferred are oligonucleotides having morpholino backbone structures of the above-referenced U.S. Pat. No. 5,034,506.

Modified oligonucleotides may also contain one or more substituted sugar moieties. Preferred oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly preferred are O[(CH₂)ₙO]ₘCH₃, O(CH₂)ₙ, OCH₃, O(CH₂)ₙNH₂, O(CH₂)ₙCH₃, O(CH₂)ₙONH₂, and O(CH₂ₙON[(CH₂)ₙCH₃)]₂ where n and m are from 1 to about 10. Other preferred oligonucleotides comprise one of the following at the 2′ position: C₁ to C₁₀, (lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. A preferred modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further preferred modification includes 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N (CH₂)₂, also described in examples herein below.

Other preferred modifications include 2′-methoxy (2′-OCH₃), 2′-aminopropoxy (2′-OCH₂CH₂CH₂NH₂), and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, each of which is herein incorporated by reference in its entirety.

Oligonucleotides may also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B. ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., eds, Antisense Research and Applications, CRC Press, Boca Raton, 1993, pp. 276-278) and are presently preferred base substitutions, even more particularly when combined with 2′-O-methoxyethyl sugar modifications.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,12′, 5,596,091; 5,614,617; 5,750,692, and 5,681,941, each of which is herein incorporated by reference.

Another modification of the oligonucleotides of the invention involves chemically linking to the oligonucleotide one or more moieties or conjugates, which enhance the activity, cellular distribution, or cellular uptake of the oligonucleotide. Such moieties include but are not limited to lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937).

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

It is not necessary for all positions in a given compound to be uniformly modified, and in fact more than one of the aforementioned modifications may be incorporated in a single compound or even at a single nucleoside within an oligonucleotide. The present invention also includes antisense compounds, which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease, which cleaves the RNA strand of RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference in its entirety.

The antisense compounds used in accordance with this invention may be conveniently, and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The antisense compounds of the invention are synthesized in vitro and do not include antisense compositions of biological origin, or genetic vector constructs designed to direct the in vivo synthesis of antisense molecules. The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The antisense compounds of the invention encompass any pharmaceutically acceptable salts, esters, or salts of such esters, or any other compound which, upon administration to an animal including a human, is capable of providing (directly or indirectly) the biologically active metabolite or residue thereof. Accordingly, for example, the disclosure is also drawn to prodrugs and pharmaceutically acceptable salts of the compounds of the invention, pharmaceutically acceptable salts of such prodrugs, and other bioequivalents.

The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE [(S-acetyl-2-thioethyl)phosphate] derivatives according to the methods disclosed in WO 93/24510 to Gosselin et al., published Dec. 9, 1993 or in WO 94/26764 to Imbach et al.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci., 1977, 66, 119). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Preferred acid salts are the hydrochlorides, acetates, salicylates, nitrates, and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 20 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfoic acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium, and quaternary ammonium cations. Carbonates or hydrogen carbonates are also possible.

For oligonucleotides, preferred examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine.

The antisense compounds of the present invention can be utilized for diagnostics, therapeutics, prophylaxis, and as research reagents and kits. For therapeutics, an animal, preferably a human, suspected of having a disease or disorder, which can be treated by modulating the expression of FXR, is treated by administering antisense compounds in accordance with this invention. The compounds of the invention can be utilized in pharmaceutical compositions by adding an effective amount of an antisense compound to a suitable pharmaceutically acceptable diluent or carrier. Use of the antisense compounds and methods of the invention may also be useful prophylactically, e.g., to prevent or delay infection, inflammation, or tumor formation, for example.

The antisense compounds of the invention are useful for research and diagnostics, because these compounds hybridize to nucleic acids encoding FXR, enabling sandwich and other assays to easily be constructed to exploit this fact. Hybridization of the antisense oligonucleotides of the invention with a nucleic acid encoding FXR can be detected by means known in the art. Such means may include conjugation of an enzyme to the oligonucleotide, radiolabelling of the oligonucleotide or any other suitable detection means. Kits using such detection means for detecting the level of FXR in a sample may also be prepared.

The present invention also includes pharmaceutical compositions and formulations, which include the antisense compounds of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer, intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration.

Pharmaceutical compositions and formulations for topical administration may include transdermal patches, ointments, lotions, creams, gels, drops, suppositories, sprays, liquids, and powders. Conventional pharmaceutical carriers, aqueous, powder or oily bases, thickeners and the like may be necessary or desirable. Coated condoms, gloves, and the like may also be useful.

Compositions and formulations for oral administration include powders or granules, suspensions, or solutions in water or non-aqueous media, capsules, sachets, or tablets. Thickeners, flavoring agents, diluents, emulsifiers, dispersing aids, or binders may be desirable.

Compositions and formulations for parenteral, intrathecal or intraventricular administration may include sterile aqueous solutions, which may also contain buffers, diluents and other suitable additives such as, but not limited to, penetration enhancers, carrier compounds and other pharmaceutically acceptable carriers or excipients.

Pharmaceutical compositions of the present invention include, but are not limited to, solutions, emulsions, and liposome-containing formulations. These compositions may be generated from a variety of components that include, but are not limited to, preformed liquids, self-emulsifying solids and self-emulsifying semisolids.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, liquid syrups, soft gels, suppositories, and enemas. The compositions of the present invention may also be formulated as suspensions in aqueous, non-aqueous or mixed media. Aqueous suspensions may further contain substances, which increase the viscosity of the suspension including, for example, sodium carboxymethylcellulose, sorbitol, and/or dextran. The suspension may also contain stabilizers.

In one embodiment of the present invention the pharmaceutical compositions may be formulated and used as foams. Pharmaceutical foams include formulations such as, but not limited to, emulsions, microemulsions, creams, jellies, and liposomes. While basically similar in nature these formulations vary in the components and the consistency of the final product. The preparation of such compositions and formulations is generally known to those skilled in the pharmaceutical and formulation arts and may be applied to the formulation of the compositions of the present invention. Emulsions

The compositions of the present invention may be prepared and formulated as emulsions. Emulsions are typically heterogenous systems of one liquid dispersed in another in the form of droplets usually exceeding 0.1 μm in diameter. (Idson, in Pharmaceutical Dosage Forms, Lieberman, Rieger and Banker (Eds.), 1988, Marcel Dekker, Inc., New York, N.Y., volume 1, p. 199; Rosoff, in Pharmaceutical Dosage Forms, Lieberman, Rieger and Banker (Eds.), 1988, Marcel Dekker, Inc., New York, N.Y., Volume 1, p. 245; Block in Pharmaceutical Dosage Forms, Lieberman, Rieger and Banker (Eds.), 1988, Marcel Dekker, Inc., New York, N.Y., volume 2, p. 335; Higuchi et al., in Remington's Pharmaceutical Sciences, Mack Publishing Co., Easton, Pa., 1985, p. 301). Emulsions are often biphasic systems comprising of two immiscible liquid phases intimately mixed and dispersed with each other. In general, emulsions may be either water-in-oil (w/o) or of the oil-in-water (o/w) variety. When an aqueous phase is finely divided into and dispersed as minute droplets into a bulk oily phase the resulting composition is called a water-in-oil (w/o) emulsion. Alternatively, when an oily phase is finely divided into and dispersed as minute droplets into a bulk aqueous phase the resulting composition is called an oil-in-water (o/w) emulsion. Emulsions may contain additional components in addition to the dispersed phases and the active drug, which may be present as a solution in either the aqueous phase, oily phase or itself as a separate phase. Pharmaceutical excipients such as emulsifiers, stabilizers, dyes, and anti-oxidants may also be present in emulsions as needed. Pharmaceutical emulsions may also be multiple emulsions that are comprised of more than two phases such as, for example, in the case of oil-in-water-in-oil (o/w/o) and water-in-oil-in-water (w/o/w) emulsions. Such complex formulations often provide certain advantages that simple binary emulsions do not. Multiple emulsions in which individual oil droplets of an o/w emulsion enclose small water droplets constitute a w/o/w emulsion. Likewise a system of oil droplets enclosed in globules of water stabilized in an oily continuous provides an o/w/o emulsion.

Emulsions are characterized by little or no thermodynamic stability. Often, the dispersed or discontinuous phase of the emulsion is well dispersed into the external or continuous phase and maintained in this form through the means of emulsifiers or the viscosity of the formulation. Either of the phases of the emulsion may be a semisolid or a solid, as is the case of emulsion-style ointment bases and creams. Other means of stabilizing emulsions entail the use of emulsifiers that may be incorporated into either phase of the emulsion. Emulsifiers may broadly be classified into four categories: synthetic surfactants, naturally occurring emulsifiers, absorption bases, and finely dispersed solids (Idson, in Pharmaceutical Dosaqe Forms, Lieberman, Rieger and Banker (Eds.), 1988, Marcel Dekker, Inc., New York, N.Y., volume 1, p. 199).

Synthetic surfactants, also known as surface active agents, have found wide applicability in the formulation of emulsions and have been reviewed in the literature (Rieger, in Pharmaceutical Dosage Forms, Lieberman, Rieger and Banker (Eds.), 1988, Marcel Dekker, Inc., New York, N.Y., volume 1, p. 285; Idson, in Pharmaceutical Dosage Forms, Lieberman, Rieger and Banker (Eds.), Marcel Dekker, Inc., New York, N.Y., 1988, volume 1, p. 199). Surfactants are typically amphiphilic and comprise a hydrophilic and a hydrophobic portion. The ratio of the hydrophilic to the hydrophobic nature of the surfactant has been termed the hydrophile/lipophile balance (HLB) and is a valuable tool in categorizing and selecting surfactants in the preparation of formulations. Surfactants may be classified into different classes based on the nature of the hydrophilic group: nonionic, anionic, cationic, and amphoteric (Rieger, in Pharmaceutical Dosage Forms, Lieberman, Rieger and Banker (Eds.), 1988, Marcel Dekker, Inc., New York, N.Y., volume 1, p. 285).

Naturally occurring emulsifiers used in emulsion formulations include lanolin, beeswax, phosphatides, lecithin, and acacia. Absorption bases possess hydrophilic properties such that they can soak up water to form w/o emulsions yet retain their semisolid consistencies, such as anhydrous lanolin and hydrophilic petrolatum. Finely divided solids have also been used as good emulsifiers especially in combination with surfactants and in viscous preparations. These include polar inorganic solids, such as heavy metal hydroxides, nonswelling clays such as bentonite, attapulgite, hectorite, kaolin, montmorillonite, colloidal aluminum silicate and colloidal magnesium aluminum silicate, pigments and nonpolar solids such as carbon or glyceryl tristearate.

A large variety of non-emulsifying materials are also included in emulsion formulations and contribute to the properties of emulsions. These include fats, oils, waxes, fatty acids, fatty alcohols, fatty esters, humectants, hydrophilic colloids, preservatives, and antioxidants (Block, in Pharmaceutical Dosage Forms, Lieberman, Rieger and Banker (Eds.), 1988, Marcel Dekker, Inc., New York, N.Y., volume 1, p. 335; Idson, in Pharmaceutical Dosage Forms, Lieberman, Rieger and Banker (Eds.), 1988, Marcel Dekker, Inc., New York, N.Y., volume 1, p. 199).

Hydrophilic colloids or hydrocolloids include naturally occurring gums and synthetic polymers such as polysaccharides (for example, acacia, agar, alginic acid, carrageenan, guar gum, karaya gum, and tragacanth), cellulose derivatives (for example, carboxymethylcellulose and carboxypropylcellulose), and synthetic polymers (for example, carbomers, cellulose ethers, and carboxyvinyl polymers). These disperse or swell in water to form colloidal solutions that stabilize emulsions by forming strong interfacial films around the dispersed phase droplets and by increasing the viscosity of the external phase.

Since emulsions often contain a number of ingredients such as carbohydrates, proteins, sterols, and phosphatides that may readily support the growth of microbes, these formulations often incorporate preservatives. Commonly used preservatives included in emulsion formulations include methyl paraben, propyl paraben, quaternary ammonium salts, benzalkonium chloride, esters of p-hydroxybenzoic acid, and boric acid. Antioxidants are also commonly added to emulsion formulations to prevent deterioration of the formulation. Antioxidants used may be free radical scavengers such as tocopherols, alkyl gallate, butylated hydroxyanisole, butylated hydroxytoluene, or reducing agents such as ascorbic acid and sodium metabisulfite, and antioxidant synergists such as citric acid, tartaric acid, and lecithin.

The application of emulsion formulations via dermatological, oral, and parenteral routes and methods for their manufacture have been reviewed in the literature (Idson, in Pharmaceutical Dosage Forms, Lieberman, Rieger and Banker (Eds.), 1988, Marcel Dekker, Inc., New York, N.Y., volume 1, p. 199). Emulsion formulations for oral delivery have been very widely used because of reasons of ease of formulation, efficacy from an absorption and bioavailability standpoint. (Rosoff, in Pharmaceutical Dosage Forms, Lieberman, Rieger and Banker (Eds.), 1988, Marcel Dekker, Inc., New York, N.Y., volume 1, p. 245; Idson, in Pharmaceutical Dosage Forms, Lieberman, Rieger and Banker (Eds.), 1988, Marcel Dekker, Inc., New York, N.Y., volume 1, p. 199). Mineral-oil base laxatives, oil-soluble vitamins, and high fat nutritive preparations are among the materials that have commonly been administered orally as o/w emulsions.

In one embodiment of the present invention, the compositions of oligonucleotides and nucleic acids are formulated as microemulsions. A microemulsion may be defined as a system of water, oil, and amphiphile, which is a single optically isotropic, and thermodynamically stable liquid solution (Rosoff, in Pharmaceutical Dosage Forms, Lieberman, Rieger and Banker (Eds.), 1988, Marcel Dekker, Inc., New York, N.Y., volume 1, p. 245). Typically microemulsions are systems that are prepared by first dispersing an oil in an aqueous surfactant solution and then adding a sufficient amount of a fourth component, generally an intermediate chain-length alcohol to form a transparent system. Therefore, microemulsions have also been described as thermodynamically stable, isotropically clear dispersions of two immiscible liquids that are stabilized by interfacial films of surface-active molecules (Leung and Shah, in: Controlled Release of Drugs: Polymers and Aggregate Systems, Rosoff, M., Ed., 1989, VCH Publishers, New York, pages 1852-5). Microemulsions commonly are prepared via a combination of three to five components that include oil, water, surfactant, cosurfactant, and electrolyte. Whether the microemulsion is of the water-in-oil (w/o) or an oil-in-water (o/w) type is dependent on the properties of the oil and surfactant used and on the structure and geometric packing of the polar heads and hydrocarbon tails of the surfactant molecules (Schott, in Remington's Pharmaceutical Sciences, Mack Publishing Co., Easton, Pa., 1985, p. 271).

The phenomenological approach utilizing phase diagrams has been extensively studied and has yielded a comprehensive knowledge, to one skilled in the art, of how to formulate microemulsions (Rosoff, in Pharmaceutical Dosage Forms, Lieberman, Rieger and Banker (Eds.), 1988, Marcel Dekker, Inc., New York, N.Y., volume 1, p. 245; Block, in Pharmaceutical Dosage Forms, Lieberman, Rieger and Banker (Eds.), 1988, Marcel Dekker, Inc., New York, N.Y., volume 1, p. 335). Compared to conventional emulsions, microemulsions offer the advantage of solubilizing water-insoluble drugs in a formulation of thermodynamically stable droplets that are formed spontaneously.

Surfactants used in the preparation of microemulsions include, but are not limited to, ionic surfactants, non-ionic surfactants, Brij 96, polyoxyethylene oleyl ethers, polyglycerol fatty acid esters, tetraglycerol monolaurate (ML310), tetraglycerol monooleate (MO310), hexaglycerol monooleate (PO310), hexaglycerol pentaoleate (PO500), decaglycerol monocaprate (MCA750), decaglycerol monooleate (MO750), decaglycerol sequioleate (S0750), decaglycerol decaoleate (DAO750), alone or in combination with cosurfactants. The cosurfactant, usually a short-chain alcohol such as ethanol, 1-propanol, and 1-butanol, serves to increase the interfacial fluidity by penetrating into the surfactant film and consequently creating a disordered film because of the void space generated among surfactant molecules. Microemulsions may, however, be prepared without the use of cosurfactants and alcohol-free self-emulsifying microemulsion systems are known in the art. The aqueous phase may typically be, but is not limited to, water, an aqueous solution of the drug, glycerol, PEG300, PEG400, polyglycerols, propylene glycols, and derivatives of ethylene glycol. The oil phase may include, but is not limited to, materials such as Captex 300, Captex 355, Capmul MCM, fatty acid esters, medium chain (C8-C12) mono, di, and triglycerides, polyoxyethylated glyceryl fatty acid esters, fatty alcohols, polyglycolized glycerides, saturated polyglycolized C8-C10 glycerides, vegetable oils and silicone oil.

Microemulsions are particularly of interest from the standpoint of drug solubilization and the enhanced absorption of drugs. Lipid based microemulsions (both o/w and w/o) have been proposed to enhance the oral bioavailability of drugs, including peptides (Constantinides et al., Pharmaceutical Research, 1994, 11, 1385-1390; Ritschel, Meth. Find. Exp. Clin. Pharmacol., 1993, 13, 205). Microemulsions afford advantages of improved drug solubilization, protection of drug from enzymatic hydrolysis, possible enhancement of drug absorption due to surfactant-induced alterations in membrane fluidity and permeability, ease of preparation, ease of oral administration over solid dosage forms, improved clinical potency, and decreased toxicity (Constantinides et al., Pharmaceutical Research, 1994, 11, 1385; Ho et al., J. Pharm. Sci., 1996, 85, 138-143). Often microemulsions may form spontaneously when their components are brought together at ambient temperature. This may be particularly advantageous when formulating thermolabile drugs, peptides, or oligonucleotides. Microemulsions have also been effective in the transdermal delivery of active components in both cosmetic and pharmaceutical applications. It is expected that the microemulsion compositions and formulations of the present invention will facilitate the increased systemic absorption of oligonucleotides and nucleic acids from the gastrointestinal tract, as well as improve the local cellular uptake of oligonucleotides and nucleic acids within the gastrointestinal tract, vagina, buccal cavity and other areas of administration.

Microemulsions of the present invention may also contain additional components and additives such as sorbitan monostearate (Grill 3), Labrasol, and penetration enhancers to improve the properties of the formulation and to enhance the absorption of the oligonucleotides and nucleic acids of the present invention. Penetration enhancers used in the microemulsions of the present invention may be classified as belonging to one of five broad categories—surfactants, fatty acids, bile salts, chelating agents, and non-chelating non-surfactants (Lee et al., Critical Reviews in Therapeutic Drug Carrier Systems, 1991, p. 92). Each of these classes has been discussed above.

Liposomes

There are many organized surfactant structures besides microemulsions that have been studied and used for the formulation of drugs. These include monolayers, micelles, bilayers, and vesicles. Vesicles, such as liposomes, have attracted great interest because of their specificity and the duration of action they offer from the standpoint of drug delivery. As used in the present invention, the term “liposome” means a vesicle composed of amphiphilic lipids arranged in a spherical bilayer or bilayers.

Liposomes are unilamellar or multilamellar vesicles which have a membrane formed from a lipophilic material and an aqueous interior. The aqueous portion contains the composition to be delivered. Cationic liposomes possess the advantage of being able to fuse to the cell wall. Noncationic liposomes, although not able to fuse as efficiently with the cell wall, are taken up by macrophages in vivo.

In order to cross intact mammalian skin, lipid vesicles must pass through a series of fine pores, each with a diameter less than 50 nm, under the influence of a suitable transdermal gradient. Therefore, it is desirable to use a liposome, which is highly deformable and able to pass through such fine pores.

Further advantages of liposomes include; liposomes obtained from natural phospholipids are biocompatible and biodegradable; liposomes can incorporate a wide range of water and lipid soluble drugs; liposomes can protect encapsulated drugs in their internal compartments from metabolism and degradation (Rosoff, in Pharmaceutical Dosage Forms, Lieberman, Rieger and Banker (Eds.), 1988, Marcel Dekker, Inc., New York, N.Y., volume 1, P. 245). Important considerations in the preparation of liposome formulations are the lipid surface charge, vesicle size, and the aqueous volume of the liposomes.

Liposomes are useful for the transfer and delivery of active ingredients to the site of action. Because the liposomal membrane is structurally similar to biological membranes, when liposomes are applied to a tissue, the liposomes start to merge with the cellular membranes. As the merging of the liposome and cell progresses, the liposomal contents are emptied into the cell where the active agent may act.

Liposomal formulations have been the focus of extensive investigation as the mode of delivery for many drugs. There is growing evidence that for topical administration, liposomes present several advantages over other formulations. Such advantages include reduced side-effects related to high systemic absorption of the administered drug, increased accumulation of the administered drug at the desired target, and the ability to administer a wide variety of drugs, both hydrophilic and hydrophobic, into the skin.

Several reports have detailed the ability of liposomes to deliver agents including high-molecular weight DNA into the skin. Compounds including analgesics, antibodies, hormones, and high-molecular weight DNAs have been administered to the skin. The majority of applications resulted in the targeting of the upper epidermis.

Liposomes fall into two broad classes. Cationic liposomes are positively charged liposomes, which interact with the negatively charged DNA molecules to form a stable complex. The positively charged DNA/liposome complex binds to the negatively charged cell surface and is internalized in an endosome. Due to the acidic pH within the endosome, the liposomes are ruptured, releasing their contents into the cell cytoplasm (Wang et al., Biochem. Biophys. Res. Commun., 1987, 147, 980-985)

Liposomes, which are pH-sensitive or negatively charged, entrap DNA rather than complex with it. Since both the DNA and the lipid are similarly charged, repulsion rather than complex formation occurs. Nevertheless, some DNA is entrapped within the aqueous interior of these liposomes. pH-sensitive liposomes have been used to deliver DNA encoding the thymidine kinase gene to cell monolayers in culture. Expression of the exogenous gene was detected in the target cells (Zhou et al., Journal of Controlled Release, 1992, 19, 269-274).

One major type of liposomal composition includes phospholipids other than naturally derived phosphatidylcholine. Neutral liposome compositions, for example, can be formed from dimyristoyl phosphatidylcholine (DMPC) or dipalmitoyl phosphatidylcholine (DPPC). Anionic liposome compositions generally are formed from dimyristoyl phosphatidylglycerol, while anionic fusogenic liposomes are formed primarily from dioleoyl phosphatidylethanolamine (DOPE). Another type of liposomal composition is formed from phosphatidylcholine (PC) such as, for example, soybean PC, and egg PC. Another type is formed from mixtures of phospholipid and/or phosphatidylcholine and/or cholesterol.

Several studies have assessed the topical delivery of liposomal drug formulations to the skin. Application of liposomes containing interferon to guinea pig skin resulted in a reduction of skin herpes sores while delivery of interferon via other means (e.g. as a solution or as an emulsion) were ineffective (Weiner et al., Journal of Drug Targeting, 1992, 2, 405-410). Further, an additional study tested the efficacy of interferon administered as part of a liposomal formulation to the administration of interferon using an aqueous system, and concluded that the liposomal formulation was superior to aqueous administration (du Plessis et al., Antiviral Research, 1992, 18, 259-265).

Non-ionic liposomal systems have also been examined to determine their utility in the delivery of drugs to the skin, in particular systems comprising non-ionic surfactant and cholesterol. Non-ionic liposomal formulations comprising Novasome™ I (glyceryl dilaurate/cholesterol/polyoxyethylene-10-stearyl ether) and Novasome™ II (glyceryl distearate/cholesterol/polyoxyethylene-10-stearyl ether) were used to deliver cyclosporin-A into the dermis of mouse skin. Results indicated that such non-ionic liposomal systems were effective in facilitating the deposition of cyclosporin-A into different layers of the skin (Hu et al., S.T.P. Pharma. Sci., 1994, 4, 6, 466).

Liposomes also include “sterically stabilized” liposomes, a term that, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome (A) comprises one or more glycolipids, such as monosialoganglioside GM1, or (B) is derivatized with one or more hydrophilic polymers, such as a polyethylene glycol (PEG) moiety. While not wishing to be bound by any particular theory, it is thought in the art that, at least for sterically stabilized liposomes containing gangliosides, sphingomyelin, or PEG-derivatized lipids, the enhanced circulation half-life of these sterically stabilized liposomes derives from a reduced uptake into cells of the reticuloendothelial system (RES) (Allen et al., FEBS Letters, 1987, 223, 42; Wu et al., Cancer Research, 1993, 53, 3765).

Various liposomes comprising one or more glycolipids are known in the art. Papahadjopoulos et al. (Ann. N.Y. Acad. Sci., 1987, 507, 64) reported the ability of monosialoganglioside GM1, galactocerebroside sulfate, and phosphatidylinositol to improve blood half-lives of liposomes. These findings were expounded upon by Gabizon et al. (Proc. Natl. Acad. Sci. U.S.A., 1988, 85, 6949), U.S. Pat. No. 4,837,028 and WO 88/04924, both to Allen et al., disclose liposomes comprising (1) sphingomyelin and (2) the ganglioside Gjor a galactocerebroside sulfate ester. U.S. Pat. No. 5,543,152 (Webb et al.) discloses liposomes comprising sphingomyelin. Liposomes comprising 1,2-sn-dimyristoylphosphatidylcholine are disclosed in WO 97/13499 (Lim et al.).

Many liposomes comprising lipids derivatized with one or more hydrophilic polymers, and methods of preparation thereof, are known in the art. Sunamoto et al. (Bull. Chem. Soc. Jpn., 1980, 53, 2778) described liposomes comprising a nonionic detergent, 2C1215G, which contains a PEG moiety. Illum et al. (FEBS Lett., 1984, 167, 79) noted that hydrophilic coating of polystyrene particles with polymeric glycols results in significantly enhanced blood half-lives. Synthetic phospholipids modified by the attachment of carboxylic groups of polyalkylene glycols (e.g., PEG) are described by Sears (U.S. Pat. Nos. 4,426,330 and 4,534,899). Klibanov et al. (FEBS Lett., 1990, 268, 235) described experiments demonstrating that liposomes comprising phosphatidylethanolamine (PE) derivatized with PEG or PEG stearate have significant increases in blood circulation half-lives. Blume et al. (Biochimica et Biophysica Acta, 1990, 1029, 91) extended such observations to other PEG derivatized phospholipids, e.g., DSPE-PEG, formed from the combination of distearoylpbosphatidylethanolamine (DSPE) and PEG. Liposomes having covalently bound PEG moieties on their external surface are described in European Patent No. EP 0 445 131 B1 and WO 90/04384 to Fisher. Liposome compositions containing 1-20 mole percent of PE derivatized with PEG, and methods of use thereof, are described by Woodle et al. (U.S. Pat. Nos. 5,013,556 and 5,356,633) and Martin et al. (U.S. Pat. No. 5,213,804 and European Patent No. EP 0 496 813 B1). Liposomes comprising a number of other lipid-polymer conjugates are disclosed in WO 91/05545 and U.S. Pat. No. 5,225,212 (both to Martin et al.) and in WO 94/20073 (Zalipsky et al.) Liposomes comprising PEG-modified ceramide lipids are described in WO 96/10391 (Choi et al.). U.S. Pat. No. 5,540,935 (Miyazaki et al.) and U.S. Pat. No. 5,556,948 (Tagawa et al.) describe PEG-containing liposomes that can be further derivatized with functional moieties on their surfaces.

A limited number of liposomes comprising nucleic acids are known in the art. WO 96/40062 to Thierry et al. discloses methods for encapsulating high molecular weight nucleic acids in liposomes. U.S. Pat. No. 5,264,221 to Tagawa et al. discloses protein-bonded liposomes and asserts that the contents of such liposomes may include an antisense RNA. U.S. Pat. No. 5,665,710 to Rahman et al. describes certain methods of encapsulating oligodeoxynucleotides in liposomes. WO 97/04787 to Love et al. discloses liposomes comprising antisense oligonucleotides targeted to the raf gene.

Transfersomes are yet another type of liposomes, and are highly deformable lipid aggregates which are attractive candidates for drug delivery vehicles. Transfersomes may be described as lipid droplets that are so highly deformable that they are easily able to penetrate through pores that are smaller than the droplet. Transfersomes are adaptable to the environment in which they are used, e.g. they are self-optimizing (adaptive to the shape of pores in the skin), self-repairing, frequently reach their targets without fragmenting, and often self-loading. To make transfersomes it is possible to add surface edge-activators, usually surfactants, to a standard liposomal composition. Transfersomes have been used to deliver serum albumin to the skin. The transfersome-mediated delivery of serum albumin has been shown to be as effective as subcutaneous injection of a solution containing serum albumin.

Surfactants find wide application in formulations such as emulsions (including microemulsions) and liposomes. The most common way of classifying and ranking the properties of the many different types of surfactants, both natural and synthetic, is by the use of the hydrophile/lipophile balance (HLB). The nature of the hydrophilic group (also known as the “head”) provides the most useful means for categorizing the different surfactants used in formulations (Rieger, in Pharmaceutical Dosage Forms, Marcel Dekker, Inc., New York, N.Y., 1988, p. 285)

If the surfactant molecule is not ionized, it is classified as a nonionic surfactant. Nonionic surfactants find wide application in pharmaceutical and cosmetic products and are usable over a wide range of pH values. In general their HLB values range from 2 to about 18 depending on their structure. Nonionic surfactants include nonionic esters such as ethylene glycol esters, propylene glycol esters, glyceryl esters, polyglyceryl esters, sorbitan esters, sucrose esters, and ethoxylated esters. Nonionic alkanolamides and ethers such as fatty alcohol ethoxylates, propoxylated alcohols, and ethoxylated/propoxylated block polymers are also included in this class. The polyoxyethylene surfactants are the most popular members of the nonionic surfactant class.

If the surfactant molecule carries a negative charge when it is dissolved or dispersed in water, the surfactant is classified as anionic. Anionic surfactants include carboxylates such as soaps, acyl lactylates, acyl amides of amino acids, esters of sulfuric acid such as alkyl sulfates and ethoxylated alkyl sulfates, sulfonates such as alkyl benzene sulfonates, acyl isethionates, acyl taurates and sulfosuccinates, and phosphates. The most important members of the anionic surfactant class are the alkyl sulfates and the soaps.

If the surfactant molecule carries a positive charge when it is dissolved or dispersed in water, the surfactant is classified as cationic. Cationic surfactants include quaternary ammonium salts and ethoxylated amines. The quaternary ammonium salts are the most used members of this class.

If the surfactant molecule has the ability to carry either a positive or negative charge, the surfactant is classified as amphoteric. Amphoteric surfactants include acrylic acid derivatives, substituted alkylamides, N-alkylbetaines, and phosphatides.

The use of surfactants in drug products, formulations and in emulsions has been reviewed (Rieger, in Pharmaceutical Dosage Forms, Marcel Dekker, Inc., New York, N.Y., 1988, p. 285). Penetration Enhancers

In one embodiment, the present invention employs various penetration enhancers to effect the efficient delivery of nucleic acids particularly oligonucleotides, to the skin of animals. Most drugs are present in solution in both ionized and nonionized forms. However, usually only lipid soluble or lipophilic drugs readily cross cell membranes. It has been discovered that even non-lipophilic drugs may cross cell membranes if the membrane to be crossed is treated with a penetration enhancer. In addition to aiding the diffusion of non-lipophilic drugs across cell membranes, penetration enhancers also enhance the permeability of lipophilic drugs.

Penetration enhancers may be classified as belonging to one of five broad categories, i.e., surfactants, fatty acids, bile salts, chelating agents, and non-chelating nonsurfactants (Lee et al., Critical Reviews in Therapeutic Drug Carrier Systems, 1991, p. 92). Each of the above mentioned classes of penetration enhancers are described below in greater detail.

Surfactants: In connection with the present invention, surfactants (or “surface-active agents”) are chemical entities which, when dissolved in an aqueous solution, reduce the surface tension of the solution or the interfacial tension between the aqueous solution and another liquid, with the result that absorption of oligonucleotides through the mucosa is enhanced. In addition to bile salts and fatty acids, these penetration enhancers include, for example, sodium lauryl sulfate, polyoxyethylene-9-lauryl ether and polyoxyethylene-20-cetyl ether) (Lee et al., Critical Reviews in Therapeutic Drug Carrier Systems, 1991, p. 92); and perfluorochemical emulsions, such as FC-43. Takahashi et al., J. Pharm. Pharmacol., 1988, 40, 252).

Fatty acids: Various fatty acids and their derivatives which act as penetration enhancers include, for example, oleic acid, lauric acid, capric acid (n-decanoic acid), myristic acid, palmitic acid, stearic acid, linoleic acid, linolenic acid, dicaprate, tricaprate, monoolein (1-monooleoyl-.rac-glycerol), dilaurin, caprylic acid, arachidonic acid, glycerol 1-monocaprate, 1-dodecylazacycloheptan-2-one, acylcarnitines, acylcholines, C1-10 alkyl esters thereof (e.g., methyl, isopropyl and t-butyl), and mono- and di-glycerides thereof (i.e., oleate, laurate, caprate, myristate, palmitate, stearate, linoleate, etc.) (Lee et al., Critical Reviews in Therapeutic Drug Carrier Systems, 1991, p. 92; Muranishi, Critical Reviews in Therapeutic Drug Carrier Systems, 1990, 7, 1-33; El Hariri et al., J. Pharm. Pharmacol., 1992, 44, 651-654).

Bile salts: The physiological role of bile includes the facilitation of dispersion and absorption of lipids and fat-soluble vitamins (Brunton, Chapter 38 in: Goodman & Gilman's The Pharmacological Basis of Therapeutics, 9th Ed., Hardman et al. Eds. McGraw-Hill, New York, 1996, pp. 934-935). Various natural bile salts, and their synthetic derivatives, act as penetration enhancers. Thus the term “bile salts” includes any of the naturally occurring components of bile as well as any of their synthetic derivatives. The bile salts of the invention include, for example, cholic acid (or its pharmaceutically acceptable sodium salt, sodium cholate), dehydrocholic acid (sodium dehydrocholate), deoxycholic acid (sodium deoxycholate), glucholic acid (sodium glucholate), glycholic acid (sodium glycocholate), glycodeoxycholic acid (sodium glycodeoxycholate), taurocholic acid (sodium taurocholate), taurodeoxycholic acid (sodium taurodeoxycholate), chenodeoxycholic acid (sodium chenodeoxycholate), ursodeoxycholic acid (UDCA), sodium tauro-24,25-dihydro-fusidate (STDHF), sodium glycodihydrofusidate′ and polyoxyethylene-9-lauryl ether (POE) (Lee et al., Critical Reviews in Therapeutic Drug Carrier Systems, 1991, page 92; Swinyard, Chapter 39 In: Remington's Pharmaceutical Sciences, 18th Ed., Gennaro, ed., Mack Publishing Co., Easton, Pa., 1990, pages 782-783; Muranishi, Critical Reviews in Therapeutic Drug Carrier Systems, 1990, 7, 1-33; Yamamoto et al., J. Pharm. Exp. Ther., 1992, 263, 25; Yamashita et al., J. Pharm. Sci., 1990, 79, 579-583).

Chelating Agents: Chelating agents, as used in connection with the present invention, can be defined as compounds that remove metallic ions from solution by forming complexes therewith, with the result that absorption of oligonucleotides through the mucosa is enhanced. With regards to their use as penetration enhancers in the present invention, chelating agents have the added advantage of also serving as DNase inhibitors, as most characterized DNA nucleases require a divalent metal ion for catalysis and are thus inhibited by chelating agents (Jarrett, J. Chromatogr., 1993, 618, 315-339). Chelating agents of the invention include but are not limited to disodium ethylenediaminetetraacetate (EDTA), citric acid, salicylates (e.g., sodium salicylate, 5-methoxysalicylate and homovanilate), N-acyl derivatives of collagen, laureth-9 and N-amino acyl derivatives of beta-diketones (enamines)(Lee et al., Critical Reviews in Therapeutic Drug Carrier Systems, 1991, page 92; Muranishi, Critical Reviews in Therapeutic Drug Carrier Systems, 1990, 7, 1-33; Buur et al., J. Control Rel., 1990, 14, 43-51).

Non-chelating non-surfactants: As used herein, nonchelating non-surfactant penetration enhancing compounds can be defined as compounds that demonstrate insignificant activity as chelating agents or as surfactants but that nonetheless enhance absorption of oligonucleotides through the alimentary mucosa (Muranishi, Critical Reviews in Therapeutic Drug Carrier Systems, 1990, 7, 1-33). This class of penetration enhancers includes, for example, unsaturated cyclic ureas, 1-alkyl- and 1-alkenylazacyclo-alkanone derivatives (Lee et al., Critical Reviews in Therapeutic Drug Carrier Systems, 1991, page 92); and non-steroidal anti-inflammatory agents such as diclofenac sodium, indomethacin, and phenylbutazone (Yamashita et al., J. Pharm. Pharmacol., 1987, 39, 621-626).

Agents that enhance uptake of oligonucleotides at the cellular level may also be added to the pharmaceutical and other compositions of the present invention. For example, cationic lipids, such as lipofectin (Junichi et al, U.S. Pat. No. 5,705,188), cationic glycerol derivatives, and polycationic molecules, such as polylysine (Lollo et al., PCT Application WO 97/30731), are also known to enhance the cellular uptake of oligonucleotides.

Other agents may be utilized to enhance the penetration of the administered nucleic acids, including glycols such as ethylene glycol and propylene glycol, pyrrols such as 2-pyrrol, azones, and terpenes such as limonene and menthone.

Carriers

Certain compositions of the present invention also incorporate carrier compounds in the formulation. As used herein, “carrier compound” or “carrier” can refer to a nucleic acid, or analog thereof, which is inert (i.e., does not possess biological activity per se) but is recognized as a nucleic acid by in vivo processes that reduce the bioavailability of a nucleic acid having biological activity by, for example, degrading the biologically active nucleic acid or promoting its removal from circulation. The coadministration of a nucleic acid and a carrier compound, typically with an excess of the latter substance, can result in a substantial reduction of the amount of nucleic acid recovered in the liver, kidney or other extracirculatory reservoirs, presumably due to competition between the carrier compound and the nucleic acid for a common receptor. For example, the recovery of a partially phosphorothioate oligonucleotide in hepatic tissue can be reduced when it is coadministered with polyinosinic acid, dextran sulfate, polycytidic acid or 4-acetamido-4′isothiocyano-stilbene-2,2′disulfonic acid (Miyao et al., Antisense Res. Dev., 1995, 5, 115-121; Takakura et al., Antisense & Nucl. Acid Drug Dev., 1996, 6, 177-183).

Excipients

In contrast to a carrier compound, a “pharmaceutical carrier” or “excipient” is a pharmaceutically acceptable solvent, suspending agent or any other pharmacologically inert vehicle for delivering one or more nucleic acids to an animal. The excipient may be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc.).

Pharmaceutically acceptable organic or inorganic excipient suitable for non-parenteral administration, which does not deleteriously react with nucleic acids, can also be used to formulate the compositions of the present invention. Suitable pharmaceutically acceptable carriers include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone and the like.

Formulations for topical administration of nucleic acids may include sterile and non-sterile aqueous solutions, non-aqueous solutions in common solvents such as alcohols, or solutions of the nucleic acids in liquid or solid oil bases. The solutions may also contain buffers, diluents, and other suitable additives. Pharmaceutically acceptable organic or inorganic excipients suitable for non-parenteral administration that do not deleteriously react with nucleic acids can be used.

Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

Other Components

The compositions of the present invention may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the compositions of the present invention, such as dyes, flavoring agents, preservatives, antioxidants, opacifiers, thickening agents and stabilizers. However, such materials, when added, should not unduly interfere with the biological activities of the components of the compositions of the present invention. The formulations can be sterilized and, if desired, mixed with auxiliary agents, e.g., lubricants, preservatives, stabilizers, wetting agents, emulsifiers, salts for influencing osmotic pressure, buffers, colorings, flavorings and/or aromatic substances and the like which do not deleteriously interact with the nucleic acid(s) of the formulation.

Aqueous suspensions may contain substances that increase the viscosity of the suspension including, for example, sodium carboxymethylcellulose, sorbitol, and/or dextran. The suspension may also contain stabilizers.

Certain embodiments of the invention provide pharmaceutical compositions containing (a) one or more antisense compounds and (b) one or more other chemotherapeutic agents which function by a non-antisense mechanism. Examples of such chemotherapeutic agents include, but are not limited to, anticancer drugs such as daunorubicin, dactinomycin, doxorubicin, bleomycin, mitomycin, nitrogen mustard, chlorambucil, melphalan, cyclophosphamide, 6-mercaptopurine, 6-thioguanine, cytarabine (CA), 5-fluorouracil (5-FU), floxuridine (5-FUdR), methotrexate (MTX), colchicine, vincristine, vinblastine, etoposide, teniposide, cisplatin and diethylstilbestrol (DES). See, generally, The Merck Manual of Diagnosis and Therapy, 15th Ed., Berkow et al., eds., 1987, Rahway, N.J., pages 1206-1228). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. See, generally, The Merck Manual of Diagnosis and Therapy, 15th Ed., Berkow et al., eds., 1987, Rahway, N.J., pages 2499-2506 and 46-49, respectively) other non-antisense chemotherapeutic agents are also within the scope of this invention. Two or more combined compounds may be used together or sequentially.

In another related embodiment, compositions of the invention may contain one or more antisense compounds, particularly oligonucleotides, targeted to a first nucleic acid and one or more additional antisense compounds targeted to a second nucleic acid target. Numerous examples of antisense compounds are known in the art. Two or more combined compounds may be used together or sequentially.

The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC50s found to be effective in in vitro and in vivo animal models. In general, dosage is from 0.01 μg to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 μg to 100 g per kg of body weight, once or more daily, to once every 20 years.

While the present invention has been described with specificity in accordance with certain of its preferred embodiments, the following examples serve only to illustrate the invention and are not intended to limit the same.

### EXAMPLES

### Example 1

Nucleoside Phosphoramidites for Oligonucleotide Synthesis Deoxy and 2′-Alkoxy Amidites

2′-Deoxy and 2′-methoxy beta-cyanoethyldiisopropyl phosphoramidites are available from commercial sources (e.g. Chemgenes, Needham Mass. or Glen Research, Inc. Sterling Va.). Other 2′-O-alkoxy substituted nucleoside amidites are prepared as described in U.S. Pat. No. 5,506,351, herein incorporated by reference. For oligonucleotides synthesized using 2′-alkoxy amidites, the standard cycle for unmodified oligonucleotides is utilized, except the wait step after pulse delivery of tetrazole and base is increased to 360 seconds.

Oligonucleotides containing 5-methyl-2′-deoxycytidine (5-Me-C) nucleotides are synthesized according to published methods [Sanghvi, et. al., Nucleic Acids Research, 1993, 21, 3197-3203] using commercially available phosphoramidites (Glen Research, Sterling Va. or ChemGenes, Needham Mass.).

### 2′-Fluoro amidites

### 2′-Fluorodeoxyadenosine amidites

2′-fluoro oligonucleotides are synthesized as described previously [Kawasaki, et. al., J. Med. Chem., 1993, 36, 831-841] and U.S. Pat. No. 5,670,633, herein incorporated by reference. Briefly, the protected nucleoside N6-benzoyl-2′-deoxy-2′-fluoroadenosine is synthesized utilizing commercially available 9-beta-D-arabinofuranosyladenine as starting material and by modifying literature procedures whereby the 2′-alpha-fluoro atom is introduced by an S_{N}2-displacement of a 2′-beta-trityl group. Thus N6-benzoyl-9-beta-D-arabinofuranosyladenine is selectively protected in moderate yield as the 3′,5′-ditetrahydropyranyl (THP) intermediate. Deprotection of the THP and N6-benzoyl groups is accomplished using standard methodologies and standard methods are used to obtain the 5′-dimethoxytrityl-(DMT) and 5′-DMT-3′-phosphoramidite intermediates.

### 2′-Fluorodeoxyguanosine

The synthesis of 2′-deoxy-2′-fluoroguanosine is accomplished using tetraisopropyldisiloxanyl (TPDS) protected 9-beta-D-arabinofuranosylguanine as starting material, and conversion to the intermediate diisobutyrylarabinofuranosylguanosine. Deprotection of the TPDS group is followed by protection of the hydroxyl group with THP to give diisobutyryl di-THP protected arabinofuranosylguanine. Selective O-deacylation and triflation is followed by treatment of the crude product with fluoride, then deprotection of the THP groups. Standard methodologies are used to obtain the 5′-DMT- and 5′-DMT-3′-phosphoramidites.

### 2′-Fluorouridine

Synthesis of 2′-deoxy-2′-fluorouridine is accomplished by the modification of a literature procedure in which 2,2′anhydro-1-beta-D-arabinofuranosyluracil is treated with 70% hydrogen fluoride-pyridine. Standard procedures are used to obtain the 5′-DMT and 5′-DMT-3′-phosphoramidites.

### 2′-Fluorodeoxycytidine

2′-deoxy-2′-fluorocytidine is synthesized via amination of 2′-deoxy-2′-fluorouridine, followed by selective protection to give N4-benzoyl-2′-deoxy-2′-fluorocytidine. Standard procedures are used to obtain the 5′-DMT and 5′-DMT-3′phosphoramidites.

### 2′-O-(2-Methoxyethyl) modified amidites

2′-O-Methoxyethyl-substituted nucleoside amidites are prepared as follows, or alternatively, as per the methods of Martin, P., Helvetica Chimica Acta, 1995, 78, 486-504.

### 2,2′-Anhydro[1-(beta-D-arabinofuranosyl)-5-methyluridine]

5-Methyluridine (ribosylthymine, commercially available through Yamasa, Choshi, Japan) (72.0 g, 0.279 M), diphenylcarbonate (90.0 g, 0.420 M) and sodium bicarbonate (2.0 g, 0.024 M) are added to DMF (300 mL). The mixture is heated to reflux, with stirring, allowing the evolved carbon dioxide gas to be released in a controlled manner. After 1 hour, the slightly darkened solution is concentrated under reduced pressure. The resulting syrup is poured into diethylether (2.5 L), with stirring. The product formed a gum. The ether is decanted and the residue is dissolved in a minimum amount of methanol (ca. 400 mL). The solution is poured into fresh ether (2.5 L) to yield a stiff gum. The ether is decanted and the gum is dried in a vacuum oven (60° C. at 1 mm Hg for 24 h) to give a solid that is crushed to a light tan powder. The material is used as is for further reactions (or it can be purified further by column chromatography using a gradient of methanol in ethyl acetate (10-25%) to give a white solid.

### 2′-O-Methoxyethyl-5-methyluridine

2,2′-Anhydro-5-methyluridine (195 g, 0.81 M), tris(2-methoxyethyl)borate (231 g, 0.98 M) and 2-methoxyethanol (1.2 L) are added to a 2 L stainless steel pressure vessel and placed in a pre-heated oil bath at 160° C. After heating for 48 hours at 155-160° C., the vessel is opened and the solution evaporated to dryness and triturated with MeOH (200 mL). The residue is suspended in hot acetone (1 L). The insoluble salts are filtered, washed with acetone (150 mL) and the filtrate evaporated. The residue (280 g) is dissolved in CH₃CN (600 mL) and evaporated. A silica gel column (3 kg) is packed in CH₂Cl₂/acetone/MeOH (20:5:3) containing 0.5% Et₃NH. The residue is dissolved in CH₂Cl₂ (250 mL) and adsorbed onto silica (150 g) prior to loading onto the column. The product is eluted with the packing solvent to give the title product. Additional material can be obtained by reworking impure fractions.

### 2′-O-Methoxyethyl-5′-O-dimethoxytrityl-5-methyluridine

2′-O-Methoxyethyl-5-methyluridine (160 g, 0.506 M) is co-evaporated with pyridine (250 mL) and the dried residue dissolved in pyridine (1.3 L). A first aliquot of dimethoxytrityl chloride (94.3 g, 0.278 M) is added and the mixture stirred at room temperature for one hour. A second aliquot of dimethoxytrityl chloride (94.3 g, 0.278 M) is added and the reaction stirred for an additional one hour. Methanol (170 mL) is then added to stop the reaction. The solvent is evaporated and triturated with CH₃CN (200 mL) The residue is dissolved in CHCl (1.5 L) and extracted with 2×500 mL of saturated NaHCO₃ and 2×500 mL of saturated NaCl. The organic phase is dried over Na₂SO₄, filtered, and evaporated. The residue is purified on a 3.5 kg silica gel column, packed and eluted with EtOAc/hexane/acetone (5:5:1) containing 0-5% Et₃NH. The pure fractions are evaporated to give the title product.

### 3′-O-Acetyl-2′-O-methoxyethyl-5′-O-dimethoxytrityl-5-methyluridine

2′-O-Methoxyethyl-5′-O-dimethoxytrityl-5-methyluridine (106 g, 0.167 M), DMF/pyridine (750 mL of a 3:1 mixture prepared from 562 mL of DMF and 188 mL of pyridine) and acetic anhydride (24.38 mL, 0.258 M) are combined and stirred at room temperature for 24 hours. The reaction is monitored by TLC by first quenching the TLC sample with the addition of MeOH. Upon completion of the reaction, as judged by TLC, MeOH (50 mL) is added and the mixture evaporated at 35° C. The residue is dissolved in CHCl3 (800 mL) and extracted with 2×200 mL of saturated sodium bicarbonate and 2×200 mL of saturated NaCl. The water layers are back extracted with 200 mL of CHCl₃. The combined organics are dried with sodium sulfate and evaporated to a residue. The residue is purified on a 3.5 kg silica gel column and eluted using EtOAc/hexane (4:1). Pure product fractions are evaporated to yield the title compounds.

### 3′-O-Acetyl-2′-O-methoxyethyl-5′-O-dimethoxytrityl-5-methyl-4-triazoleuridine

A first solution is prepared by dissolving 3′-O-acetyl-2′-O-methoxyethyl-5′-O-dimethoxytrityl-5-methyluridine (96 g, 0.144 M) in CH₃CN (700 mL) and set aside. Triethylamine (189 mL, 1.44 M) is added to a solution of triazole (90 g, 1.3 M) in CH₃CN (1 L), cooled to −5° C. and stirred for 0.5 h using an overhead stirrer. POCl₃ is added dropwise, over a 30 minute period, to the stirred solution maintained at 0-10° C., and the resulting mixture stirred for an additional 2 hours. The first solution is added dropwise, over a 45 minute period, to the latter solution. The resulting reaction mixture is stored overnight in a cold room. Salts are filtered from the reaction mixture and the solution is evaporated. The residue is dissolved in EtOAc (1 L) and the insoluble solids are removed by filtration. The filtrate is washed with 1×300 mL of NaHCO₃ and 2×300 mL of saturated NaCl, dried over sodium sulfate and evaporated. The residue is triturated with EtOAc to give the title compound.

### 2′-O-Methoxyethyl-5′-O-dimethoxytrityl-5-methylcytidine

A solution of 3′-O-acetyl-2′-O-methoxyethyl-5′-O-dimethoxytrityl-5-methyl-4-triazoleuridine (103 g, 0.141 M) in dioxane (500 mL) and NH₄OH (30 mL) is stirred at room temperature for 2 hours. The dioxane solution is evaporated and the residue azeotroped with MeOH (2×200 mL). The residue is dissolved in MeOH (300 mL) and transferred to a 2-liter stainless steel pressure vessel. MeOH (400 mL) saturated with NH₃ gas is added and the vessel heated to 100° C. for 2 hours (TLC showed complete conversion). The vessel contents are evaporated to dryness and the residue is dissolved in EtOAc (500 mL) and washed once with saturated NaCl (200 mL). The organics are dried over sodium sulfate and the solvent is evaporated to give the title compound.

### N4-Benzoyl-2′-O-methoxyethyl-5′-O-dimethoxytrityl-5-methylcytidine

2′-O-Methoxyethyl-5′-O-dimethoxytrityl-5-methylcytidine (85 g, 0.134 M) is dissolved in DMF (800 mL) and benzoic anhydride (37.2 g, 0.165 M) is added with stirring. After stirring for 3 hours, TLC showed the reaction to be approximately 95% complete. The solvent is evaporated and the residue azeotroped with MeOH (200 mL). The residue is dissolved in CHCl₃ (700 mL) and extracted with saturated NaHCO, (2×300 mL) and saturated NaCl (2×300 mL), dried over MgSO₄ and evaporated to give a residue. The residue is chromatographed on a 1.5 kg silica column using EtOAc/hexane (1:1) containing 0-5% Et₃NH as the eluting solvent. The pure product fractions are evaporated to give the title compound.

### N4-Benzoyl-2′-O-methoxyethyl-5′-O-dimethoxytrityl-5-methylcytidine-3′-amidite

N4-Benzoyl-2′-O-methoxyethyl-5′-O-dimethoxytrityl-5-methylcytidine (74 g, 0.10 M) is dissolved in CH₂Cl₂ (1 L) Tetrazole diisopropylamine (7.1 g) and 2-cyanoethoxy-tetra(isopropyl)phosphite (40.5 mL, 0.123 M) are added with stirring, under a nitrogen atmosphere. The resulting mixture is stirred for 20 hours at room temperature (TLC showed the reaction to be 95% complete). The reaction mixture is extracted with saturated NaHCO₃ (1×300 mL) and saturated NaCl (3×300 mL). The aqueous washes are back-extracted with CH₂Cl₂ (300 mL), and the extracts are combined, dried over MgSO₄, and concentrated. The residue obtained is chromatographed on a 1.5 kg silica column using EtOAc/hexane (3:1) as the eluting solvent. The pure fractions were combined to give the title compound.

### 2′-O-(Aminooxyethyl) nucleoside amidites and 2′-O-(dimethylaminooxyethyl) nucleoside amidites

### 2′-(Dimethylaminooxyethoxy) nucleoside amidites

2′-(Dimethylaminooxyethoxy) nucleoside amidites [also known in the art as 2′-O-(dimethylaminooxyethyl) nucleoside amidites] are prepared as described in the following paragraphs. Adenosine, cytidine and guanosine nucleoside amidites are prepared similarly to the thymidine (5-methyluridine) except the exocyclic amines are protected with a benzoyl moiety in the case of adenosine and cytidine and with isobutyryl in the case of guanosine.

### 5′-O-tert-Butyldiphenylsilyl-O²-2′-anhydro-5-methyluridine

O²-2′-anhydro-5-methyluridine (Pro. Bio. Sint., Varese, Italy, 100.0 g, 0.4′6 mmol), dimethylaminopyridine (0.66 g, 0.013 eq, 0.0054 mmol) are dissolved in dry pyridine (500 ml) at ambient temperature under an argon atmosphere and with mechanical stirring tert-Butyldiphenylchlorosilane (125.8 g, 119.0 mL, 1.1 eq, 0.458 mmol) is added in one portion. The reaction is stirred for 16 h at ambient temperature. TLC (Rf 0.22, ethyl acetate) indicated a complete reaction. The solution is concentrated under reduced pressure to a thick oil. This is partitioned between dichloromethane (1 L) and saturated sodium bicarbonate (2×1 L) and brine (1 L). The organic layer is dried over sodium sulfate and concentrated under reduced pressure to a thick oil. The oil is dissolved in a 1:1 mixture of ethyl acetate and ethyl ether (600 mL) and the solution is cooled to −10° C. The resulting crystalline product is collected by filtration, washed with ethyl ether (3×200 mL), and dried (40° C., 1 mm Hg, 24 h) to a white solid.

### 5′-O-tert-Butyldiphenylsilyl-2′-O-(2-hydroxyethyl)-5-methyluridine

In a 2 L stainless steel, unstirred pressure reactor is added borane in tetrahydrofuran (1.0 M, 2.0 eq, 622 mL). In the fume hood and with manual stirring, ethylene glycol (350 mL, excess) is added cautiously at first until the evolution of hydrogen gas subsides. 5′-O-tert-Butyldiphenylsilyl-O²-2′anhydro-5-methyluridine (149 g, 0.3′1 mol) and sodium bicarbonate (0.074 g, 0.003 eq) are added with manual stirring. The reactor is sealed and heated in an oil bath until an internal temperature of 160° C. is reached and then maintained for 16 h (pressure<100 psig). The reaction vessel is cooled to ambient and opened. TLC (Rf 0.67 for desired product and Rf 0.82 for ara-T side product, ethyl acetate) indicated about 70% conversion to the product. In order to avoid additional side product formation, the reaction is stopped, concentrated under reduced pressure (10 to 1 mm, Hg) in a warm water bath (40-100° C.) with the more extreme conditions used to remove the ethylene glycol. [Alternatively, once the low boiling solvent is gone, the remaining solution can be partitioned between ethyl acetate and water. The product will be in the organic phase.] The residue is purified by column chromatography (2 kg silica gel, ethyl acetate-hexanes gradient 1:1 to 4:1). The appropriate fractions are combined, stripped, and dried to product as a white crisp foam, contaminated starting material, and pure reusable starting material.

### 2′-O-([2-phthalimidoxy)ethyl]-5′-t-butyldiphenylsilyl-5-methyluridine

5′-O-tert-Butyldiphenylsilyl-2′-O-(2-hydroxyethyl)-5-methyluridine (20 g, 36.98 mmol) is mixed with triphenylphosphine (11.63 g, 44.36 mmol) and N-hydroxyphthalimide (7.24 g, 44.36 mmol). It is then dried over P₂O₅ under high vacuum for two days at 40° C. The reaction mixture is flushed with argon and dry THF (369.8 mL, Aldrich, sure seal bottle) is added to get a clear solution. Diethyl-azodicarboxylate (6.98 mL, 44.36 mmol) is added dropwise to the reaction mixture. The rate of addition is maintained such that resulting deep red coloration is just discharged before adding the next drop. After the addition is complete, the reaction is stirred for 4 hrs. By that time TLC showed the completion of the reaction (ethylacetate:hexane, 60:40). The solvent is evaporated in vacuum. Residue obtained is placed on a flash column and eluted with ethyl acetate:hexane (60:40), to get 2′-O-([2-phthalimidoxy)ethyl]-5′-t-butyldiphenylsilyl-5-methyluridine as white foam.

### 5′-O-tert-butyldiphenylsilyl-2′-O-[(2-formadoximinooxy)ethyl]-5-methyluridine

2′-O-([2-phthalimidoxy)ethyl]-5′-t-butyldiphenylsilyl-5-methyluridine (3.1 g, 4.5 mmol) is dissolved in dry CH₂Cl₂ (4.5 mL) and methylhydrazine (300 mL, 4.64 mmol) is added dropwise at −10° C. to 0° C. After 1 h the mixture is filtered, the filtrate is washed with ice cold CH₂Cl₂ and the combined organic phase is washed with water, brine and dried over anhydrous Na₂SO₄. The solution is concentrated to get 2′-O(aminooxyethyl)thymidine, which is then dissolved in MeOH (67.5 mL). To this formaldehyde (20% aqueous solution, w/w, 1.1 eq.) is added and the resulting mixture is stirred for 1 h. Solvent is removed under vacuum; residue chromatographed to get 5′-O-tert-butyldiphenylsilyl-2′-O-[(2-formadoximinooxy)ethyl]-5-methyluridine as white foam.

### 5′-O-tert-Butyldiphenylsilyl-2′-O-[N,N-dimethylaminooxyethyl]-5-methyluridine

5′-O-tert-butyldiphenylsilyl-2′-O-[(2-formadoximinooxy)ethyl]-5-methyluridine (1.77 g, 3.12 mmol) is dissolved in a solution of 1M pyridinium p-toluenesulfonate (PPTS) in dry MeOH (30.6 mL). Sodium cyanoborohydride (0.39 g, 6.13 mmol) is added to this solution at 10° C. under inert atmosphere. The reaction mixture is stirred for 10 minutes at 10° C. After that the reaction vessel is removed from the ice bath and stirred at room temperature for 2 h, the reaction monitored by TLC (5% MeOH in CH₂Cl₂). Aqueous NaHCO₃ solution (5%, 10 mL) is added and extracted with ethyl acetate (2×20 mL). Ethyl acetate phase is dried over anhydrous Na₂SO₄, evaporated to dryness. Residue is dissolved in a solution of 1M PPTS in MeOH (30.6 mL). Formaldehyde (20% w/w, 30 mL, 3.37 mmol) is added and the reaction mixture is stirred at room temperature for 10 minutes. Reaction mixture cooled to 10° C. in an ice bath, sodium cyanoborohydride (0.39 g, 6.13 mmol) is added, and reaction mixture stirred at 10° C. for 10 minutes. After 10 minutes, the reaction mixture is removed from the ice bath and stirred at room temperature for 2 hrs. To the reaction mixture 5% NaHCO₃ (25 mL) solution is added and extracted with ethyl acetate (2×25 mL). Ethyl acetate layer is dried over anhydrous Na₂SO₄ and evaporated to dryness. The residue obtained is purified by flash column chromatography and eluted with 5% MeOH in CH₂Cl₂ to get 5′-O-tertbutyldiphenylsilyl-2′-O-[N,N-dimethylaminooxyethyl]-5-methyluridine as a white foam.

### 2′-O-(dimethylaminooxyethyl)-5-methyluridine

Triethylamine trihydrofluoride (3.91 mL, 24.0 mmol) is dissolved in dry THF and triethylamine (1.67 mL, 12 mmol, dry, kept over KOH). This mixture of triethylamine-2HF is then added to 5′-O-tert-butyldiphenylsilyl-2′-O-[N,N-dimethylaminooxyethyl]-5-methyluridine (1.40 g, 2.4 mmol) and stirred at room temperature for 24 hrs. Reaction is monitored by TLC (5% MeOH in CH₂Cl₂). Solvent is removed under vacuum and the residue placed on a flash column and eluted with 10% MeOH in CH₂Cl₂ to get 2′-O-(dimethylaminooxyethyl)-5-methyluridine.

### 5′-O-DMT-2′-O-(dimethylaminooxyethyl)-5-methyluridine

2′-O-(dimethylaminooxyethyl)-5-methyluridine (750 mg, 2.17 mmol) is dried over P₂O₅ under high vacuum overnight at 40° C. It is then co-evaporated with anhydrous pyridine (20 mL). The residue obtained is dissolved in pyridine (11 mL) under argon atmosphere. 4-dimethylaminopyridine (26.5 mg, 2.60 mmol), 4,4′-dimethoxytrityl chloride (880 mg, 2.60 mmol) is added to the mixture and the reaction mixture is stirred at room temperature until all of the starting material disappeared. Pyridine is removed under vacuum and the residue chromatographed and eluted with 10% MeOH in CH₂Cl₂ (containing a few drops of pyridine) to get 5′-O-DMT-2′-0(dimethylamino-oxyethyl)-5-methyluridine.

### 5′-O-DMT-2′-O-(2-N,N-dimethylaminooxyethyl)-5-methyluridine-3′-[(2-cyanoethyl)-N,N-diisopropylphosphoramidite]

5′-O-DMT-2′-O-(dimethylaminooxyethyl)-5-methyluridine (1.08 g, 1.67 mmol) is co-evaporated with toluene (20 mL). To the residue N,N-diisopropylamine tetrazonide (0.29 g, 1.67 mmol) is added and dried over P20, under high vacuum overnight at 40° C. Then the reaction mixture is dissolved in anhydrous acetonitrile (8.4 mL) and 2-cyanoethyl-N,N,N¹,N¹-tetraisopropylphosphoramidite (2.12 mL, 6.08 mmol) is added. The reaction mixture is stirred at ambient temperature for 4 hrs under inert atmosphere. The progress of the reaction is monitored by TLC (hexane:ethyl acetate 1:1). The solvent is evaporated, then the residue is dissolved in ethyl acetate (70 mL) and washed with 5% aqueous NaHCO₃ (40 mL). Ethyl acetate layer is dried over anhydrous Na₂SO₄ and concentrated. Residue obtained is chromatographed (ethyl acetate as eluent) to get 5′-O-DMT-2′-O-(2-N,N-dimethylaminooxyethyl)-5-methyluridine-3′-[(2-cyanoethyl)-N,N-diisopropylphosphoramidite] as a foam.

### 2′-(Aminooxyethoxy) nucleoside amidites

2′-(Aminooxyethoxy) nucleoside amidites [also known in the art as 2′-O-(aminooxyethyl) nucleoside amidites] are prepared as described in the following paragraphs. Adenosine, cytidine and thymidine nucleoside amidites are prepared similarly.

### N2-isobutyryl-6-O-diphenylcarbamoyl-2′-O-(2-ethylacetyl)-5′-O-(4,4′-dimethoxytrityl)guanosine-3′-[(2-cyanoethyl)-N,N-diisopropylphosphoramidite]

The 2′-O-aminooxyethyl guanosine analog may be obtained by selective 2′-O-alkylation of diaminopurine riboside. Multigram quantities of diaminopurine riboside may be purchased from Schering AG (Berlin) to provide 2′-O-(2-ethylacetyl)diaminopurine riboside along with a minor amount of the 3′-O-isomer. 2′-O-(2-ethylacetyl)diaminopurine riboside may be resolved and converted to 2′-O-(2ethylacetyl)guanosine by treatment with adenosine deaminase. (McGee, D. P. C., Cook, P. D., Guinosso, C. J., WO 94/02501 A1 940203.) Standard protection procedures should afford 2′-O-(2-ethylacetyl)-5′-O-(4,4′-dimethoxytrityl)guanosine and 2-N-isobutyryl-6-O-diphenylcarbamoyl-2′-O-(2-ethylacetyl)-5′-O-(4,4′-dimethoxytrityl)guanosine which may be reduced to provide 2-N-isobutyryl-6-O-diphenylcarbamoyl-2′-O-(2-ethylacetyl)-5′-O-(4,4′-dimethoxytrityl)guanosine. As before the hydroxyl group may be displaced by N-hydroxyphthalimide via a Mitsunobu reaction, and the protected nucleoside may phosphitylated as usual to yield 2-N-isobutyryl-6-O-diphenylcarbamoyl-2′-O-(2-ethylacetyl)-5′-O-(4,4′-dimethoxytrityl)guanosine-3′-[(2-cyanoethyl)-N,N-diisopropylphosphoramiditel.

### 2′-dimethylaminoethoxyethoxy (2′-DMAEOE) nucleoside amidites

2′-dimethylaminoethoxyethoxy nucleoside amidites (also known in the art as 2′-O-dimethylaminoethoxyethyl, i.e., 2′O—CH₂—O—CH₂—N(CH₂)₂, or 2′-DMAEOE nucleoside amidites) are prepared as follows. Other nucleoside amidites are prepared similarly.

### 2′-O-[2(2-N,N-dimethylaminoethoxy)ethyl]-5-methyl uridine

2[2-(Dimethylamino)ethoxylethanol (Aldrich, 6.66 g, 50 mmol) is slowly added to a solution of borane in tetrahydrofuran (1 M, 10 mL, 10 mmol) with stirring in a 100 mL bomb. Hydrogen gas evolves as the solid dissolves. O²-,2′-anhydro-5-methyluridine (1.2 g, 5 mmol), and sodium bicarbonate (2.5 mg) are added and the bomb is sealed, placed in an oil bath, and heated to 155° C. for 26 hours. The bomb is cooled to room temperature and opened. The crude solution is concentrated and the residue partitioned between water (200 mL) and hexanes (200 mL). The excess phenol is extracted into the hexane layer. The aqueous layer is extracted with ethyl acetate (3×200 mL) and the combined organic layers are washed once with water, dried over anhydrous sodium sulfate, and concentrated. The residue is columned on silica gel using methanol/methylene chloride 1:20 (which has 2% triethylamine) as the eluent. As the column fractions are concentrated a colorless solid forms which is collected to give the title compound as a white solid.

### 5′-O-dimethoxytrityl-2′-O-[2(2-N,N-dimethylaminoethoxy)ethyl)]-5-methyl uridine

To 0.5 g (1.3 mmol) of 2′-O-[2(2-N,N-dimethylaminoethoxy)ethyl)1-5-methyl uridine in anhydrous pyridine (8 mL), triethylamine (0.36 mL) and dimethoxytrityl chloride (DMT-Cl, 0.87 g, 2 eq.) are added and stirred for 1 hour. The reaction mixture is poured into water (200 mL) and extracted with CH₂Cl₂ (2×200 mL). The combined CH₂Cl₂ layers are washed with saturated NaHCO₃ solution, followed by saturated NaCl solution, and dried over anhydrous sodium sulfate. Evaporation of the solvent followed by silica gel chromatography using MeOH: CH₂Cl₂:Et₃N (20:1, v/v, with 1% triethylamine) gives the title compound.

### 5′-O-Dimethoxytrityl-2′-O-[2(2-N,N-dimethylaminoethoxy)ethyl)]-5-methyl uridine-3′-O-(cyanoethyl-N,N-diisopropyl)phosphoramidite

Diisopropylaminotetrazolide (0.6 g) and 2-cyanoethoxyN,N-diisopropyl phosphoramidite (1.1 mL, 2 eq.) are added to a solution of 5′-O-dimethoxytrityl-2′-O-[2(2-N,N-dimethylaminoethoxy)ethyl)]-5-methyluridine (2.17 g, 3 mmol) dissolved in CH₂Cl₂ (20 mL) under an atmosphere of argon. The reaction mixture is stirred overnight and the solvent evaporated. The resulting residue is purified by silica gel flash column chromatography with ethyl acetate as the eluent to give the title compound.

### Example 2

Oligonucleotide Synthesis

Unsubstituted and substituted phosphodiester (P═O) oligonucleotides are synthesized on an automated DNA synthesizer (Applied Biosystems model 380B) using standard phosphoramidite chemistry with oxidation by iodine.

Phosphorothioates (P═S) are synthesized as for the phosphodiester oligonucleotides except the standard oxidation bottle is replaced by 0.2 M solution of 3H-1,2-benzodithiole-3-one 1,1-dioxide in acetonitrile for the stepwise thiation of the phosphite linkages. The thiation wait step is increased to 68 sec and is followed by the capping step. After cleavage from the CPG column and deblocking in concentrated ammonium hydroxide at 55° C. (18 h), the oligonucleotides are purified by precipitating twice with 2.5 volumes of ethanol from a 0.5 M NaCl solution. Phosphinate oligonucleotides are prepared as described in U.S. Pat. No. 5,508,270, herein incorporated by reference.

Alkyl phosphonate oligonucleotides are prepared as described in U.S. Pat. No. 4,469,863, herein incorporated by reference.

3′-Deoxy-3′-methylene phosphonate oligonucleotides are prepared as described in U.S. Pat. No. 5,610,289 or 5,625,050, herein incorporated by reference.

Phosphoramidite oligonucleotides are prepared as described in U.S. Pat. No. 5,256,775 or U.S. Pat. No. 5,366,878, herein incorporated by reference.

Alkylphosphonothioate oligonucleotides are prepared as described in WO 94/17093 and WO 94/02499 herein incorporated by reference.

3′-Deoxy-3′-amino phosphoramidate oligonucleotides are prepared as described in U.S. Pat. No. 5,476,925, herein incorporated by reference.

Phosphotriester oligonucleotides are prepared as described in U.S. Pat. No. 5,023,243, herein incorporated by reference.

Borano phosphate oligonucleotides are prepared as described in U.S. Pat. Nos. 5,130,302 and 5,177,198, both herein incorporated by reference.

### Example 3

Oligonucleoside Synthesis

Methylenemethylimino linked oligonucleosides, also identified as MMI linked oligonucleosides, methylenedimethylhydrazo linked oligonucleosides, also identified as MDH linked oligonucleosides, and methylenecarbonylamino linked oligonucleosides, also identified as amide-3 linked oligonucleosides, and methyleneaminocarbonyl linked oligonucleosides, also identified as amide-4 linked oligonucleosides, as well as mixed backbone compounds having, for instance, alternating MMI and P═O or P═S linkages are prepared as described in U.S. Pat. Nos. 5,378,825; 5,386,023; 5,489,677; 5,602,240; and 5,610,289, all of which are herein incorporated by reference.

Formacetal and thioformacetal linked oligonucleosides are prepared as described in U.S. Pat. Nos. 5,264,562 and 5,264,564, herein incorporated by reference.

Ethylene oxide linked oligonucleosides are prepared as described in U.S. Pat. No. 5,223,618, herein incorporated by reference.

### Example 4

PNA Synthesis

Peptide nucleic acids (PNAs) are prepared in accordance with any of the various procedures referred to in Peptide Nucleic Acids (PNA): Synthesis, Properties and Potential Applications, Bioorganic & Medicinal Chemistry, 1996, 4, 523. They may also be prepared in accordance with U.S. Pat. Nos. 5,539,082; 5,700,922; and 5,719,262, herein incorporated by reference.

### Example 5

Synthesis of Chimeric Oligonucleotides

Chimeric oligonucleotides, oligonucleosides, or mixed oligonucleotides/oligonucleosides of the invention can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers”.

### [2′-O-Me]-[2′-deoxy]-[2′-O-Me]Chimeric Phosphorothioate Oligonucleotides

Chimeric oligonucleotides having 2′-O-alkyl phosphorothioate and 2′-deoxy phosphorothioate oligonucleotide segments are synthesized using an Applied Biosystems automated DNA synthesizer Model 380B, as above. Oligonucleotides are synthesized using the automated synthesizer and 2′-deoxy-5′-dimethoxytrityl-3′-O-phosphoramidite for the DNA portion and 5′-dimethoxytrityl-2′-O-methyl-3′-O-phosphoramidite for 5′ and 3′ wings. The standard synthesis cycle is modified by increasing the wait step after the delivery of tetrazole and base to 600 s repeated four times for RNA and twice for 2′-O-methyl. The fully protected oligonucleotide is cleaved from the support and the phosphate group is deprotected in 3:1 ammonia/ethanol at room temperature overnight then lyophilized to dryness. Treatment in methanolic ammonia for 24 hrs at room temperature is then done to deprotect all bases and sample is again lyophilized to dryness. The pellet is resuspended in 1M TBAF in THF for 24 hrs at room temperature to deprotect the 2′ positions. The reaction is then quenched with 1M TEAA and the sample is then reduced to ½ volume by rotovac before being desalted on a G25 size exclusion column. The oligo recovered is then analyzed spectrophotometrically for yield and for purity by capillary electrophoresis and by mass spectrometry.

### [2′-O-(2-Methoxyethyl)]-[2′-deoxy]-[2′-O-(Methoxyethyl)]Chimeric Phosphorothioate Oligonucleotides

[2′-O-(2-methoxyethyl)]-[2′-deoxy]-[-2′-O-(methoxyethyl)]chimeric phosphorothioate oligonucleotides are prepared as per the procedure above for the 2′-O-methyl chimeric oligonucleotide, with the substitution of phorothioate oligonucleotides are prepared as per the procedure above for 2′-O-(methoxyethyl) amidites for the 2′-O-methyl amidites.

### [2′-O-(2-Methoxyethyl)Phosphodiester]-[2′-deoxy Phosphorothioate]-[2′-O-(2-Methoxyethyl)]Phosphodiester]Chimeric Oligonucleotides

[2′-O-(2-methoxyethyl phosphodiester]-[2′-deoxy phosphorothioate]-[2′-O-(methcixyethyl)phosphodiester]chimeric oligonucleotides are prepared as per the above procedure for the 2′-O-methyl chimeric oligonucleotide with the substitution of 2′-O-(methoxyethyl) amidites for the 2′-O-methyl amidites, oxidization with iodine to generate the phosphodiester internucleotide linkages within the wing portions of the chimeric structures and sulfurization utilizing 3,H-1,2 benzodithiole-3-one 1,1 dioxide (Beaucage Reagent) to generate the phosphorothioate internucleotide linkages for the center gap.

Other chimeric oligonucleotides, chimeric oligonucleosides, and mixed chimeric oligonucleotides/oligonucleosides are synthesized according to U.S. Pat. No. 5,623,065, herein incorporated by reference.

### Example 6

Oligonucleotide Isolation

After cleavage from the controlled pore glass column (Applied Biosystems) and deblocking in concentrated ammonium hydroxide at 55° C. for 18 hours, the oligonucleotides or oligonucleosides are purified by precipitation twice out of 0.5 M NaCl with 2.5 volumes ethanol. Synthesized oligonucleotides are analyzed by polyacrylamide gel electrophoresis on denaturing gels and judged to be at least 85% full-length material. The relative amounts of phosphorothioate and phosphodiester linkages obtained in synthesis are periodically checked by “P nuclear magnetic resonance spectroscopy, and for some studies oligonucleotides are purified by HPLC, as described by Chiang et al., J. Biol. Chem. 1991, 266, 18162-18171.

### Example 7

Oligonucleotide Synthesis—96 Well Plate Format

Oligonucleotides are synthesized via solid phase P(III) phosphoramidite chemistry on an automated synthesizer capable of assembling 96 sequences simultaneously in a standard 96 well format. Phosphodiester internucleotide linkages are afforded by oxidation with aqueous iodine. Phosphorothioate internucleotide linkages are generated by sulfurization utilizing 3,H-1,2 benzodithiole-3-one 1,1 dioxide (Beaucage Reagent) in anhydrous acetonitrile. Standard base-protected beta-cyanoethyldiisopropyl phosphoramidites can be purchased from commercial vendors (e.g. PE-Applied Biosystems, Foster City, Calif., or Pharmacia, Piscataway, N.J.). Non-standard nucleosides are synthesized as per known literature or patented methods. They are utilized as base protected betacyanoethyldiisopropyl phosphoramidites.

Oligonucleotides are cleaved from support and deprotected with concentrated NH₄OH at elevated temperature (55-60° C.) for 12-16 hours and the released product then dried in vacuo. The dried product is then re-suspended in sterile water to afford a master plate from which all analytical and test plate samples are then diluted utilizing robotic pipettors.

### Example 8

Oligonucleotide Analysis—96 Well Plate Format

The concentration of oligonucleotide in each well is assessed by dilution of samples and UV absorption spectroscopy. The full-length integrity of the individual products is evaluated by capillary electrophoresis (CE) in either the 96 well format (Beckman P/ACE™ MDQ) or, for individually prepared samples, on a commercial CE apparatus (e.g., Beckman P/ACE™ 5000, ABI 270). Base and backbone composition is confirmed by mass analysis of the compounds utilizing electrospray-mass spectroscopy. All assay test plates are diluted from the master plate using single and multi-channel robotic pipettors. Plates are judged to be acceptable if at least 85% of the compounds on the plate are at least 85% full length.

### Example 9

Cell Culture and Oligonucleotide Treatment

The effect of antisense compounds on target nucleic acid expression can be tested in any of a variety of cell types provided that the target nucleic acid is present at measurable levels. This can be routinely determined using, for example, PCR or Northern blot analysis. The following 6 cell types are provided for illustrative purposes, but other cell types can be routinely used, provided that the target is expressed in the cell type chosen. This can be readily determined by methods routine in the art, for example Northern blot analysis, Ribonuclease protection assays, or RT-PCR.

T-24 Cells:

The human transitional cell bladder carcinoma cell line T-24 is obtained from the American Type Culture Collection (ATCC) (Manassas, Va.). T-24 cells are routinely cultured in complete McCoy's 5A basal media (Gibco/Life Technologies, Gaithersburg, Md.) supplemented with 10% fetal calf serum (Gibco/Life Technologies, Gaithersburg, Md.), penicillin 100 units per mL, and streptomycin 100 micrograms per mL (Gibco/Life Technologies, Gaithersburg, Md.). Cells are routinely passaged by trypsinization and dilution when they reached 90% confluence. Cells are seeded into 96-well plates (Falcon-Primaria #3872) at a density of 7000 cells/well for use in RT-PCR analysis.

For Northern blotting or other analysis, cells may be seeded onto 100 mm or other standard tissue culture plates and treated similarly, using appropriate volumes of medium and oligonucleotide.

A549 Cells:

The human lung carcinoma cell line A549 can be obtained from the American Type Culture Collection (ATCC) (Manassas, Va.). A549 cells are routinely cultured in DMEM basal media (Gibco/Life Technologies, Gaithersburg, Md.) supplemented with 10% fetal calf serum (Gibco/Life Technologies, Gaithersburg, Md.), penicillin 100 units per mL, and streptomycin 100 micrograms per mL (Gibco/Life Technologies, Gaithersburg, Md.). Cells are routinely passaged by trypsinization and dilution when they reached 90% confluence.

NHDF Cells:

Human neonatal dermal fibroblast (NHDF) can be obtained from the Clonetics Corporation (Walkersville Md.). NHDFs are routinely maintained in Fibroblast Growth Medium (Clonetics Corporation, Walkersville Md.) supplemented as recommended by the supplier. Cells are maintained for up to 10 passages as recommended by the supplier.

HEK Cells:

Human embryonic keratinocytes (HEK) can be obtained from the Clonetics Corporation (Walkersville Md.). HEKs are routinely maintained in Keratinocyte Growth Medium (Clonetics Corporation, Walkersville Md.) formulated as recommended by the supplier. Cells are routinely maintained for up to 10 passages as recommended by the supplier.

MCF-7 Cells:

The human breast carcinoma cell line MCF-7 is obtained from the American Type Culture Collection (Manassas, Va.). MCF-7 cells are routinely cultured in DMEM low glucose (Gibco/Life Technologies, Gaithersburg, Md.) supplemented with 10% fetal calf serum (Gibco/Life Technologies, Gaithersburg, Md.). Cells are routinely passaged by trypsinization and dilution when they reached 90% confluence. Cells are seeded into 96-well plates (Falcon-Primaria #3872) at a density of 7000 cells/well for use in RT-PCR analysis.

For Northern blotting or other analyses, cells may be seeded onto 100 mm or other standard tissue culture plates and treated similarly, using appropriate volumes of medium and oligonucleotide.

LA4 Cells:

The mouse lung epithelial cell line LA4 is obtained from the American Type Culture Collection (Manassas, Va.). LA4 cells are routinely cultured in F12K medium (Gibco/Life Technologies, Gaithersburg, Md.) supplemented with 15% fetal calf serum (Gibco/Life Technologies, Gaithersburg, Md.). Cells are routinely passaged by trypsinization and dilution when they reached 90% confluence. Cells are seeded into 96-well plates (Falcon-Primaria #3872) at a density of 3000-6000 cells/well for use in RT-PCR analysis.

For Northern blotting or other analyses, cells may be seeded onto 100 mm or other standard tissue culture plates and treated similarly, using appropriate volumes of medium and oligonucleotide.

Treatment with Antisense Compounds:

When cells reached 80% confluence, they are treated with oligonucleotide. For cells grown in 96-well plates, wells are washed once with 200 μL OPTI-MEM™-1 reduced-serum medium (Gibco BRL) and then treated with 130 μL of OPTI-MEMTM™-1 containing 3.75 μg/mL LIPOFECTIN™ (Gibco BRL) and the desired concentration of oligonucleotide. After 4-7 hours of treatment, the medium is replaced with fresh medium. Cells are harvested 16-24 hours after oligonucleotide treatment.

The concentration of oligonucleotide used varies from cell line to cell line. To determine the optimal oligonucleotide concentration for a particular cell line, the cells are treated with a positive control oligonucleotide at a range of concentrations.

### Example 10

Analysis of Oligonucleotide Inhibition of FXR Expression

Antisense modulation of FXR expression can be assayed in a variety of ways known in the art. For example, FXR mRNA levels can be quantitated by, e.g., Northern blot analysis, competitive polymerase chain reaction (PCR), or real-time PCR (RT-PCR). Real-time quantitative PCR is presently preferred. RNA analysis can be performed on total cellular RNA or poly(A)+ mRNA. Methods of RNA isolation are taught in, for example, Ausubel, F. M. et al., Current Protocols in Molecular Biology, Volume 1, pp. 4.1.1-4.2.9 and 4.5.1-4.5.3, John Wiley & Sons, Inc., 1993. Northern blot analysis is routine in the art and is taught in, for example, Ausubel, F. M. et al., Current Protocols in Molecular Biology, Volume 1, pp. 4.2.1-4.2.9, John Wiley & Sons, Inc., 1996. Real-time quantitative (PCR) can be conveniently accomplished using the commercially available ABI PRISM™ 7700 Sequence Detection System, available from PE-Applied Biosystems, Foster City, Calif. and used according to manufacturer's instructions. Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed as multiplexable. Other methods of PCR are also known in the art.

Protein levels of FXR can be quantitated in a variety of ways well known in the art, such as immunoprecipitation, Western blot analysis (immunoblotting), ELISA or fluorescence-activated cell sorting (FACS). Antibodies directed to FXR can be identified and obtained from a variety of sources, such as the MSRS catalog of antibodies (Aerie Corporation, Birmingham, Mich.), or can be prepared via conventional antibody generation methods. Methods for preparation of polyclonal antisera are taught in, for example, Ausubel, F. M. et al., Current Protocols in Molecular Biology, Volume 2, pp. 11.12.1-11.12.9, John Wiley & Sons, Inc., 1997. Preparation of monoclonal antibodies is taught in, for example, Ausubel, F. M. et al., Current Protocols in Molecular Biology, Volume 2, pp. 11.4.1-11.11.5, John Wiley Sons, Inc., 1997.

Immunoprecipitation methods are standard in the art and can be found at, for example, Ausubel, F. M. et al., Current Protocols in Molecular Biology, Volume 2, pp. 10.16.110.16.11, John Wiley & Sons, Inc., 1998. Western blot (immunoblot) analysis is standard in the art and can be found at, for example, Ausubel, F. M. et al., Current Protocols in Molecular Biology, Volume 2, pp. 10.8.1-10.8.21, John Wiley Sons, Inc., 1997. Enzyme-linked immunosorbent assays (ELISA) are standard in the art and can be found at, for example, Ausubel, F. M. et al., Current Protocols in Molecular Biology, Volume 2, pp. 11.2.1-11.2.22, John Wiley & Sons, Inc., 1991.

### Example 11

Poly(A)+ mRNA Isolation

Poly(A)+ mRNA is isolated according to Miura et al., Clin. Chem., 1996, 42, 1758-1764. Other methods for poly(A)+ mRNA isolation are taught in, for example, Ausubel, F. M. et al., Current Protocols in Molecular Biology, Volume 1, pp. 4.5.1-4.5.3, John Wiley & Sons, Inc., 1993. Briefly, for cells grown on 96-well plates, growth medium is removed from the cells and each well is washed with 200 μL cold PBS. 60 μL lysis buffer (10 mM Tris-HCl, pH 7.6, 1 mM EDTA, 0.5 M NaCl, 0.5% NP-40, 20 mM vanadyl-ribonucleoside complex) is added to each well, the plate is gently agitated and then incubated at room temperature for five minutes. 55 μL of lysate is transferred to Oligo d(T) coated 96-well plates (AGCT Inc., Irvine Calif.). Plates are incubated for 60 minutes at room temperature, washed 3 times with 200 μL of wash buffer (10 mM Tris-HCl pH 7.6, 1 mM EDTA, 0.3 M NaCl). After the final wash, the plate is blotted on paper towels to remove excess wash buffer and then air-dried for 5 minutes. 60 pL of elution buffer (5 mM Tris-HCl pH 7.6), preheated to 70° C. is added to each well, the plate is incubated on a 90° C. hot plate for 5 minutes, and the eluate is then transferred to a fresh 96-well plate.

Cells grown on 100 mm or other standard plates may be treated similarly, using appropriate volumes of all solutions.

### Example 12

Total RNA Isolation

Total mRNA is isolated using an RNEASY 96™ kit and buffers purchased from Qiagen Inc. (Valencia Calif.) following the manufacturer's recommended procedures. Briefly, for cells grown on 96-well plates, growth medium is removed from the cells and each well is washed with 200 μL cold PBS. 100 μL Buffer RLT is added to each well and the plate vigorously agitated for 20 seconds. 100 μL of 70% ethanol is then added to each well and the contents mixed by pipetting three times up and down. The samples are then transferred to the RNEASY 96™ well plate attached to a QIAVAC™ manifold fitted with a waste collection tray and attached to a vacuum source. Vacuum is applied for 15 seconds. 1 mL of Buffer RW1 is added to each well of the RNEASY 96™ plate and the vacuum again applied for 15 seconds. 1 mL of Buffer RPE is then added to each well of the RNEASY 96™ plate and the vacuum applied for a period of 15 seconds. The Buffer RPE wash is then repeated and the vacuum is applied for an additional 10 minutes. The plate is then removed from the QIAVAC™ manifold and blotted dry on paper towels. The plate is then re-attached to the QIAVAC™ manifold fitted with a collection tube rack containing 1.2 mL collection tubes. RNA is then eluted by pipetting 60 μL water into each well, incubating one minute, and then applying the vacuum for 30 seconds. The elution step is repeated with additional 60 μL water.

The repetitive pipetting and elution steps may be automated using a QIAGEN Bio-Robot 9604 (Qiagen, Inc., Valencia Calif.). Essentially, after lysing of the cells on the culture plate, the plate is transferred to the robot deck where the pipetting, DNase treatment and elution steps are carried out.

### Example 13

Real-Time Quantitative PCR Analysis of FXR mRNA Levels

Quantitation of FXR mRNA levels is determined by real-time quantitative PCR using the ABI PRISM™ 7700 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR, in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., JOE, FAM™, or VIC, obtained from either Operon Technologies Inc., Alameda, Calif. or PE-Applied Biosystems, Foster City, Calif.) is attached to the 5′ end of the probe and a quencher dye (e.g., TAMRA, obtained from either Operon Technologies Inc., Alameda, Calif. or PE-Applied Biosystems, Foster City, Calif.) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM™ 7700 Sequence Detection System. In each assay, a series of parallel reactions containing serial dilutions of mRNA from untreated control samples generates a standard curve that is used to quantitate the percent inhibition after antisense oligonucleotide treatment of test samples.

PCR reagents can be obtained from PE-Applied Biosystems, Foster City, Calif. RT-PCR reactions are carried out by adding 25 μL PCR cocktail (1×TAQMAN™ buffer A, 5.5 MM MgCl₂, 300 μM each of dATP, dCTP and dGTP, 600 μM of dUTP, 100 nM each of forward primer, reverse primer, and probe, 20 Units RNAse inhibitor, 1.25 Units AMPLITAQ GOLD™, and 12.5 Units MuLV reverse transcriptase) to 96 well plates containing 25 μL poly(A) mRNA solution. The RT reaction is carried out by incubation for 30 minutes at 48° C. Following a 10 minute incubation at 95° C. to activate the AMPLITAQ GOLD™, 40 cycles of a two-step PCR protocol are carried out: 95° C. for 15 seconds (denaturation) followed by 60° C. for 1.5 minutes (annealing/extension).

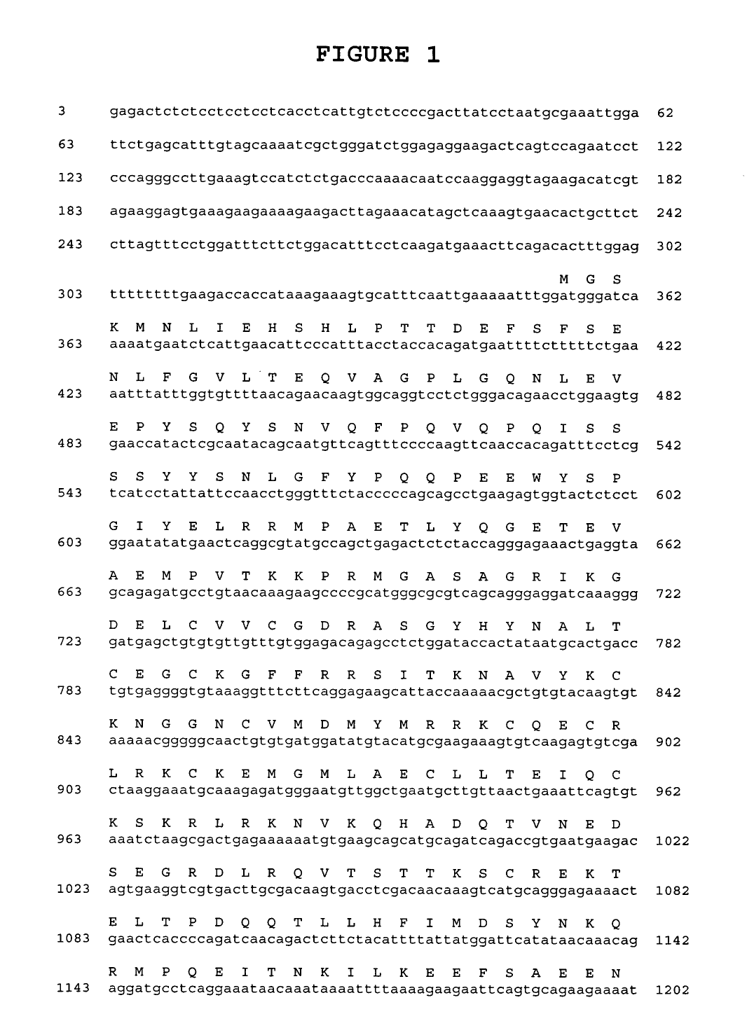

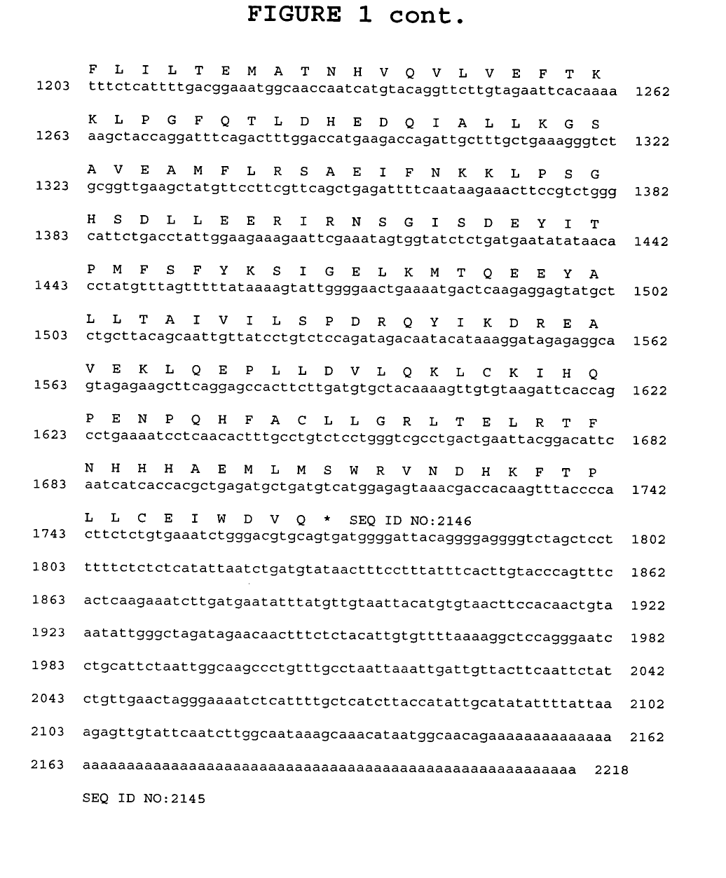

Probes and primers to human FXR were designed to hybridize to a human FXR sequence, using published sequence, information (NM_{—}005123, incorporated herein as FIG. 1). For human FXR the PCR primers were:

forward primer: CTGGGTCGCCTGACTGAATT SEQ ID NO:2139

reverse primer: GGTCGTTTACTCTCCATGACATCA SEQ ID NO:2140 and the PCR

probe is: FAM™-CGGACATTCAATCATCACCACGCTGAG SEQ ID NO:2141-TAMRA where FAM™ (PE-Applied Biosystems, Foster City, Calif.) is the fluorescent reporter dye) and TAMRA (PE-Applied Biosystems, Foster City, Calif.) is the quencher dye. For human cyclophilin the PCR primers were: forward

primer: CCCACCGTGTTCTTCGACAT SEQ ID NO:2142

reverse primer: TTTCTGCTGTCTTTGGGACCTT SEQ ID NO:2143 and the PCR

probe is: 5′ JOE-CGCGTCTCCTTTGAGCTGTTTGCA SEQ ID NO: 2144-TAMRA 3′ where JOE (PE-Applied Biosystems, Foster City, Calif.) is the fluorescent reporter dye) and TAMRA (PE-Applied Biosystems, Foster City, Calif.) is the quencher dye.

### Example 14

Antisense Inhibition of Human FXR Expression by Chimeric Phosphorothioate Oligonucleotides having 2′-MOE Wings and a Deoxy Gap

In accordance with the present invention, a series of oligonucleotides are designed to target different regions of the human FXR RNA, using published sequences (NM_{—}005123, incorporated herein as FIG. 1). The oligonucleotides are shown in Table 1. “Position” indicates the first (5′-most) nucleotide number on the particular target sequence to which the oligonucleotide binds. The indicated parameters for each oligo were predicted using RNAstructure 3.7 by David H. Mathews, Michael Zuker, and Douglas H. Turner. The parameters are described either as free energy (The energy that is released when a reaction occurs. The more negative the number, the more likely the reaction will occur. All free energy units are in kcal/mol. or melting temperature (the temperature at which two anneal strands of polynucleic acid separate. The higher the temperature, greater the affinity between the 2 strands.) When designing an antisense oligonucleotide (oligomers) that will bind with high affinity, it is desirable to consider the structure of the target RNA strand and the antisense oligomer. Specifically, for an oligomer to bind tightly (in the table described as ‘duplex formation’), it should be complementary to a stretch of target RNA that has little self-structure (in the table the free energy of which is described as ‘target structure’). Also, the oligomer should have little self-structure, either intramolecular (in the table the free energy of which is described as ‘intramolecular oligo’) or bimolecular (in the table the free energy of which is described as ‘intermolecular oligo’). Breaking up any self-structure amounts to a binding penalty. All compounds in Table 1 are chimeric oligonucleotides (“gapmers”) 20 nucleotides in length, composed of a central “gap” region consisting of ten 2′deoxynucleotides, which is flanked on both sides (5′ and 3′ directions) by four-nucleotide “wings”. The wings are composed of 2′-methoxyethyl (2′-MOE) nucleotides. The internucleoside (backbone) linkages are phosphorothioate (P═S) throughout the oligonucleotide. Cytidine residues in the 2′-MOE wings are 5-methylcytidines. All cytidine residues are 5-methylcytidines.

TABLE 1

| | | | | | | | |
|---|---|---|---|---|---|---|---|
| | | | | | | | kcal/ |
| | | | kcal/ | | kcal/ | kcal/ | mol |
| | | kcal/ | mol | | mol | Intra- | Inter- |
| | | mol | duplex | deg C. | target | mole- | mole- |
| | | total | forma- | Tm of | struc- | cular | cular |
| position | oligo | binding | tion | Duplex | ture | oligo | oligo |

| | | | | | | | |
|---|---|---|---|---|---|---|---|
| 1132 | AGGCATCCTCTGTTTGTTAT | −21.6 | −24.7 | 73.8 | −3.1 | 0 | −4 |
| | SEQ. ID. NO:1 | | | | | | |
| 1136 | CCTGAGGCATCCTCTGTTTG | −21.6 | −27.2 | 77.4 | −3.1 | −2.5 | −7.9 |
| | SEQ. ID. NO:2 | | | | | | |
| 682 | CGCGCCCATGCGGGGCTTCT | −21.5 | −34.2 | 84.9 | −8.2 | −4.5 | −11.3 |
| | SEQ. ID. NO:3 | | | | | | |
| 684 | GACGCGCCCATGCGGGGCTT | −21.5 | −33.7 | 83.3 | −8.2 | −4 | −11.8 |
| | SEQ. ID. NO:4 | | | | | | |
| 1131 | GGCATCCTCTGTTTGTTATA | −21.3 | −24.4 | 72.8 | −3.1 | 0 | −4 |
| | SEQ. ID. NO:5 | | | | | | |
| 882 | CGACACTCTTGACACTTTCT | −21 | −22.9 | 67 | −1.9 | 0 | −2.1 |
| | SEQ. ID. NO:6 | | | | | | |
| 685 | TGACGCGCCCATGCGGGGCT | −20.9 | −33.6 | 82.7 | −8.2 | −4.5 | −11.8 |
| | SEQ. ID. NO:7 | | | | | | |
| 681 | GCGCCCATGCGGGGCTTCTT | −20.8 | −33.5 | 85.9 | −8.2 | −4.5 | −11.3 |
| | SEQ. ID. NO:8 | | | | | | |
| 683 | ACGCGCCCATGCGGGGCTTC | −20.8 | −33.5 | 83.7 | −8.2 | −4.5 | −11.8 |
| | SEQ. ID. NO:9 | | | | | | |
| 686 | CTGACGCGCCCATGCGGGGC | −20.8 | −33.6 | 82.7 | −9.1 | −3.7 | −11.1 |
| | SEQ. ID. NO:10 | | | | | | |
| 1135 | CTGAGGCATCCTCTGTTTGT | −20.8 | −26.4 | 77.4 | −3.1 | −2.5 | −7.9 |
| | SEQ. ID. NO:11 | | | | | | |
| 678 | CCCATGCGGGGCTTCTTTGT | −20.7 | −30.4 | 81.7 | −8.2 | −1.4 | −6.8 |
| | SEQ. ID. NO:12 | | | | | | |
| 848 | CCATCACACAGTTGCCCCCG | −20.5 | −31.5 | 80.3 | −11 | 0 | −3 |
| | SEQ. ID. NO:13 | | | | | | |
| 883 | TCGACACTCTTGACACTTTC | −20.5 | −22.4 | 66.6 | −1.9 | 0 | −4.2 |
| | SEQ. ID. NO:14 | | | | | | |
| 845 | TCACACAGTTGCCCCCGTTT | −20.4 | −30.2 | 80.1 | −9.8 | 0 | −3 |
| | SEQ. ID. NO:15 | | | | | | |
| 1133 | GAGGCATCCTCTGTTTGTTA | −20.4 | −25.3 | 75.3 | −3.1 | −1.8 | −7.1 |
| | SEQ. ID. NO:16 | | | | | | |
| 881 | GACACTCTTGACACTTTCTT | −20.3 | −22.2 | 67.1 | −1.9 | 0 | −2.3 |
| | SEQ. ID. NO:17 | | | | | | |
| 884 | GTCGACACTCTTGACACTTT | −20.3 | −23.2 | 68.3 | −1.9 | −0.7 | −8.8 |
| | SEQ. ID. NO:18 | | | | | | |
| 844 | CACACAGTTGCCCCCGTTTT | −20.1 | −29.9 | 78.8 | −9.8 | 0 | −3 |
| | SEQ. ID. NO:19 | | | | | | |
| 1130 | GCATCCTCTGTTTGTTATAT | −20.1 | −23.2 | 70 | −3.1 | 0 | 3.4 |
| | SEQ. ID. NO:20 | | | | | | |
| 1138 | TTCCTGAGGCATCCTCTGTT | −20.1 | −27.6 | 79.5 | −5.7 | −1.8 | −7.2 |
| | SEQ. ID. NO:21 | | | | | | |
| 219 | GCAGTGTTCACTTTGAGCTA | −20 | −24.4 | 73.6 | −3.9 | −0.1 | −7.9 |
| | SEQ. ID. NO:22 | | | | | | |
| 1134 | TGAGGCATCCTCTGTTTGTT | −20 | −25.6 | 75.7 | −3.1 | −2.5 | −7.9 |
| | SEQ. ID. NO:23 | | | | | | |
| 220 | AGCAGTGTTCACTTTGAGCT | −19.9 | −24.7 | 74.6 | −3.9 | −0.8 | −8 |
| | SEQ. ID. NO:24 | | | | | | |
| 1143 | GTTATTTCCTGAGGCATCCT | −19.8 | −26.1 | 75.6 | −5.7 | −0.3 | −5.4 |
| | SEQ. ID. NO:25 | | | | | | |
| 677 | CCATGCGGGGCTTCTTTGTT | −19.7 | −28.5 | 78.7 | −8.2 | −0.3 | −4.3 |
| | SEQ. ID. NO:26 | | | | | | |
| 847 | CATCACACAGTTGCCCCCGT | −19.7 | −30.7 | 80.3 | −11 | 0 | −3 |
| | SEQ. ID. NO:27 | | | | | | |
| 885 | AGTCGACACTCTTGACACTT | −19.6 | −23.1 | 68.2 | −1.9 | −1.5 | −9.5 |
| | SEQ. ID. NO:28 | | | | | | |
| 1144 | TGTTATTTCCTGAGGCATCC | −19.5 | −25.2 | 73.4 | −5.7 | 0 | −5.4 |
| | SEQ. ID. NO:29 | | | | | | |
| 315 | TGCACTTTCTTTATGGTGGT | −19.4 | −23.8 | 71.5 | −3.7 | −0.5 | −4.7 |
| | SEQ. ID. NO:30 | | | | | | |
| 846 | ATCACACAGTTGCCCCCGTT | −19.4 | −30.1 | 79.7 | −10.7 | 0 | −3 |
| | SEQ. ID. NO:31 | | | | | | |
| 906 | CCCATCTCTTTGCATTTCCT | −19.4 | −27.5 | 77.2 | −8.1 | 0 | −5.1 |
| | SEQ. ID. NO:32 | | | | | | |
| 1139 | TTTCCTGAGGCATCCTCTGT | −19.4 | −27.6 | 79.5 | −5.7 | −2.5 | −7.9 |
| | SEQ. ID. NO:33 | | | | | | |
| 1655 | GTAATTCAGTCAGGCGACCC | −19.4 | −26.3 | 73.9 | −5.5 | −1.3 | −5.4 |
| | SEQ. ID. NO:34 | | | | | | |
| 886 | TAGTCGACACTCTTGACACT | −19.2 | −22.7 | 67.2 | −1.9 | −1.5 | −9.5 |
| | SEQ. ID. NO:35 | | | | | | |
| 314 | GCACTTTCTTTATGGTGGTC | −19.1 | −24.2 | 73.4 | −4.4 | −0.5 | −4.5 |
| | SEQ. ID. NO:36 | | | | | | |
| 680 | CGCCCATGCGGGGCTTCTTT | −19.1 | −31.8 | 82.2 | −8.2 | −4.5 | −11.3 |
| | SEQ. ID. NO:37 | | | | | | |
| 907 | TCCCATCTCTTTGCATTTCC | −18.9 | −27 | 76.9 | −8.1 | 0 | −5.1 |
| | SEQ. ID. NO:38 | | | | | | |
| 679 | GCCCATGCGGGGCTTCTTTG | −18.8 | −31 | 82.5 | −8.2 | −4 | −11 |
| | SEQ. ID. NO:39 | | | | | | |
| 2138 | TTTTTTTTTCTGTTGCCATT | −18.8 | −22 | 66.8 | −3.2 | 0 | −3 |
| | SEQ. ID. NO:40 | | | | | | |
| 221 | AAGCAGTGTTCACTTTGAGC | −18.7 | −23.1 | 69.8 | −3.9 | 0 | −7.9 |
| | SEQ. ID. NO:41 | | | | | | |
| 1979 | GCCAATTAGAATGCAGGATT | −18.7 | −21.9 | 63.6 | −3.2 | 0 | −5.5 |
| | SEQ. ID. NO:42 | | | | | | |
| 2134 | TTTTTCTGTTGCCATTATGT | −18.7 | −22.5 | 68 | −3.8 | 0 | −3 |
| | SEQ. ID. NO:43 | | | | | | |
| 687 | GCTGACGCGCCCATGCGGGG | −18.6 | −33.6 | 82.7 | −12.2 | −2.8 | −11.1 |
| | SEQ. ID. NO:44 | | | | | | |
| 699 | TTGATCCTCCCTGCTGACGC | −18.6 | −29.4 | 79 | −10.3 | −0.1 | −4.5 |
| | SEQ. ID. NO:45 | | | | | | |
| 843 | ACACAGTTGCCCCCGTTTTT | −18.6 | −29.3 | 78.2 | −10.7 | 0 | −3 |
| | SEQ. ID. NO:46 | | | | | | |
| 917 | CAGCCAACATTCCCATCTCT | −18.6 | −27.2 | 74.9 | −8.6 | 0 | −3.2 |
| | SEQ. ID. NO:47 | | | | | | |
| 313 | CACTTTCTTTATGGTGGTCT | −18.4 | −23.3 | 70.9 | −4.9 | 0 | −3.9 |
| | SEQ. ID. NO:48 | | | | | | |
| 887 | TTAGTCGACACTCTTGACAC | −18.4 | −21.9 | 65.6 | −1.9 | −1.5 | −9.5 |
| | SEQ. ID. NO:49 | | | | | | |
| 984 | TCTGCATGCTGCTTCACATT | −18.4 | −25.4 | 73.9 | −5.2 | −1.8 | −9.7 |
| | SEQ. ID. NO:50 | | | | | | |
| 2137 | TTTTTTTTCTGTTGCCATTA | −18.4 | −21.6 | 65.8 | −3.2 | 0 | −3 |
| | SEQ. ID. NO:51 | | | | | | |
| 216 | GTGTTCACTTTGAGCTATGT | −18.3 | −23.1 | 70.8 | −3.9 | −0.8 | −5.1 |
| | SEQ. ID. NO:52 | | | | | | |
| 1129 | CATCCTCTGTTTGTTATATG | −18.3 | −21.4 | 65.4 | −3.1 | 0 | −2.4 |
| | SEQ. ID. NO:53 | | | | | | |
| 1982 | CTTGCCAATTAGAATGCAGG | −18.3 | −22.2 | 64.1 | −3.2 | −0.5 | −5.5 |
| | SEQ. ID. NO:54 | | | | | | |
| 2136 | TTTTTTTCTGTTGCCATTAT | −18.3 | −21.5 | 65.4 | −3.2 | 0 | −3 |
| | SEQ. ID. NO:55 | | | | | | |
| 608 | GCATACGCCTGAGTTCATAT | −18.2 | −24.6 | 70.2 | −6.4 | 0 | −3.4 |
| | SEQ. ID. NO:56 | | | | | | |
| 849 | TCCATCACACAGTTGCCCCC | −18.2 | −31.1 | 82.4 | −12.9 | 0 | −3 |
| | SEQ. ID. NO:57 | | | | | | |
| 889 | CCTTAGTCGACACTCTTGAC | −18.2 | −23.9 | 69.6 | −5 | 0 | −8.7 |
| | SEQ. ID. NO:58 | | | | | | |
| 890 | TCCTTAGTCGACACTCTTGA | −18.2 | −24.1 | 70.6 | −5 | 0 | −9.5 |
| | SEQ. ID. NO:59 | | | | | | |
| 1128 | ATCCTCTGTTTGTTATATGA | −18.2 | −21.3 | 65.6 | −3.1 | 0 | −2.4 |
| | SEQ. ID. NO:60 | | | | | | |
| 1140 | ATTTCCTGAGGCATCCTCTG | −18.2 | −26.4 | 75.8 | −5.7 | −2.5 | −7.9 |
| | SEQ. ID. NO:61 | | | | | | |
| 2135 | TTTTTTCTGTTGCCATTATG | −18.2 | −21.4 | 65 | −3.2 | 0 | −3 |
| | SEQ. ID. NO:62 | | | | | | |
| 691 | CCCTGCTGACGCGCCCATGC | −18.1 | −34.1 | 84 | −14.7 | −1.2 | −8.2 |
| | SEQ. ID. NO:63 | | | | | | |
| 918 | TCAGCCAACATTCCCATCTC | −18.1 | −26.7 | 74.6 | −8.6 | 0 | −3.2 |
| | SEQ. ID. NO:64 | | | | | | |
| 983 | CTGCATGCTGCTTCACATTT | −18.1 | −25.1 | 72.6 | −5.2 | −1.8 | −9.7 |
| | SEQ. ID. NO:65 | | | | | | |
| 1122 | TGTTTGTTATATGAATCCAT | −18.1 | −19.1 | 59.1 | −0.9 | 0 | −2.6 |
| | SEQ. ID. NO:66 | | | | | | |
| 916 | AGCCAACATTCCCATCTCTT | −18 | −26.6 | 74.2 | −8.6 | 0 | −3.2 |
| | SEQ. ID. NO:67 | | | | | | |
| 981 | GCATGCTGCTTCACATTTTT | −18 | −24.4 | 71.5 | −5.2 | −1.1 | −8.9 |
| | SEQ. ID. NO:68 | | | | | | |
| 1137 | TCCTGAGGCATCCTCTGTTT | −18 | −27.6 | 79.5 | −7.1 | −2.5 | 7.9 |
| | SEQ. ID. NO:69 | | | | | | |
| 1651 | TTCAGTCAGGCGACCCAGGA | −18 | −28.6 | 78.8 | −9.2 | −1.3 | −5.9 |
| | SEQ. ID. NO:70 | | | | | | |
| 1980 | TGCCAATTAGAATGCAGGAT | −18 | −21.8 | 63.2 | −3.2 | −0.3 | −5.5 |
| | SEQ. ID. NO:71 | | | | | | |
| 1981 | TTGCCAATTAGAATGCAGGA | −18 | −21.9 | 63.5 | −3.2 | −0.5 | −5.5 |
| | SEQ. ID. NO:72 | | | | | | |
| 607 | CATACGCCTGAGTTCATATA | −17.9 | −22.5 | 65.5 | −4.6 | 0 | −3.3 |
| | SEQ. ID. NO:73 | | | | | | |
| 1141 | TATTTCCTGAGGCATCCTCT | −17.9 | −26.1 | 75.4 | −5.7 | −2.5 | −7.9 |
| | SEQ. ID. NO:74 | | | | | | |
| 1142 | TTATTTCCTGAGGCATCCTC | −17.9 | −25.3 | 73.8 | −5.7 | −1.7 | −6.9 |
| | SEQ. ID. NO:75 | | | | | | |
| 218 | CAGTGTTCACTTTGAGCTAT | −17.8 | −22.6 | 68.9 | −3.9 | −0.8 | −6.8 |
| | SEQ. ID. NO:76 | | | | | | |
| 807 | TTTTTGGTAATGCTTCTCCT | −17.8 | −23.2 | 69.1 | −5.4 | 0 | −3.6 |
| | SEQ. ID. NO:77 | | | | | | |
| 842 | CACAGTTGCCCCCGTTTTTA | −17.8 | −28.8 | 77.1 | −11 | 0 | −3 |
| | SEQ. ID. NO:78 | | | | | | |
| 919 | TTCAGCCAACATTCCCATCT | −17.8 | −26.4 | 73.4 | −8.6 | 0 | −3.2 |
| | SEQ. ID. NO:79 | | | | | | |
| 1654 | TAATTCAGTCAGGCGACCCA | −17.8 | −25.8 | 71.7 | −6.6 | −1.3 | −5.4 |
| | SEQ. ID. NO:80 | | | | | | |
| 2133 | TTTTCTGTTGCCATTATGTT | −17.8 | −22.5 | 68 | −4.7 | 0 | −3 |
| | SEQ. ID. NO:81 | | | | | | |
| 850 | ATCCATCACACAGTTGCCCC | −17.7 | −29.1 | 79 | −11.4 | 0 | −3 |
| | SEQ. ID. NO:82 | | | | | | |
| 1796 | ATGAGAGAGAAAAAGGAGCT | −17.7 | −18.1 | 55.9 | 0 | 0 | −5 |
| | SEQ. ID. NO:83 | | | | | | |
| 880 | ACACTCTTGACACTTTCTTC | −17.6 | −22 | 67.4 | −4.4 | 0 | −2.3 |
| | SEQ. ID. NO:84 | | | | | | |
| 1941 | CACAATGTAGAGAAAGTTGT | −17.6 | −18.1 | 56.7 | 0 | −0.2 | −4.4 |
| | SEQ. ID. NO:85 | | | | | | |
| 222 | GAAGCAGTGTTCACTTTGAG | −17.5 | −21.9 | 66.7 | −3.9 | 0 | −7.9 |
| | SEQ. ID. NO:86 | | | | | | |
| 316 | ATGCACTTTCTTTATGGTGG | −17.5 | −22.6 | 68 | −4.4 | −0.5 | −5.5 |
| | SEQ. ID. NO:87 | | | | | | |
| 878 | ACTCTTGACACTTTCTTCGC | −17.5 | −23.7 | 70.1 | −6.2 | 0 | −2.7 |
| | SEQ. ID. NO:88 | | | | | | |
| 905 | CCATCTCTTTGCATTTCCTT | −17.5 | −25.6 | 73.9 | −8.1 | 0 | −5.1 |
| | SEQ. ID. NO:89 | | | | | | |
| 980 | CATGCTGCTTCACATTTTTT | −17.5 | −22.7 | 67.5 | −5.2 | 0 | −6 |
| | SEQ. ID. NO:90 | | | | | | |
| 1127 | TCCTCTGTTTGTTATATGAA | −17.5 | −20.6 | 63.3 | −3.1 | 0 | −2.4 |
| | SEQ. ID. NO:91 | | | | | | |
| 1299 | CCTTTCAGCAAAGCAATCTG | −17.5 | −22.4 | 64.8 | −4 | −0.8 | −4.7 |
| | SEQ. ID. NO:92 | | | | | | |
| 1722 | GGGGTAAACTTGTGGTCGTT | −17.5 | −24.4 | 70.7 | −6.9 | 0 | −3.4 |
| | SEQ. ID. NO:93 | | | | | | |
| 1723 | TGGGGTAAACTTGTGGTCGT | −17.4 | −24.3 | 70.1 | −6.9 | 0 | −3 |
| | SEQ. ID. NO:94 | | | | | | |
| 1724 | GTGGGGTAAACTTGTGGTCG | −17.4 | −24.3 | 70.1 | −6.9 | 0 | −2.5 |
| | SEQ. ID. NO:95 | | | | | | |
| 605 | TACGCCTGAGTTCATATATT | −17.3 | −21.9 | 64.7 | −4.6 | 0 | −3.6 |
| | SEQ. ID. NO:96 | | | | | | |
| 692 | TCCCTGCTGACGCGCCCATG | −17.3 | −32.7 | 81.7 | −14.7 | −0.5 | −7.7 |
| | SEQ. ID. NO:97 | | | | | | |
| 841 | ACAGTTGCCCCCGTTTTTAC | −17.3 | −28.3 | 76.7 | −11 | 0 | −3 |
| | SEQ. ID. NO:98 | | | | | | |
| 915 | GCCAACATTCCCATCTCTTT | −17.3 | −26.7 | 74.2 | −9.4 | 0 | −2 |
| | SEQ. ID. NO:99 | | | | | | |
| 982 | TGCATGCTGCTTCACATTTT | −17.3 | −24.3 | 71 | −5.2 | −1.8 | −9.7 |
| | SEQ. ID. NO:100 | | | | | | |
| 215 | TGTTCACTTTGAGCTATGTT | −17.2 | −22 | 67.6 | −3.9 | −0.8 | −5.1 |
| | SEQ. ID. NO:101 | | | | | | |
| 606 | ATACGCCTGAGTTCATATAT | −17.2 | −21.8 | 64.3 | −4.6 | 0 | −3.3 |
| | SEQ. ID. NO:102 | | | | | | |
| 979 | ATGCTGCTTCACATTTTTTC | −17.2 | −22.4 | 67.9 | −5.2 | 0 | −6 |
| | SEQ. ID. NO:103 | | | | | | |
| 217 | AGTGTTCACTTTGAGCTATG | −17.1 | −21.9 | 67.5 | −3.9 | −0.8 | −6.6 |
| | SEQ. ID. NO:104 | | | | | | |
| 312 | ACTTTCTTTATGGTGGTCTT | −17.1 | −22.7 | 70 | −5.6 | 0 | −2.2 |
| | SEQ. ID. NO:105 | | | | | | |
| 838 | GTTGCCCCCGTTTTTACACT | −17.1 | −29.2 | 78.2 | −11.4 | −0.4 | −3.4 |
| | SEQ. ID. NO:106 | | | | | | |
| 1067 | GTTCAGTTTTCTCCCTGCAT | −17.1 | −27 | 79.1 | −9.9 | 0 | −4.9 |
| | SEQ. ID. NO:107 | | | | | | |
| 1068 | AGTTCAGTTTTCTCCCTGCA | −17.1 | −27 | 79.5 | −9.9 | 0 | −4.7 |
| | SEQ. ID. NO:108 | | | | | | |
| 1126 | CCTCTGTTTGTTATATGAAT | −17.1 | −20.2 | 61.8 | −3.1 | 0 | −2.4 |
| | SEQ. ID. NO:109 | | | | | | |
| 1983 | GCTTGCCAATTAGAATGCAG | −17.1 | −22.8 | 65.6 | −5 | −0.5 | −5.5 |
| | SEQ. ID. NO:110 | | | | | | |
| 665 | TCTTTGTTACAGGCATCTCT | −17 | −23.7 | 72.2 | −6.7 | 0 | −4.2 |
| | SEQ. ID. NO:111 | | | | | | |
| 895 | GCATTTCCTTAGTCGACACT | −17 | −24.8 | 71.6 | −6.9 | 0 | −9.5 |
| | SEQ. ID. NO:112 | | | | | | |
| 899 | CTTTGCATTTCCTTAGTCGA | −17 | −23.9 | 69.9 | −6.9 | 0 | −5.1 |
| | SEQ. ID. NO:113 | | | | | | |
| 1940 | ACAATGTAGAGAAAGTTGTT | −17 | −17.5 | 55.7 | 0.9 | −0.2 | −4 |
| | SEQ. ID. NO:114 | | | | | | |
| 46 | GAATCCAATTTCGCATTAGG | −16.9 | −21.2 | 61.7 | −4.3 | 0 | −3.7 |
| | SEQ. ID. NO:115 | | | | | | |
| 575 | ACCACTCTTCAGGCTGCTGG | −16.9 | −28.3 | 80.2 | −9.9 | −1.4 | −6.1 |
| | SEQ. ID. NO:116 | | | | | | |
| 808 | GTTTTTGGTAATGCTTCTCC | −16.9 | −23.5 | 70.5 | −6.6 | 0 | −3.6 |
| | SEQ. ID. NO:117 | | | | | | |
| 920 | ATTCAGCCAACATTCCCATC | −16.9 | −25.5 | 71.4 | −8.6 | 0 | −2.4 |
| | SEQ. ID. NO:118 | | | | | | |
| 985 | ATCTGCATGCTGCTTCACAT | −16.9 | −25.3 | 73.5 | −6.6 | −1.8 | −9.7 |
| | SEQ. ID. NO:119 | | | | | | |
| 2132 | TTTCTGTTGCCATTATGTTT | −16.9 | −22.5 | 68 | −5.6 | 0 | −3 |
| | SEQ. ID. NO:120 | | | | | | |
| 214 | GTTCACTTTGAGCTATGTTT | −16.8 | −22.1 | 68.2 | −4.8 | −0.1 | −5.1 |
| | SEQ. ID. NO:121 | | | | | | |
| 698 | TGATCCTCCCTGCTGACGCG | −16.8 | −30.1 | 78.3 | −12 | −1.2 | −7.4 |
| | SEQ. ID. NO:122 | | | | | | |
| 891 | TTCCTTAGTCGACACTCTTG | −16.8 | −23.6 | 69.6 | −5.9 | 0 | −9.5 |
| | SEQ. ID. NO:123 | | | | | | |
| 900 | TCTTTGCATTTCCTTAGTCG | −16.8 | −23.7 | 70.2 | −6.9 | 0 | −5.1 |
| | SEQ. ID. NO:124 | | | | | | |
| 978 | TGCTGCTTCACATTTTTTCT | −16.7 | −23.3 | 70 | −6.6 | 0 | −6 |
| | SEQ. ID. NO:125 | | | | | | |
| 1145 | TTGTTATTTCCTGAGGCATC | −16.7 | −23.3 | 69.9 | −6.6 | 0 | −5 |
| | SEQ. ID. NO:126 | | | | | | |
| 1942 | ACACAATGTAGAGAAAGTTG | −16.7 | −17.1 | 54.3 | 0 | 0 | −4.4 |
| | SEQ. ID. NO:127 | | | | | | |
| 1051 | GCATGACTTTGTTGTCGAGG | −16.6 | −23.9 | 70 | −6 | −1.2 | −5.2 |
| | SEQ. ID. NO:128 | | | | | | |
| 1725 | AGTGGGGTAAACTTGTGGTC | −16.6 | −23.5 | 70.4 | −6.9 | 0 | −2.6 |
| | SEQ. ID. NO:129 | | | | | | |
| 43 | TCCAATTTCGCATTAGGATA | −16.5 | −21.6 | 63.2 | −4.3 | −0.6 | −4.8 |
| | SEQ. ID. NO:130 | | | | | | |
| 571 | CTCTTCAGGCTGCTGGGGGT | −16.5 | −30 | 86.2 | −12.5 | −0.9 | −6.1 |
| | SEQ. ID. NO:131 | | | | | | |
| 676 | CATGCGGGGCTTCTTTGTTA | −16.5 | −26.2 | 74.6 | −9.1 | −0.3 | −4.1 |
| | SEQ. ID. NO:132 | | | | | | |
| 877 | CTCTTGACACTTTCTTCGCA | −16.5 | −24.2 | 70.7 | −7.7 | 0 | −3.6 |
| | SEQ. ID. NO:133 | | | | | | |
| 1656 | CGTAATTCAGTCAGGCGACC | −16.5 | −25.1 | 70.3 | −7.2 | −1.3 | −5.1 |
| | SEQ. ID. NO:134 | | | | | | |
| 1797 | TATGAGAGAGAAAAAGGAGC | −16.5 | −16.9 | 53.5 | 0 | 0 | −2.8 |
| | SEQ. ID. NO:135 | | | | | | |
| 223 | AGAAGCAGTGTTCACTTTGA | −16.4 | −21.9 | 66.7 | −4.8 | −0.4 | −7.8 |
| | SEQ. ID. NO:136 | | | | | | |
| 1653 | AATTCAGTCAGGCGACCCAG | −16.4 | −26.1 | 72.5 | −8.3 | −1.3 | −5.4 |
| | SEQ. ID. NO:137 | | | | | | |
| 1795 | TGAGAGAGAAAAAGGAGCTA | −16.4 | −17.8 | 55.3 | −1.3 | 0 | −5.1 |
| | SEQ. ID. NO:138 | | | | | | |
| 49 | TCAGAATCCAATTTCGCATT | −16.3 | −21.4 | 62.4 | −4.4 | −0.4 | −3.6 |
| | SEQ. ID. NO:139 | | | | | | |
| 704 | CCCCTTTGATCCTCCCTGCT | −16.3 | −33 | 85.7 | −16.7 | 0 | −4.3 |
| | SEQ. ID. NO:140 | | | | | | |
| 914 | CCAACATTCCCATCTCTTTG | −16.3 | −24.9 | 70 | −8.6 | 0 | −2.5 |
| | SEQ. ID. NO:141 | | | | | | |
| 1053 | CTGCATGACTTTGTTGTCGA | −16.3 | −23.6 | 69 | −6 | −1.2 | −7.6 |
| | SEQ. ID. NO:142 | | | | | | |
| 1376 | ATAGGTCAGAATGCCCAGAC | −16.3 | −24.4 | 70 | −6.6 | −1.4 | −5.8 |
| | SEQ. ID. NO:143 | | | | | | |
| 1781 | GAGCTAGACCCCTCCCCTGT | −16.3 | −33.2 | 87.1 | −16.9 | 0 | −5.3 |
| | SEQ. ID. NO:144 | | | | | | |
| 42 | CCAATTTCGCATTAGGATAA | −16.2 | −20.5 | 59.9 | −4.3 | 0 | −3.6 |
| | SEQ. ID. NO:145 | | | | | | |
| 44 | ATCCAATTTCGCATTAGGAT | −16.2 | −21.9 | 63.7 | −4.3 | −1.3 | −6.2 |
| | SEQ. ID. NO:146 | | | | | | |
| 441 | GGACCTGCCACTTGTTCTGT | −16.2 | −28.4 | 80.2 | −11.7 | −0.2 | −3 |
| | SEQ. ID. NO:147 | | | | | | |
| 604 | ACGCCTGAGTTCATATATTC | −16.2 | −22.6 | 66.8 | −6.4 | 0 | −3.6 |
| | SEQ. ID. NO:148 | | | | | | |
| 666 | TTCTTTGTTACAGGCATCTC | −16.2 | −22.9 | 70.4 | −6.7 | 0 | −4.2 |
| | SEQ. ID. NO:149 | | | | | | |
| 695 | TCCTCCCTGCTGACGCGCCC | −16.2 | −35.3 | 87.5 | −17.8 | −1.2 | −7.7 |
| | SEQ. ID. NO:150 | | | | | | |
| 839 | AGTTGCCCCCGTTTTTACAC | −16.2 | −28.3 | 76.7 | −11.4 | −0.4 | −3.4 |
| | SEQ. ID. NO:151 | | | | | | |
| 999 | TCATTCACGGTCTGATCTGC | −16.2 | −24.7 | 72.5 | −8.5 | 0 | −4.9 |
| | SEQ. ID. NO:152 | | | | | | |
| 1069 | GAGTTCAGTTTTCTCCCTGC | −16.2 | −26.9 | 79.9 | −10.7 | 0 | −4.4 |
| | SEQ. ID. NO:153 | | | | | | |
| 662 | TTGTTACAGGCATCTCTGCT | −16.1 | −25 | 74.4 | −6.7 | −2.2 | −8.7 |
| | SEQ. ID. NO:154 | | | | | | |
| 896 | TGCATTTCCTTAGTCGACAC | −16.1 | −23.9 | 69.5 | −6.9 | 0 | −9.5 |
| | SEQ. ID. NO:155 | | | | | | |
| 38 | TTTCGCATTAGGATAAGTCG | −16 | −20.9 | 62 | −4.3 | −0.3 | −3.9 |
| | SEQ. ID. NO:156 | | | | | | |
| 663 | TTTGTTACAGGCATCTCTGC | −16 | −24.2 | 72.7 | −6.7 | −1.4 | −8.5 |
| | SEQ. ID. NO:157 | | | | | | |
| 703 | CCCTTTGATCCTCCCTGCTG | −16 | −31 | 82.3 | −15 | 0 | −4.3 |
| | SEQ. ID. NO:158 | | | | | | |
| 897 | TTGCATTTCCTTAGTCGACA | −16 | −23.8 | 69.3 | −6.9 | 0 | −9.5 |
| | SEQ. ID. NO:159 | | | | | | |
| 1050 | CATGACTTTGTTGTCGAGGT | −16 | −23.3 | 69 | −6 | −1.2 | −5.2 |
| | SEQ. ID. NO:160 | | | | | | |
| 1052 | TGCATGACTTTGTTGTCGAG | −16 | −22.7 | 67.3 | −6 | −0.5 | −7.6 |
| | SEQ. ID. NO:161 | | | | | | |
| 45 | AATCCAATTTCGCATTAGGA | −15.9 | −21.2 | 61.7 | −4.3 | −0.9 | −5.4 |
| | SEQ. ID. NO:162 | | | | | | |
| 664 | CTTTGTTACAGGCATCTCTG | −15.9 | −23.3 | 70.2 | −6.7 | −0.4 | −4.4 |
| | SEQ. ID. NO:163 | | | | | | |
| 700 | TTTGATCCTCCCTGCTGACG | −15.9 | −27.7 | 75.2 | −11.8 | 0 | −4.3 |
| | SEQ. ID. NO:164 | | | | | | |
| 806 | TTTTGGTAATGCTTCTCCTG | −15.9 | −23.1 | 68.6 | −7.2 | 0 | −3.6 |
| | SEQ. ID. NO:165 | | | | | | |
| 1054 | CCTGCATGACTTTGTTGTCG | −15.9 | −25 | 71.3 | −7.8 | −1.2 | −7.6 |
| | SEQ. ID. NO:166 | | | | | | |
| 1121 | GTTTGTTATATGAATCCATA | −15.9 | −18.8 | 58.6 | −1.9 | −0.8 | −3.4 |
| | SEQ. ID. NO:167 | | | | | | |
| 1123 | CTGTTTGTTATATGAATCCA | −15.9 | −20 | 61.1 | −4.1 | 0 | −2.4 |
| | SEQ. ID. NO:168 | | | | | | |
| 1686 | AGCATCTCAGCGTGGTGATG | −15.9 | −25.7 | 74.4 | −8.8 | −0.9 | −6.2 |
| | SEQ. ID. NO:169 | | | | | | |
| 1721 | GGGTAAACTTGTGGTCGTTT | −15.9 | −23.3 | 68.4 | −6.9 | −0.1 | −4.2 |
| | SEQ. ID. NO:170 | | | | | | |
| 1943 | AACACAATGTAGAGAAAGTT | −15.9 | −16.4 | 52.5 | 0 | −0.2 | −4.4 |
| | SEQ. ID. NO:171 | | | | | | |
| 39 | ATTTCGCATTAGGATAAGTC | −15.8 | −20.1 | 61.5 | −4.3 | 0 | −3.1 |
| | SEQ. ID. NO:172 | | | | | | |
| 576 | TACCACTCTTCAGGCTGCTG | −15.8 | −26.8 | 76.9 | −9.9 | −1 | −6.1 |
| | SEQ. ID. NO:173 | | | | | | |
| 898 | TTTGCATTTCCTTAGTCGAC | −15.8 | −23.2 | 68.5 | −6.9 | 0 | −8.2 |
| | SEQ. ID. NO:174 | | | | | | |
| 1300 | CCCTTTCAGCAAAGCAATCT | −15.8 | −24.4 | 68.4 | −7.7 | −0.8 | −4.7 |
| | SEQ. ID. NO:175 | | | | | | |
| 1650 | TCAGTCAGGCGACCCAGGAG | −15.8 | −28.5 | 78.7 | −11.3 | −1.3 | −5.9 |
| | SEQ. ID. NO:176 | | | | | | |
| 48 | CAGAATCCAATTTCGCATTA | −15.7 | −20.7 | 60.5 | −4.3 | −0.4 | −3.6 |
| | SEQ. ID. NO:177 | | | | | | |
| 888 | CTTAGTCGACACTCTTGACA | −15.7 | −22.6 | 67 | −5.3 | −1.5 | −9.5 |
| | SEQ. ID. NO:178 | | | | | | |
| 892 | TTTCCTTAGTCGACACTCTT | −15.7 | −23.7 | 70.1 | −7.1 | 0 | −9.5 |
| | SEQ. ID. NO:179 | | | | | | |
| 1049 | ATGACTTTGTTGTCGAGGTC | −15.7 | −23 | 69.5 | −6 | −1.2 | −5.2 |
| | SEQ. ID. NO:180 | | | | | | |
| 1673 | GGTGATGATTGAATGTCCGT | −15.7 | −23.2 | 67 | −7.5 | 0 | −2.8 |
| | SEQ. ID. NO:181 | | | | | | |
| 2047 | ATGAGATTTTCCCTAGTTCA | −15.7 | −22.9 | 68.4 | −7.2 | 0 | −3.8 |
| | SEQ. ID. NO:182 | | | | | | |
| 37 | TTCGCATTAGGATAAGTCGG | −15.6 | −22 | 64.2 | −5.6 | −0.6 | −3.9 |
| | SEQ. ID. NO:183 | | | | | | |
| 440 | GACCTGCCACTTGTTCTGTT | −15.6 | −27.3 | 77.9 | −11.7 | 0 | −2.3 |
| | SEQ. ID. NO:184 | | | | | | |
| 690 | CCTGCTGACGCGCCCATGCG | −15.6 | −32.9 | 80.5 | −14.7 | −2.6 | −9.6 |
| | SEQ. ID. NO:185 | | | | | | |
| 1043 | TTGTTGTCGAGGTCACTTGT | −15.6 | −24.3 | 72.9 | −8.7 | 0 | −4.9 |
| | SEQ. ID. NO:186 | | | | | | |
| 1926 | GTTGTTCTATCTAGCCCAAT | −15.6 | −24.4 | 71.5 | −8.8 | 0 | −3.7 |
| | SEQ. ID. NO:187 | | | | | | |
| 212 | TCACTTTGAGCTATGTTTCT | −15.5 | −22.1 | 68 | −6.6 | 0 | −5.1 |
| | SEQ. ID. NO:188 | | | | | | |
| 1375 | TAGGTCAGAATGCCCAGACG | −15.5 | −25.2 | 70.1 | −8.2 | −1.4 | −5.9 |
| | SEQ. ID. NO:189 | | | | | | |
| 837 | TTGCCCCCGTTTTTACACTT | −15.4 | −28.1 | 75.3 | −12 | −0.4 | −3.4 |
| | SEQ. ID. NO:190 | | | | | | |
| 851 | TATCCATCACACAGTTGCCC | −15.4 | −26.8 | 75 | −11.4 | 0 | −3 |
| | SEQ. ID. NO:191 | | | | | | |
| 1001 | CTTCATTCACGGTCTGATCT | −15.4 | −23.9 | 70.6 | −8.5 | 0 | −4.9 |
| | SEQ. ID. NO:192 | | | | | | |
| 1305 | GCAGACCCTTTCAGCAAAGC | −15.4 | −26.4 | 73.5 | −10.1 | −0.8 | −5 |
| | SEQ. ID. NO:193 | | | | | | |
| 1377 | AATAGGTCAGAATGCCCAGA | −15.4 | −23.5 | 67.2 | −6.6 | −1.4 | −4.5 |
| | SEQ. ID. NO:194 | | | | | | |
| 1780 | AGCTAGACCCCTCCCCTGTA | −15.4 | −32.3 | 85.3 | −16.9 | 0 | −4.3 |
| | SEQ. ID. NO:195 | | | | | | |
| 317 | AATGCACTTTCTTTATGGTG | −15.3 | −20.7 | 63 | −4.9 | −0.1 | −5.5 |
| | SEQ. ID. NO:196 | | | | | | |
| 577 | GTACCACTCTTCAGGCTGCT | −15.2 | −28 | 80.8 | −12.8 | 0 | −6.1 |
| | SEQ. ID. NO:197 | | | | | | |
| 840 | CAGTTGCCCCCGTTTTTACA | −15.2 | −28.8 | 77.1 | −12.9 | −0.4 | −2.7 |
| | SEQ. ID. NO:198 | | | | | | |
| 904 | CATCTCTTTGCATTTCCTTA | −15.2 | −23.3 | 69.6 | −8.1 | 0 | −5.1 |
| | SEQ. ID. NO:199 | | | | | | |
| 1042 | TGTTGTCGAGGTCACTTGTC | −15.2 | −24.6 | 74.3 | −9.4 | 0 | −4.4 |
| | SEQ. ID. NO:100 | | | | | | |
| 1146 | TTTGTTATTTCCTGAGGCAT | −15.2 | −23 | 68.7 | −7.8 | 0 | −4 |
| | SEQ. ID. NO:201 | | | | | | |
| 50 | CTCAGAATCCAATTTCGCAT | −15.1 | −22.2 | 63.9 | −6.4 | −0.4 | −3.6 |
| | SEQ. ID. NO:202 | | | | | | |
| 697 | GATCCTCCCTGCTGACGCGC | −15.1 | −31.9 | 82.5 | −15.5 | −1.2 | −7.7 |
| | SEQ. ID. NO:203 | | | | | | |
| 990 | GTCTGATCTGCATGCTGCTT | −15.1 | −26.4 | 77.5 | −9.5 | −1.8 | −9.7 |
| | SEQ. ID. NO:204 | | | | | | |
| 1944 | AAACACAATGTAGAGAAAGT | −15.1 | −15.6 | 50.6 | 0 | −0.2 | −4.4 |
| | SEQ. ID. NO:205 | | | | | | |
| 47 | AGAATCCAATTTCGCATTAG | −15 | −20 | 59.5 | −4.3 | −0.4 | −3.6 |
| | SEQ. ID. NO:206 | | | | | | |
| 572 | ACTCTTCAGGCTGCTGGGGG | −15 | −29 | 83 | −12.5 | −1.4 | −6.1 |
| | SEQ. ID. NO:207 | | | | | | |
| 805 | TTTGGTAATGCTTCTCCTGA | −15 | −23.6 | 69.6 | −8.6 | 0 | −3.6 |
| | SEQ. ID. NO:208 | | | | | | |
| 986 | GATCTGCATGCTGCTTCACA | −15 | −25.9 | 74.9 | −9.7 | −1.1 | −9 |
| | SEQ. ID. NO:209 | | | | | | |
| 1048 | TGACTTTGTTGTCGAGGTCA | −15 | −23.7 | 70.7 | −6.9 | −1.8 | −6.7 |
| | SEQ. ID. NO:210 | | | | | | |
| 1782 | GGAGCTAGACCCCTCCCCTG | −15 | −33.2 | 86.1 | −16.9 | −1.2 | −6.4 |
| | SEQ. ID. NO:211 | | | | | | |
| 2046 | TGAGATTTTCCCTAGTTCAA | −15 | −22.2 | 66.2 | −7.2 | 0 | −3.8 |
| | SEQ. ID. NO:212 | | | | | | |
| 667 | CTTCTTTGTTACAGGCATCT | −14.9 | −23.4 | 70.8 | −8.5 | 0 | −4.2 |
| | SEQ. ID. NO:213 | | | | | | |
| 1652 | ATTCAGTCAGGCGACCCAGG | −14.9 | −28 | 77.4 | −12.1 | −0.9 | −5.4 |
| | SEQ. ID. NO:214 | | | | | | |
| 1675 | GTGGTGATGATTGAATGTCC | −14.9 | −22.4 | 66.6 | −7.5 | 0 | −2.8 |
| | SEQ. ID. NO:215 | | | | | | |
| 211 | CACTTTGAGCTATGTTTCTA | −14.8 | −21.4 | 65.8 | −6.6 | 0 | −5.1 |
| | SEQ. ID. NO:216 | | | | | | |
| 879 | CACTCTTGACACTTTCTTCG | −14.8 | −22.6 | 67 | −7.8 | 0 | −2.4 |
| | SEQ. ID. NO:217 | | | | | | |
| 1894 | GGAAGTTACACATGTAATTA | −14.8 | −17.9 | 56.3 | −3.1 | 0.1 | −6.6 |
| | SEQ. ID. NO:218 | | | | | | |
| 40 | AATTTCGCATTAGGATAAGT | −14.7 | −19 | 58.1 | −4.3 | 0 | −3.9 |
| | SEQ. ID. NO:219 | | | | | | |
| 1726 | AAGTGGGGTAAACTTGTGGT | −14.7 | −22.4 | 66.4 | −7.1 | −0.3 | −3.6 |
| | SEQ. ID. NO:220 | | | | | | |
| 1779 | GCTAGACCCCTCCCCTGTAA | −14.7 | −31.6 | 82.4 | −16.9 | 0 | −4.1 |
| | SEQ. ID. NO:221 | | | | | | |
| 1798 | ATATGAGAGAGAAAAAGGAG | −14.7 | −15.1 | 49.7 | 0 | 0 | −1.8 |
| | SEQ. ID. NO:222 | | | | | | |
| 1927 | AGTTGTTCTATCTAGCCCAA | −14.7 | −24.4 | 71.8 | −9.7 | 0 | −3.7 |
| | SEQ. ID. NO:223 | | | | | | |
| 1928 | AAGTTGTTCTATCTAGCCCA | −14.7 | −24.4 | 71.8 | −9.7 | 0 | −3.7 |
| | SEQ. ID. NO:224 | | | | | | |
| 225 | AGAGAACCAGTGTTCACTTT | −14.6 | −21.9 | 67.1 | −6.6 | −0.4 | −6.8 |
| | SEQ. ID. NO:225 | | | | | | |
| 688 | TGCTGACGCGCCCATGCGGG | −14.6 | −32.4 | 80.3 | −13.9 | −3.9 | −10.9 |
| | SEQ. ID. NO:226 | | | | | | |
| 901 | CTCTTTGCATTTCCTTAGTC | −14.6 | −23.8 | 72.2 | −9.2 | 0 | −4.8 |
| | SEQ. ID. NO:227 | | | | | | |
| 988 | CTGATCTGCATGCTGCTTCA | −14.6 | −25.9 | 75 | −9.5 | −1.8 | −9.7 |
| | SEQ. ID. NO:228 | | | | | | |
| 1378 | CAATAGGTCAGAATGCCCAG | −14.6 | −23.6 | 67.1 | −8.2 | −0.6 | −3.7 |
| | SEQ. ID. NO:229 | | | | | | |
| 1984 | GGCTTGCCAATTAGAATGCA | −14.6 | −24 | 67.8 | −8.3 | −1 | −7.9 |
| | SEQ. ID. NO:230 | | | | | | |
| 1000 | TTCATTCACGGTCTGATCTG | −14.5 | −23 | 68.4 | −8.5 | 0 | −4.9 |
| | SEQ. ID. NO:231 | | | | | | |
| 1044 | TTTGTTGTCGAGGTCACTTG | −14.5 | −23.2 | 69.7 | −8.7 | 0 | −4.9 |
| | SEQ. ID. NO:232 | | | | | | |
| 1153 | AATTTTATTTGTTATTTCCT | −14.5 | −18 | 57.3 | −3.5 | 0 | −2.3 |
| | SEQ. ID. NO:233 | | | | | | |
| 1674 | TGGTGATGATTGAATGTCCG | −14.5 | −22 | 63.8 | −7.5 | 0 | −3.5 |
| | SEQ. ID. NO:234 | | | | | | |
| 1895 | TGGAAGTTACACATGTAATT | −14.5 | −18.2 | 56.8 | −3.1 | −0.3 | −7.1 |
| | SEQ. ID. NO:235 | | | | | | |
| 1939 | CAATGTAGAGAAAGTTGTTC | −14.5 | −17.7 | 56.5 | −2.7 | −0.1 | −2.8 |
| | SEQ. ID. NO:236 | | | | | | |
| 1948 | TTTAAAACACAATGTAGAGA | −14.5 | −15 | 49.5 | 0 | −0.2 | −5.1 |
| | SEQ. ID. NO:237 | | | | | | |
| 1978 | CCAATTAGAATGCAGGATTC | −14.5 | −20.5 | 61 | −5 | −0.9 | −5.5 |
| | SEQ. ID. NO:238 | | | | | | |
| 318 | AAATGCACTTTCTTTATGGT | −14.4 | −20 | 61 | −5.6 | 0 | −5.5 |
| | SEQ. ID. NO:239 | | | | | | |
| 701 | CTTTGATCCTCCCTGCTGAC | −14.3 | −27.8 | 77.4 | −13.5 | 0 | −4.3 |
| | SEQ. ID. NO:240 | | | | | | |
| 989 | TCTGATCTGCATGCTGCTTC | −14.3 | −25.6 | 75.6 | −9.5 | −1.8 | −9.7 |
| | SEQ. ID. NO:241 | | | | | | |
| 1304 | CAGACCCTTTCAGCAAAGCA | −14.3 | −25.3 | 70.4 | −10.1 | −0.8 | −4.7 |
| | SEQ. ID. NO:242 | | | | | | |
| 1590 | CACAACTTTTGTAGCACATC | −14.3 | −21 | 63.4 | −5.7 | −0.9 | −6.7 |
| | SEQ. ID. NO:243 | | | | | | |
| 1649 | CAGTCAGGCGACCCAGGAGA | −14.3 | −28.7 | 78.3 | −13 | −1.3 | −5.9 |
| | SEQ. ID. NO:244 | | | | | | |
| 1783 | AGGAGCTAGACCCCTCCCCT | −14.3 | −33.2 | 86.7 | −16.9 | −2 | −7.6 |
| | SEQ. ID. NO:245 | | | | | | |
| 41 | CAATTTCGCATTAGGATAAG | −14.2 | −18.5 | 56.5 | −4.3 | 0 | −3.9 |
| | SEQ. ID. NO:246 | | | | | | |
| 311 | CTTTCTTTATGGTGGTCTTC | −14.2 | −22.9 | 71.2 | −8.7 | 0 | −1.5 |
| | SEQ. ID. NO:247 | | | | | | |
| 661 | TGTTACAGGCATCTCTGCTA | −14.2 | −24.6 | 73.4 | −8.2 | −2.2 | −7.5 |
| | SEQ. ID. NO:248 | | | | | | |
| 693 | CTCCCTGCTGACGCGCCCAT | −14.2 | −33.6 | 83.6 | −18.1 | −1.2 | −7.7 |
| | SEQ. ID. NO:249 | | | | | | |
| 876 | TCTTGACACTTTCTTCGCAT | −14.2 | −23.3 | 68.7 | −9.1 | 0 | −3.6 |
| | SEQ. ID. NO:250 | | | | | | |
| 893 | ATTTCCTTAGTCGACACTCT | −14.2 | −23.6 | 69.7 | −8.5 | 0 | −9.5 |
| | SEQ. ID. NO:251 | | | | | | |
| 991 | GGTCTGATCTGCATGCTGCT | −14.2 | −27.5 | 79.8 | −11.5 | −1.8 | −9.7 |
| | SEQ. ID. NO:252 | | | | | | |
| 1124 | TCTGTTTGTTATATGAATCC | −14.2 | −19.7 | 61.3 | −5.5 | 0 | −2.4 |
| | SEQ. ID. NO:253 | | | | | | |
| 1672 | GTGATGATTGAATGTCCGTA | −14.2 | −21.7 | 63.9 | −7.5 | 0 | −2.6 |
| | SEQ. ID. NO:254 | | | | | | |
| 603 | CGCCTGAGTTCATATATTCC | −14.1 | −24.4 | 69.9 | −10.3 | 0 | −3.6 |
| | SEQ. ID. NO:255 | | | | | | |
| 739 | AGAGGCTCTGTCTCCACAAA | −14.1 | −24.9 | 72.1 | −9.6 | −1.1 | −5.1 |
| | SEQ. ID. NO:256 | | | | | | |
| 1251 | GGTAGCTTTTTTGTGAATTC | −14.1 | −20.9 | 64.9 | −6.8 | 0 | −5.9 |
| | SEQ. ID. NO:257 | | | | | | |
| 1591 | ACACAACTTTTGTAGCACAT | −14.1 | −20.8 | 62.5 | −5.7 | −0.9 | −6.7 |
| | SEQ. ID. NO:258 | | | | | | |
| 977 | GCTGCTTCACATTTTTTCTC | −14 | −23.7 | 71.9 | −9.7 | 0 | −5.2 |
| | SEQ. ID. NO:259 | | | | | | |
| 1227 | AGAACCTGTACATGATTGGT | −14 | −21.9 | 64.8 | −7.4 | −0.1 | −6.8 |
| | SEQ. ID. NO:260 | | | | | | |
| 1799 | AATATGAGAGAGAAAAAGGA | −14 | −14.4 | 48 | 0 | 0 | −2.7 |
| | SEQ. ID. NO:261 | | | | | | |
| 1426 | AGGTGTTATATATTCATCAG | −13.9 | −19.1 | 61 | −5.2 | 0 | −5.2 |
| | SEQ. ID. NO:262 | | | | | | |
| 1687 | CAGCATCTCAGCGTGGTGAT | −13.9 | −26.4 | 75.7 | −11.5 | −0.9 | −4.4 |
| | SEQ. ID. NO:263 | | | | | | |
| 1720 | GGTAAACTTGTGGTCGTTTA | −13.9 | −21.8 | 65.2 | −6.9 | −0.9 | −5 |
| | SEQ. ID. NO:264 | | | | | | |
| 1947 | TTAAAACACAATGTAGAGAA | −13.9 | −14.2 | 47.6 | 0 | 0.3 | −4.4 |
| | SEQ. ID. NO:265 | | | | | | |
| 2122 | CATTATGTTTGCTTTATTGC | −13.9 | −20.4 | 62.9 | −6.5 | 0 | −3.6 |
| | SEQ. ID. NO:266 | | | | | | |
| 226 | AAGAGAAGCAGTGTTCACTT | −13.8 | −21.1 | 64.4 | −6.6 | −0.4 | −7.5 |
| | SEQ. ID. NO:267 | | | | | | |
| 963 | TTTCTCAGTCGCTTAGATTT | −13.8 | −22.3 | 68.1 | −8.5 | 0 | −3.1 |
| | SEQ. ID. NO:268 | | | | | | |
| 964 | TTTTCTCAGTCGCTTAGATT | −13.8 | −22.3 | 68.1 | −8.5 | 0 | −3.1 |
| | SEQ. ID. NO:269 | | | | | | |
| 965 | TTTTTCTCAGTCGCTTAGAT | −13.8 | −22.3 | 68.1 | −8.5 | 0 | −3.1 |
| | SEQ. ID. NO:270 | | | | | | |
| 1147 | ATTTGTTATTTCCTGAGGCA | −13.8 | −23 | 68.7 | −9.2 | 0 | −4 |
| | SEQ. ID. NO:271 | | | | | | |
| 1220 | GTACATGATTGGTTGCCATT | −13.8 | −23.6 | 69 | −9.1 | −0.4 | −5.9 |
| | SEQ. ID. NO:272 | | | | | | |
| 1221 | TGTACATGATTGGTTGCCAT | −13.8 | −23.5 | 68.5 | −9 | −0.4 | −6.6 |
| | SEQ. ID. NO:273 | | | | | | |
| 1223 | CCTGTACATGATTGGTTGCC | −13.8 | −25.7 | 73 | −11.9 | 0 | −6.1 |
| | SEQ. ID. NO:274 | | | | | | |
| 1250 | GTAGCTTTTTTGTGAATTCT | −13.8 | −20.6 | 64.3 | −6.8 | 0 | −6.9 |
| | SEQ. ID. NO:275 | | | | | | |
| 1648 | AGTCAGGCGACCCAGGAGAC | −13.8 | −28.2 | 77.9 | −13 | −1.3 | −6.6 |
| | SEQ. ID. NO:276 | | | | | | |
| 1690 | CATCAGCATCTCAGCGTGGT | −13.8 | −26.9 | 77.5 | −12.6 | −0.1 | −4.1 |
| | SEQ. ID. NO:277 | | | | | | |
| 738 | GAGGCTCTGTCTCCACAAAC | −13.7 | −25.1 | 72.4 | −10.8 | −0.3 | −4.1 |
| | SEQ. ID. NO:278 | | | | | | |
| 1061 | TTTTCTCCCTGCATGACTTT | −13.7 | −25.3 | 72.9 | −11.6 | 0 | −4.9 |
| | SEQ. ID. NO:279 | | | | | | |
| 1365 | TGCCCAGACGGAAGTTTCTT | −13.7 | −26 | 72.2 | −11.4 | −0.8 | −5 |
| | SEQ. ID. NO:280 | | | | | | |
| 2127 | GTTGCCATTATGTTTGCTTT | −13.7 | −23.9 | 70.7 | −10.2 | 0 | −3.6 |
| | SEQ. ID. NO:281 | | | | | | |
| 51 | GCTCAGAATCCAATTTCGCA | −13.6 | −24 | 67.8 | −10.4 | 0.4 | −4 |
| | SEQ. ID. NO:282 | | | | | | |
| 612 | GCTGGCATACGCCTGAGTTC | −13.6 | −28.1 | 78.4 | −11.6 | −2.9 | −8.1 |
| | SEQ. ID. NO:283 | | | | | | |
| 1055 | CCCTGCATGACTTTGTTGTC | −13.6 | −26.2 | 75.1 | −12.1 | −0.1 | −4.9 |
| | SEQ. ID. NO:284 | | | | | | |
| 1060 | TTTCTCCCTGCATGACTTTG | −13.6 | −25.2 | 72.4 | −11.6 | 0 | −4.9 |
| | SEQ. ID. NO:285 | | | | | | |
| 1063 | AGTTTTCTCCCTGCATGACT | −13.6 | −26.3 | 76 | −12.7 | 0 | −4.9 |
| | SEQ. ID. NO:286 | | | | | | |
| 1066 | TTCAGTTTTCTCCCTGCATG | −13.6 | −25.8 | 75.2 | −12.2 | 0 | −5.7 |
| | SEQ. ID. NO:287 | | | | | | |
| 1366 | ATGCCCAGACGGAAGTTTCT | −13.6 | −25.9 | 71.8 | −11.4 | −0.8 | −5 |
| | SEQ. ID. NO:288 | | | | | | |
| 1427 | TAGGTGTTATATATTCATCA | −13.6 | −18.8 | 60.1 | −5.2 | 0 | −5.2 |
| | SEQ. ID. NO:289 | | | | | | |
| 1647 | GTCAGGCGACCCAGGAGACA | −13.6 | −28.9 | 78.6 | −14.3 | −0.9 | −6.5 |
| | SEQ. ID. NO:290 | | | | | | |
| 2123 | CCATTATGTTTGCTTTATTG | −13.6 | −20.6 | 62.5 | −7 | 0 | −3.6 |
| | SEQ. ID. NO:291 | | | | | | |
| 442 | AGGACCTGCCACTTGTTCTG | −13.5 | −27.2 | 76.9 | −12.6 | −1 | −3.6 |
| | SEQ. ID. NO:292 | | | | | | |
| 908 | TTCCCATCTCTTTGCATTTC | −13.5 | −25.1 | 73.6 | −11.6 | 0 | −5.1 |
| | SEQ. ID. NO:293 | | | | | | |
| 909 | ATTCCCATCTCTTTGCATTT | −13.5 | −24.7 | 71.9 | −11.2 | 0 | −5.1 |
| | SEQ. ID. NO:294 | | | | | | |
| 1580 | GTAGCACATCAAGAAGTGGC | −13.5 | −22.8 | 67.7 | −8.4 | −0.8 | −6.4 |
| | SEQ. ID. NO:295 | | | | | | |
| 1589 | ACAACTTTTGTAGCACATCA | −13.5 | −21 | 63.4 | −6.6 | −0.7 | −6.7 |
| | SEQ. ID. NO:296 | | | | | | |
| 1657 | CCGTAATTCAGTCAGGCGAC | −13.5 | −25.1 | 70.3 | −10.6 | −0.9 | −4.7 |
| | SEQ. ID. NO:297 | | | | | | |
| 36 | TCGCATTAGGATAAGTCGGG | −13.4 | −23.1 | 66.3 | −8.9 | −0.6 | −3.9 |
| | SEQ. ID. NO:298 | | | | | | |
| 213 | TTCACTTTGAGCTATGTTTC | −13.4 | −21.3 | 66.3 | −7.9 | 0 | −5.1 |
| | SEQ. ID. NO:299 | | | | | | |
| 705 | TCCCCTTTGATCCTCCCTGC | −13.4 | −32.5 | 85.7 | −19.1 | 0 | −4.3 |
| | SEQ. ID. NO:300 | | | | | | |
| 974 | GCTTCACATTTTTTCTCAGT | −13.4 | −22.9 | 70.4 | −9.5 | 0 | −2.8 |
| | SEQ. ID. NO:301 | | | | | | |
| 1034 | AGGTCACTTGTCGCAAGTCA | −13.4 | −25.2 | 73.9 | −9.8 | −2 | −10.6 |
| | SEQ. ID. NO:302 | | | | | | |
| 1064 | CAGTTTTCTCCCTGCATGAC | −13.4 | −26.1 | 75.1 | −12.7 | 0 | −5.4 |
| | SEQ. ID. NO:303 | | | | | | |
| 1364 | GCCCAGACGGAAGTTTCTTA | −13.4 | −25.7 | 71.8 | −11.4 | −0.8 | −5.1 |
| | SEQ. ID. NO:304 | | | | | | |
| 1430 | ACATAGGTGTTATATATTCA | −13.4 | −18.6 | 59.2 | −4.7 | −0.2 | −5.7 |
| | SEQ. ID. NO:305 | | | | | | |
| 1809 | ACATCAGATTAATATGAGAG | −13.4 | −16.6 | 53.7 | −3.2 | 0 | −7.4 |
| | SEQ. ID. NO:306 | | | | | | |
| 224 | GAGAAGCAGTGTTCACTTTG | −13.3 | −21.9 | 66.7 | −7.9 | −0.4 | −6.8 |
| | SEQ. ID. NO:307 | | | | | | |
| 609 | GGCATACGCCTGAGTTCATA | −13.3 | −25.8 | 72.8 | −10.3 | −2.2 | −7.4 |
| | SEQ. ID. NO:308 | | | | | | |
| 809 | CGTTTTTGGTAATGCTTCTC | −13.3 | −22.3 | 66.8 | −9 | 0 | −3.6 |
| | SEQ. ID. NO:309 | | | | | | |
| 1047 | GACTTTGTTGTCGAGGTCAC | −13.3 | −23.9 | 71.5 | −9.4 | −1.1 | −5.6 |
| | SEQ. ID. NO:310 | | | | | | |
| 2045 | GAGATTTTCCCTAGTTCAAC | −13.3 | −22.4 | 66.9 | −9.1 | 0 | −3.6 |
| | SEQ. ID. NO:311 | | | | | | |
| 2124 | GCCATTATGTTTGCTTTATT | −13.3 | −22.4 | 66.9 | −9.1 | 0 | −3.6 |
| | SEQ. ID. NO:312 | | | | | | |
| 2126 | TTGCCATTATGTTTGCTTTA | −13.3 | −22.4 | 66.8 | −9.1 | 0 | −3.6 |
| | SEQ. ID. NO:313 | | | | | | |
| 613 | AGCTGGCATACGCCTGAGTT | −13.2 | −27.7 | 77 | −11.6 | −2.9 | −9.3 |
| | SEQ. ID. NO:314 | | | | | | |
| 696 | ATCCTCCCTGCTGACGCGCC | −13.2 | −33.3 | 84.4 | −18.8 | −1.2 | −7.7 |
| | SEQ. ID. NO:315 | | | | | | |
| 923 | AGCATTCAGCCAACATTCCC | −13.2 | −26.9 | 74.3 | −12.7 | −0.9 | −4.1 |
| | SEQ. ID. NO:316 | | | | | | |
| 1058 | TCTCCCTGCATGACTTTGTT | −13.2 | −26.3 | 75.5 | −13.1 | 0 | −4.9 |
| | SEQ. ID. NO:317 | | | | | | |
| 1249 | TAGCTTTTTTGTGAATTCTA | −13.2 | −19.1 | 60.3 | −5.9 | 0 | −6.9 |
| | SEQ. ID. NO:318 | | | | | | |
| 1301 | ACCCTTTCAGCAAAGCAATC | −13.2 | −23.7 | 67.1 | −9.6 | −0.8 | −4.7 |
| | SEQ. ID. NO:319 | | | | | | |
| 1579 | TAGCACATCAAGAAGTGGCT | −13.2 | −22.5 | 66.4 | −8.4 | −0.8 | −6.4 |
| | SEQ. ID. NO:320 | | | | | | |
| 1945 | AAAACACAATGTAGAGAAAG | −13.2 | −13.7 | 46.5 | 0 | −0.2 | −4.2 |
| | SEQ. ID. NO:321 | | | | | | |
| 2125 | TGCCATTATGTTTGCTTTAT | −13.2 | −22.3 | 66.4 | −9.1 | 0 | −3.6 |
| | SEQ. ID. NO:322 | | | | | | |
| 689 | CTGCTGACGCGCCCATGCGG | −13.1 | −32.1 | 79.7 | −16 | −3 | −10 |
| | SEQ. ID. NO:323 | | | | | | |
| 694 | CCTCCCTGCTGACGCGCCCA | −13.1 | −35.6 | 86.6 | −21.2 | −1.2 | −7.7 |
| | SEQ. ID. NO:324 | | | | | | |
| 1062 | GTTTTCTCCCTGCATGACTT | −13.1 | −26.4 | 76 | −13.3 | 0 | −4.9 |
| | SEQ. ID. NO:325 | | | | | | |
| 1226 | GAACCTGTACATGATTGGTT | −13.1 | −22 | 64.9 | −7.6 | −1.2 | −9 |
| | SEQ. ID. NO:326 | | | | | | |
| 1252 | TGGTAGCTTTTTTGTGAATT | −13.1 | −20.5 | 63.3 | −7.4 | 0 | −4.6 |
| | SEQ. ID. NO:327 | | | | | | |
| 1679 | CAGCGTGGTGATGATTGAAT | −13.1 | −22.1 | 64.2 | −9 | 0 | −4.1 |
| | SEQ. ID. NO:328 | | | | | | |
| 1800 | TAATATGAGAGAGAAAAAGG | −13.1 | −13.5 | 46.3 | 0 | 0 | −2.7 |
| | SEQ. ID. NO:329 | | | | | | |
| 1810 | TACATCAGATTAATATGAGA | −13.1 | −16.3 | 53 | −3.2 | 0 | −7.4 |
| | SEQ. ID. NO:330 | | | | | | |
| 2120 | TTATGTTTGCTTTATTGCCA | −13.1 | −22.4 | 66.8 | −9.3 | 0 | −3.6 |
| | SEQ. ID. NO:331 | | | | | | |
| 709 | CTCATCCCCTTTGATCCTCC | −13 | −29.8 | 81 | −16.8 | 0 | −4.3 |
| | SEQ. ID. NO:332 | | | | | | |
| 913 | CAACATTCCCATCTCTTTGC | −13 | −24.7 | 70.5 | −11.7 | 0 | −2.6 |
| | SEQ. ID. NO:333 | | | | | | |
| 1039 | TGTCGAGGTCACTTGTCGCA | −13 | −26.6 | 76 | −12.7 | −0.7 | −5.7 |
| | SEQ. ID. NO:334 | | | | | | |
| 1057 | CTCCCTGCATGACTTTGTTG | −13 | −25.9 | 73.6 | −12.9 | 0 | −4.8 |
| | SEQ. ID. NO:335 | | | | | | |
| 1059 | TTCTCCCTGCATGACTTTGT | −13 | −26.3 | 75.5 | −13.3 | 0 | −4.9 |
| | SEQ. ID. NO:336 | | | | | | |
| 1152 | ATTTTATTTGTTATTTCCTG | −13 | −18.7 | 59.2 | −5.7 | 0 | −0.7 |
| | SEQ. ID. NO:337 | | | | | | |
| 1224 | ACCTGTACATGATTGGTTGC | −13 | −23.9 | 69.9 | −10.9 | 0 | −6.2 |
| | SEQ. ID. NO:338 | | | | | | |
| 1247 | GCTTTTTTGTGAATTCTACA | −13 | −20.3 | 62.6 | −6.8 | 0 | −8.1 |
| | SEQ. ID. NO:339 | | | | | | |
| 1292 | GCAAAGCAATCTGGTCTTCA | −13 | −23.1 | 67.7 | −10.1 | 0 | −3.7 |
| | SEQ. ID. NO:340 | | | | | | |
| 1298 | CTTTCAGCAAAGCAATCTGG | −13 | −21.6 | 63.6 | −7.7 | −0.7 | −4.4 |
| | SEQ. ID. NO:341 | | | | | | |
| 1425 | GGTGTTATATATTCATCAGA | −13 | −19.7 | 62.2 | −6.7 | 0 | −4.5 |
| | SEQ. ID. NO:342 | | | | | | |
| 1535 | TATCCTTTATGTATTGTCTA | −13 | −20.1 | 63 | −7.1 | 0 | −1.2 |
| | SEQ. ID. NO:343 | | | | | | |
| 203 | GCTATGTTTCTAAGTCTTCT | −12.9 | −22 | 68.7 | −9.1 | 0 | −2.8 |
| | SEQ. ID. NO:344 | | | | | | |
| 675 | ATGCGGGGCTTCTTTGTTAC | −12.9 | −25.7 | 74.1 | −12.2 | −0.3 | −4.1 |
| | SEQ. ID. NO:345 | | | | | | |
| 710 | GCTCATCCCCTTTGATCCTC | −12.9 | −29.6 | 82 | −16.7 | 0 | −4.3 |
| | SEQ. ID. NO:346 | | | | | | |
| 994 | CACGGTCTGATCTGCATGCT | −12.9 | −26.5 | 74.9 | −12.7 | 0 | −9.7 |
| | SEQ. ID. NO:347 | | | | | | |
| 1045 | CTTTGTTGTCGAGGTCACTT | −12.9 | −24.1 | 72 | −11.2 | 0 | −4.9 |
| | SEQ. ID. NO:348 | | | | | | |
| 1154 | AAATTTTATTTGTTATTTCC | −12.9 | −16.4 | 53.4 | −3.5 | 0 | −4.3 |
| | SEQ. ID. NO:349 | | | | | | |
| 1303 | AGACCCTTTCAGCAAAGCAA | −12.9 | −23.9 | 67.2 | −10.1 | −0.7 | −4.7 |
| | SEQ. ID. NO:350 | | | | | | |
| 1428 | ATAGGTGTTATATATTCATC | −12.9 | −18.1 | 58.7 | −5.2 | 0 | −4 |
| | SEQ. ID. NO:351 | | | | | | |
| 1592 | TACACAACTTTTGTAGCACA | −12.9 | −20.5 | 61.9 | −6.6 | −0.9 | −6.6 |
| | SEQ. ID. NO:352 | | | | | | |
| 1814 | GTTATACATCAGATTAATAT | −12.9 | −16.1 | 52.9 | −3.2 | 0 | −4.7 |
| | SEQ. ID. NO:353 | | | | | | |
| 1946 | TAAAACACAATGTAGAGAAA | −12.9 | −13.4 | 45.9 | 0 | −0.2 | −4.4 |
| | SEQ. ID. NO:354 | | | | | | |
| 1949 | TTTTAAAACACAATGTAGAG | −12.9 | −14.5 | 48.6 | −1 | −0.2 | −6 |
| | SEQ. ID. NO:355 | | | | | | |
| 2015 | GAAGTAACAATCAATTTAAT | −12.9 | −13.9 | 47.2 | −0.9 | 0 | −2.9 |
| | SEQ. ID. NO:356 | | | | | | |
| 2016 | TGAAGTAACAATCAATTTAA | −12.9 | −13.9 | 47.2 | −0.9 | 0 | −2.9 |
| | SEQ. ID. NO:357 | | | | | | |
| 2017 | TTGAAGTAACAATCAATTTA | −12.9 | −14.7 | 49.1 | −0.9 | −0.5 | −3.8 |
| | SEQ. ID. NO:358 | | | | | | |
| 34 | GCATTAGGATAAGTCGGGGA | −12.8 | −23.7 | 68.4 | −10.3 | −0.3 | −3.7 |
| | SEQ. ID. NO:359 | | | | | | |
| 227 | TAAGAGAAGCAGTGTTCACT | −12.8 | −20.7 | 63.5 | −7.9 | 0.4 | −6.6 |
| | SEQ. ID. NO:360 | | | | | | |
| 702 | CCTTTGATCCTCCCTGCTGA | −12.8 | −29.6 | 80.2 | −16.8 | 0 | −3.6 |
| | SEQ. ID. NO:361 | | | | | | |
| 852 | ATATCCATCACACAGTTGCC | −12.8 | −24.8 | 71.4 | −12 | 0 | −3 |
| | SEQ. ID. NO:362 | | | | | | |
| 1120 | TTTGTTATATGAATCCATAA | −12.8 | −16.9 | 53.8 | −3 | −1 | −3.6 |
| | SEQ. ID. NO:363 | | | | | | |
| 1248 | AGCTTTTTTGTGAATTCTAC | −12.8 | −19.6 | 61.5 | −6.8 | 0 | −6.9 |
| | SEQ. ID. NO:364 | | | | | | |
| 1370 | CAGAATGCCCAGACGGAAGT | −12.8 | −25 | 68.3 | −11.4 | −0.6 | −4.2 |
| | SEQ. ID. NO:365 | | | | | | |
| 1374 | AGGTCAGAATGCCCAGACGG | −12.8 | −26.7 | 73.1 | −12.4 | −1.4 | −5.9 |
| | SEQ. ID. NO:366 | | | | | | |
| 95 | GGACTGAGTCTTCCTCTCCA | −12.7 | −27.8 | 80.7 | −13.5 | −1.6 | −6.1 |
| | SEQ. ID. NO:367 | | | | | | |
| 125 | GATGGACTTTCAAGGCCCTG | −12.7 | −26 | 72.6 | −13.3 | 0 | −7.1 |
| | SEQ. ID. NO:368 | | | | | | |
| 660 | GTTACAGGCATCTCTGCTAC | −12.7 | −24.8 | 74.2 | −9.9 | −2.2 | −6.6 |
| | SEQ. ID. NO:369 | | | | | | |
| 836 | TGCCCCCGTTTTTACACTTG | −12.7 | −28 | 74.8 | −14.6 | −0.4 | −3.4 |
| | SEQ. ID. NO:370 | | | | | | |
| 903 | ATCTCTTTGCATTTCCTTAG | −12.7 | −22.6 | 68.6 | −9.9 | 0 | −5.1 |
| | SEQ. ID. NO:371 | | | | | | |
| 1033 | GGTCACTTGTCGCAAGTCAC | −12.7 | −25.4 | 74.2 | −10.5 | −2.2 | −10.8 |
| | SEQ. ID. NO:372 | | | | | | |
| 1056 | TCCCTGCATGACTTTGTTGT | −12.7 | −26.2 | 75.1 | −13.5 | 0 | −4.9 |
| | SEQ. ID. NO:373 | | | | | | |
| 1784 | AAGGAGCTAGACCCCTCCCC | −12.7 | −31.6 | 82.3 | −16.9 | −2 | −7.6 |
| | SEQ. ID. NO:374 | | | | | | |
| 2117 | TGTTTGCTTTATTGCCAAGA | −12.7 | −22.5 | 66.4 | −9.8 | 0 | −3.4 |
| | SEQ. ID. NO:375 | | | | | | |
| 362 | GTTCAATGAGATTCATTTTT | −12.6 | −18.5 | 58.7 | −4.2 | −1.7 | −6.2 |
| | SEQ. ID. NO:376 | | | | | | |
| 363 | TGTTCAATGAGATTCATTTT | −12.6 | −18.4 | 58.2 | −4.2 | −1.5 | −6 |
| | SEQ. ID. NO:377 | | | | | | |
| 438 | CCTGCCACTTGTTCTGTTAA | −12.6 | −25.5 | 72.8 | −12.9 | 0 | −3 |
| | SEQ. ID. NO:378 | | | | | | |
| 578 | AGTACCACTCTTCAGGCTGC | −12.6 | −27.1 | 79.1 | −14.5 | 0 | −5.2 |
| | SEQ. ID. NO:379 | | | | | | |
| 995 | TCACGGTCTGATCTGCATGC | −12.6 | −26 | 74.7 | −12.7 | 0 | −8.7 |
| | SEQ. ID. NO:380 | | | | | | |
| 1040 | TTGTCGAGGTCACTTGTCGC | −12.6 | −26 | 75.3 | −12.7 | −0.4 | −5.4 |
| | SEQ. ID. NO:381 | | | | | | |
| 1228 | AAGAACCTGTACATGATTGG | −12.6 | −20 | 59.7 | −7.4 | 0 | −6.1 |
| | SEQ. ID. NO:382 | | | | | | |
| 1718 | TAAACTTGTGGTCGTTTACT | −12.6 | −20.5 | 62 | −7.1 | −0.6 | −4.7 |
| | SEQ. ID. NO:383 | | | | | | |
| 1792 | GAGAGAAAAAGGAGCTAGAC | −12.6 | −18 | 55.9 | −5.4 | 0 | −5.1 |
| | SEQ. ID. NO:384 | | | | | | |
| 2118 | ATGTTTGCTTTATTGCCAAG | −12.6 | −21.9 | 65 | −9.3 | 0 | −3.6 |
| | SEQ. ID. NO:385 | | | | | | |
| 309 | TTCTTTATGGTGGTCTTCAA | −12.5 | −21.9 | 67.4 | −9.4 | 0 | −3.3 |
| | SEQ. ID. NO:386 | | | | | | |
| 494 | ACTGAACATTGCTGTATTGC | −12.5 | −21.5 | 64.3 | −9 | 0 | −3.9 |
| | SEQ. ID. NO:387 | | | | | | |
| 574 | CCACTCTTCAGGCTGCTGGG | −12.5 | −29.3 | 82.2 | −15.3 | −1.4 | −6.1 |
| | SEQ. ID. NO:388 | | | | | | |
| 611 | CTGGCATACGCCTGAGTTCA | −12.5 | −27 | 75.2 | −11.6 | −2.9 | −7.9 |
| | SEQ. ID. NO:389 | | | | | | |
| 736 | GGCTCTGTCTCCACAAACAA | −12.5 | −24.5 | 69.6 | −12 | 0.1 | −3.8 |
| | SEQ. ID. NO:390 | | | | | | |
| 1041 | GTTGTCGAGGTCACTTGTCG | −12.5 | −25.4 | 74.3 | −12.9 | 0.4 | −4.9 |
| | SEQ. ID. NO:391 | | | | | | |
| 1811 | ATACATCAGATTAATATGAG | −12.5 | −15.7 | 51.7 | −3.2 | 0 | −6.9 |
| | SEQ. ID. NO:392 | | | | | | |
| 2018 | ATTGAAGTAACAATCAATTT | −12.5 | −15 | 49.6 | −0.9 | −1.4 | −5.5 |
| | SEQ. ID. NO:393 | | | | | | |
| 364 | ATGTTCAATGAGATTCATTT | −12.4 | −18.3 | 57.9 | −4.2 | −1.7 | −6.2 |
| | SEQ. ID. NO:394 | | | | | | |
| 668 | GCTTCTTTGTTACAGGCATC | −12.4 | −24.3 | 73.3 | −11.9 | 0 | −4.2 |
| | SEQ. ID. NO:395 | | | | | | |
| 1112 | ATGAATCCATAATAAAATGT | −12.4 | −14.8 | 48.5 | −2.4 | 0 | −2.8 |
| | SEQ. ID. NO:396 | | | | | | |
| 1534 | ATCCTTTATGTATTGTCTAT | −12.4 | −20.4 | 63.6 | −8 | 0 | −0.9 |
| | SEQ. ID. NO:397 | | | | | | |
| 1689 | ATCAGCATCTCAGCGTGGTG | −12.4 | −26.2 | 76.2 | −12.6 | −1.1 | −4.1 |
| | SEQ. ID. NO:398 | | | | | | |
| 1790 | GAGAAAAAGGAGCTAGACCC | −12.4 | −21.4 | 61.7 | −9 | 0 | −5.8 |
| | SEQ. ID. NO:399 | | | | | | |
| 1896 | GTGGAAGTTACACATGTAAT | −12.4 | −19.3 | 59.5 | −6 | −0.8 | −7.1 |
| | SEQ. ID. NO:400 | | | | | | |
| 1899 | GTTGTGGAAGTTACACATGT | −12.4 | −21.6 | 65.7 | −7.5 | −1.7 | −6.1 |
| | SEQ. ID. NO:401 | | | | | | |
| 2014 | AAGTAACAATCAATTTAATT | −12.4 | −13.4 | 46.3 | −0.9 | 0 | −2.9 |
| | SEQ. ID. NO:402 | | | | | | |
| 2044 | AGATTTTCCCTAGTTCAACA | −12.4 | −22.5 | 66.7 | −10.1 | 0 | −3.6 |
| | SEQ. ID. NO:403 | | | | | | |
| 93 | ACTGAGTCTTCCTCTCCAGA | −12.3 | −26.6 | 78.3 | −13 | −1.2 | −4.9 |
| | SEQ. ID. NO:404 | | | | | | |
| 96 | TGGACTGAGTCTTCCTCTCC | −12.3 | −27.1 | 79.4 | −13.5 | −1.2 | −6.9 |
| | SEQ. ID. NO:405 | | | | | | |
| 126 | AGATGGACTTTCAAGGCCCT | −12.3 | −26 | 73 | −13.7 | 0 | −7.1 |
| | SEQ. ID. NO:406 | | | | | | |
| 142 | GATTGTTTTGGGTCAGAGAT | −12.3 | −22.1 | 67.7 | −9.8 | 0 | −2.7 |
| | SEQ. ID. NO:407 | | | | | | |
| 602 | GCCTGAGTTCATATATTCCA | −12.3 | −24.3 | 71 | −12 | 0 | −3.6 |
| | SEQ. ID. NO:408 | | | | | | |
| 1002 | TCTTCATTCACGGTCTGATC | −12.3 | −23.4 | 70.2 | −11.1 | 0 | −3.9 |
| | SEQ. ID. NO:409 | | | | | | |
| 1253 | CTGGTAGCTTTTTTGTGAAT | −12.3 | −21.3 | 64.9 | −9 | 0 | −4.3 |
| | SEQ. ID. NO:410 | | | | | | |
| 1306 | CGCAGACCCTTTCAGCAAAG | −12.3 | −25.4 | 69.4 | −12 | −1 | −4.8 |
| | SEQ. ID. NO:411 | | | | | | |
| 1371 | TCAGAATGCCCAGACGGAAG | −12.3 | −24.2 | 66.7 | −11.4 | −0.1 | −3.5 |
| | SEQ. ID. NO:412 | | | | | | |
| 1670 | GATGATTGAATGTCCGTAAT | −12.3 | −19.8 | 59 | −7.5 | 0 | −2.6 |
| | SEQ. ID. NO:413 | | | | | | |
| 1671 | TGATGATTGAATGTCCGTAA | −12.3 | −19.8 | 58.9 | −7.5 | 0 | −2.6 |
| | SEQ. ID. NO:414 | | | | | | |
| 1794 | GAGAGAGAAAAAGGAGCTAG | −12.3 | −17.8 | 55.6 | −5.5 | 0 | −5.1 |
| | SEQ. ID. NO:415 | | | | | | |
| 1964 | GGATTCCCTGGAGCCTTTTA | −12.3 | −27.7 | 77.2 | −15.4 | 0 | −4.6 |
| | SEQ. ID. NO:416 | | | | | | |
| 1967 | GCAGGATTCCCTGGAGCCTT | −12.3 | −30.3 | 82.8 | −15 | −3 | −9.1 |
| | SEQ. ID. NO:417 | | | | | | |
| 2119 | TATGTTTGCTTTATTGCCAA | −12.3 | −21.6 | 64.2 | −9.3 | 0 | −3.6 |
| | SEQ. ID. NO:418 | | | | | | |
| 2131 | TTCTGTTGCCATTATGTTTG | −12.3 | −22.4 | 67.4 | −10.1 | 0 | −3 |
| | SEQ. ID. NO:419 | | | | | | |
| 439 | ACCTGCCACTTGTTCTGTTA | −12.2 | −26.4 | 75.9 | −14.2 | 0 | −3 |
| | SEQ. ID. NO:420 | | | | | | |
| 485 | TGCTGTATTGCGAGTATGGT | −12.2 | −24.2 | 70.9 | −11.1 | −0.7 | −4.1 |
| | SEQ. ID. NO:421 | | | | | | |
| 804 | TTGGTAATGCTTCTCCTGAA | −12.2 | −22.8 | 66.9 | −10.6 | 0 | −3.2 |
| | SEQ. ID. NO:422 | | | | | | |
| 975 | TGCTTCACATTTTTTCTCAG | −12.2 | −21.7 | 66.7 | −9.5 | 0 | −3.6 |
| | SEQ. ID. NO:423 | | | | | | |
| 993 | ACGGTCTGATCTGCATGCTG | −12.2 | −25.8 | 73.6 | −12.7 | 0 | −9.7 |
| | SEQ. ID. NO:424 | | | | | | |
| 1368 | GAATGCCCAGACGGAAGTTT | −12.2 | −24.5 | 67.6 | −11.4 | −0.8 | −4.4 |
| | SEQ. ID. NO:425 | | | | | | |
| 1578 | AGCACATCAAGAAGTGGCTC | −12.2 | −23.2 | 68.5 | −10.1 | −0.8 | −6.4 |
| | SEQ. ID. NO:426 | | | | | | |
| 1588 | CAACTTTTGTAGCACATCAA | −12.2 | −20.1 | 60.7 | −7.4 | −0.1 | −5.6 |
| | SEQ. ID. NO:427 | | | | | | |
| 1668 | TGATTGAATGTCCGTAATTC | −12.2 | −19.7 | 59.4 | −7.5 | 0.4 | −5.2 |
| | SEQ. ID. NO:428 | | | | | | |
| 1812 | TATACATCAGATTAATATGA | −12.2 | −15.4 | 51 | −3.2 | 0 | −7.2 |
| | SEQ. ID. NO:429 | | | | | | |
| 1950 | CTTTTAAAACACAATGTAGA | −12.2 | −15.4 | 50.3 | −2.7 | −0.2 | −6.2 |
| | SEQ. ID. NO:430 | | | | | | |
| 1968 | TGCAGGATTCCCTGGAGCCT | −12.2 | −30.2 | 82.1 | −15 | −3 | −9.1 |
| | SEQ. ID. NO:431 | | | | | | |
| 118 | TTTCAAGGCCCTGGGAGGAT | −12.1 | −27.3 | 75.6 | −14.4 | −0.6 | −8.3 |
| | SEQ. ID. NO:432 | | | | | | |
| 210 | ACTTTGAGCTATGTTTCTAA | −12.1 | −20 | 62.2 | −7.9 | 0 | −5.1 |
| | SEQ. ID. NO:433 | | | | | | |
| 310 | TTTCTTTATGGTGGTCTTCA | −12.1 | −22.7 | 70.3 | −10.6 | 0 | −3.1 |
| | SEQ. ID. NO:434 | | | | | | |
| 671 | GGGGCTTCTTTGTTACAGGC | −12.1 | −26.8 | 78.8 | −14.7 | 0 | −3.7 |
| | SEQ. ID. NO:435 | | | | | | |
| 810 | GCGTTTTTGGTAATGCTTCT | −12.1 | −23.7 | 69.6 | −10.9 | −0.5 | −3.9 |
| | SEQ. ID. NO:436 | | | | | | |
| 1369 | AGAATGCCCAGACGGAAGTT | −12.1 | −24.4 | 67.5 | −11.4 | −0.8 | −3.9 |
| | SEQ. ID. NO:437 | | | | | | |
| 1482 | GCATACTCCTCTTGAGTCAT | −12.1 | −24.9 | 73.9 | −11.1 | −1.7 | −6.8 |
| | SEQ. ID. NO:438 | | | | | | |
| 1581 | TGTAGCACATCAAGAAGTGG | −12.1 | −21 | 63.3 | −8.4 | −0.1 | −5.7 |
| | SEQ. ID. NO:439 | | | | | | |
| 1719 | GTAAACTTGTGGTCGTTTAC | −12.1 | −20.8 | 63.2 | −6.9 | −1.8 | −6 |
| | SEQ. ID. NO:440 | | | | | | |
| 1815 | AGTTATACATCAGATTAATA | −12.1 | −16.1 | 53 | −4 | 0 | −4.7 |
| | SEQ. ID. NO:441 | | | | | | |
| 987 | TGATCTGCATGCTGCTTCAC | −12 | −25.2 | 73.6 | −11.4 | −1.8 | −9.7 |
| | SEQ. ID. NO:442 | | | | | | |
| 997 | ATTCACGGTCTGATCTGCAT | −12 | −24.3 | 70.8 | −12.3 | 0 | −4.9 |
| | SEQ. ID. NO:443 | | | | | | |
| 1213 | ATTGGTTGCCATTTCCGTCA | −12 | −26.8 | 75.1 | −14.1 | −0.4 | −4.6 |
| | SEQ. ID. NO:444 | | | | | | |
| 1225 | AACCTGTACATGATTGGTTG | −12 | −21.4 | 63.5 | −8.5 | −0.8 | −8.2 |
| | SEQ. ID. NO:445 | | | | | | |
| 1276 | TTCATGGTCCAAAGTCTGAA | −12 | −21.7 | 64.3 | −9.7 | 0 | −5 |
| | SEQ. ID. NO:446 | | | | | | |
| 1277 | CTTCATGGTCCAAAGTCTGA | −12 | −23.3 | 68.5 | −11.3 | 0 | −5 |
| | SEQ. ID. NO:447 | | | | | | |
| 1295 | TCAGCAAAGCAATCTGGTCT | −12 | −23 | 67.6 | −10.1 | −0.7 | −4.4 |
| | SEQ. ID. NO:448 | | | | | | |
| 1312 | TTCAACCGCAGACCCTTTCA | −12 | −27 | 72.9 | −15 | 0 | −3.6 |
| | SEQ. ID. NO:449 | | | | | | |
| 1367 | AATGCCCAGACGGAAGTTTC | −12 | −24.3 | 67.8 | −11.4 | −0.8 | −4.4 |
| | SEQ. ID. NO:450 | | | | | | |
| 1536 | CTATCCTTTATGTATTGTCT | −12 | −21.3 | 65.7 | −9.3 | 0 | −1.2 |
| | SEQ. ID. NO:451 | | | | | | |
| 1801 | TTAATATGAGAGAGAAAAAG | −12 | −12.4 | 44.3 | 0 | 0 | −2.7 |
| | SEQ. ID. NO:452 | | | | | | |
| 360 | TCAATGAGATTCATTTTTGA | −11.9 | −17.8 | 56.5 | −4.2 | −1.7 | −7.2 |
| | SEQ. ID. NO:453 | | | | | | |
| 674 | TGCGGGGCTTCTTTGTTACA | −11.9 | −26.4 | 75.2 | −13.9 | −0.3 | −4.1 |
| | SEQ. ID. NO:454 | | | | | | |
| 910 | CATTCCCATCTCTTTGCATT | −11.9 | −25.3 | 72.7 | −13.4 | 0 | −5.1 |
| | SEQ. ID. NO:455 | | | | | | |
| 1148 | TATTTGTTATTTCCTGAGGC | −11.9 | −22 | 66.8 | −10.1 | 0 | −3.6 |
| | SEQ. ID. NO:456 | | | | | | |
| 1429 | CATAGGTGTTATATATTCAT | −11.9 | −18.4 | 58.6 | −6.5 | 0 | −3.9 |
| | SEQ. ID. NO:457 | | | | | | |
| 1553 | GCTTCTCTACTGCCTCTCTA | −11.9 | −27.2 | 80.5 | −15.3 | 0 | −3.1 |
| | SEQ. ID. NO:458 | | | | | | |
| 1665 | TTGAATGTCCGTAATTCAGT | −11.9 | −21 | 62.6 | −7.5 | −1.6 | −6.4 |
| | SEQ. ID. NO:459 | | | | | | |
| 1953 | AGCCTTTTAAAACACAATGT | −11.9 | −18.9 | 56.9 | −7 | 0 | −6.2 |
| | SEQ. ID. NO:460 | | | | | | |
| 167 | TTCTACGATGTCTTCTACCT | −11.8 | −23.4 | 69.2 | −11.6 | 0 | −3 |
| | SEQ. ID. NO:461 | | | | | | |
| 922 | GCATTCAGCCAACATTCCCA | −11.8 | −27.6 | 75.1 | −15.3 | −0.1 | −3.5 |
| | SEQ. ID. NO:462 | | | | | | |
| 1222 | CTGTACATGATTGGTTGCCA | −11.8 | −24.4 | 70.5 | −12.1 | −0.2 | −6.5 |
| | SEQ. ID. NO:463 | | | | | | |
| 1297 | TTTCAGCAAAGCAATCTGGT | −11.8 | −21.9 | 64.8 | −9.6 | −0.2 | −4.1 |
| | SEQ. ID. NO:464 | | | | | | |
| 1373 | GGTCAGAATGCCCAGACGGA | −11.8 | −27.3 | 74.1 | −14.2 | −1.2 | −5.2 |
| | SEQ. ID. NO:465 | | | | | | |
| 1669 | ATGATTGAATGTCCGTAATT | −11.8 | −19.3 | 58.1 | −7.5 | 0 | −3 |
| | SEQ. ID. NO:466 | | | | | | |
| 98 | TCTGGACTGAGTCTTCCTCT | −11.7 | −26 | 77.7 | −13 | −1.2 | −6.9 |
| | SEQ. ID. NO:467 | | | | | | |
| 308 | TCTTTATGGTGGTCTTCAAA | −11.7 | −21.1 | 64.6 | −9.4 | 0 | −3.3 |
| | SEQ. ID. NO:468 | | | | | | |
| 966 | TTTTTTCTCAGTCGCTTAGA | −11.7 | −22.4 | 68.5 | −10.7 | 0 | −3.1 |
| | SEQ. ID. NO:469 | | | | | | |
| 1065 | TCAGTTTTCTCCCTGCATGA | −11.7 | −26.3 | 76.2 | −14.6 | 0 | −5.7 |
| | SEQ. ID. NO:470 | | | | | | |
| 1254 | CCTGGTAGCTTTTTTGTGAA | −11.7 | −23.3 | 68.8 | −11.6 | 0 | −4.6 |
| | SEQ. ID. NO:471 | | | | | | |
| 1294 | CAGCAAAGCAATCTGGTCTT | −11.7 | −22.7 | 66.4 | −10.1 | −0.7 | −4.4 |
| | SEQ. ID. NO:472 | | | | | | |
| 1379 | CCAATAGGTCAGAATGCCCA | −11.7 | −25.6 | 70.3 | −12.4 | −1.4 | −4.5 |
| | SEQ. ID. NO:473 | | | | | | |
| 1813 | TTATACATCAGATTAATATG | −11.7 | −14.9 | 50 | −3.2 | 0 | −5.9 |
| | SEQ. ID. NO:474 | | | | | | |
| 1938 | AATGTACAGAAAGTTGTTCT | −11.7 | −17.9 | 57.2 | −4.9 | −1.2 | −3.9 |
| | SEQ. ID. NO:475 | | | | | | |
| 2130 | TCTGTTGCCATTATGTTTGC | −11.7 | −24.1 | 71.5 | −12.4 | 0 | −3 |
| | SEQ. ID. NO:476 | | | | | | |
| 127 | GAGATGGACTTTCAAGGCCC | −11.6 | −25.7 | 72.4 | −14.1 | 0 | −7.1 |
| | SEQ. ID. NO:477 | | | | | | |
| 737 | AGGCTCTGTCTCCACAAACA | −11.6 | −25.2 | 72.2 | −13.1 | −0.2 | −3.8 |
| | SEQ. ID. NO:478 | | | | | | |
| 835 | GCCCCCGTTTTTACACTTGT | −11.6 | −29.2 | 78.2 | −16.9 | −0.4 | −3.1 |
| | SEQ. ID. NO:479 | | | | | | |
| 992 | CGGTCTGATCTGCATGCTGC | −11.6 | −27.4 | 77.4 | −14.6 | −1 | −9.7 |
| | SEQ. ID. NO:480 | | | | | | |
| 1014 | CGACCTTCACTGTCTTCATT | −11.6 | −24.6 | 71.1 | −12.3 | −0.5 | −3.7 |
| | SEQ. ID. NO:481 | | | | | | |
| 1565 | GTGGCTCCTGAAGCTTCTCT | −11.6 | −27.7 | 80.3 | −14 | −2.1 | −10.8 |
| | SEQ. ID. NO:482 | | | | | | |
| 1583 | TTTGTAGCACATCAAGAAGT | −11.6 | −20 | 61.5 | −8.4 | 0 | −5.1 |
| | SEQ. ID. NO:483 | | | | | | |
| 1793 | AGAGAGAAAAAGGAGCTAGA | −11.6 | −17.8 | 55.6 | −6.2 | 0 | −5.1 |
| | SEQ. ID. NO:484 | | | | | | |
| 1925 | TTGTTCTATCTAGCCCAATA | −11.6 | −22.9 | 67.6 | −11.3 | 0 | −3.7 |
| | SEQ. ID. NO:485 | | | | | | |
| 446 | CCAGAGGACCTGCCACTTGT | −11.5 | −29.1 | 79.3 | −16.7 | −0.7 | −4.6 |
| | SEQ. ID. NO:486 | | | | | | |
| 1275 | TCATGGTCCAAAGTCTGAAA | −11.5 | −20.9 | 61.9 | −9.4 | 0 | −5 |
| | SEQ. ID. NO:487 | | | | | | |
| 1593 | TTACACAACTTTTGTAGCAC | −11.5 | −19.9 | 61 | −7.4 | −0.9 | −5.8 |
| | SEQ. ID. NO:488 | | | | | | |
| 1683 | ATCTCAGCGTGGTGATGATT | −11.5 | −23.9 | 70.3 | −11.4 | −0.9 | −5.2 |
| | SEQ. ID. NO:489 | | | | | | |
| 1691 | ACATCAGCATCTCAGCGTGG | −11.5 | −25.9 | 74.5 | −13.4 | −0.9 | −4.2 |
| | SEQ. ID. NO:490 | | | | | | |
| 1759 | TCCCCATCACTGCACGTCCC | −11.5 | −32.4 | 83.8 | −20.9 | 0 | −4.8 |
| | SEQ. ID. NO:491 | | | | | | |
| 1778 | CTAGACCCCTCCCCTGTAAT | −11.5 | −29.8 | 78.3 | −18.3 | 0 | −3 |
| | SEQ. ID. NO:492 | | | | | | |
| 1913 | GCCCAATATTTACAGTTGTG | −11.5 | −22.8 | 66.4 | −11.3 | 0 | −4.1 |
| | SEQ. ID. NO:493 | | | | | | |
| 2116 | GTTTGCTTTATTGCCAAGAT | −11.5 | −22.5 | 66.5 | −11 | 0 | −3.6 |
| | SEQ. ID. NO:494 | | | | | | |
| 92 | CTGAGTCTTCCTCTCCAGAT | −11.4 | −26.4 | 77.6 | −13.7 | −1.2 | −4.3 |
| | SEQ. ID. NO:495 | | | | | | |
| 361 | TTCAATGAGATTCATTTTTG | −11.4 | −17.3 | 55.5 | −4.2 | −1.7 | −6.2 |
| | SEQ. ID. NO:496 | | | | | | |
| 1293 | AGCAAAGCAATCTGGTCTTC | −11.4 | −22.4 | 66.8 | −10.1 | −0.7 | −4.4 |
| | SEQ. ID. NO:497 | | | | | | |
| 1667 | GATTGAATGTCCGTAATTCA | −11.4 | −20.4 | 60.6 | −7.5 | −1.4 | −6 |
| | SEQ. ID. NO:498 | | | | | | |
| 1806 | TCAGATTAATATGAGAGAGA | −11.4 | −16.9 | 54.6 | −5.5 | 0 | −6.5 |
| | SEQ. ID. NO:499 | | | | | | |
| 2013 | AGTAACAATCAATTTAATTA | −11.4 | −13.8 | 47.3 | −2.4 | 0 | −3.7 |
| | SEQ. ID. NO:500 | | | | | | |
| 99 | TTCTGGACTGAGTCTTCCTC | −11.3 | −25.2 | 75.9 | −13 | −0.7 | −6.9 |
| | SEQ. ID. NO:501 | | | | | | |
| 141 | ATTGTTTTGGGTCACAGATG | −11.3 | −21.5 | 66.1 | −9.6 | −0.3 | −3.5 |
| | SEQ. ID. NO:502 | | | | | | |
| 573 | CACTCTTCAGGCTGCTGGGG | −11.3 | −28.5 | 81.3 | −15.7 | −1.4 | −6.1 |
| | SEQ. ID. NO:503 | | | | | | |
| 614 | CAGCTGGCATACGCCTGAGT | −11.3 | −28.3 | 77.7 | −14.8 | −2.2 | −9.9 |
| | SEQ. ID. NO:504 | | | | | | |
| 1119 | TTGTTATATGAATCCATAAT | −11.3 | −16.8 | 53.5 | −4.4 | −1 | −3.6 |
| | SEQ. ID. NO:505 | | | | | | |
| 1212 | TTGGTTGCCATTTCCGTCAA | −11.3 | −26.1 | 72.7 | −14.1 | −0.4 | −4.6 |
| | SEQ. ID. NO:506 | | | | | | |
| 1954 | GAGCCTTTTAAAACACAATG | −11.3 | −18.3 | 55.4 | −7 | 0 | −6 |
| | SEQ. ID. NO:507 | | | | | | |
| 2121 | ATTATGTTTGCTTTATTGCC | −11.3 | −21.7 | 65.5 | −10.4 | 0 | −3.6 |
| | SEQ. ID. NO:508 | | | | | | |
| 117 | TTCAAGGCCCTGGGAGGATT | −11.2 | −27.3 | 75.6 | −15.3 | −0.6 | −8.3 |
| | SEQ. ID. NO:509 | | | | | | |
| 437 | CTGCCACTTGTTCTGTTAAA | −11.2 | −22.8 | 66.9 | −11.6 | 0 | −3 |
| | SEQ. ID. NO:510 | | | | | | |
| 610 | TGGCATACGCCTGAGTTCAT | −11.2 | −26.1 | 73.2 | −12 | −2.9 | −7.9 |
| | SEQ. ID. NO:511 | | | | | | |
| 976 | CTGCTTCACATTTTTTCTCA | −11.2 | −22.6 | 68.5 | −11.4 | 0 | −3.6 |
| | SEQ. ID. NO:512 | | | | | | |
| 1046 | ACTTTGTTGTCGAGGTCACT | −11.2 | −24.2 | 72.2 | −13 | 0 | −4.9 |
| | SEQ. ID. NO:513 | | | | | | |
| 1070 | TGAGTTCAGTTTTCTCCCTG | −11.2 | −25.1 | 74.9 | −13.3 | −0.3 | −4.3 |
| | SEQ. ID. NO:514 | | | | | | |
| 1216 | ATGATTGGTTGCCATTTCCG | −11.2 | −25.1 | 70.2 | −13.2 | −0.4 | −4.6 |
| | SEQ. ID. NO:515 | | | | | | |
| 1219 | TACATGATTGGTTGCCATTT | −11.2 | −22.5 | 66.1 | −10.6 | −0.4 | −5.9 |
| | SEQ. ID. NO:516 | | | | | | |
| 1255 | TCCTGGTAGCTTTTTTGTGA | −11.2 | −24.4 | 72.9 | −13.2 | 0 | −4.6 |
| | SEQ. ID. NO:517 | | | | | | |
| 1291 | CAAAGCAATCTGGTCTTCAT | −11.2 | −21.3 | 63.5 | −10.1 | 0 | −4.1 |
| | SEQ. ID. NO:518 | | | | | | |
| 1431 | AACATAGGTGTTATATATTC | −11.2 | −17.2 | 55.8 | −4.7 | −1.2 | −7 |
| | SEQ. ID. NO:519 | | | | | | |
| 1554 | AGCTTCTCTACTGCCTCTCT | −11.2 | −27.5 | 81.5 | −16.3 | 0 | −4.3 |
| | SEQ. ID. NO:520 | | | | | | |
| 1586 | ACTTTTGTAGCACATCAAGA | −11.2 | −20.7 | 63.1 | −8.4 | −1 | −6.9 |
| | SEQ. ID. NO:521 | | | | | | |
| 1680 | TCAGCGTGGTGATGATTGAA | −11.2 | −22.5 | 65.7 | −10.4 | −0.7 | −4.6 |
| | SEQ. ID. NO:522 | | | | | | |
| 1684 | CATCTCAGCGTGGTGATGAT | −11.2 | −24.5 | 71.1 | −12.3 | −0.9 | −5.6 |
| | SEQ. ID. NO:523 | | | | | | |
| 1900 | AGTTGTGGAAGTTACACATG | −11.2 | −20.4 | 62.7 | −7.5 | −1.7 | −5.9 |
| | SEQ. ID. NO:524 | | | | | | |
| 67 | CGATTTTGCTACAAATGCTC | −11.1 | −20.7 | 61 | −8.8 | −0.6 | −5.2 |
| | SEQ. ID. NO:525 | | | | | | |
| 486 | TTGCTGTATTGCGAGTATGG | −11.1 | −23.1 | 67.9 | −11.1 | −0.7 | −4.1 |
| | SEQ. ID. NO:526 | | | | | | |
| 672 | CGGGGCTTCTTTGTTACAGG | −11.1 | −25.8 | 73.9 | −14.7 | 0 | −3.7 |
| | SEQ. ID. NO:527 | | | | | | |
| 1215 | TGATTGGTTGCCATTTCCGT | −11.1 | −26.3 | 73.5 | −14.5 | −0.4 | −4.6 |
| | SEQ. ID. NO:528 | | | | | | |
| 1543 | TGCCTCTCTATCCTTTATGT | −11.1 | −25.4 | 74.7 | −14.3 | 0 | −3 |
| | SEQ. ID. NO:529 | | | | | | |
| 1688 | TCAGCATCTCAGCGTGGTGA | −11.1 | −26.8 | 77.6 | −13.9 | −1.8 | −4.2 |
| | SEQ. ID. NO:530 | | | | | | |
| 1716 | AACTTGTGGTCGTTTACTCT | −11.1 | −22.8 | 68.3 | −11.7 | 0 | −3 |
| | SEQ. ID. NO:531 | | | | | | |
| 1952 | GCCTTTTAAAACACAATGTA | −11.1 | −18.6 | 56.2 | −7 | −0.2 | −6.2 |
| | SEQ. ID. NO:532 | | | | | | |
| 33 | CATTAGGATAAGTCGGGGAG | −11 | −21.9 | 64.5 | −10.3 | −0.3 | −3 |
| | SEQ. ID. NO:533 | | | | | | |
| 35 | CGCATTAGGATAAGTCGGGG | −11 | −23.9 | 67.3 | −12.3 | −0.3 | −3.9 |
| | SEQ. ID. NO:534 | | | | | | |
| 64 | TTTTGCTACAAATGCTCAGA | −11 | −20.6 | 61.9 | −8.8 | −0.6 | −5.2 |
| | SEQ. ID. NO:535 | | | | | | |
| 66 | GATTTTGCTACAAATGCTCA | −11 | −20.6 | 61.7 | −8.8 | −0.6 | −5.2 |
| | SEQ. ID. NO:536 | | | | | | |
| 140 | TTGTTTTGGGTCAGAGATGG | −11 | −22.7 | 68.9 | −10.8 | −0.7 | −3.6 |
| | SEQ. ID. NO:537 | | | | | | |
| 1660 | TGTCCGTAATTCAGTCAGGC | −11 | −25.1 | 73.2 | −14.1 | 0 | −3.4 |
| | SEQ. ID. NO:538 | | | | | | |
| 1717 | AAACTTGTGGTCGTTTACTC | −11 | −21.2 | 64 | −10.2 | 0 | −4.1 |
| | SEQ. ID. NO:539 | | | | | | |
| 601 | CCTGAGTTCATATATTCCAG | −10.9 | −22.5 | 66.9 | −11.6 | 0 | −3.6 |
| | SEQ. ID. NO:540 | | | | | | |
| 670 | GGGCTTCTTTGTTACAGGCA | −10.9 | −26.3 | 77.1 | −14.7 | −0.4 | −4.2 |
| | SEQ. ID. NO:541 | | | | | | |
| 970 | CACATTTTTTCTCAGTCGCT | −10.9 | −23.6 | 70.1 | −12.7 | 0 | −3.1 |
| | SEQ. ID. NO:542 | | | | | | |
| 1585 | CTTTTGTAGCACATCAAGAA | −10.9 | −19.8 | 60.5 | −8.4 | −0.1 | −5.4 |
| | SEQ. ID. NO:543 | | | | | | |
| 1595 | TCTTACACAACTTTTGTAGC | −10.9 | −20.3 | 62.7 | −8.4 | −0.9 | −4.4 |
| | SEQ. ID. NO:544 | | | | | | |
| 1791 | AGAGAAAAAGGAGCTAGACC | −10.9 | −19.4 | 58.3 | −8.5 | 0 | −5.4 |
| | SEQ. ID. NO:545 | | | | | | |
| 1841 | AACTGGGTACAAGTGAAATA | −10.9 | −18 | 55.6 | −7.1 | 0 | −6 |
| | SEQ. ID. NO:546 | | | | | | |
| 1912 | CCCAATATTTACAGTTGTGG | −10.9 | −22.2 | 64.8 | −11.3 | 0 | −4.1 |
| | SEQ. ID. NO:547 | | | | | | |
| 1955 | GGAGCCTTTTAAAACACAAT | −10.9 | −19.5 | 57.8 | −8.6 | 0 | −6.2 |
| | SEQ. ID. NO:548 | | | | | | |
| 2128 | TGTTGCCATTATGTTTGCTT | −10.9 | −23.8 | 70.2 | −12.9 | 0 | −3.6 |
| | SEQ. ID. NO:549 | | | | | | |
| 100 | ATTCTGGACTGAGTCTTCCT | −10.8 | −24.8 | 74 | −13 | −0.9 | −6.2 |
| | SEQ. ID. NO:550 | | | | | | |
| 112 | GGCCCTGGGAGGATTCTGGA | −10.8 | −29.9 | 82 | −18.3 | −0.6 | −8.3 |
| | SEQ. ID. NO:551 | | | | | | |
| 735 | GCTCTGTCTCCACAAACAAC | −10.8 | −23.5 | 67.7 | −12.2 | −0.1 | −2.9 |
| | SEQ. ID. NO:552 | | | | | | |
| 875 | CTTGACACTTTCTTCGCATG | −10.8 | −22.9 | 67 | −12.1 | 0 | −4.5 |
| | SEQ. ID. NO:553 | | | | | | |
| 962 | TTCTCAGTCGCTTAGATTTA | −10.8 | −21.9 | 67.1 | −11.1 | 0 | −3.1 |
| | SEQ. ID. NO:554 | | | | | | |
| 1261 | CTGAAATCCTGGTAGCTTTT | −10.8 | −22.5 | 66.2 | −11.7 | 0 | −4.7 |
| | SEQ. ID. NO:555 | | | | | | |
| 1582 | TTGTAGCACATCAAGAAGTG | −10.8 | −19.9 | 61 | −8.4 | −0.4 | −5.7 |
| | SEQ. ID. NO:556 | | | | | | |
| 1646 | TCAGGCGACCCAGGAGACAG | −10.8 | −27.7 | 75.5 | −15.9 | −0.9 | −5.4 |
| | SEQ. ID. NO:557 | | | | | | |
| 1682 | TCTCAGCGTGGTGATGATTG | −10.8 | −23.9 | 70.1 | −12.1 | −0.9 | −4.8 |
| | SEQ. ID. NO:558 | | | | | | |
| 1816 | AAGTTATACATCAGATTAAT | −10.8 | −15.7 | 51.8 | −4.9 | 0 | −4.6 |
| | SEQ. ID. NO:559 | | | | | | |
| 1965 | AGGATTCCCTGGAGCCTTTT | −10.8 | −28 | 78.1 | −16.3 | −0.7 | −6 |
| | SEQ. ID. NO:560 | | | | | | |
| 1977 | CAATTAGAATGCAGGATTCC | −10.8 | −20.5 | 61 | −8.3 | −1.3 | −5.8 |
| | SEQ. ID. NO:561 | | | | | | |
| 119 | CTTTCAAGGCCCTGGGAGGA | −10.7 | −28.2 | 77.6 | −16.7 | −0.6 | −8.3 |
| | SEQ. ID. NO:562 | | | | | | |
| 164 | TACGATGTCTTCTACCTCCT | −10.7 | −25.3 | 72.5 | −14.6 | 0 | −3.5 |
| | SEQ. ID. NO:563 | | | | | | |
| 570 | TCTTCAGGCTGCTGGGGGTA | −10.7 | −28.8 | 83.5 | −16.6 | −1.4 | −6.1 |
| | SEQ. ID. NO:564 | | | | | | |
| 812 | CAGCGTTTTTGGTAATGCTT | −10.7 | −23.1 | 67.4 | −10.9 | −1.4 | −5.5 |
| | SEQ. ID. NO:565 | | | | | | |
| 1111 | TGAATCCATAATAAAATGTA | −10.7 | −14.5 | 48 | −3.8 | 0 | −2.8 |
| | SEQ. ID. NO:566 | | | | | | |
| 1211 | TGGTTGCCATTTCCGTCAAA | −10.7 | −25.3 | 70.1 | −14.1 | −0.2 | −4.2 |
| | SEQ. ID. NO:567 | | | | | | |
| 1229 | CAAGAACCTGTACATGATTG | −10.7 | −19.5 | 58.5 | −8.8 | 0 | −6.1 |
| | SEQ. ID. NO:568 | | | | | | |
| 1264 | AGTCTGAAATCCTGGTAGCT | −10.7 | −23.8 | 70.2 | −13.1 | 0 | −4.6 |
| | SEQ. ID. NO:569 | | | | | | |
| 1311 | TCAACCGCAGACCCTTTCAG | −10.7 | −26.9 | 72.8 | −16.2 | 0 | −3.6 |
| | SEQ. ID. NO:570 | | | | | | |
| 1394 | TTCGAATTCTTTCTTCCAAT | −10.7 | −20.6 | 61.6 | −9.1 | −0.6 | −6.4 |
| | SEQ. ID. NO:571 | | | | | | |
| 1566 | AGTGGCTCCTGAAGCTTCTC | −10.7 | −26.8 | 78.6 | −14 | −2.1 | −10.8 |
| | SEQ. ID. NO:572 | | | | | | |
| 1616 | GAGGATTTTCAGGCTGGTGA | −10.7 | −24.7 | 73.2 | −14 | 0 | −3.9 |
| | SEQ. ID. NO:573 | | | | | | |
| 1666 | ATTGAATGTCCGTAATTCAG | −10.7 | −19.8 | 59.6 | −7.5 | −1.6 | −6.4 |
| | SEQ. ID. NO:574 | | | | | | |
| 1714 | CTTGTGGTCGTTTACTCTCC | −10.7 | −25.7 | 75.7 | −15 | 0 | −3.3 |
| | SEQ. ID. NO:575 | | | | | | |
| 1789 | AGAAAAAGGAGCTAGACCCC | −10.7 | −22.8 | 64 | −12.1 | 0 | −5.8 |
| | SEQ. ID. NO:576 | | | | | | |
| 1931 | AGAAAGTTGTTCTATCTAGC | −10.7 | −19.6 | 62 | −7.9 | −0.9 | −5.4 |
| | SEQ. ID. NO:577 | | | | | | |
| 307 | CTTTATGGTGGTCTTCAAAA | −10.6 | −20 | 61 | −9.4 | 0 | −2.9 |
| | SEQ. ID. NO:578 | | | | | | |
| 1071 | GTGAGTTCAGTTTTCTCCCT | −10.6 | −26.3 | 78.9 | −15.1 | −0.3 | −3.6 |
| | SEQ. ID. NO:579 | | | | | | |
| 1307 | CCGCAGACCCTTTCAGCAAA | −10.6 | −27.4 | 72.5 | −15.7 | −1 | −4.1 |
| | SEQ. ID. NO:580 | | | | | | |
| 1386 | CTTTCTTCCAATAGGTCAGA | −10.6 | −22.7 | 68.2 | −11.4 | −0.5 | −3.8 |
| | SEQ. ID. NO:581 | | | | | | |
| 1388 | TTCTTTCTTCCAATAGGTCA | −10.6 | −22.6 | 68.5 | −11.4 | −0.3 | −3.6 |
| | SEQ. ID. NO:582 | | | | | | |
| 1395 | TTTCGAATTCTTTCTTCCAA | −10.6 | −20.7 | 61.9 | −9.3 | −0.6 | −6.7 |
| | SEQ. ID. NO:583 | | | | | | |
| 1483 | AGCATACTCCTCTTGAGTCA | −10.6 | −24.9 | 74.2 | −12.8 | −1.4 | −7.5 |
| | SEQ. ID. NO:584 | | | | | | |
| 1727 | GAAGTGGGGTAAACTTGTGG | −10.6 | −21.8 | 64.5 | −10.2 | −0.9 | −4.1 |
| | SEQ. ID. NO:585 | | | | | | |
| 1802 | ATTAATATGAGAGAGAAAAA | −10.6 | −12.4 | 44.2 | −1.8 | 0 | −3.8 |
| | SEQ. ID. NO:586 | | | | | | |
| 1937 | ATGTAGAGAAAGTTGTTCTA | −10.6 | −18.3 | 58.6 | −6.2 | −1.4 | −4.6 |
| | SEQ. ID. NO:587 | | | | | | |
| 32 | ATTAGGATAAGTCGGGGAGA | −10.5 | −21.8 | 64.7 | −11.3 | 0.1 | −3 |
| | SEQ. ID. NO:588 | | | | | | |
| 101 | GATTCTGGACTGAGTCTTCC | −10.5 | −24.5 | 73.4 | −13 | −0.9 | −5.9 |
| | SEQ. ID. NO:589 | | | | | | |
| 568 | TTCAGGCTGCTGGGGGTAGA | −10.5 | −28.1 | 81.2 | −16.1 | −1.4 | −5.4 |
| | SEQ. ID. NO:590 | | | | | | |
| 811 | AGCGTTTTTGGTAATGCTTC | −10.5 | −22.8 | 67.8 | −10.9 | −1.3 | −5.3 |
| | SEQ. ID. NO:591 | | | | | | |
| 894 | CATTTCCTTAGTCGACACTC | −10.5 | −23.4 | 68.9 | −12 | 0 | −9.5 |
| | SEQ. ID. NO:592 | | | | | | |
| 924 | AAGCATTCAGCCAACATTCC | −10.5 | −24.2 | 68.5 | −12.7 | −0.9 | −4.1 |
| | SEQ. ID. NO:593 | | | | | | |
| 1210 | GGTTGCCATTTCCGTCAAAA | −10.5 | −24.6 | 68.1 | −14.1 | 0 | −3.1 |
| | SEQ. ID. NO:594 | | | | | | |
| 1313 | CTTCAACCGCAGACCCTTTC | −10.5 | −27.2 | 73.6 | −16.7 | 0 | −3.6 |
| | SEQ. ID. NO:595 | | | | | | |
| 1387 | TCTTTCTTCCAATAGGTCAG | −10.5 | −22.5 | 68.4 | −11.4 | −0.3 | −3.6 |
| | SEQ. ID. NO:596 | | | | | | |
| 1396 | ATTTCGAATTCTTTCTTCCA | −10.5 | −21.4 | 64 | −10.4 | −0.1 | −6.7 |
| | SEQ. ID. NO:597 | | | | | | |
| 1584 | TTTTGTAGCACATCAAGAAG | −10.5 | −18.9 | 58.7 | −8.4 | 0 | −5.1 |
| | SEQ. ID. NO:598 | | | | | | |
| 1603 | CTGGTGAATCTTACACAACT | −10.5 | −20.5 | 61.5 | −8.4 | −1.6 | −4.8 |
| | SEQ. ID. NO:599 | | | | | | |
| 1763 | GTAATCCCCATCACTGCACG | −10.5 | −27 | 72.7 | −16.5 | 0 | −4.8 |
| | SEQ. ID. NO:600 | | | | | | |
| 1985 | GGGCTTGCCAATTAGAATGC | −10.5 | −24.5 | 69.2 | −12.2 | −1.8 | −8.5 |
| | SEQ. ID. NO:601 | | | | | | |
| 2061 | GTAAGATGAGCAAAATGAGA | −10.5 | −17 | 53.5 | −6.5 | 0 | −4.1 |
| | SEQ. ID. NO:602 | | | | | | |
| 65 | ATTTTGCTACAAATGCTCAG | −10.4 | −20 | 60.6 | −8.8 | −0.6 | −5.2 |
| | SEQ. ID. NO:603 | | | | | | |
| 122 | GGACTTTCAAGGCCCTGGGA | −10.4 | −28.4 | 77.8 | −17.5 | 0 | −8.3 |
| | SEQ. ID. NO:604 | | | | | | |
| 673 | GCGGGGCTTCTTTGTTACAG | −10.4 | −26.4 | 75.7 | −16 | 0 | −3.4 |
| | SEQ. ID. NO:605 | | | | | | |
| 971 | TCACATTTTTTCTCAGTCGC | −10.4 | −23.1 | 69.7 | −12.7 | 0 | −2.7 |
| | SEQ. ID. NO:606 | | | | | | |
| 1118 | TGTTATATGAATCCATAATA | −10.4 | −16.4 | 52.6 | −5.3 | −0.5 | −3.6 |
| | SEQ. ID. NO:607 | | | | | | |
| 1481 | CATACTCCTCTTGAGTCATT | −10.4 | −23.2 | 69.7 | −11.1 | −1.7 | −5.8 |
| | SEQ. ID. NO:608 | | | | | | |
| 1540 | CTCTCTATCCTTTATGTATT | −10.4 | −21.4 | 66.1 | −11 | 0 | −1.2 |
| | SEQ. ID. NO:609 | | | | | | |
| 1901 | CAGTTGTGGAAGTTACACAT | −10.4 | −21.1 | 64 | −9 | −1.7 | −5.9 |
| | SEQ. ID. NO:610 | | | | | | |
| 1908 | ATATTTACAGTTGTGGAAGT | −10.4 | −19.3 | 60.6 | −8.9 | 0 | −3.4 |
| | SEQ. ID. NO:611 | | | | | | |
| 1963 | GATTCCCTGGAGCCTTTTAA | −10.4 | −25.8 | 72.3 | −15.4 | 0 | −4.5 |
| | SEQ. ID. NO:612 | | | | | | |
| 2060 | TAAGATGAGCAAAATGAGAT | −10.4 | −15.8 | 50.8 | −5.4 | 0 | −4.1 |
| | SEQ. ID. NO:613 | | | | | | |
| 741 | CCAGAGGCTCTGTCTCCACA | −10.3 | −29 | 82.1 | −17.1 | −1.5 | −8 |
| | SEQ. ID. NO:614 | | | | | | |
| 969 | ACATTTTTTCTCAGTCGCTT | −10.3 | −23 | 69.3 | −12.7 | 0 | −3.1 |
| | SEQ. ID. NO:615 | | | | | | |
| 998 | CATTCACGGTCTGATCTGCA | −10.3 | −25 | 72 | −14.7 | 0 | −4.9 |
| | SEQ. ID. NO:616 | | | | | | |
| 1029 | ACTTGTCGCAAGTCACGACC | −10.3 | −25.5 | 71 | −12.4 | −2.8 | −10.6 |
| | SEQ. ID. NO:617 | | | | | | |
| 1302 | GACCCTTTCAGCAAAGCAAT | −10.3 | −23.9 | 66.9 | −12.7 | −0.8 | −4.7 |
| | SEQ. ID. NO:618 | | | | | | |
| 1382 | CTTCCAATAGGTCAGAATGC | −10.3 | −22.3 | 65.8 | −11.4 | −0.3 | −3.6 |
| | SEQ. ID. NO:619 | | | | | | |
| 1533 | TCCTTTATGTATTGTCTATC | −10.3 | −20.8 | 65.3 | −10.5 | 0 | −0.9 |
| | SEQ. ID. NO:620 | | | | | | |
| 1805 | CAGATTAATATGAGAGAGAA | −10.3 | −15.8 | 51.6 | −5.5 | 0 | −5.4 |
| | SEQ. ID. NO:621 | | | | | | |
| 1893 | GAAGTTACACATGTAATTAC | −10.3 | −16.9 | 54.3 | −6 | −0.3 | −7.3 |
| | SEQ. ID. NO:622 | | | | | | |
| 1924 | TGTTCTATCTAGCCCAATAT | −10.3 | −22.8 | 67.2 | −12.5 | 0 | −3.7 |
| | SEQ. ID. NO:623 | | | | | | |
| 2043 | GATTTTCCCTAGTTCAACAG | −10.3 | −22.5 | 66.7 | −12.2 | 0 | −3.6 |
| | SEQ. ID. NO:624 | | | | | | |
| 149 | CTCCTTGGATTGTTTTGGGT | −10.2 | −25.3 | 74.1 | −15.1 | 0 | −4.6 |
| | SEQ. ID. NO:625 | | | | | | |
| 237 | TCCAGGAAACTAAGAGAAGC | −10.2 | −19.9 | 59.4 | −9.1 | −0.3 | −4.7 |
| | SEQ. ID. NO:626 | | | | | | |
| 365 | AATGTTCAATGAGATTCATT | −10.2 | −17.5 | 55.6 | −5.7 | −1.5 | −5.9 |
| | SEQ. ID. NO:627 | | | | | | |
| 567 | TCAGGCTGCTGGGGGTAGAA | −10.2 | −27.3 | 78.1 | −15.6 | −1.4 | −6.1 |
| | SEQ. ID. NO:628 | | | | | | |
| 793 | TCTCCTGAAGAAACCTTTAC | −10.2 | −20.9 | 61.7 | −10.7 | 0 | −2.8 |
| | SEQ. ID. NO:629 | | | | | | |
| 1003 | GTCTTCATTCACGGTCTGAT | −10.2 | −24.2 | 72.1 | −14 | 0 | −3.5 |
| | SEQ. ID. NO:630 | | | | | | |
| 1113 | TATGAATCCATAATAAAATG | −10.2 | −13.3 | 45.6 | −2.4 | −0.5 | −3.3 |
| | SEQ. ID. NO:631 | | | | | | |
| 1349 | TCTTATTGAAAATCTCAGCT | −10.2 | −18.8 | 58.5 | −8.1 | −0.1 | −4.3 |
| | SEQ. ID. NO:632 | | | | | | |
| 1474 | CTCTTGAGTCATTTTCAGTT | −10.2 | −21.9 | 68.6 | −11.7 | 0 | −5.8 |
| | SEQ. ID. NO:633 | | | | | | |
| 1475 | CCTCTTGAGTCATTTTCAGT | −10.2 | −23.8 | 72.3 | −13.1 | −0.2 | −5.5 |
| | SEQ. ID. NO:634 | | | | | | |
| 1951 | CCTTTTAAAACACAATGTAG | −10.2 | −16.8 | 52.7 | −6.1 | −0.2 | −6.2 |
| | SEQ. ID. NO:635 | | | | | | |
| 1972 | AGAATGCAGGATTCCCTGGA | −10.2 | −25.4 | 71.3 | −12.2 | −3 | −8.5 |
| | SEQ. ID. NO:636 | | | | | | |
| 600 | CTGAGTTCATATATTCCAGG | −10.1 | −21.7 | 65.7 | −11.6 | 0 | −3.6 |
| | SEQ. ID. NO:637 | | | | | | |
| 1259 | GAAATCCTGGTAGCTTTTTT | −10.1 | −21.8 | 65 | −11.7 | 0 | −4.7 |
| | SEQ. ID. NO:638 | | | | | | |
| 1262 | TCTGAAATCCTGGTAGCTTT | −10.1 | −22.8 | 67.3 | −12.7 | 0 | −4.7 |
| | SEQ. ID. NO:639 | | | | | | |
| 1278 | TCTTCATGGTCCAAAGTCTG | −10.1 | −23.1 | 68.8 | −13 | 0 | −4.7 |
| | SEQ. ID. NO:640 | | | | | | |
| 1617 | TGAGGATTTTCAGGCTGGTG | −10.1 | −24.1 | 71.6 | −14 | 0 | −3.8 |
| | SEQ. ID. NO:641 | | | | | | |
| 1661 | ATGTCCGTAATTCAGTCAGG | −10.1 | −23.3 | 68.8 | −13.2 | 0 | −3.3 |
| | SEQ. ID. NO:642 | | | | | | |
| 1773 | CCCCTCCCCTGTAATCCCCA | −10.1 | −35.5 | 86.8 | −25.4 | 0 | −1.5 |
| | SEQ. ID. NO:643 | | | | | | |
| 1932 | GAGAAAGTTGTTCTATCTAG | −10.1 | −18.4 | 59 | −6.8 | −1.4 | −5.9 |
| | SEQ. ID. NO:644 | | | | | | |
| 1933 | AGAGAAAGTTGTTCTATCTA | −10.1 | −18.4 | 59 | −6.8 | −1.4 | −5.5 |
| | SEQ. ID. NO:645 | | | | | | |
| 1989 | AACAGGGCTTGCCAATTAGA | −10.1 | −23.6 | 67.2 | −12.2 | −1.2 | −7.7 |
| | SEQ. ID. NO:646 | | | | | | |
| 2009 | ACAATCAATTTAATTAGGCA | −10.1 | −17.3 | 54.3 | −7.2 | 0 | −4.1 |
| | SEQ. ID. NO:647 | | | | | | |
| 2129 | CTGTTGCCATTATGTTTGCT | −10.1 | −24.6 | 71.8 | −14.5 | 0 | −3.6 |
| | SEQ. ID. NO:648 | | | | | | |
| 52 | TGCTCAGAATCCAATTTCGC | −10 | −23.3 | 66.6 | −12.6 | −0.4 | −4 |
| | SEQ. ID. NO:649 | | | | | | |
| 124 | ATGGACTTTCAAGGCCCTGG | −10 | −26.6 | 73.8 | −16.6 | 0 | −7.1 |
| | SEQ. ID. NO:650 | | | | | | |
| 205 | GAGCTATGTTTCTAAGTCTT | −10 | −21.3 | 66.6 | −11.3 | 0 | −5.1 |
| | SEQ. ID. NO:651 | | | | | | |
| 359 | CAATGAGATTCATTTTTGAT | −10 | −17.4 | 55.2 | −5.7 | −1.7 | −6.2 |
| | SEQ. ID. NO:652 | | | | | | |
| 447 | CCCAGAGGACCTGCCACTTG | −10 | −29.9 | 79.2 | −18.8 | −1 | −4.9 |
| | SEQ. ID. NO:653 | | | | | | |
| 579 | GAGTACCACTCTTCAGGCTG | −10 | −25.9 | 75.9 | −14.4 | −1.4 | −6.5 |
| | SEQ. ID. NO:654 | | | | | | |
| 711 | AGCTCATCCCCTTTGATCCT | −10 | −29.2 | 80.5 | −19.2 | 0 | −4.3 |
| | SEQ. ID. NO:655 | | | | | | |
| 794 | TTCTCCTGAAGAAACCTTTA | −10 | −20.8 | 61.5 | −9.9 | −0.8 | −3.6 |
| | SEQ. ID. NO:656 | | | | | | |
| 973 | CTTCACATTTTTTCTCAGTC | −10 | −21.5 | 67.5 | −11.5 | 0 | −2.5 |
| | SEQ. ID. NO:657 | | | | | | |
| 1260 | TGAAATCCTGGTAGCTTTTT | −10 | −21.7 | 64.6 | −11.7 | 0 | −4.7 |
| | SEQ. ID. NO:658 | | | | | | |
| 1285 | AATCTGGTCTTCATGGTCCA | −10 | −25 | 73.6 | −15 | 0 | −4.7 |
| | SEQ. ID. NO:659 | | | | | | |
| 1363 | CCCAGACGGAAGTTTCTTAT | −10 | −23.9 | 67.7 | −13.4 | −0.2 | −5.1 |
| | SEQ. ID. NO:660 | | | | | | |
| 1563 | GGCTCCTGAAGCTTCTCTAC | −10 | −26.4 | 76.8 | −14.3 | −2.1 | −10.8 |
| | SEQ. ID. NO:661 | | | | | | |
| 1681 | CTCAGCGTGGTGATGATTGA | −10 | −24.1 | 69.9 | −13.1 | −0.9 | −4.8 |
| | SEQ. ID. NO:662 | | | | | | |
| 1685 | GCATCTCAGCGTGGTGATGA | −10 | −26.3 | 75.5 | −14.9 | −1.3 | −6.7 |
| | SEQ. ID. NO:663 | | | | | | |
| 1788 | GAAAAAGGAGCTAGACCCCT | −10 | −23.7 | 65.5 | −13.7 | 0 | −5.8 |
| | SEQ. ID. NO:664 | | | | | | |
| 68 | GCGATTTTGCTACAAATGCT | −9.9 | −22.1 | 63.5 | −10.8 | −1.3 | −6.5 |
| | SEQ. ID. NO:665 | | | | | | |
| 129 | CAGAGATGGACTTTCAAGGC | −9.9 | −22.4 | 66.5 | −12 | −0.1 | −4.1 |
| | SEQ. ID. NO:666 | | | | | | |
| 206 | TGAGCTATGTTTCTAAGTCT | −9.9 | −21.2 | 66.1 | −11.3 | 0 | −5.1 |
| | SEQ. ID. NO:667 | | | | | | |
| 487 | ATTGCTGTATTGCGAGTATG | −9.9 | −21.9 | 65.3 | −11.1 | −0.7 | −4.1 |
| | SEQ. ID. NO:668 | | | | | | |
| 1218 | ACATGATTGGTTGCCATTTC | −9.9 | −23.2 | 68.2 | −12.6 | −0.4 | −5.9 |
| | SEQ. ID. NO:669 | | | | | | |
| 1263 | GTCTGAAATCCTGGTAGCTT | −9.9 | −23.9 | 70.3 | −14 | 0 | −4.7 |
| | SEQ. ID. NO:670 | | | | | | |
| 1274 | CATGGTCCAAAGTCTGAAAT | −9.9 | −20.5 | 60.6 | −10.6 | 0 | −3.9 |
| | SEQ. ID. NO:671 | | | | | | |
| 1310 | CAACCGCAGACCCTTTCAGC | −9.9 | −28.3 | 75.2 | −18.4 | 0 | −3.6 |
| | SEQ. ID. NO:672 | | | | | | |
| 1389 | ATTCTTTCTTCCAATAGGTC | −9.9 | −21.9 | 67.3 | −11.4 | −0.3 | −3.6 |
| | SEQ. ID. NO:673 | | | | | | |
| 1619 | GTTGAGGATTTTCAGGCTGG | −9.9 | −24.2 | 72.2 | −14.3 | 0 | −5.8 |
| | SEQ. ID. NO:674 | | | | | | |
| 1621 | GTGTTGAGGATTTTCAGGCT | −9.9 | −24.2 | 73 | −14.3 | 0 | −5.8 |
| | SEQ. ID. NO:675 | | | | | | |
| 1898 | TTGTGGAAGTTACACATGTA | −9.9 | −20.1 | 61.8 | −8.5 | −1.7 | −6.5 |
| | SEQ. ID. NO:676 | | | | | | |
| 111 | GCCCTGGGAGGATTCTGGAC | −9.8 | −28.9 | 80 | −18.3 | −0.6 | −8.3 |
| | SEQ. ID. NO:677 | | | | | | |
| 200 | ATGTTTCTAAGTCTTCTTTT | −9.8 | −19.9 | 63.7 | −9.5 | −0.3 | −2.7 |
| | SEQ. ID. NO:678 | | | | | | |
| 599 | TGAGTTCATATATTCCAGGA | −9.8 | −21.4 | 65.1 | −11.6 | 0 | −4.9 |
| | SEQ. ID. NO:679 | | | | | | |
| 813 | ACAGCGTTTTTGGTAATGCT | −9.8 | −23.2 | 67.7 | −12 | −1.3 | −5.3 |
| | SEQ. ID. NO:680 | | | | | | |
| 874 | TTGACACTTTCTTCGCATGT | −9.8 | −23.2 | 68.3 | −13.4 | 0 | −4.8 |
| | SEQ. ID. NO:681 | | | | | | |
| 1004 | TGTCTTCATTCACGGTCTGA | −9.8 | −24.2 | 71.9 | −14.4 | 0 | −3.5 |
| | SEQ. ID. NO:682 | | | | | | |
| 1031 | TCACTTGTCGCAAGTCACGA | −9.8 | −24.4 | 69.5 | −12.4 | −2.2 | −10.8 |
| | SEQ. ID. NO:683 | | | | | | |
| 1114 | ATATGAATCCATAATAAAAT | −9.8 | −13.3 | 45.6 | −2.4 | −1 | −3.8 |
| | SEQ. ID. NO:684 | | | | | | |
| 1271 | GGTCCAAAGTCTGAAATCCT | −9.8 | −23.1 | 66.4 | −13.3 | 0 | −3 |
| | SEQ. ID. NO:685 | | | | | | |
| 1348 | CTTATTGAAAATCTCAGCTG | −9.8 | −18.4 | 57.1 | −8.1 | 0 | −8 |
| | SEQ. ID. NO:686 | | | | | | |
| 1537 | TCTATCCTTTATGTATTGTC | −9.8 | −20.8 | 65.3 | −11 | 0 | −1.2 |
| | SEQ. ID. NO:687 | | | | | | |
| 1545 | ACTGCCTCTCTATCCTTTAT | −9.8 | −25.3 | 73.9 | −15.5 | 0 | −3 |
| | SEQ. ID. NO:688 | | | | | | |
| 1601 | GGTGAATCTTACACAACTTT | −9.8 | −19.8 | 60.3 | −8.4 | −1.6 | −4.8 |
| | SEQ. ID. NO:689 | | | | | | |
| 1807 | ATCAGATTAATATGAGAGAG | −9.8 | −16.3 | 53.3 | −6.5 | 0 | −7 |
| | SEQ. ID. NO:690 | | | | | | |
| 1897 | TGTGGAAGTTACACATGTAA | −9.8 | −19.3 | 59.4 | −7.9 | −1.5 | −6.9 |
| | SEQ. ID. NO:691 | | | | | | |
| 1930 | GAAAGTTGTTCTATCTAGCC | −9.8 | −21.6 | 65.8 | −11.3 | −0.1 | −3.9 |
| | SEQ. ID. NO:692 | | | | | | |
| 2059 | AAGATGAGCAAAATGAGATT | −9.8 | −16.2 | 51.6 | −6.4 | 0 | −4.1 |
| | SEQ. ID. NO:693 | | | | | | |
| 63 | TTTGCTACAAATGCTCAGAA | −9.7 | −19.8 | 59.6 | −9.4 | −0.4 | −5.2 |
| | SEQ. ID. NO:694 | | | | | | |
| 102 | GGATTCTGGACTGAGTCTTC | −9.7 | −23.7 | 72.3 | −13 | −0.9 | −5.9 |
| | SEQ. ID. NO:695 | | | | | | |
| 143 | GGATTGTTTTGGGTCAGAGA | −9.7 | −23.3 | 70.5 | −13.6 | 0 | −3.4 |
| | SEQ. ID. NO:696 | | | | | | |
| 163 | ACGATGTCTTCTACCTCCTT | −9.7 | −25.7 | 73.5 | −16 | 0 | −3.5 |
| | SEQ. ID. NO:697 | | | | | | |
| 228 | CTAAGAGAAGCAGTGTTCAC | −9.7 | −20.7 | 63.5 | −10.3 | −0.4 | −6.8 |
| | SEQ. ID. NO:698 | | | | | | |
| 319 | GAAATGCACTTTCTTTATGG | −9.7 | −19.4 | 59.3 | −8.7 | −0.9 | −8.4 |
| | SEQ. ID. NO:699 | | | | | | |
| 734 | CTCTGTCTCCACAAACAACA | −9.7 | −22.4 | 64.8 | −12.2 | −0.1 | −2.9 |
| | SEQ. ID. NO:700 | | | | | | |
| 902 | TCTCTTTGCATTTCCTTAGT | −9.7 | −23.8 | 72.2 | −14.1 | 0 | −5.1 |
| | SEQ. ID. NO:701 | | | | | | |
| 1125 | CTCTGTTTGTTATATGAATC | −9.7 | −18.6 | 59.3 | −8.9 | 0 | −2.4 |
| | SEQ. ID. NO:702 | | | | | | |
| 1155 | AAAATTTTATTTGTTATTTC | −9.7 | −13.7 | 47.7 | −3.5 | −0.2 | −6.3 |
| | SEQ. ID. NO:703 | | | | | | |
| 1256 | ATCCTGGTAGCTTTTTTGTG | −9.7 | −23.8 | 71.5 | −14.1 | 0 | −4.7 |
| | SEQ. ID. NO:704 | | | | | | |
| 1372 | GTCAGAATGCCCAGACGGAA | −9.7 | −25.4 | 69.4 | −15 | −0.4 | −4.8 |
| | SEQ. ID. NO:705 | | | | | | |
| 1432 | AAACATAGGTGTTATATATT | −9.7 | −16.1 | 52.6 | −4.7 | −1.7 | −7.4 |
| | SEQ. ID. NO:706 | | | | | | |
| 1602 | TGGTCAATCTTACACAACTT | −9.7 | −19.7 | 59.9 | −8.4 | −1.6 | −4.8 |
| | SEQ. ID. NO:707 | | | | | | |
| 1764 | TGTAATCCCCATCACTGCAC | −9.7 | −26.2 | 72.6 | −16.5 | 0 | −4.8 |
| | SEQ. ID. NO:708 | | | | | | |
| 168 | CTTCTACGATGTCTTCTACC | −9.6 | −23.4 | 69.2 | −13.8 | 0 | −3.5 |
| | SEQ. ID. NO:709 | | | | | | |
| 445 | CAGAGGACCTGCCACTTGTT | −9.6 | −27.2 | 76.2 | −16.5 | −1 | −4.9 |
| | SEQ. ID. NO:710 | | | | | | |
| 659 | TTACAGGCATCTCTGCTACC | −9.6 | −25.6 | 74.4 | −13.8 | −2.2 | −5.6 |
| | SEQ. ID. NO:711 | | | | | | |
| 1015 | ACGACCTTCACTGTCTTCAT | −9.6 | −24.7 | 71.3 | −14.4 | −0.5 | −3.7 |
| | SEQ. ID. NO:712 | | | | | | |
| 1030 | CACTTGTCGCAAGTCACGAC | −9.6 | −24.2 | 68.5 | −12.4 | −2.2 | −10.8 |
| | SEQ. ID. NO:713 | | | | | | |
| 1094 | GTAGAAGAGTCTGTTGATCT | −9.6 | −21.1 | 66.3 | −11 | −0.2 | −5.3 |
| | SEQ. ID. NO:714 | | | | | | |
| 1214 | GATTGGTTGCCATTTCCGTC | −9.6 | −26.7 | 75.3 | −16.4 | −0.4 | −4.6 |
| | SEQ. ID. NO:715 | | | | | | |
| 1380 | TCCAATAGGTCAGAATGCCC | −9.6 | −25.3 | 70.7 | −14.2 | −1.4 | −5 |
| | SEQ. ID. NO:716 | | | | | | |
| 1988 | ACAGGGCTTGCCAATTAGAA | −9.6 | −23.6 | 67.2 | −12.2 | −1.8 | −8.5 |
| | SEQ. ID. NO:717 | | | | | | |
| 2058 | AGATGAGCAAAATGAGATTT | −9.6 | −17 | 53.6 | −7.4 | 0 | −4.1 |
| | SEQ. ID. NO:718 | | | | | | |
| 2115 | TTTGCTTTATTGCCAAGATT | −9.6 | −21.4 | 63.6 | −11.8 | 0 | −3.6 |
| | SEQ. ID. NO:719 | | | | | | |
| 128 | AGAGATGGACTTTCAAGGCC | −9.5 | −23.7 | 69.1 | −14.2 | 0 | −6.4 |
| | SEQ. ID. NO:720 | | | | | | |
| 443 | GAGGACCTGCCACTTGTTCT | −9.5 | −27.8 | 78.5 | −17.2 | −1 | −4 |
| | SEQ. ID. NO:721 | | | | | | |
| 489 | ACATTGCTGTATTGCGAGTA | −9.5 | −22.8 | 67.2 | −13.3 | 0 | −4.1 |
| | SEQ. ID. NO:722 | | | | | | |
| 1258 | AAATCCTGGTAGCTTTTTTG | −9.5 | −21.2 | 63.6 | −11.7 | 0 | −4.7 |
| | SEQ. ID. NO:723 | | | | | | |
| 1279 | GTCTTCATGGTCCAAAGTCT | −9.5 | −24.3 | 72.4 | −14.8 | 0 | −4.2 |
| | SEQ. ID. NO:724 | | | | | | |
| 1284 | ATCTGGTCTTCATGGTCCAA | −9.5 | −25 | 73.6 | −15 | −0.2 | −4.7 |
| | SEQ. ID. NO:725 | | | | | | |
| 1546 | TACTGCCTCTCTATCCTTTA | −9.5 | −25 | 73.4 | −15.5 | 0 | −3 |
| | SEQ. ID. NO:726 | | | | | | |
| 1659 | GTCCGTAATTCAGTCAGGCG | −9.5 | −25.9 | 73.3 | −16.4 | 0 | −4 |
| | SEQ. ID. NO:727 | | | | | | |
| 1902 | ACAGTTGTGGAAGTTACACA | −9.5 | −21.3 | 64.6 | −10.5 | −1.2 | −6.1 |
| | SEQ. ID. NO:728 | | | | | | |
| 1907 | TATTTACAGTTGTGGAAGTT | −9.5 | −19.4 | 61 | −9.9 | 0 | −3.1 |
| | SEQ. ID. NO:729 | | | | | | |
| 1923 | GTTCTATCTAGCCCAATATT | −9.5 | −22.9 | 67.7 | −13.4 | 0 | −3.8 |
| | SEQ. ID. NO:730 | | | | | | |
| 1936 | TGTAGAGAAAGTTGTTCTAT | −9.5 | −18.3 | 58.6 | −7.9 | −0.8 | −4.4 |
| | SEQ. ID. NO:731 | | | | | | |
| 1246 | CTTTTTTGTGAATTCTACAA | −9.4 | −17.8 | 56.4 | −7.4 | −0.2 | −9.8 |
| | SEQ. ID. NO:732 | | | | | | |
| 1350 | TTCTTATTGAAAATCTCAGC | −9.4 | −18 | 56.8 | −8.1 | −0.1 | −3.1 |
| | SEQ. ID. NO:733 | | | | | | |
| 1594 | CTTACACAACTTTTGTAGCA | −9.4 | −20.6 | 62.4 | −10.3 | −0.7 | −5.8 |
| | SEQ. ID. NO:734 | | | | | | |
| 1598 | GAATCTTACACAACTTTTGT | −9.4 | −18.7 | 58.1 | −8.4 | −0.7 | −3.9 |
| | SEQ. ID. NO:735 | | | | | | |
| 1600 | GTGAATCTTACACAACTTTT | −9.4 | −18.7 | 58.1 | −8.4 | −0.8 | −4.3 |
| | SEQ. ID. NO:736 | | | | | | |
| 1914 | AGCCCAATATTTACAGTTGT | −9.4 | −22.8 | 66.8 | −13.4 | 0 | −3.9 |
| | SEQ. ID. NO:737 | | | | | | |
| 1987 | CAGGGCTTGCCAATTAGAAT | −9.4 | −23.4 | 66.6 | −12.2 | −1.8 | −8.5 |
| | SEQ. ID. NO:738 | | | | | | |
| 151 | ACCTCCTTGGATTGTTTTGG | −9.3 | −25.1 | 72.3 | −15.1 | −0.5 | −4.6 |
| | SEQ. ID. NO:739 | | | | | | |
| 166 | TCTACGATGTCTTCTACCTC | −9.3 | −23.7 | 70.4 | −14.4 | 0 | −3.5 |
| | SEQ. ID. NO:740 | | | | | | |
| 274 | GTCTGAAGTTTCATCTTGAG | −9.3 | −20.9 | 65.4 | −11.6 | 0 | −4.7 |
| | SEQ. ID. NO:741 | | | | | | |
| 275 | TGTCTGAAGTTTCATCTTGA | −9.3 | −20.9 | 65 | −11.6 | 0 | −4.7 |
| | SEQ. ID. NO:742 | | | | | | |
| 580 | AGAGTACCACTCTTCAGGCT | −9.3 | −25.9 | 76.4 | −14.4 | −2.2 | −8 |
| | SEQ. ID. NO:743 | | | | | | |
| 657 | ACAGGCATCTCTGCTACCTC | −9.3 | −27.1 | 78.5 | −15.6 | −2.2 | −5.6 |
| | SEQ. ID. NO:744 | | | | | | |
| 658 | TACAGGCATCTCTGCTACCT | −9.3 | −26.4 | 76.1 | −15.6 | −1.4 | −5.6 |
| | SEQ. ID. NO:745 | | | | | | |
| 834 | CCCCCGTTTTTACACTTGTA | −9.3 | −27.1 | 73.6 | −17.8 | 0.1 | −4.3 |
| | SEQ. ID. NO:746 | | | | | | |
| 1209 | GTTGCCATTTCCGTCAAAAT | −9.3 | −23.4 | 65.7 | −14.1 | 0 | −3 |
| | SEQ. ID. NO:747 | | | | | | |
| 1217 | CATGATTGGTTGCCATTTCC | −9.3 | −25 | 71.3 | −15 | −0.4 | −4.6 |
| | SEQ. ID. NO:748 | | | | | | |
| 1268 | CCAAAGTCTGAAATCCTGGT | −9.3 | −22.7 | 64.8 | −13.4 | 0 | −4.6 |
| | SEQ. ID. NO:749 | | | | | | |
| 1269 | TCCAAAGTCTGAAATCCTGG | −9.3 | −21.9 | 63.2 | −12.6 | 0 | −4 |
| | SEQ. ID. NO:750 | | | | | | |
| 1362 | CCAGACGGAAGTTTCTTATT | −9.3 | −22 | 64.5 | −11.8 | −0.8 | −5.1 |
| | SEQ. ID. NO:751 | | | | | | |
| 1393 | TCGAATTCTTTCTTCCAATA | −9.3 | −20.2 | 60.7 | −10.1 | −0.6 | −6.4 |
| | SEQ. ID. NO:752 | | | | | | |
| 1433 | TAAACATAGGTGTTATATAT | −9.3 | −15.7 | 51.7 | −4.7 | −1.7 | −7.2 |
| | SEQ. ID. NO:753 | | | | | | |
| 1772 | CCCTCCCCTGTAATCCCCAT | −9.3 | −33.5 | 83.7 | −24.2 | 0 | −1.6 |
| | SEQ. ID. NO:754 | | | | | | |
| 1851 | TCTTGAGTGAAACTGGGTAC | −9.3 | −21 | 63.7 | −11 | −0.5 | −5.2 |
| | SEQ. ID. NO:755 | | | | | | |
| 1863 | TTCATCAAGATTTCTTGAGT | −9.3 | −19.6 | 61.7 | −7.9 | −2.4 | −11.2 |
| | SEQ. ID. NO:756 | | | | | | |
| 1973 | TAGAATGCAGGATTCCCTGG | −9.3 | −24.5 | 69.5 | −12.2 | −3 | −8.5 |
| | SEQ. ID. NO:757 | | | | | | |
| 2019 | AATTGAAGTAACAATCAATT | −9.3 | −14.2 | 47.7 | −2.7 | −2.2 | −7.1 |
| | SEQ. ID. NO:758 | | | | | | |
| 2108 | TATTGCCAAGATTGAATACA | −9.3 | −18.8 | 57 | −9.5 | 0 | −3.7 |
| | SEQ. ID. NO:759 | | | | | | |
| 616 | CTCAGCTGGCATACGCCTGA | −9.2 | −28.4 | 77.6 | −16.3 | −2.9 | −9.9 |
| | SEQ. ID. NO:760 | | | | | | |
| 740 | CAGAGGCTCTGTCTCCACAA | −9.2 | −26.3 | 75.7 | −15.9 | −1.1 | −7.2 |
| | SEQ. ID. NO:761 | | | | | | |
| 1149 | TTATTTGTTATTTCCTGAGG | −9.2 | −20.3 | 62.8 | −11.1 | 0 | −3.5 |
| | SEQ. ID. NO:762 | | | | | | |
| 1637 | CCAGGAGACAGGCAAAGTGT | −9.2 | −24.7 | 70.4 | −15.5 | 0 | −4 |
| | SEQ. ID. NO:763 | | | | | | |
| 1840 | ACTGGGTACAAGTGAAATAA | −9.2 | −18 | 55.6 | −8.8 | 0 | −5 |
| | SEQ. ID. NO:764 | | | | | | |
| 2008 | CAATCAATTTAATTAGGCAA | −9.2 | −16.4 | 52.1 | −7.2 | 0 | −4.1 |
| | SEQ. ID. NO:765 | | | | | | |
| 669 | GGCTTCTTTGTTACAGGCAT | −9.1 | −25.1 | 74.3 | −15.3 | −0.4 | −4.2 |
| | SEQ. ID. NO:766 | | | | | | |
| 1032 | GTCACTTGTCGCAAGTCACG | −9.1 | −25 | 71.5 | −13.7 | −2.2 | −10.8 |
| | SEQ. ID. NO:767 | | | | | | |
| 1265 | AAGTCTGAAATCCTGGTAGC | −9.1 | −22.2 | 65.9 | −13.1 | 0 | −4.6 |
| | SEQ. ID. NO:768 | | | | | | |
| 1347 | TTATTGAAAATCTCAGCTGA | −9.1 | −18.1 | 56.5 | −8.1 | −0.1 | −9.8 |
| | SEQ. ID. NO:769 | | | | | | |
| 1596 | ATCTTACACAACTTTTGTAG | −9.1 | −18.5 | 58.4 | −8.4 | −0.9 | −4.3 |
| | SEQ. ID. NO:770 | | | | | | |
| 1599 | TGAATCTTACACAACTTTTG | −9.1 | −17.5 | 55.1 | −8.4 | 0 | −2.9 |
| | SEQ. ID. NO:771 | | | | | | |
| 1850 | CTTGAGTGAAACTGGGTACA | −9.1 | −21.3 | 63.4 | −11 | −1.1 | −6.3 |
| | SEQ. ID. NO:772 | | | | | | |
| 1853 | TTTCTTGAGTGAAACTGGGT | −9.1 | −21.3 | 64.4 | −11 | −1.1 | −5.1 |
| | SEQ. ID. NO:773 | | | | | | |
| 1962 | ATTCCCTGGAGCCTTTTAAA | −9.1 | −24.5 | 68.8 | −15.4 | 0 | −4.5 |
| | SEQ. ID. NO:774 | | | | | | |
| 2104 | GCCAAGATTGAATACAACTC | −9.1 | −19.8 | 59 | −9.8 | −0.8 | −3.7 |
| | SEQ. ID. NO:775 | | | | | | |
| 84 | TCCTCTCCAGATCCCAGCGA | −9 | −30.6 | 82 | −21.6 | 0 | −4.5 |
| | SEQ. ID. NO:776 | | | | | | |
| 132 | GGTCAGAGATGGACTTTCAA | −9 | −22.2 | 66.8 | −12 | −1.1 | −5 |
| | SEQ. ID. NO:777 | | | | | | |
| 201 | TATGTTTCTAAGTCTTCTTT | −9 | −19.5 | 62.7 | −9.9 | −0.3 | −2.7 |
| | SEQ. ID. NO:778 | | | | | | |
| 488 | CATTGCTGTATTGCGAGTAT | −9 | −22.6 | 66.6 | −12.7 | −0.7 | −4.1 |
| | SEQ. ID. NO:779 | | | | | | |
| 493 | CTGAACATTGCTGTATTGCG | −9 | −22.1 | 64 | −12.2 | −0.7 | −4.5 |
| | SEQ. ID. NO:780 | | | | | | |
| 1156 | TAAAATTTTATTTGTTATTT | −9 | −13 | 46.1 | −3.5 | −0.2 | −7.5 |
| | SEQ. ID. NO:781 | | | | | | |
| 1541 | CCTCTCTATCCTTTATGTAT | −9 | −23.3 | 69.7 | −14.3 | 0 | −1.2 |
| | SEQ. ID. NO:782 | | | | | | |
| 1622 | AGTGTTGAGGATTTTCAGGC | −9 | −23.3 | 71.2 | −14.3 | 0 | −5.6 |
| | SEQ. ID. NO:783 | | | | | | |
| 1715 | ACTTGTGGTCGTTTACTCTC | −9 | −23.9 | 72.5 | −14.9 | 0 | −3.3 |
| | SEQ. ID. NO:784 | | | | | | |
| 1803 | GATTAATATGAGAGAGAAAA | −9 | −13.7 | 46.9 | −4.7 | 0 | −4.7 |
| | SEQ. ID. NO:785 | | | | | | |
| 110 | CCCTGGGAGGATTCTGGACT | −8.9 | −28 | 77.6 | −18.3 | −0.6 | −7.2 |
| | SEQ. ID. NO:786 | | | | | | |
| 853 | CATATCCATCACACAGTTGC | −8.9 | −23.5 | 68.8 | −14.6 | 0 | −2.6 |
| | SEQ. ID. NO:787 | | | | | | |
| 1016 | CACGACCTTCACTGTCTTCA | −8.9 | −25.4 | 72.4 | −15.8 | −0.5 | −3.7 |
| | SEQ. ID. NO:788 | | | | | | |
| 1038 | GTCGAGGTCACTTGTCGCAA | −8.9 | −25.9 | 73.7 | −16.3 | −0.4 | −5.4 |
| | SEQ. ID. NO:789 | | | | | | |
| 1157 | TTAAAATTTTATTTGTTATT | −8.9 | −13 | 46.1 | −3.5 | −0.2 | −8 |
| | SEQ. ID. NO:790 | | | | | | |
| 1158 | TTTAAAATTTTATTTGTTAT | −8.9 | −13 | 46.1 | −3.5 | −0.2 | −8 |
| | SEQ. ID. NO:791 | | | | | | |
| 1270 | GTCCAAAGTCTGAAATCCTG | −8.9 | −21.9 | 63.8 | −13 | 0 | −3 |
| | SEQ. ID. NO:792 | | | | | | |
| 1308 | ACCGCAGACCCTTTCAGCAA | −8.9 | −28.3 | 75.2 | −18.3 | −1 | −4.1 |
| | SEQ. ID. NO:793 | | | | | | |
| 1476 | TCCTCTTGAGTCATTTTCAG | −8.9 | −23 | 70.4 | −13.6 | −0.2 | −5.8 |
| | SEQ. ID. NO:794 | | | | | | |
| 1539 | TCTCTATCCTTTATGTATTG | −8.9 | −20.5 | 63.9 | −11.6 | 0 | −1.2 |
| | SEQ. ID. NO:795 | | | | | | |
| 1757 | CCCATCACTGCACGTCCCAG | −8.9 | −30.7 | 80.1 | −21.3 | −0.1 | −7 |
| | SEQ. ID. NO:796 | | | | | | |
| 1804 | AGATTAATATGAGAGAGAAA | −8.9 | −14.4 | 48.6 | −5.5 | 0 | −4.7 |
| | SEQ. ID. NO:797 | | | | | | |
| 1976 | AATTAGAATGCAGGATTCCC | −8.9 | −21.8 | 63.4 | −12.2 | −0.5 | −5.8 |
| | SEQ. ID. NO:798 | | | | | | |
| 94 | GACTGAGTCTTCCTCTCCAG | −8.8 | −26.6 | 78.3 | −16.5 | −1.2 | −5.3 |
| | SEQ. ID. NO:799 | | | | | | |
| 366 | GAATGTTCAATGAGATTCAT | −8.8 | −18 | 56.6 | −8.3 | −0.8 | −7 |
| | SEQ. ID. NO:800 | | | | | | |
| 619 | AGTCTCAGCTGGCATACGCC | −8.8 | −28.5 | 80.1 | −17.6 | −2.1 | −9.3 |
| | SEQ. ID. NO:801 | | | | | | |
| 652 | CATCTCTGCTACCTCAGTTT | −8.8 | −25.3 | 75 | −16.5 | 0.4 | −3.6 |
| | SEQ. ID. NO:802 | | | | | | |
| 1283 | TCTGGTCTTCATGGTCCAAA | −8.8 | −24.3 | 71.1 | −15 | −0.2 | −4.7 |
| | SEQ. ID. NO:803 | | | | | | |
| 1309 | AACCGCAGACCCTTTCAGCA | −8.8 | −28.3 | 75.2 | −18.4 | −1 | −4.1 |
| | SEQ. ID. NO:804 | | | | | | |
| 1383 | TCTTCCAATAGGTCAGAATG | −8.8 | −20.9 | 63.1 | −11.4 | −0.4 | −3.7 |
| | SEQ. ID. NO:805 | | | | | | |
| 1549 | CTCTACTGCCTCTCTATCCT | −8.8 | −27.3 | 79.1 | −18.5 | 0 | −3 |
| | SEQ. ID. NO:806 | | | | | | |
| 1956 | TGGAGCCTTTTAAAACACAA | −8.8 | −19.5 | 57.7 | −10.7 | 0 | −6.2 |
| | SEQ. ID. NO:807 | | | | | | |
| 1959 | CCCTGGAGCCTTTTAAAACA | −8.8 | −24.2 | 66.6 | −15.4 | 0 | −6.2 |
| | SEQ. ID. NO:808 | | | | | | |
| 2049 | AAATGAGATTTTCCCTAGTT | −8.8 | −20.4 | 61.3 | −11.6 | 0 | −3.8 |
| | SEQ. ID. NO:809 | | | | | | |
| 150 | CCTCCTTGGATTGTTTTGGG | −8.7 | −26.1 | 74.3 | −17.4 | 0 | −4.6 |
| | SEQ. ID. NO:810 | | | | | | |
| 171 | CTCCTTCTACGATGTCTTCT | −8.7 | −24.8 | 72.8 | −16.1 | 0 | −3.5 |
| | SEQ. ID. NO:811 | | | | | | |
| 436 | TGCCACTTGTTCTGTTAAAA | −8.7 | −21.2 | 62.8 | −12.5 | 0 | −3 |
| | SEQ. ID. NO:812 | | | | | | |
| 645 | GCTACCTCAGTTTCTCCCTG | −8.7 | −28.6 | 81.3 | −19.9 | 0 | −3.2 |
| | SEQ. ID. NO:813 | | | | | | |
| 646 | TGCTACCTCAGTTTCTCCCT | −8.7 | −28.6 | 81.3 | −19.9 | 0 | −3.6 |
| | SEQ. ID. NO:814 | | | | | | |
| 647 | CTGCTACCTCAGTTTCTCCC | −8.7 | −28.6 | 81.3 | −19.9 | 0 | −3.6 |
| | SEQ. ID. NO:815 | | | | | | |
| 743 | ATCCAGAGGCTCTGTCTCCA | −8.7 | −28.5 | 82.2 | −18.2 | −1.5 | −8 |
| | SEQ. ID. NO:816 | | | | | | |
| 795 | CTTCTCCTGAAGAAACCTTT | −8.7 | −22 | 63.9 | −11.7 | −1.5 | −5.3 |
| | SEQ. ID. NO:817 | | | | | | |
| 803 | TGGTAATGCTTCTCCTGAAG | −8.7 | −22.7 | 66.8 | −12.2 | −1.8 | −6.1 |
| | SEQ. ID. NO:818 | | | | | | |
| 996 | TTCACGGTCTGATCTGCATG | −8.7 | −24.3 | 70.7 | −15.6 | 0 | −4.9 |
| | SEQ. ID. NO:819 | | | | | | |
| 1106 | CCATAATAAAATGTAGAAGA | −8.7 | −14.7 | 48.4 | −6 | 0 | −2.8 |
| | SEQ. ID. NO:820 | | | | | | |
| 1230 | ACAAGAACCTGTACATGATT | −8.7 | −19.7 | 59.1 | −11 | 0 | −6.1 |
| | SEQ. ID. NO:821 | | | | | | |
| 1272 | TGGTCCAAAGTCTGAAATCC | −8.7 | −22.2 | 64.4 | −13.5 | 0 | −3.5 |
| | SEQ. ID. NO:822 | | | | | | |
| 1280 | GGTCTTCATGGTCCAAAGTC | −8.7 | −24.6 | 73.1 | −15.9 | 0 | −4.7 |
| | SEQ. ID. NO:823 | | | | | | |
| 1538 | CTCTATCCTTTATGTATTGT | −8.7 | −21.3 | 65.7 | −12.6 | 0 | −1.2 |
| | SEQ. ID. NO:824 | | | | | | |
| 1562 | GCTCCTGAAGCTTCTCTACT | −8.7 | −26.1 | 76.2 | −15.8 | −1.3 | −10.8 |
| | SEQ. ID. NO:825 | | | | | | |
| 1620 | TGTTGAGGATTTTCAGGCTG | −8.7 | −23 | 69.3 | −14.3 | 0 | −5.8 |
| | SEQ. ID. NO:826 | | | | | | |
| 1676 | CGTGGTGATGATTGAATGTC | −8.7 | −21.2 | 63.2 | −12.5 | 0 | −2.8 |
| | SEQ. ID. NO:827 | | | | | | |
| 1758 | CCCCATCACTGCACGTCCCA | −8.7 | −32.7 | 83 | −24 | 0 | −4.8 |
| | SEQ. ID. NO:828 | | | | | | |
| 1762 | TAATCCCCATCACTGCACGT | −8.7 | −27 | 72.7 | −18.3 | 0 | −4.8 |
| | SEQ. ID. NO:829 | | | | | | |
| 1852 | TTCTTGAGTGAAACTGGGTA | −8.7 | −20.9 | 63.5 | −11 | −1.1 | −4.4 |
| | SEQ. ID. NO:830 | | | | | | |
| 1957 | CTGGAGCCTTTTAAAACACA | −8.7 | −21.1 | 61.3 | −12.4 | 0 | −6.2 |
| | SEQ. ID. NO:831 | | | | | | |
| 2010 | AACAATCAATTTAATTAGGC | −8.7 | −15.9 | 51.3 | −7.2 | 0 | −4.1 |
| | SEQ. ID. NO:832 | | | | | | |
| 83 | CCTCTCCAGATCCCAGCGAT | −8.6 | −30.2 | 80.2 | −21.6 | 0 | −4.5 |
| | SEQ. ID. NO:833 | | | | | | |
| 86 | CTTCCTCTCCAGATCCCAGC | −8.6 | −30.2 | 83.6 | −21.6 | 0 | −4.5 |
| | SEQ. ID. NO:834 | | | | | | |
| 103 | AGGATTCTGGACTGAGTCTT | −8.6 | −23.3 | 70.8 | −13.7 | −0.9 | −5.9 |
| | SEQ. ID. NO:835 | | | | | | |
| 139 | TGTTTTGGGTCAGAGATGGA | −8.6 | −23.2 | 70 | −13.7 | −0.7 | −3.6 |
| | SEQ. ID. NO:836 | | | | | | |
| 444 | AGAGGACCTGCCACTTGTTC | −8.6 | −26.9 | 76.8 | −17.2 | −1 | −3.9 |
| | SEQ. ID. NO:837 | | | | | | |
| 569 | CTTCAGGCTGCTGGGGGTAG | −8.6 | −28.4 | 81.9 | −18.3 | −1.4 | −6.1 |
| | SEQ. ID. NO:838 | | | | | | |
| 742 | TCCAGAGGCTCTGTCTCCAC | −8.6 | −28.7 | 83 | −18.5 | −1.5 | −8 |
| | SEQ. ID. NO:839 | | | | | | |
| 921 | CATTCAGCCAACATTCCCAT | −8.6 | −25.8 | 71 | −17.2 | 0 | −3.2 |
| | SEQ. ID. NO:840 | | | | | | |
| 1273 | ATGGTCCAAAGTCTGAAATC | −8.6 | −20.2 | 60.7 | −11.6 | 0 | −3.9 |
| | SEQ. ID. NO:841 | | | | | | |
| 1290 | AAAGCAATCTGGTCTTCATG | −8.6 | −20.6 | 62.2 | −12 | 0 | −4.1 |
| | SEQ. ID. NO:842 | | | | | | |
| 1296 | TTCAGCAAAGCAATCTGGTC | −8.6 | −22.2 | 66 | −12.7 | −0.7 | −4.4 |
| | SEQ. ID. NO:843 | | | | | | |
| 1424 | GTGTTATATATTCATCAGAG | −8.6 | −18.5 | 59.6 | −9.9 | 0 | −4 |
| | SEQ. ID. NO:844 | | | | | | |
| 1544 | CTGCCTCTCTATCCTTTATG | −8.6 | −25.1 | 73.1 | −16.5 | 0 | −3 |
| | SEQ. ID. NO:845 | | | | | | |
| 1618 | TTGAGGATTTTCAGGCTGGT | −8.6 | −24.2 | 72.2 | −15.6 | 0 | −5.8 |
| | SEQ. ID. NO:846 | | | | | | |
| 1677 | GCGTGGTGATGATTGAATGT | −8.6 | −22.6 | 65.8 | −14 | 0 | −3.5 |
| | SEQ. ID. NO:847 | | | | | | |
| 1844 | TGAAACTGGGTACAAGTGAA | −8.6 | −18.9 | 57.3 | −10.3 | 0 | −6 |
| | SEQ. ID. NO:848 | | | | | | |
| 1858 | CAAGATTTCTTGAGTGAAAC | −8.6 | −17.4 | 55.1 | −7.9 | −0.8 | −8.1 |
| | SEQ. ID. NO:849 | | | | | | |
| 1974 | TTAGAATGCAGGATTCCCTG | −8.6 | −23.4 | 67.3 | −12.2 | −2.6 | −7.2 |
| | SEQ. ID. NO:850 | | | | | | |
| 2100 | AGATTGAATACAACTCTTTA | −8.6 | −16.8 | 54 | −7.1 | −1 | −3.6 |
| | SEQ. ID. NO:851 | | | | | | |
| 62 | TTGCTACAAATGCTCAGAAT | −8.5 | −19.7 | 59.2 | −10.5 | −0.4 | −3.6 |
| | SEQ. ID. NO:852 | | | | | | |
| 85 | TTCCTCTCCAGATCCCAGCG | −8.5 | −30.1 | 81.1 | −21.6 | 0 | −4.5 |
| | SEQ. ID. NO:853 | | | | | | |
| 148 | TCCTTGGATTGTTTTGGGTC | −8.5 | −24.8 | 73.8 | −16.3 | 0 | −4.3 |
| | SEQ. ID. NO:854 | | | | | | |
| 165 | CTACGATGTCTTCTACCTCC | −8.5 | −25.3 | 72.5 | −16.8 | 0 | −3.5 |
| | SEQ. ID. NO:855 | | | | | | |
| 175 | TTCACTCCTTCTACGATGTC | −8.5 | −23.9 | 70.6 | −15.4 | 0 | −3.5 |
| | SEQ. ID. NO:856 | | | | | | |
| 176 | TTTCACTCCTTCTACGATGT | −8.5 | −23.6 | 69.3 | −15.1 | 0 | −3.5 |
| | SEQ. ID. NO:857 | | | | | | |
| 351 | TTCATTTTTGATCCCATCCA | −8.5 | −24.4 | 69.8 | −15 | −0.8 | −4.3 |
| | SEQ. ID. NO:858 | | | | | | |
| 484 | GCTGTATTGCGAGTATGGTT | −8.5 | −24.3 | 71.5 | −15.8 | 0 | −4.1 |
| | SEQ. ID. NO:859 | | | | | | |
| 581 | GAGAGTACCACTCTTCAGGC | −8.5 | −25.6 | 75.7 | −14.4 | −2.7 | −8.6 |
| | SEQ. ID. NO:860 | | | | | | |
| 1009 | TTCACTGTCTTCATTCACGG | −8.5 | −23.4 | 69.4 | −14.9 | 0 | −3.5 |
| | SEQ. ID. NO:861 | | | | | | |
| 1564 | TGGCTCCTGAAGCTTCTCTA | −8.5 | −26.2 | 76 | −15.6 | −2.1 | −10.8 |
| | SEQ. ID. NO:862 | | | | | | |
| 1615 | AGGATTTTCAGGCTGGTGAA | −8.5 | −23.4 | 69.3 | −14.3 | −0.3 | −5.4 |
| | SEQ. ID. NO:863 | | | | | | |
| 1753 | TCACTGCACGTCCCAGATTT | −8.5 | −26.8 | 74.4 | −17.6 | −0.5 | −7.5 |
| | SEQ. ID. NO:864 | | | | | | |
| 1890 | GTTACACATGTAATTACAAC | −8.5 | −17.2 | 54.6 | −7.5 | −0.3 | −10.3 |
| | SEQ. ID. NO:865 | | | | | | |
| 1960 | TCCCTGGAGCCTTTTAAAAC | −8.5 | −23.9 | 66.9 | −15.4 | 0 | −6.2 |
| | SEQ. ID. NO:866 | | | | | | |
| 60 | GCTACAAATGCTCAGAATCC | −8.4 | −22 | 64 | −13.6 | 0 | −3.6 |
| | SEQ. ID. NO:867 | | | | | | |
| 302 | TGGTGGTCTTCAAAAAAAAC | −8.4 | −16.6 | 52.3 | −8.2 | 0 | −2.9 |
| | SEQ. ID. NO:868 | | | | | | |
| 643 | TACCTCAGTTTCTCCCTGGT | −8.4 | −28.3 | 81.1 | −19.9 | 0.3 | −4.8 |
| | SEQ. ID. NO:869 | | | | | | |
| 1006 | ACTGTCTTCATTCACGGTCT | −8.4 | −24.7 | 73.4 | −16.3 | 0 | −3.5 |
| | SEQ. ID. NO:870 | | | | | | |
| 1008 | TCACTGTCTTCATTCACGGT | −8.4 | −24.5 | 72.5 | −16.1 | 0 | −3.5 |
| | SEQ. ID. NO:871 | | | | | | |
| 1080 | TGATCTGGGGTGAGTTCAGT | −8.4 | −24.9 | 75.3 | −16 | −0.2 | −4.9 |
| | SEQ. ID. NO:872 | | | | | | |
| 1314 | GCTTCAACCGCAGACCCTTT | −8.4 | −28.6 | 76.1 | −20.2 | 0 | −3.6 |
| | SEQ. ID. NO:873 | | | | | | |
| 1547 | CTACTGCCTCTCTATCCTTT | −8.4 | −26.2 | 76 | −17.8 | 0 | −2.3 |
| | SEQ. ID. NO:874 | | | | | | |
| 1597 | AATCTTACACAACTTTTGTA | −8.4 | −17.8 | 56.2 | −8.4 | −0.9 | −4.3 |
| | SEQ. ID. NO:875 | | | | | | |
| 1692 | GACATCAGCATCTCAGCGTG | −8.4 | −25.3 | 73.2 | −15.9 | −0.9 | −4.1 |
| | SEQ. ID. NO:876 | | | | | | |
| 1713 | TTGTGGTCGTTTACTCTCCA | −8.4 | −25.5 | 74.8 | −16.6 | −0.2 | −3.7 |
| | SEQ. ID. NO:877 | | | | | | |
| 1817 | AAAGTTATACATCAGATTAA | −8.4 | −15 | 50 | −6.6 | 0 | −3.4 |
| | SEQ. ID. NO:878 | | | | | | |
| 1842 | AAACTGGGTACAAGTGAAAT | −8.4 | −17.6 | 54.4 | −9.2 | 0 | −6 |
| | SEQ. ID. NO:879 | | | | | | |
| 1961 | TTCCCTGGAGCCTTTTAAAA | −8.4 | −23.8 | 66.7 | −15.4 | 0 | −6 |
| | SEQ. ID. NO:880 | | | | | | |
| 2048 | AATGAGATTTTCCCTAGTTC | −8.4 | −21.5 | 64.9 | −13.1 | 0 | −3.8 |
| | SEQ. ID. NO:881 | | | | | | |
| 91 | TGAGTCTTCCTCTCCAGATC | −8.3 | −25.9 | 77.4 | −16.3 | −1.2 | −5.9 |
| | SEQ. ID. NO:882 | | | | | | |
| 120 | ACTTTCAAGGCCCTGGGAGG | −8.3 | −27.8 | 76.8 | −18.9 | −0.2 | −8.3 |
| | SEQ. ID. NO:883 | | | | | | |
| 174 | TCACTCCTTCTACGATGTCT | −8.3 | −24.7 | 72.2 | −16.4 | 0 | −3.5 |
| | SEQ. ID. NO:884 | | | | | | |
| 481 | GTATTGCGAGTATGGTTCCA | −8.3 | −24.7 | 71.8 | −16.4 | 0 | −5.3 |
| | SEQ. ID. NO:885 | | | | | | |
| 495 | AACTGAACATTGCTGTATTG | −8.3 | −19 | 58.2 | −10 | −0.5 | −3.9 |
| | SEQ. ID. NO:886 | | | | | | |
| 1117 | GTTATATGAATCCATAATAA | −8.3 | −15.7 | 51 | −6.3 | −1 | −4.2 |
| | SEQ. ID. NO:887 | | | | | | |
| 1337 | TCTCAGCTGAACGAAGGAAC | −8.3 | −21.2 | 62 | −11.8 | 0 | −10.1 |
| | SEQ. ID. NO:888 | | | | | | |
| 1529 | TTATGTATTGTCTATCTGGA | −8.3 | −20.1 | 63.3 | −11.8 | 0 | −2.7 |
| | SEQ. ID. NO:889 | | | | | | |
| 1552 | CTTCTCTACTGCCTCTCTAT | −8.3 | −25.4 | 75.7 | −17.1 | 0 | −3 |
| | SEQ. ID. NO:890 | | | | | | |
| 1587 | AACTTTTGTAGCACATCAAG | −8.3 | −19.4 | 59.7 | −10.3 | −0.6 | −6.4 |
| | SEQ. ID. NO:891 | | | | | | |
| 1645 | CAGGCGACCCAGGAGACAGG | −8.3 | −28.5 | 76.4 | −19.2 | −0.9 | −5.4 |
| | SEQ. ID. NO:892 | | | | | | |
| 1662 | AATGTCCGTAATTCAGTCAG | −8.3 | −21.4 | 63.9 | −13.1 | 0 | −3 |
| | SEQ. ID. NO:893 | | | | | | |
| 1846 | AGTGAAACTGGGTACAAGTG | −8.3 | −20.2 | 61.1 | −11.1 | −0.6 | −6.6 |
| | SEQ. ID. NO:894 | | | | | | |
| 1990 | AAACAGGGCTTGCCAATTAG | −8.3 | −22.3 | 63.9 | −12.2 | −1.8 | −8.5 |
| | SEQ. ID. NO:895 | | | | | | |
| 2063 | TGGTAAGATGAGCAAAATGA | −8.3 | −17.6 | 54.5 | −9.3 | 0 | −4.1 |
| | SEQ. ID. NO:896 | | | | | | |
| 97 | CTGGACTGAGTCTTCCTCTC | −8.2 | −26 | 77.7 | −16.5 | −1.2 | −6.9 |
| | SEQ. ID. NO:897 | | | | | | |
| 169 | CCTTCTACGATGTCTTCTAC | −8.2 | −23.4 | 69.2 | −15.2 | 0 | −3.5 |
| | SEQ. ID. NO:898 | | | | | | |
| 303 | ATGGTGGTCTTCAAAAAAAA | −8.2 | −16.4 | 51.8 | −8.2 | 0 | −3.3 |
| | SEQ. ID. NO:899 | | | | | | |
| 653 | GCATCTCTGCTACCTCAGTT | −8.2 | −27 | 79.2 | −17 | −1.8 | −5.6 |
| | SEQ. ID. NO:900 | | | | | | |
| 865 | TCTTCGCATGTACATATCCA | −8.2 | −23.7 | 68.7 | −15 | 0 | −8 |
| | SEQ. ID. NO:901 | | | | | | |
| 1010 | CTTCACTGTCTTCATTCACG | −8.2 | −23.1 | 68.8 | −14.9 | 0 | −3 |
| | SEQ. ID. NO:902 | | | | | | |
| 1257 | AATCCTGGTAGCTTTTTTGT | −8.2 | −23.1 | 69.2 | −14.9 | 0 | −4.7 |
| | SEQ. ID. NO:903 | | | | | | |
| 1343 | TGAAAATCTCAGCTGAACGA | −8.2 | −19.1 | 57 | −9.8 | 0 | −10.1 |
| | SEQ. ID. NO:904 | | | | | | |
| 1754 | ATCACTGCACGTCCCAGATT | −8.2 | −26.7 | 74 | −17.8 | −0.5 | −7.5 |
| | SEQ. ID. NO:905 | | | | | | |
| 1966 | CAGGATTCCCTGGAGCCTTT | −8.2 | −28.6 | 78.8 | −18.1 | −2.3 | −7.8 |
| | SEQ. ID. NO:906 | | | | | | |
| 1975 | ATTAGAATGCAGGATTCCCT | −8.2 | −23.4 | 67.4 | −13.8 | −1.3 | −6 |
| | SEQ. ID. NO:907 | | | | | | |
| 130 | TCAGAGATGGACTTTCAAGG | −8.1 | −21 | 63.8 | −12 | −0.7 | −4.8 |
| | SEQ. ID. NO:908 | | | | | | |
| 131 | GTCAGAGATGGACTTTCAAG | −8.1 | −21 | 64.4 | −12 | −0.7 | −4.4 |
| | SEQ. ID. NO:909 | | | | | | |
| 566 | CAGGCTGCTGGGGGTAGAAA | −8.1 | −26.2 | 73.8 | −17.2 | −0.8 | −6.1 |
| | SEQ. ID. NO:910 | | | | | | |
| 615 | TCAGCTGGCATACGCCTGAG | −8.1 | −27.5 | 76 | −16.5 | −2.9 | −9.9 |
| | SEQ. ID. NO:911 | | | | | | |
| 617 | TCTCAGCTGGCATACGCCTG | −8.1 | −28.2 | 78 | −17.2 | −2.9 | −9.8 |
| | SEQ. ID. NO:912 | | | | | | |
| 707 | CATCCCCTTTGATCCTCCCT | −8.1 | −31.4 | 82.6 | −23.3 | 0 | −4.3 |
| | SEQ. ID. NO:913 | | | | | | |
| 712 | CAGCTCATCCCCTTTGATCC | −8.1 | −29 | 79.6 | −20.9 | 0 | −4.4 |
| | SEQ. ID. NO:914 | | | | | | |
| 751 | ATAGTGGTATCCAGAGGCTC | −8.1 | −25 | 74.9 | −16.1 | −0.6 | −4.6 |
| | SEQ. ID. NO:915 | | | | | | |
| 814 | CACAGCGTTTTTGGTAATGC | −8.1 | −23 | 66.9 | −14.2 | −0.5 | −4.1 |
| | SEQ. ID. NO:916 | | | | | | |
| 1013 | GACCTTCACTGTCTTCATTC | −8.1 | −24.2 | 72.8 | −16.1 | 0 | −3.6 |
| | SEQ. ID. NO:917 | | | | | | |
| 1159 | TTTTAAAATTTTATTTGTTA | −8.1 | −13.1 | 46.3 | −5 | 0.3 | −8 |
| | SEQ. ID. NO:918 | | | | | | |
| 1384 | TTCTTCCAATAGGTCAGAAT | −8.1 | −21 | 63.5 | −11.4 | −1.4 | −4.7 |
| | SEQ. ID. NO:919 | | | | | | |
| 1385 | TTTCTTCCAATAGGTCAGAA | −8.1 | −21.1 | 63.9 | −11.4 | −1.5 | −4.8 |
| | SEQ. ID. NO:920 | | | | | | |
| 1765 | CTGTAATCCCCATCACTGCA | −8.1 | −26.9 | 73.9 | −18.8 | 0 | −4.7 |
| | SEQ. ID. NO:921 | | | | | | |
| 1777 | TAGACCCCTCCCCTGTAATC | −8.1 | −29.3 | 78.1 | −21.2 | 0 | −2 |
| | SEQ. ID. NO:922 | | | | | | |
| 1845 | GTGAAACTGGGTACAAGTGA | −8.1 | −20.8 | 62.2 | −12.7 | 0 | −6 |
| | SEQ. ID. NO:923 | | | | | | |
| 1892 | AAGTTACACATGTAATTACA | −8.1 | −17 | 54.3 | −7.9 | −0.3 | −9.9 |
| | SEQ. ID. NO:924 | | | | | | |
| 1997 | ATTAGGCAAACAGGGCTTGC | −8.1 | −24 | 68.9 | −15 | −0.8 | −7.2 |
| | SEQ. ID. NO:925 | | | | | | |
| 2012 | GTAACAATCAATTTAATTAG | −8.1 | −13.8 | 47.3 | −5.7 | 0 | −4.1 |
| | SEQ. ID. NO:926 | | | | | | |
| 2099 | GATTGAATACAACTGTTTAA | −8.1 | −16.1 | 52.1 | −7.1 | −0.8 | −3.7 |
| | SEQ. ID. NO:927 | | | | | | |
| 2107 | ATTGCCAAGATTGAATACAA | −8.1 | −18.4 | 55.7 | −9.5 | −0.6 | −4.2 |
| | SEQ. ID. NO:928 | | | | | | |
| 236 | CCAGGAAACTAAGAGAAGCA | −8 | −20.2 | 59.3 | −11.6 | −0.3 | −4.7 |
| | SEQ. ID. NO:929 | | | | | | |
| 911 | ACATTCCCATCTCTTTGCAT | −8 | −25.4 | 72.9 | −17.4 | 0 | −5.1 |
| | SEQ. ID. NO:930 | | | | | | |
| 933 | TCAGTTAACAAGCATTCAGC | −8 | −21.1 | 64 | −12.4 | −0.5 | −8.3 |
| | SEQ. ID. NO:931 | | | | | | |
| 961 | TCTCAGTCGCTTAGATTTAC | −8 | −22 | 67.3 | −14 | 0 | −3.1 |
| | SEQ. ID. NO:932 | | | | | | |
| 1095 | TGTAGAAGAGTCTGTTGATC | −8 | −20.2 | 64 | −11.7 | −0.2 | −5.8 |
| | SEQ. ID. NO:933 | | | | | | |
| 1345 | ATTGAAAATCTCAGCTGAAC | −8 | −17.8 | 55.4 | −8.1 | −0.1 | −11.6 |
| | SEQ. ID. NO:934 | | | | | | |
| 1766 | CCTGTAATCCCCATCACTGC | −8 | −28.2 | 76.3 | −20.2 | 0 | −2.6 |
| | SEQ. ID. NO:935 | | | | | | |
| 1860 | ATCAAGATTTCTTGAGTGAA | −8 | −18.3 | 57.8 | −7.9 | −2.4 | −11.2 |
| | SEQ. ID. NO:936 | | | | | | |
| 1903 | TACAGTTGTGGAAGTTACAC | −8 | −20.3 | 62.8 | −11.6 | −0.4 | −4.2 |
| | SEQ. ID. NO:937 | | | | | | |
| 277 | AGTGTCTGAAGTTTCATCTT | −7.9 | −21.5 | 67.5 | −13.6 | 0 | −4.7 |
| | SEQ. ID. NO:938 | | | | | | |
| 350 | TCATTTTTGATCCCATCCAA | −7.9 | −23.6 | 67.3 | −15 | −0.5 | −4.3 |
| | SEQ. ID. NO:939 | | | | | | |
| 455 | GGTTCTGTCCCAGAGGACCT | −7.9 | −29.6 | 83.3 | −18.7 | −3 | −9.7 |
| | SEQ. ID. NO:940 | | | | | | |
| 477 | TGCGAGTATGGTTCCACTTC | −7.9 | −25.3 | 73.3 | −17.4 | 0 | −5.8 |
| | SEQ. ID. NO:941 | | | | | | |
| 792 | CTCCTGAAGAAACCTTTACA | −7.9 | −21.2 | 61.5 | −13.3 | 0 | −2.8 |
| | SEQ. ID. NO:942 | | | | | | |
| 912 | AACATTCCCATCTCTTTGCA | −7.9 | −24.7 | 70.5 | −16.8 | 0 | −4.8 |
| | SEQ. ID. NO:943 | | | | | | |
| 960 | CTCAGTCGCTTAGATTTACA | −7.9 | −22.3 | 66.9 | −14.4 | 0 | −3.1 |
| | SEQ. ID. NO:944 | | | | | | |
| 1555 | AAGCTTCTCTACTGCCTCTC | −7.9 | −25.9 | 76.6 | −18 | 0 | −6.2 |
| | SEQ. ID. NO:945 | | | | | | |
| 1571 | CAAGAAGTGGCTCCTGAAGC | −7.9 | −24 | 68.7 | −14.7 | −1.3 | −4.8 |
| | SEQ. ID. NO:946 | | | | | | |
| 1572 | TCAAGAAGTGGCTCCTGAAG | −7.9 | −22.6 | 66 | −14.7 | 0 | −3.7 |
| | SEQ. ID. NO:947 | | | | | | |
| 1573 | ATCAAGAAGTGGCTCCTGAA | −7.9 | −22.6 | 65.8 | −14.7 | 0 | −3.7 |
| | SEQ. ID. NO:948 | | | | | | |
| 1614 | GGATTTTCAGGCTGGTGAAT | −7.9 | −23.4 | 69 | −15 | −0.2 | −5.4 |
| | SEQ. ID. NO:949 | | | | | | |
| 1728 | AGAAGTGGGGTAAACTTGTG | −7.9 | −20.6 | 62.2 | −11.7 | −0.9 | −4.1 |
| | SEQ. ID. NO:950 | | | | | | |
| 1854 | ATTTCTTGAGTGAAACTGGG | −7.9 | −20.1 | 61.2 | −11 | −1.1 | −5.5 |
| | SEQ. ID. NO:951 | | | | | | |
| 1909 | AATATTTACAGTTGTGGAAG | −7.9 | −17.4 | 55.5 | −9.5 | 0 | −3.8 |
| | SEQ. ID. NO:952 | | | | | | |
| 1929 | AAAGTTGTTCTATCTAGCCC | −7.9 | −23 | 68.2 | −15.1 | 0 | −3.7 |
| | SEQ. ID. NO:953 | | | | | | |
| 2057 | GATGAGCAAAATGAGATTTT | −7.9 | −17.1 | 53.8 | −8.3 | −0.7 | −4.1 |
| | SEQ. ID. NO:954 | | | | | | |
| 152 | TACCTCCTTGGATTGTTTTG | −7.8 | −23.6 | 69.1 | −15.1 | −0.5 | −4.6 |
| | SEQ. ID. NO:955 | | | | | | |
| 864 | CTTCGCATGTACATATCCAT | −7.8 | −23.3 | 67.2 | −15 | 0 | −8 |
| | SEQ. ID. NO:956 | | | | | | |
| 873 | TGACACTTTCTTCGCATGTA | −7.8 | −22.8 | 67.4 | −15 | 0 | −4.8 |
| | SEQ. ID. NO:957 | | | | | | |
| 1011 | CCTTCACTGTCTTCATTCAC | −7.8 | −24.3 | 72.6 | −16.5 | 0 | −2.4 |
| | SEQ. ID. NO:958 | | | | | | |
| 1281 | TGGTCTTCATGGTCCAAAGT | −7.8 | −24.2 | 71.2 | −15.9 | −0.1 | −4.7 |
| | SEQ. ID. NO:959 | | | | | | |
| 1643 | GGCGACCCAGGAGACAGGCA | −7.8 | −30.3 | 80.2 | −22 | −0.2 | −4.2 |
| | SEQ. ID. NO:960 | | | | | | |
| 1847 | GAGTGAAACTGGGTACAAGT | −7.8 | −20.8 | 62.5 | −11.8 | −1.1 | −7 |
| | SEQ. ID. NO:961 | | | | | | |
| 1859 | TCAAGATTTCTTGAGTGAAA | −7.8 | −17.6 | 55.8 | −7.9 | −1.9 | −10.3 |
| | SEQ. ID. NO:962 | | | | | | |
| 1971 | GAATGCAGGATTCCCTGGAG | −7.8 | −25.4 | 71.3 | −15.3 | −2.3 | −8.5 |
| | SEQ. ID. NO:963 | | | | | | |
| 2007 | AATCAATTTAATTAGGCAAA | −7.8 | −15 | 49.2 | −7.2 | 0 | −4.1 |
| | SEQ. ID. NO:964 | | | | | | |
| 2042 | ATTTTCCCTAGTTCAACAGA | −7.8 | −22.5 | 66.7 | −14.7 | 0 | −3.6 |
| | SEQ. ID. NO:965 | | | | | | |
| 2103 | CCAAGATTGAATACAACTCT | −7.8 | −18.9 | 57 | −9.8 | −1.2 | −4 |
| | SEQ. ID. NO:966 | | | | | | |
| 114 | AAGGCCCTGGGAGGATTCTG | −7.7 | −27.4 | 75.9 | −19.1 | −0.1 | −8.3 |
| | SEQ. ID. NO:967 | | | | | | |
| 115 | CAAGGCCCTGGGAGGATTCT | −7.7 | −28.1 | 77.1 | −19.6 | −0.6 | −7.6 |
| | SEQ. ID. NO:968 | | | | | | |
| 301 | GGTGGTCTTCAAAAAAAACT | −7.7 | −17.5 | 54.1 | −9.8 | 0 | −2.6 |
| | SEQ. ID. NO:969 | | | | | | |
| 752 | TATAGTGGTATCCAGAGGCT | −7.7 | −24.3 | 72.5 | −16.1 | −0.1 | −4.1 |
| | SEQ. ID. NO:970 | | | | | | |
| 931 | AGTTAACAAGCATTCAGCCA | −7.7 | −22.7 | 66.3 | −14 | −0.9 | −8.7 |
| | SEQ. ID. NO:971 | | | | | | |
| 1755 | CATCACTGCACGTCCCAGAT | −7.7 | −27.3 | 74.7 | −19.6 | 0.4 | −6.6 |
| | SEQ. ID. NO:972 | | | | | | |
| 2064 | ATGGTAAGATGAGCAAAATG | −7.7 | −17 | 53.3 | −9.3 | 0 | −4.1 |
| | SEQ. ID. NO:973 | | | | | | |
| 90 | GAGTCTTCCTCTCCAGATCC | −7.6 | −27.9 | 81.4 | −19.6 | −0.5 | −5.5 |
| | SEQ. ID. NO:974 | | | | | | |
| 234 | AGGAAACTAAGAGAAGCAGT | −7.6 | −18.7 | 57.5 | −10.6 | −0.2 | −4.4 |
| | SEQ. ID. NO:975 | | | | | | |
| 327 | TTTCAATTGAAATGCACTTT | −7.6 | −17.7 | 55.2 | −8.2 | −0.1 | −11.9 |
| | SEQ. ID. NO:976 | | | | | | |
| 478 | TTGCGAGTATGGTTCCACTT | −7.6 | −25 | 72 | −17.4 | 0 | −5.8 |
| | SEQ. ID. NO:977 | | | | | | |
| 482 | TGTATTGCGAGTATGGTTCC | −7.6 | −25 | 70.5 | −16.4 | 0 | −4.1 |
| | SEQ. ID. NO:978 | | | | | | |
| 490 | AACATTGCTGTATTGCGAGT | −7.6 | −22.4 | 65.6 | −13.9 | −0.7 | −5 |
| | SEQ. ID. NO:979 | | | | | | |
| 644 | CTACCTCAGTTTCTCCCTGG | −7.6 | −28 | 79.5 | −19.9 | −0.2 | −4 |
| | SEQ. ID. NO:980 | | | | | | |
| 1072 | GGTGAGTTCAGTTTTCTCCC | −7.6 | −26.6 | 79.6 | −18.4 | −0.3 | 3.6 |
| | SEQ. ID. NO:981 | | | | | | |
| 1904 | TTACAGTTGTGGAAGTTACA | −7.6 | −20.2 | 62.5 | −12.6 | 0 | −4.2 |
| | SEQ. ID. NO:982 | | | | | | |
| 1996 | TTAGGCAAACAGGGCTTGCC | −7.6 | −26 | 72.5 | −15 | −3.4 | −98 |
| | SEQ. ID. NO:983 | | | | | | |
| 265 | TTCATCTTGAGGAAATGTCC | −7.5 | −21.2 | 63.8 | −12.6 | −1 | −5.2 |
| | SEQ. ID. NO:984 | | | | | | |
| 824 | TACACTTGTACACAGCGTTT | −7.5 | −22.5 | 66.4 | −15 | 0 | −6.3 |
| | SEQ. ID. NO:985 | | | | | | |
| 825 | TTACACTTGTACACAGCGTT | −7.5 | −22.5 | 66.4 | −15 | 0 | −5.9 |
| | SEQ. ID. NO:986 | | | | | | |
| 826 | TTTACACTTGTACACAGCGT | −7.5 | −22.5 | 66.4 | −15 | 0 | −6.3 |
| | SEQ. ID. NO:987 | | | | | | |
| 1110 | GAATCCATAATAAAATGTAG | −7.5 | −14.5 | 48.1 | −7 | 0 | −2.7 |
| | SEQ. ID. NO:988 | | | | | | |
| 1336 | CTCAGCTGAACGAAGGAACA | −7.5 | −21.5 | 61.8 | −12.9 | 0 | −10.1 |
| | SEQ. ID. NO:989 | | | | | | |
| 1342 | GAAAATCTCAGCTGAACGAA | −7.5 | −18.4 | 55.3 | −9.8 | 0 | −10.1 |
| | SEQ. ID. NO:990 | | | | | | |
| 1346 | TATTGAAAATGTGAGCTGAA | −7.5 | −17.3 | 54.3 | −8.1 | −0.1 | −11.6 |
| | SEQ. ID. NO:991 | | | | | | |
| 1606 | AGGCTGGTGAATCTTACACA | −7.5 | −23.1 | 68.1 | −14 | −1.6 | −5.4 |
| | SEQ. ID. NO:992 | | | | | | |
| 1609 | TTCAGGCTGGTGAATCTTAC | −7.5 | −22.7 | 68.3 | −14.7 | −0.2 | −5.2 |
| | SEQ. ID. NO:993 | | | | | | |
| 1678 | AGCGTGGTGATGATTGAATG | −7.5 | −21.4 | 63 | −13.9 | 0 | −4.1 |
| | SEQ. ID. NO:994 | | | | | | |
| 1922 | TTCTATCTAGCCCAATATTT | −7.5 | −21.8 | 64.8 | −14.3 | 0 | −4.1 |
| | SEQ. ID. NO:995 | | | | | | |
| 2020 | GAATTGAAGTAACAATCAAT | −7.5 | −14.7 | 48.6 | −5.5 | −1.7 | −6.1 |
| | SEQ. ID. NO:996 | | | | | | |
| 2098 | ATTGAATACAACTCTTTAAT | −7.5 | −15.5 | 50.8 | −7.1 | −0.8 | −4 |
| | SEQ. ID. NO:997 | | | | | | |
| 199 | TGTTTCTAAGTCTTCTTTTC | −7.4 | −20.3 | 65.4 | −12.3 | −0.3 | −2.7 |
| | SEQ. ID. NO:998 | | | | | | |
| 202 | CTATGTTTCTAAGTCTTCTT | −7.4 | −20.3 | 64.5 | −12.4 | −0.1 | −2.7 |
| | SEQ. ID. NO:999 | | | | | | |
| 207 | TTGAGCTATGTTTCTAAGTC | −7.4 | −20.4 | 64.3 | −13 | 0 | −5.1 |
| | SEQ. ID. NO:1000 | | | | | | |
| 232 | GAAACTAAGAGAAGCAGTGT | −7.4 | −18.7 | 57.7 | −11.3 | 0 | −4.2 |
| | SEQ. ID. NO:1001 | | | | | | |
| 328 | TTTTCAATTGAAATGCACTT | −7.4 | −17.7 | 55.2 | −8.2 | −0.4 | −12.4 |
| | SEQ. ID. NO:1002 | | | | | | |
| 329 | TTTTTCAATTGAAATGCACT | −7.4 | −17.7 | 55.2 | −8.2 | −0.4 | −12.4 |
| | SEQ. ID. NO:1003 | | | | | | |
| 733 | TCTGTCTCCACAAACAACAC | −7.4 | −21.7 | 63.5 | −13.8 | −0.1 | −2.9 |
| | SEQ. ID. NO:1004 | | | | | | |
| 744 | TATCCAGAGGCTCTGTCTCC | −7.4 | −27.5 | 80.5 | −18.5 | −1.5 | −8 |
| | SEQ. ID. NO:1005 | | | | | | |
| 1012 | ACCTTCACTGTCTTCATTCA | −7.4 | −24.3 | 72.6 | −16.9 | 0 | −2.6 |
| | SEQ. ID. NO:1006 | | | | | | |
| 1019 | AGTCACGACCTTCACTGTCT | −7.4 | −25.8 | 74.7 | −17.7 | −0.5 | −4.7 |
| | SEQ. ID. NO:1007 | | | | | | |
| 1935 | GTAGAGAAAGTTGTTCTATC | −7.4 | −18.7 | 60.2 | −9.8 | −1.4 | −4.5 |
| | SEQ. ID. NO:1008 | | | | | | |
| 2091 | ACAACTCTTTAATAAAATAT | −7.4 | −13.1 | 45.7 | −5.7 | 0 | −3.7 |
| | SEQ. ID. NO:1009 | | | | | | |
| 183 | TTTCTTCTTTCACTCCTTCT | −7.3 | −24 | 73.3 | −16.7 | 0 | 0 |
| | SEQ. ID. NO:1010 | | | | | | |
| 198 | GTTTCTAAGTCTTCTTTTCT | −7.3 | −21.2 | 67.8 | −13.3 | −0.3 | −2.7 |
| | SEQ. ID. NO:1011 | | | | | | |
| 240 | AAATCCAGGAAACTAAGAGA | −7.3 | −17.4 | 53.7 | −9.5 | −0.3 | −5.7 |
| | SEQ. ID. NO:1012 | | | | | | |
| 306 | TTTATGGTGGTCTTCAAAAA | −7.3 | −18.4 | 57.1 | −11.1 | 0 | −3.3 |
| | SEQ. ID. NO:1013 | | | | | | |
| 321 | TTGAAATGCACTTTCTTTAT | −7.3 | −18.3 | 57.1 | −9.4 | −1.6 | −9.2 |
| | SEQ. ID. NO:1014 | | | | | | |
| 322 | ATTGAAATGCACTTTCTTTA | −7.3 | −18.3 | 57.1 | −9.4 | −1.6 | −9.2 |
| | SEQ. ID. NO:1015 | | | | | | |
| 650 | TCTCTGCTACCTCAGTTTCT | −7.3 | −25.9 | 77.8 | −18.1 | −0.2 | −3.5 |
| | SEQ. ID. NO:1016 | | | | | | |
| 863 | TTCGCATGTACATATCCATC | −7.3 | −22.8 | 66.8 | −15 | 0 | −7.8 |
| | SEQ. ID. NO:1017 | | | | | | |
| 1381 | TTCCAATAGGTCAGAATGCC | −7.3 | −23.4 | 67.5 | −15 | −1 | −4.6 |
| | SEQ. ID. NO:1018 | | | | | | |
| 1567 | AAGTGGCTCCTGAAGCTTCT | −7.3 | −25.7 | 74.2 | −16.8 | −1.3 | −10.8 |
| | SEQ. ID. NO:1019 | | | | | | |
| 1636 | CAGGAGACAGGCAAAGTGTT | −7.3 | −22.8 | 67 | −15.5 | 0 | −4 |
| | SEQ. ID. NO:1020 | | | | | | |
| 1658 | TCCGTAATTCAGTCAGGCGA | −7.3 | −25.3 | 71.3 | −18 | 0 | −4 |
| | SEQ. ID. NO:1021 | | | | | | |
| 1891 | AGTTACACATGTAATTACAA | −7.3 | −17 | 54.3 | −8.5 | −0.3 | −10.3 |
| | SEQ. ID. NO:1022 | | | | | | |
| 74 | ATCCCAGCGATTTTGCTACA | −7.2 | −25.9 | 71.8 | −17.2 | −1.4 | −5.1 |
| | SEQ. ID. NO:1023 | | | | | | |
| 87 | TCTTCCTCTCCAGATCCCAG | −7.2 | −28.8 | 81 | −21.3 | 0 | −4.5 |
| | SEQ. ID. NO:1024 | | | | | | |
| 158 | GTCTTCTACCTCCTTGGATT | −7.2 | −26 | 76.1 | −18.1 | −0.5 | −4.6 |
| | SEQ. ID. NO:1025 | | | | | | |
| 357 | ATGAGATTCATTTTTGATCC | −7.2 | −19.8 | 61.2 | −11.7 | −0.8 | −5.3 |
| | SEQ. ID. NO:1026 | | | | | | |
| 358 | AATGAGATTCATTTTTGATC | −7.2 | −17.1 | 55.2 | −8.3 | −1.5 | −6.9 |
| | SEQ. ID. NO:1027 | | | | | | |
| 379 | GGTAGGTAAATGGGAATGTT | −7.2 | −20.4 | 61.6 | −13.2 | 0 | −2.5 |
| | SEQ. ID. NO:1028 | | | | | | |
| 959 | TCAGTCGCTTAGATTTACAC | −7.2 | −21.6 | 65.5 | −14.4 | 0 | −3.1 |
| | SEQ. ID. NO:1029 | | | | | | |
| 1351 | TTTCTTATTGAAAATCTCAG | −7.2 | −16.3 | 53.2 | −8.1 | −0.9 | −4.1 |
| | SEQ. ID. NO:1030 | | | | | | |
| 1392 | CGAATTCTTTCTTCCAATAG | −7.2 | −19.8 | 59.6 | −11.8 | −0.6 | −6.4 |
| | SEQ. ID. NO:1031 | | | | | | |
| 1434 | CTAAACATAGGTGTTATATA | −7.2 | −16.6 | 53.7 | −7.7 | −1.7 | −5.9 |
| | SEQ. ID. NO:1032 | | | | | | |
| 1576 | CACATCAAGAAGTGGCTCCT | −7.2 | −24.3 | 69.7 | −16.6 | −0.1 | −5.1 |
| | SEQ. ID. NO:1033 | | | | | | |
| 1610 | TTTCAGGCTGGTGAATCTTA | −7.2 | −22.6 | 68.1 | −14.7 | −0.5 | −5.7 |
| | SEQ. ID. NO:1034 | | | | | | |
| 1638 | CCCAGGAGACAGGCAAAGTG | −7.2 | −25.5 | 70.7 | −18.3 | 0 | −4 |
| | SEQ. ID. NO:1035 | | | | | | |
| 1839 | CTGGGTACAAGTGAAATAAA | −7.2 | −17.1 | 53.4 | −9.9 | 0 | −5.2 |
| | SEQ. ID. NO:1036 | | | | | | |
| 1857 | AAGATTTCTTGAGTGAAACT | −7.2 | −17.6 | 55.7 | −9.4 | −0.9 | −5.7 |
| | SEQ. ID. NO:1037 | | | | | | |
| 1864 | ATTCATCAAGATTTCTTGAG | −7.2 | −18.4 | 58.4 | −9.3 | −1.9 | −10.7 |
| | SEQ. ID. NO:1038 | | | | | | |
| 2050 | AAAATGAGATTTTCCCTAGT | −7.2 | −19.6 | 59.1 | −11.5 | −0.7 | −5 |
| | SEQ. ID. NO:1039 | | | | | | |
| 2062 | GGTAAGATGAGCAAAATGAG | −7.2 | −17.6 | 54.7 | −10.4 | 0 | −4.1 |
| | SEQ. ID. NO:1040 | | | | | | |
| 23 | AGTCGGGGAGACAATGAGGT | −7.1 | −24.4 | 70.3 | −15.2 | −2.1 | −5 |
| | SEQ. ID. NO:1041 | | | | | | |
| 53 | ATGCTCAGAATCCAATTTCG | −7.1 | −21.5 | 62.6 | −13.7 | −0.4 | −4 |
| | SEQ. ID. NO:1042 | | | | | | |
| 56 | CAAATGCTCAGAATCCAATT | −7.1 | −19.5 | 57.9 | −12.4 | 0 | −2.9 |
| | SEQ. ID. NO:1043 | | | | | | |
| 229 | ACTAAGAGAAGCAGTGTTCA | −7.1 | −20.7 | 63.5 | −12.9 | −0.4 | −6.8 |
| | SEQ. ID. NO:1044 | | | | | | |
| 272 | CTGAAGTTTCATCTTGAGGA | −7.1 | −21.1 | 64.6 | −14 | 0 | −4.7 |
| | SEQ. ID. NO:1045 | | | | | | |
| 380 | TGGTAGGTAAATGGGAATGT | −7.1 | −20.3 | 61.1 | −13.2 | 0 | −1.2 |
| | SEQ. ID. NO:1046 | | | | | | |
| 1017 | TCACGACCTTCACTGTCTTC | −7.1 | −25.1 | 73 | −17.3 | −0.5 | −3.7 |
| | SEQ. ID. NO:1047 | | | | | | |
| 1232 | CTACAAGAACCTGTACATGA | −7.1 | −20.2 | 60 | −13.1 | 0 | −6.5 |
| | SEQ. ID. NO:1048 | | | | | | |
| 1236 | AATTCTACAAGAACCTGTAC | −7.1 | −18.7 | 57.4 | −10.6 | −0.9 | −5.5 |
| | SEQ. ID. NO:1049 | | | | | | |
| 1335 | TCAGCTGAACGAAGGAACAT | −7.1 | −20.6 | 60 | −12.6 | 0 | −9.8 |
| | SEQ. ID. NO:1050 | | | | | | |
| 1338 | ATCTCAGCTGAACGAAGGAA | −7.1 | −21 | 61.4 | −12.8 | 0 | −10.1 |
| | SEQ. ID. NO:1051 | | | | | | |
| 1344 | TTGAAAATCTCAGCTGAACG | −7.1 | −18.6 | 56.1 | −10.4 | −0.1 | −10.1 |
| | SEQ. ID. NO:1052 | | | | | | |
| 1712 | TGTGGTCGTTTACTCTCCAT | −7.1 | −25.4 | 74.4 | −17.6 | −0.4 | −3.9 |
| | SEQ. ID. NO:1053 | | | | | | |
| 1776 | AGACCCCTCCCCTGTAATCC | −7.1 | −31.6 | 81.9 | −24.5 | 0 | −2.1 |
| | SEQ. ID. NO:1054 | | | | | | |
| 1832 | CAAGTGAAATAAAGGAAAGT | −7.1 | −14.3 | 47.6 | −7.2 | 0 | −1.6 |
| | SEQ. ID. NO:1055 | | | | | | |
| 1986 | AGGGCTTGCCAATTAGAATG | −7.1 | −22.7 | 65.4 | −13.8 | −1.8 | −8.5 |
| | SEQ. ID. NO:1056 | | | | | | |
| 1995 | TAGGCAAACAGGGCTTGCCA | −7.1 | −26.6 | 73.2 | −15 | −4.5 | −11.1 |
| | SEQ. ID. NO:1057 | | | | | | |
| 2093 | ATACAACTCTTTAATAAAAT | −7.1 | −13.1 | 45.7 | −6 | 0 | −3.7 |
| | SEQ. ID. NO:1058 | | | | | | |
| 204 | AGCTATGTTTCTAAGTCTTC | −7 | −21.1 | 66.8 | −14.1 | 0 | −4.3 |
| | SEQ. ID. NO:1059 | | | | | | |
| 239 | AATCCAGGAAACTAAGAGAA | −7 | −17.4 | 53.7 | −9.9 | −0.1 | −5.7 |
| | SEQ. ID. NO:1060 | | | | | | |
| 492 | TGAACATTGCTGTATTGCGA | −7 | −21.8 | 63.4 | −13.9 | −0.7 | −5 |
| | SEQ. ID. NO:1061 | | | | | | |
| 1160 | CTTTTAAAATTTTATTTGTT | −7 | −14.3 | 48.8 | −6.7 | −0.2 | −8 |
| | SEQ. ID. NO:1062 | | | | | | |
| 1206 | GCCATTTCCGTCAAAATGAG | −7 | −22.7 | 63.9 | −14.1 | −1.6 | −6 |
| | SEQ. ID. NO:1063 | | | | | | |
| 1207 | TGCCATTTCCGTCAAAATGA | −7 | −22.7 | 63.6 | −14.1 | −1.6 | −6.2 |
| | SEQ. ID. NO:1064 | | | | | | |
| 1239 | GTGAATTCTACAAGAACCTG | −7 | −19.4 | 58.6 | −11.7 | −0.4 | −7.1 |
| | SEQ. ID. NO:1065 | | | | | | |
| 123 | TGGACTTTCAAGGCCCTGGG | −6.9 | −27.8 | 76.4 | −20.4 | 0 | −7.8 |
| | SEQ. ID. NO:1066 | | | | | | |
| 144 | TGGATTGTTTTGGGTCAGAG | −6.9 | −22.7 | 68.9 | −15.8 | 0 | −3.4 |
| | SEQ. ID. NO:1067 | | | | | | |
| 231 | AAACTAAGAGAAGCAGTGTT | −6.9 | −18.2 | 56.7 | −11.3 | 0 | −4.4 |
| | SEQ. ID. NO:1068 | | | | | | |
| 283 | CTCCAAAGTGTCTGAAGTTT | −6.9 | −21.6 | 64.8 | −14.7 | 0 | −3 |
| | SEQ. ID. NO:1069 | | | | | | |
| 323 | AATTGAAATGCACTTTCTTT | −6.9 | −17.9 | 55.8 | −9.4 | −1.6 | −9.2 |
| | SEQ. ID. NO:1070 | | | | | | |
| 349 | CATTTTTGATCCCATCCAAA | −6.9 | −22.5 | 63.8 | −15 | −0.3 | −4.3 |
| | SEQ. ID. NO:1071 | | | | | | |
| 454 | GTTCTGTCCCAGAGGACCTG | −6.9 | −28.4 | 80.3 | −19.2 | −2.3 | −6.5 |
| | SEQ. ID. NO:1072 | | | | | | |
| 706 | ATCCCCTTTGATCCTCCCTG | −6.9 | −30.7 | 81.4 | −23.8 | 0 | −4.3 |
| | SEQ. ID. NO:1073 | | | | | | |
| 968 | GATTTTTTCTCAGTCGCTTA | −6.9 | −22.5 | 68 | −15.6 | 0 | −3.1 |
| | SEQ. ID. NO:1074 | | | | | | |
| 1164 | TCTTCTTTTAAAATTTTATT | −6.9 | −14.7 | 49.9 | −7.3 | 0 | −8 |
| | SEQ. ID. NO:1075 | | | | | | |
| 1231 | TACAAGAACCTGTACATGAT | −6.9 | −19.3 | 58.2 | −12.4 | 0 | −6.5 |
| | SEQ. ID. NO:1076 | | | | | | |
| 1233 | TCTACAAGAACCTGTACATG | −6.9 | −20 | 60.1 | −13.1 | 0 | −6.1 |
| | SEQ. ID. NO:1077 | | | | | | |
| 1332 | GCTGAACGAAGGAACATAGC | −6.9 | −21 | 60.8 | −14.1 | 0 | −3.5 |
| | SEQ. ID. NO:1078 | | | | | | |
| 1423 | TGTTATATATTCATCAGAGA | −6.9 | −17.9 | 57.7 | −11 | 0 | −3.9 |
| | SEQ. ID. NO:1079 | | | | | | |
| 1569 | AGAAGTGGCTCCTGAAGCTT | −6.9 | −25 | 72.1 | −16 | −2.1 | −7 |
| | SEQ. ID. NO:1080 | | | | | | |
| 1613 | GATTTTCAGGCTGGTGAATC | −6.9 | −22.6 | 68 | −15 | −0.5 | −5.7 |
| | SEQ. ID. NO:1081 | | | | | | |
| 1639 | ACCCAGGAGACAGGCAAAGT | −6.9 | −25.7 | 71.4 | −18.8 | 0 | −4 |
| | SEQ. ID. NO:1082 | | | | | | |
| 1829 | GTGAAATAAAGGAAAGTTAT | −6.9 | −14.1 | 47.5 | 7.2 | 0 | −2.7 |
| | SEQ. ID. NO:1083 | | | | | | |
| 1830 | AGTGAAATAAAGGAAAGTTA | −6.9 | −14.1 | 47.6 | −7.2 | 0 | −2.6 |
| | SEQ. ID. NO:1084 | | | | | | |
| 1848 | TGAGTGAAACTGGGTACAAG | −6.9 | −19.6 | 59.4 | −11.5 | −1.1 | −7 |
| | SEQ. ID. NO:1085 | | | | | | |
| 2021 | AGAATTGAAGTAACAATCAA | −6.9 | −14.7 | 48.7 | −6.8 | −0.9 | −4.4 |
| | SEQ. ID. NO:1086 | | | | | | |
| 2053 | AGCAAAATGAGATTTTCCCT | −6.9 | −21.2 | 61.7 | −13.3 | −0.9 | −4.8 |
| | SEQ. ID. NO:1087 | | | | | | |
| 2065 | TATGGTAAGATGAGCAAAAT | −6.9 | −16.7 | 52.8 | −9.8 | 0 | −4.1 |
| | SEQ. ID. NO:1088 | | | | | | |
| 2106 | TTGCCAAGATTGAATACAAC | −6.9 | −18.6 | 56.2 | −10.8 | −0.8 | −4.5 |
| | SEQ. ID. NO:1089 | | | | | | |
| 61 | TGCTACAAATGCTCAGAATC | −6.8 | −20 | 60.2 | −12.5 | −0.4 | −3.6 |
| | SEQ. ID. NO:1090 | | | | | | |
| 73 | TCCCAGCGATTTTGCTACAA | −6.8 | −25.2 | 69.7 | −16.8 | −1.6 | −6.1 |
| | SEQ. ID. NO:1091 | | | | | | |
| 116 | TCAAGGCCCTGGGAGGATTC | −6.8 | −27.6 | 76.9 | −20 | −0.6 | −8.3 |
| | SEQ. ID. NO:1092 | | | | | | |
| 367 | GGAATGTTCAATGAGATTCA | −6.8 | −19.2 | 59.1 | −11.7 | −0.6 | −7.6 |
| | SEQ. ID. NO:1093 | | | | | | |
| 972 | TTCACATTTTTTCTCAGTCG | −6.8 | −21.4 | 65.6 | −14.6 | 0 | −2.5 |
| | SEQ. ID. NO:1094 | | | | | | |
| 1208 | TTGCCATTTCCGTCAAAATG | −6.8 | −22.2 | 62.8 | −14.1 | −1.2 | −6.2 |
| | SEQ. ID. NO:1095 | | | | | | |
| 1289 | AAGCAATCTGGTCTTCATGG | −6.8 | −22.5 | 67 | −15.7 | 0 | −4.7 |
| | SEQ. ID. NO:1096 | | | | | | |
| 1390 | AATTCTTTCTTCCAATAGGT | −6.8 | −20.8 | 63.4 | −13.4 | −0.3 | −3.6 |
| | SEQ. ID. NO:1097 | | | | | | |
| 1542 | GCCTCTCTATCCTTTATGTA | −6.8 | −25.1 | 74.2 | −18.3 | 0 | −2 |
| | SEQ. ID. NO:1098 | | | | | | |
| 1818 | GAAAGTTATACATCAGATTA | −6.8 | −16.3 | 53.1 | −9.5 | 0 | −3.4 |
| | SEQ. ID. NO:1099 | | | | | | |
| 1910 | CAATATTTACAGTTGTGGAA | −6.8 | −18.1 | 56.6 | −11.3 | 0 | −4.1 |
| | SEQ. ID. NO:1100 | | | | | | |
| 80 | CTCCAGATCCCAGCGATTTT | −6.7 | −27.2 | 74.5 | −20.5 | 0 | −4.1 |
| | SEQ. ID. NO:1101 | | | | | | |
| 82 | CTCTCCAGATCCCAGCGATT | −6.7 | −28.3 | 77.2 | −21.6 | 0 | −4.5 |
| | SEQ. ID. NO:1102 | | | | | | |
| 159 | TGTCTTCTACCTCCTTGGAT | −6.7 | −25.9 | 75.5 | −18.5 | −0.5 | −5 |
| | SEQ. ID. NO:1103 | | | | | | |
| 342 | GATCCCATCCAAATTTTTCA | −6.7 | −22.9 | 65.3 | −16.2 | 0 | −5.4 |
| | SEQ. ID. NO:1104 | | | | | | |
| 708 | TCATCCCCTTTGATCCTCCC | −6.7 | −30.9 | 82.5 | −24.2 | 0 | −4.3 |
| | SEQ. ID. NO:1105 | | | | | | |
| 862 | TCGCATGTACATATCCATCA | −6.7 | −23.4 | 67.6 | −16.2 | 0 | −8 |
| | SEQ. ID. NO:1106 | | | | | | |
| 1105 | CATAATAAAATGTAGAAGAG | −6.7 | −12.7 | 44.8 | −6 | 0 | −2.4 |
| | SEQ. ID. NO:1107 | | | | | | |
| 1238 | TGAATTCTACAAGAACCTGT | −6.7 | −19.4 | 58.6 | −11.7 | −0.9 | −6.9 |
| | SEQ. ID. NO:1108 | | | | | | |
| 1240 | TGTGAATTCTACAAGAACCT | −6.7 | −19.4 | 58.6 | −11.7 | −0.9 | −8 |
| | SEQ. ID. NO:1109 | | | | | | |
| 1282 | CTGGTCTTCATGGTCCAAAG | −6.7 | −23.9 | 69.8 | −16.7 | −0.2 | −4.7 |
| | SEQ. ID. NO:1110 | | | | | | |
| 1361 | CAGACGGAAGTTTCTTATTG | −6.7 | −20 | 60.7 | −12.4 | −0.8 | −5.1 |
| | SEQ. ID. NO:1111 | | | | | | |
| 1530 | TTTATGTATTGTCTATCTGG | −6.7 | −19.6 | 62.2 | −12.9 | 0 | −1.3 |
| | SEQ. ID. NO:1112 | | | | | | |
| 1738 | GATTTCACAGAGAAGTGGGG | −6.7 | −22.1 | 66.2 | −14.8 | −0.3 | −4.7 |
| | SEQ. ID. NO:1113 | | | | | | |
| 1739 | AGATTTCACAGAGAAGTGGG | −6.7 | −20.9 | 63.7 | −13.3 | −0.7 | −4.7 |
| | SEQ. ID. NO:1114 | | | | | | |
| 1958 | CCTGGAGCCTTTTAAAACAC | −6.7 | −22.4 | 63.7 | −15.7 | 0 | −6.2 |
| | SEQ. ID. NO:1115 | | | | | | |
| 1994 | AGGCAAACAGGGCTTGCCAA | −6.7 | −26.2 | 71.5 | −15 | −4.5 | −11.1 |
| | SEQ. ID. NO:1116 | | | | | | |
| 2041 | TTTTCCCTAGTTCAACAGAT | −6.7 | −22.5 | 66.7 | −15.8 | 0 | −3.6 |
| | SEQ. ID. NO:1117 | | | | | | |
| 2074 | TATATGCAATATGGTAAGAT | −6.7 | −16.9 | 53.8 | −9.5 | −0.5 | −5.6 |
| | SEQ. ID. NO:1118 | | | | | | |
| 2075 | ATATATGCAATATGGTAAGA | −6.7 | −16.9 | 53.8 | −9.5 | −0.5 | −5.6 |
| | SEQ. ID. NO:1119 | | | | | | |
| 2087 | CTCTTTAATAAAATATATGC | −6.7 | −14.2 | 48.1 | −7.5 | 0 | −4.2 |
| | SEQ. ID. NO:1120 | | | | | | |
| 431 | CTTGTTCTGTTAAAACACCA | −6.6 | −20.3 | 60.6 | −12.8 | −0.7 | −5.5 |
| | SEQ. ID. NO:1121 | | | | | | |
| 432 | ACTTGTTCTGTTAAAACACC | −6.6 | −19.8 | 60 | −12.3 | −0.7 | −5.5 |
| | SEQ. ID. NO:1122 | | | | | | |
| 435 | GCCACTTGTTCTGTTAAAAC | −6.6 | −21.4 | 63.5 | −14.8 | 0 | −3.3 |
| | SEQ. ID. NO:1123 | | | | | | |
| 469 | TGGTTCCACTTCCAGGTTCT | −6.6 | −27.7 | 80.6 | −20.5 | −0.3 | −4.8 |
| | SEQ. ID. NO:1124 | | | | | | |
| 598 | GAGTTCATATATTCCAGGAG | −6.6 | −21.4 | 65.5 | −14.8 | 0 | −5.3 |
| | SEQ. ID. NO:1125 | | | | | | |
| 753 | TTATAGTGGTATCCAGAGGC | −6.6 | −23.5 | 70.8 | −16.1 | −0.6 | −6.9 |
| | SEQ. ID. NO:1126 | | | | | | |
| 928 | TAACAAGCATTCAGCCAACA | −6.6 | −21.6 | 62.3 | −14 | −0.9 | −4.1 |
| | SEQ. ID. NO:1127 | | | | | | |
| 1036 | CGAGGTCACTTGTCGCAAGT | −6.6 | −25.5 | 72.3 | −16.9 | −2 | −10.6 |
| | SEQ. ID. NO:1128 | | | | | | |
| 1093 | TAGAAGAGTCTGTTGATCTG | −6.6 | −19.9 | 62.7 | −12.8 | −0.2 | −5.8 |
| | SEQ. ID. NO:1129 | | | | | | |
| 1109 | AATCCATAATAAAATGTAGA | −6.6 | −14.5 | 48.1 | −7.9 | 0 | −2.8 |
| | SEQ. ID. NO:1130 | | | | | | |
| 1843 | GAAACTGGGTACAAGTGAAA | −6.6 | −18.2 | 55.6 | −11.6 | 0 | −6 |
| | SEQ. ID. NO:1131 | | | | | | |
| 2088 | ACTCTTTAATAAAATATATG | −6.6 | −12.6 | 44.9 | −6 | 0 | −4.2 |
| | SEQ. ID. NO:1132 | | | | | | |
| 55 | AAATGCTCAGAATCCAATTT | −6.5 | −18.9 | 57 | −12.4 | 0 | −3.6 |
| | SEQ. ID. NO:1133 | | | | | | |
| 153 | CTACCTCCTTGGATTGTTTT | −6.5 | −24.5 | 71.2 | −17.3 | −0.5 | −4.4 |
| | SEQ. ID. NO:1134 | | | | | | |
| 172 | ACTCCTTCTACGATGTCTTC | −6.5 | −24.1 | 71.4 | −17.6 | 0 | −3.5 |
| | SEQ. ID. NO:1135 | | | | | | |
| 330 | ATTTTTCAATTGAAATGCAC | −6.5 | −16.8 | 53.3 | −8.2 | −0.4 | −12.4 |
| | SEQ. ID. NO:1136 | | | | | | |
| 483 | CTGTATTGCGAGTATGGTTC | −6.5 | −22.9 | 68.7 | −16.4 | 0 | −4.1 |
| | SEQ. ID. NO:1137 | | | | | | |
| 802 | GGTAATGCTTCTCCTGAAGA | −6.5 | −23.3 | 68.3 | −14.6 | −2.2 | −6.7 |
| | SEQ. ID. NO:1138 | | | | | | |
| 1005 | CTGTCTTCATTCACGGTCTG | −6.5 | −24.5 | 72.6 | −18 | 0 | −3.5 |
| | SEQ. ID. NO:1139 | | | | | | |
| 1007 | CACTGTCTTCATTCACGGTC | −6.5 | −24.5 | 72.5 | −18 | 0 | −3.5 |
| | SEQ. ID. NO:1140 | | | | | | |
| 1018 | GTCACGACCTTCACTGTCTT | −6.5 | −25.9 | 74.7 | −19.4 | 0 | −3.7 |
| | SEQ. ID. NO:1141 | | | | | | |
| 1020 | AAGTCACGACCTTCACTGTC | −6.5 | −24.2 | 70.2 | −17.7 | 0 | −4.7 |
| | SEQ. ID. NO:1142 | | | | | | |
| 1079 | GATCTGGGGTGAGTTCAGTT | −6.5 | −25 | 75.9 | −18 | −0.2 | −4.1 |
| | SEQ. ID. NO:1143 | | | | | | |
| 1096 | ATGTAGAAGAGTCTGTTGAT | −6.5 | −19.8 | 62.4 | −12.8 | −0.2 | −5.8 |
| | SEQ. ID. NO:1144 | | | | | | |
| 1245 | TTTTTTGTGAATTCTACAAG | −6.5 | −16.9 | 54.6 | −9 | −0.7 | −10.5 |
| | SEQ. ID. NO:1145 | | | | | | |
| 1477 | CTCCTCTTGAGTCATTTTCA | −6.5 | −23.9 | 72.2 | −16.9 | −0.2 | −5.8 |
| | SEQ. ID. NO:1146 | | | | | | |
| 1623 | AAGTGTTGAGGATTTTCAGG | −6.5 | −20.8 | 64.2 | −14.3 | 0 | −3.2 |
| | SEQ. ID. NO:1147 | | | | | | |
| 1631 | GACAGGCAAAGTGTTGAGGA | −6.5 | −22.7 | 66.8 | −15.3 | −0.7 | −3.9 |
| | SEQ. ID. NO:1148 | | | | | | |
| 1785 | AAAGGAGCTAGACCCCTCCC | −6.5 | −28.9 | 76.6 | −20.4 | −2 | −7.6 |
| | SEQ. ID. NO:1149 | | | | | | |
| 1808 | CATCAGATTAATATGAGAGA | −6.5 | −17 | 54.5 | −10.5 | 0 | −7 |
| | SEQ. ID. NO:1150 | | | | | | |
| 1831 | AAGTGAAATAAAGGAAAGTT | −6.5 | −13.7 | 46.6 | −7.2 | 0 | −2.3 |
| | SEQ. ID. NO:1151 | | | | | | |
| 1889 | TTACACATGTAATTACAACA | −6.5 | −16.7 | 53.1 | −9 | −0.2 | −10.3 |
| | SEQ. ID. NO:1152 | | | | | | |
| 113 | AGGCCCTGGGAGGATTCTGG | −6.4 | −29.3 | 81 | −22.1 | −0.6 | −8.3 |
| | SEQ. ID. NO:1153 | | | | | | |
| 324 | CAATTGAAATGCACTTTCTT | −6.4 | −18.5 | 56.7 | −11.1 | −0.9 | −8.5 |
| | SEQ. ID. NO:1154 | | | | | | |
| 378 | GTAGGTAAATGGGAATGTTC | −6.4 | −19.6 | 60.4 | −13.2 | 0 | −4.5 |
| | SEQ. ID. NO:1155 | | | | | | |
| 626 | GGTAGAGAGTCTCAGCTGGC | −6.4 | −26.6 | 80.6 | −18.8 | −1.1 | −10 |
| | SEQ. ID. NO:1156 | | | | | | |
| 827 | TTTTACACTTGTACACAGCG | −6.4 | −21.4 | 63.6 | −15 | 0 | −6.3 |
| | SEQ. ID. NO:1157 | | | | | | |
| 1024 | TCGCAAGTCACGACCTTCAC | −6.4 | −25.4 | 70.5 | −18.3 | −0.5 | −4.7 |
| | SEQ. ID. NO:1158 | | | | | | |
| 1267 | CAAAGTCTGAAATCCTGGTA | −6.4 | −20.4 | 60.7 | −14 | 0 | −4.6 |
| | SEQ. ID. NO:1159 | | | | | | |
| 1287 | GCAATCTGGTCTTCATGGTC | −6.4 | −24.8 | 74.4 | −18.4 | 0 | −4.7 |
| | SEQ. ID. NO:1160 | | | | | | |
| 1485 | AGAGCATACTCCTCTTGAGT | −6.4 | −24.4 | 73 | −16.4 | −1.5 | −7.1 |
| | SEQ. ID. NO:1161 | | | | | | |
| 1575 | ACATCAAGAAGTGGCTCCTG | −6.4 | −23.6 | 68.4 | −17.2 | 0 | −3.7 |
| | SEQ. ID. NO:1162 | | | | | | |
| 1605 | GGCTGGTGAATCTTACACAA | −6.4 | −22.4 | 65.6 | −15.1 | −0.8 | −5.9 |
| | SEQ. ID. NO:1163 | | | | | | |
| 1642 | GCGACCCAGGAGACAGGCAA | −6.4 | −28.4 | 75.4 | −22 | 0 | −4.2 |
| | SEQ. ID. NO:1164 | | | | | | |
| 1745 | CGTCCCAGATTTCACAGAGA | −6.4 | −25.1 | 71.1 | −18.7 | 0 | −2.7 |
| | SEQ. ID. NO:1165 | | | | | | |
| 1787 | AAAAAGGAGCTAGACCCCTC | −6.4 | −23.5 | 65.7 | −16.6 | −0.2 | −5.3 |
| | SEQ. ID. NO:1166 | | | | | | |
| 1821 | AAGGAAAGTTATACATCAGA | −6.4 | −17 | 54.2 | −10.6 | 0 | −2.9 |
| | SEQ. ID. NO:1167 | | | | | | |
| 2094 | AATACAACTCTTTAATAAAA | −6.4 | −12.4 | 44.2 | −6 | 0 | −3.7 |
| | SEQ. ID. NO:1168 | | | | | | |
| 2109 | TTATTGCCAAGATTGAATAC | −6.4 | −18.2 | 56.1 | −11.8 | 0 | −3.7 |
| | SEQ. ID. NO:1169 | | | | | | |
| 57 | ACAAATGCTCAGAATCCAAT | −6.3 | −19.6 | 58.1 | −13.3 | 0 | −3.6 |
| | SEQ. ID. NO:1170 | | | | | | |
| 79 | TCCAGATCCCAGCGATTTTG | −6.3 | −26.3 | 72.5 | −20 | 0 | −4.5 |
| | SEQ. ID. NO:1171 | | | | | | |
| 170 | TCCTTCTACGATGTCTTCTA | −6.3 | −23.6 | 70.2 | −17.3 | 0 | −3.5 |
| | SEQ. ID. NO:1172 | | | | | | |
| 173 | CACTCCTTCTACGATGTCTT | −6.3 | −24.4 | 70.9 | −18.1 | 0 | −3.5 |
| | SEQ. ID. NO:1173 | | | | | | |
| 618 | GTCTCAGCTGGCATACGCCT | −6.3 | −29.4 | 81.7 | −20.2 | −2.9 | −9.9 |
| | SEQ. ID. NO:1174 | | | | | | |
| 780 | CCTTTACACCCCTCACAGGT | −6.3 | −29.2 | 79 | −22.2 | −0.5 | −3.9 |
| | SEQ. ID. NO:1175 | | | | | | |
| 1035 | GAGGTCACTTGTCGCAAGTC | −6.3 | −25.1 | 74.1 | −16.6 | −2.2 | −10.8 |
| | SEQ. ID. NO:1176 | | | | | | |
| 1234 | TTCTACAAGAACCTGTACAT | −6.3 | −20.4 | 60.5 | −13.1 | −0.4 | −6.9 |
| | SEQ. ID. NO:1177 | | | | | | |
| 1352 | GTTTCTTATTGAAAATCTCA | −6.3 | −17.5 | 55.9 | −9.7 | −1.4 | −4.5 |
| | SEQ. ID. NO:1178 | | | | | | |
| 1391 | GAATTCTTTCTTCCAATAGG | −6.3 | −20.2 | 61.6 | −13.4 | −0.1 | −6.1 |
| | SEQ. ID. NO:1179 | | | | | | |
| 1435 | ACTAAACATAGGTGTTATAT | −6.3 | −17.1 | 54.8 | −9.1 | −1.7 | −5.8 |
| | SEQ. ID. NO:1180 | | | | | | |
| 1473 | TCTTGAGTCATTTTCAGTTC | −6.3 | −21.4 | 68.2 | −15.1 | 0 | −5.8 |
| | SEQ. ID. NO:1181 | | | | | | |
| 1548 | TCTACTGCCTCTCTATCCTT | −6.3 | −26.5 | 77.4 | −20.2 | 0 | −3 |
| | SEQ. ID. NO:1182 | | | | | | |
| 1577 | GCACATCAAGAAGTGGCTCC | −6.3 | −25.2 | 72 | −18 | −0.8 | −6.4 |
| | SEQ. ID. NO:1183 | | | | | | |
| 1693 | TGACATCAGCATCTCAGCGT | −6.3 | −25.3 | 73.2 | −18 | −0.9 | −4.1 |
| | SEQ. ID. NO:1184 | | | | | | |
| 2105 | TGCCAAGATTGAATACAACT | −6.3 | −19.4 | 57.7 | −12.2 | −0.8 | −4.5 |
| | SEQ. ID. NO:1185 | | | | | | |
| 2113 | TGCTTTATTGCCAAGATTGA | −6.3 | −21.8 | 64.1 | −15.5 | 0 | −3.7 |
| | SEQ. ID. NO:1186 | | | | | | |
| 24 | AAGTCGGGGAGACAATGAGG | −6.2 | −22.5 | 64.9 | −14.2 | −2.1 | −4.8 |
| | SEQ. ID. NO:1187 | | | | | | |
| 104 | GAGGATTCTGGACTGAGTCT | −6.2 | −23.8 | 71.8 | −16.3 | −1.2 | −6.2 |
| | SEQ. ID. NO:1188 | | | | | | |
| 147 | CCTTGGATTGTTTTGGGTCA | −6.2 | −25.1 | 73.2 | −18.9 | 0 | −2.7 |
| | SEQ. ID. NO:1189 | | | | | | |
| 266 | TTTCATCTTGAGGAAATGTC | −6.2 | −19.3 | 60.3 | −12.6 | −0.2 | −7.1 |
| | SEQ. ID. NO:1190 | | | | | | |
| 620 | GAGTCTCAGCTGGCATACGC | −6.2 | −27.1 | 77.8 | −20 | −0.4 | −9.6 |
| | SEQ. ID. NO:1191 | | | | | | |
| 642 | ACCTCAGTTTCTCCCTGGTA | −6.2 | −28.3 | 81.1 | −21.6 | −0.2 | −4.7 |
| | SEQ. ID. NO:1192 | | | | | | |
| 745 | GTATCCAGAGGCTCTGTCTC | −6.2 | −26.7 | 80.6 | −19.4 | −1 | −7.5 |
| | SEQ. ID. NO:1193 | | | | | | |
| 930 | GTTAACAAGCATTCAGCCAA | −6.2 | −22 | 63.9 | −14.8 | −0.9 | −8 |
| | SEQ. ID. NO:1194 | | | | | | |
| 1037 | TCGAGGTCACTTGTCGCAAG | −6.2 | −24.7 | 70.6 | −17.1 | −1.3 | −9.2 |
| | SEQ. ID. NO:1195 | | | | | | |
| 1612 | ATTTTCAGGCTGGTGAATCT | −6.2 | −22.9 | 68.6 | −16 | −0.5 | −5.7 |
| | SEQ. ID. NO:1196 | | | | | | |
| 1709 | GGTCGTTTACTCTCCATGAC | −6.2 | −25 | 73 | −18.8 | 0 | −4.5 |
| | SEQ. ID. NO:1197 | | | | | | |
| 1911 | CCAATATTTACAGTTGTGGA | −6.2 | −20.8 | 62.4 | −14.6 | 0 | −4.1 |
| | SEQ. ID. NO:1198 | | | | | | |
| 2026 | CAGATAGAATTGAAGTAACA | −6.2 | −16 | 51.7 | −9.8 | 0 | −3.1 |
| | SEQ. ID. NO:1199 | | | | | | |
| 2095 | GAATACAACTCTTTAATAAA | −6.2 | −13.7 | 46.8 | −7.5 | 0 | −3.4 |
| | SEQ. ID. NO:1200 | | | | | | |
| 162 | CGATGTCTTCTACCTCCTTG | −6.1 | −25.5 | 72.7 | −19.4 | 0 | −3 |
| | SEQ. ID. NO:1201 | | | | | | |
| 278 | AAGTGTCTGAAGTTTCATCT | −6.1 | −20.7 | 64.7 | −14.6 | 0 | −4.7 |
| | SEQ. ID. NO:1202 | | | | | | |
| 284 | ACTCCAAAGTGTCTGAAGTT | −6.1 | −21.7 | 65 | −15.6 | 0 | −4.7 |
| | SEQ. ID. NO:1203 | | | | | | |
| 430 | TTGTTCTGTTAAAACACCAA | −6.1 | −18.7 | 56.9 | −11.7 | −0.7 | −5.5 |
| | SEQ. ID. NO:1204 | | | | | | |
| 471 | TATGGTTCCACTTCCAGGTT | −6.1 | −26.1 | 75.7 | −19.1 | −0.7 | −5.6 |
| | SEQ. ID. NO:1205 | | | | | | |
| 649 | CTCTGCTACCTCAGTTTCTC | −6.1 | −25.9 | 77.8 | −19.3 | −0.2 | −3.6 |
| | SEQ. ID. NO:1206 | | | | | | |
| 822 | CACTTGTACACAGCGTTTTT | −6.1 | −22.8 | 67.1 | −16.7 | 0 | −6.3 |
| | SEQ. ID. NO:1207 | | | | | | |
| 870 | CACTTTCTTCGCATGTACAT | −6.1 | −22.9 | 67.3 | −16.3 | 0 | −7.6 |
| | SEQ. ID. NO:1208 | | | | | | |
| 1023 | CGCAAGTCACGACCTTCACT | −6.1 | −25.9 | 70.9 | −19.8 | 0 | −3.9 |
| | SEQ. ID. NO:1209 | | | | | | |
| 1288 | AGCAATCTGGTCTTCATGGT | −6.1 | −24.4 | 72.9 | −18.3 | 0 | −4.7 |
| | SEQ. ID. NO:1210 | | | | | | |
| 1480 | ATACTCCTCTTGAGTCATTT | −6.1 | −22.6 | 68.9 | −14.8 | −1.7 | −5.8 |
| | SEQ. ID. NO:1211 | | | | | | |
| 1489 | AAGCAGAGCATACTCCTCTT | −6.1 | −24.4 | 71.4 | −17.4 | −0.8 | −6.3 |
| | SEQ. ID. NO:1212 | | | | | | |
| 1528 | TATGTATTGTCTATCTGGAG | −6.1 | −20 | 63.2 | −13.9 | 0 | −3 |
| | SEQ. ID. NO:1213 | | | | | | |
| 1761 | AATCCCCATCACTGCACGTC | −6.1 | −27.7 | 74.8 | −21.6 | 0 | −4.8 |
| | SEQ. ID. NO:1214 | | | | | | |
| 1833 | ACAAGTGAAATAAAGGAAAG | −6.1 | −13.3 | 45.6 | −7.2 | 0 | −2.5 |
| | SEQ. ID. NO:1215 | | | | | | |
| 2022 | TAGAATTGAAGTAACAATCA | −6.1 | −15.1 | 49.8 | −8 | −0.9 | −4.4 |
| | SEQ. ID. NO:1216 | | | | | | |
| 22 | GTCGGGGAGACAATGAGGTG | −6 | −24.4 | 69.9 | −17 | −1.3 | −4.7 |
| | SEQ. ID. NO:1217 | | | | | | |
| 145 | TTGGATTGTTTTGGGTCAGA | −6 | −22.8 | 69 | −16.8 | 0 | −3.4 |
| | SEQ. ID. NO:1218 | | | | | | |
| 320 | TGAAATGCACTTTCTTTATG | −6 | −18.2 | 56.7 | −10.6 | −1.6 | −9.2 |
| | SEQ. ID. NO:1219 | | | | | | |
| 343 | TGATCCCATCCAAATTTTTC | −6 | −22.2 | 64.1 | −16.2 | 0 | −5.4 |
| | SEQ. ID. NO:1220 | | | | | | |
| 467 | GTTCCACTTCCAGGTTCTGT | −6 | −27.7 | 81.3 | −21.2 | −0.2 | −3.8 |
| | SEQ. ID. NO:1221 | | | | | | |
| 654 | GGCATCTCTGCTACCTCAGT | −6 | −28.1 | 81.6 | −19.9 | −2.2 | −7.8 |
| | SEQ. ID. NO:1222 | | | | | | |
| 1025 | GTCGCAAGTCACGACCTTCA | −6 | −26.4 | 73.2 | −18.3 | −2.1 | −6.8 |
| | SEQ. ID. NO:1223 | | | | | | |
| 1331 | CTGAACGAAGGAACATAGCT | −6 | −20.1 | 58.8 | −14.1 | 0 | −4.4 |
| | SEQ. ID. NO:1224 | | | | | | |
| 1334 | CAGCTGAACGAAGGAACATA | −6 | −19.9 | 58.2 | −13.4 | 0 | −7.6 |
| | SEQ. ID. NO:1225 | | | | | | |
| 1398 | CTATTTCGAATTCTTTCTTC | −6 | −19.3 | 60.3 | −12.5 | −0.6 | −6.7 |
| | SEQ. ID. NO:1226 | | | | | | |
| 1486 | CAGAGCATACTCCTCTTGAG | −6 | −23.9 | 70.6 | −16.4 | −1.4 | −6.9 |
| | SEQ. ID. NO:1227 | | | | | | |
| 1531 | CTTTATGTATTGTCTATCTG | −6 | −19.3 | 61.6 | −13.3 | 0 | −0.9 |
| | SEQ. ID. NO:1228 | | | | | | |
| 1663 | GAATGTCCGTAATTCAGTCA | −6 | −22 | 65 | −15.1 | −0.7 | −4.6 |
| | SEQ. ID. NO:1229 | | | | | | |
| 1710 | TGGTCGTTTACTCTCCATGA | −6 | −24.8 | 72.2 | −18.8 | 0 | −4.5 |
| | SEQ. ID. NO:1230 | | | | | | |
| 1849 | TTGAGTGAAACTGGGTACAA | −6 | −19.7 | 59.5 | −12.5 | −1.1 | −6.3 |
| | SEQ. ID. NO:1231 | | | | | | |
| 2101 | AAGATTGAATACAACTCTTT | −6 | −16.4 | 52.7 | −8.5 | −1.9 | −5.4 |
| | SEQ. ID. NO:1232 | | | | | | |
| 75 | GATCCCAGCGATTTTGCTAC | −5.9 | −25.8 | 72 | −18.3 | −1.6 | −6.5 |
| | SEQ. ID. NO:1233 | | | | | | |
| 121 | GACTTTCAAGGCCCTGGGAG | −5.9 | −27.2 | 75.6 | −20.8 | 0 | −8.3 |
| | SEQ. ID. NO:1234 | | | | | | |
| 136 | TTTGGGTCAGAGATGGACTT | −5.9 | −23.1 | 69.3 | −16.6 | −0.3 | −5.3 |
| | SEQ. ID. NO:1235 | | | | | | |
| 157 | TCTTCTACCTCCTTGGATTG | −5.9 | −24.8 | 72.4 | −18.2 | −0.5 | −4.6 |
| | SEQ. ID. NO:1236 | | | | | | |
| 345 | TTTGATCCCATCCAAATTTT | −5.9 | −21.9 | 63 | −15.5 | −0.2 | −5.4 |
| | SEQ. ID. NO:1237 | | | | | | |
| 347 | TTTTTGATCCCATCCAAATT | −5.9 | −21.9 | 63 | −15.3 | −0.5 | −3.8 |
| | SEQ. ID. NO:1238 | | | | | | |
| 476 | GCGAGTATGGTTCCACTTCC | −5.9 | −27.3 | 77.1 | −21.4 | 0 | −5.6 |
| | SEQ. ID. NO:1239 | | | | | | |
| 496 | AAACTGAACATTGCTGTATT | −5.9 | −18.3 | 56.3 | −11.7 | −0.5 | −3.9 |
| | SEQ. ID. NO:1240 | | | | | | |
| 564 | GGCTGCTGGGGGTAGAAACC | −5.9 | −27.7 | 76.5 | −20.5 | −1.2 | −8.5 |
| | SEQ. ID. NO:1241 | | | | | | |
| 627 | TGGTAGAGAGTCTCAGCTGG | −5.9 | −24.8 | 75.4 | −18.1 | −0.3 | −9.2 |
| | SEQ. ID. NO:1242 | | | | | | |
| 781 | ACCTTTACACCCCTCACAGG | −5.9 | −28.2 | 76.3 | −21.8 | −0.2 | −3.6 |
| | SEQ. ID. NO:1243 | | | | | | |
| 796 | GCTTCTCCTGAAGAAACCTT | −5.9 | −23.7 | 67.5 | −15.6 | −2.2 | −5.7 |
| | SEQ. ID. NO:1244 | | | | | | |
| 932 | CAGTTAACAAGCATTCAGCC | −5.9 | −22.7 | 66.3 | −15.8 | −0.9 | −8.7 |
| | SEQ. ID. NO:1245 | | | | | | |
| 1479 | TACTCCTCTTGAGTCATTTT | −5.9 | −22.7 | 69.3 | −15.1 | −1.7 | −5.8 |
| | SEQ. ID. NO:1246 | | | | | | |
| 1509 | GACAGGATAACAATTGCTGT | −5.9 | −20.5 | 61.3 | −13.2 | −1.3 | −8.5 |
| | SEQ. ID. NO:1247 | | | | | | |
| 1532 | CCTTTATGTATTGTCTATCT | −5.9 | −21.3 | 65.7 | −15.4 | 0 | −0.9 |
| | SEQ. ID. NO:1248 | | | | | | |
| 1574 | CATCAAGAAGTGGCTCCTGA | −5.9 | −24 | 69.1 | −18.1 | 0 | −3.7 |
| | SEQ. ID. NO:1249 | | | | | | |
| 1991 | CAAACAGGGCTTGCCAATTA | −5.9 | −23 | 64.8 | −15.3 | −1.8 | −8.5 |
| | SEQ. ID. NO:1250 | | | | | | |
| 2001 | TTTAATTAGGCAAACAGGGC | −5.9 | −20.4 | 60.8 | −14.5 | 0 | −6.9 |
| | SEQ. ID. NO:1251 | | | | | | |
| 2006 | ATCAATTTAATTAGGCAAAC | −5.9 | −15.9 | 51.3 | −10 | 0 | −4.1 |
| | SEQ. ID. NO:1252 | | | | | | |
| 2089 | AACTCTTTAATAAAATATAT | −5.9 | −11.9 | 43.4 | −6 | 0 | −3.9 |
| | SEQ. ID. NO:1253 | | | | | | |
| 2110 | TTTATTGCCAAGATTGAATA | −5.9 | −18.1 | 55.9 | −12.2 | 0 | −3.7 |
| | SEQ. ID. NO:1254 | | | | | | |
| 89 | AGTCTTCCTCTCCAGATCCC | −5.8 | −29.3 | 83.7 | −23.5 | 0 | −4.5 |
| | SEQ. ID. NO:1255 | | | | | | |
| 434 | CCACTTGTTCTGTTAAAACA | −5.8 | −20.3 | 60.6 | −14 | −0.2 | −5.4 |
| | SEQ. ID. NO:1256 | | | | | | |
| 819 | TTGTACACAGCGTTTTTGGT | −5.8 | −23.4 | 69.2 | −17.6 | 0 | −6.2 |
| | SEQ. ID. NO:1257 | | | | | | |
| 935 | TTTCAGTTAACAAGCATTCA | −5.8 | −19.5 | 60.3 | −13.7 | 0 | −6.5 |
| | SEQ. ID. NO:1258 | | | | | | |
| 1151 | TTTTATTTGTTATTTCCTGA | −5.8 | −19.3 | 60.6 | −13.5 | 0 | −1.7 |
| | SEQ. ID. NO:1259 | | | | | | |
| 1834 | TACAAGTGAAATAAAGGAAA | −5.8 | −13 | 45 | −7.2 | 0 | −2.4 |
| | SEQ. ID. NO:1260 | | | | | | |
| 1905 | TTTACAGTTGTGGAAGTTAC | −5.8 | −19.6 | 61.6 | −13.8 | 0 | −3.4 |
| | SEQ. ID. NO:1261 | | | | | | |
| 1921 | TCTATCTAGCCCAATATTTA | −5.8 | −21.4 | 63.9 | −15.6 | 0 | −4.1 |
| | SEQ. ID. NO:1262 | | | | | | |
| 565 | AGGCTGCTGGGGGTAGAAAC | −5.7 | −25.7 | 73.3 | −20 | 0 | −6.1 |
| | SEQ. ID. NO:1263 | | | | | | |
| 1317 | ATAGCTTCAACCGCAGACCC | −5.7 | −27.2 | 73.3 | −20.8 | −0.5 | −4.6 |
| | SEQ. ID. NO:1264 | | | | | | |
| 1756 | CCATCACTGCACGTCCCAGA | −5.7 | −29.3 | 78.1 | −22.9 | −0.5 | −7.5 |
| | SEQ. ID. NO:1265 | | | | | | |
| 2027 | ACAGATAGAATTGAAGTAAC | −5.7 | −15.5 | 50.9 | −9.8 | 0 | −3.1 |
| | SEQ. ID. NO:1266 | | | | | | |
| 2066 | ATATGGTAAGATGAGCAAAA | −5.7 | −16.7 | 52.8 | −11 | 0 | −4.1 |
| | SEQ. ID. NO:1267 | | | | | | |
| 2092 | TACAACTCTTTAATAAAATA | −5.7 | −12.8 | 45.1 | −7.1 | 0 | −3.7 |
| | SEQ. ID. NO:1268 | | | | | | |
| 273 | TCTGAAGTTTCATCTTGAGG | −5.6 | −20.9 | 64.7 | −15.3 | 0 | −4.7 |
| | SEQ. ID. NO:1269 | | | | | | |
| 466 | TTCCACTTCCAGGTTCTGTC | −5.6 | −26.9 | 79.4 | −20.8 | −0.2 | −3.8 |
| | SEQ. ID. NO:1270 | | | | | | |
| 651 | ATCTCTGCTACCTCAGTTTC | −5.6 | −25 | 75.6 | −18.9 | −0.2 | −3.6 |
| | SEQ. ID. NO:1271 | | | | | | |
| 656 | CAGGCATCTCTGCTACCTCA | −5.6 | −27.6 | 79 | −19.8 | −2.2 | −5.6 |
| | SEQ. ID. NO:1272 | | | | | | |
| 732 | CTGTCTCCACAAACAACACA | −5.6 | −22 | 63.2 | −15.9 | −0.1 | −2.9 |
| | SEQ. ID. NO:1273 | | | | | | |
| 936 | ATTTCAGTTAACAAGCATTC | −5.6 | −18.8 | 59 | −13.2 | 0 | −7.3 |
| | SEQ. ID. NO:1274 | | | | | | |
| 967 | ATTTTTTCTCAGTCGCTTAG | −5.6 | −21.8 | 67.1 | −16.2 | 0 | −3.1 |
| | SEQ. ID. NO:1275 | | | | | | |
| 1085 | TCTGTTGATCTGGGGTGAGT | −5.6 | −25.1 | 75.7 | −19.5 | 0 | −4.9 |
| | SEQ. ID. NO:1276 | | | | | | |
| 1086 | GTCTGTTGATCTGGGGTGAG | −5.6 | −25.1 | 75.7 | −19.5 | 0 | −4.9 |
| | SEQ. ID. NO:1277 | | | | | | |
| 1401 | CCACTATTTCGAATTCTTTC | −5.6 | −20.8 | 62.2 | −15.2 | 0 | −6.7 |
| | SEQ. ID. NO:1278 | | | | | | |
| 1510 | AGACAGGATAACAATTGCTG | −5.6 | −19.3 | 58.5 | −13.2 | −0.2 | −7 |
| | SEQ. ID. NO:1279 | | | | | | |
| 2051 | CAAAATGAGATTTTCCCTAG | −5.6 | −19.1 | 57.4 | −12.5 | −0.9 | −4.8 |
| | SEQ. ID. NO:1280 | | | | | | |
| 2056 | ATGAGCAAAATGAGATTTTC | −5.6 | −16.9 | 53.7 | −10.3 | −0.9 | −4.8 |
| | SEQ. ID. NO:1281 | | | | | | |
| 2072 | TATGCAATATGGTAAGATGA | −5.6 | −17.8 | 55.6 | −12.2 | 0 | −5.6 |
| | SEQ. ID. NO:1282 | | | | | | |
| 160 | ATGTCTTCTACCTCCTTGGA | −5.5 | −25.9 | 75.5 | −19.7 | −0.5 | −4.3 |
| | SEQ. ID. NO:1283 | | | | | | |
| 344 | TTGATCCCATCCAAATTTTT | −5.5 | −21.9 | 63 | −16.4 | 0 | −5.4 |
| | SEQ. ID. NO:1284 | | | | | | |
| 346 | TTTTGATCCCATCCAAATTT | −5.5 | −21.9 | 63 | −15.7 | −0.5 | −4.3 |
| | SEQ. ID. NO:1285 | | | | | | |
| 470 | ATGGTTCCACTTCCAGGTTC | −5.5 | −26.8 | 78.1 | −20.4 | −0.7 | −5.6 |
| | SEQ. ID. NO:1286 | | | | | | |
| 491 | GAACATTGCTGTATTGCGAG | −5.5 | −21.8 | 63.8 | −15.4 | −0.7 | −5 |
| | SEQ. ID. NO:1287 | | | | | | |
| 520 | GGAAATCTGTGGTTGAACTT | −5.5 | −20.5 | 61.7 | −15 | 0 | −3.4 |
| | SEQ. ID. NO:1288 | | | | | | |
| 630 | CCCTGGTAGAGAGTCTCAGC | −5.5 | −27.6 | 80.6 | −20.7 | −1.1 | −10 |
| | SEQ. ID. NO:1289 | | | | | | |
| 869 | ACTTTCTTCGCATGTACATA | −5.5 | −21.9 | 65.5 | −15.9 | 0 | −8 |
| | SEQ. ID. NO:1290 | | | | | | |
| 925 | CAAGCATTCAGCCAACATTC | −5.5 | −22.9 | 66.1 | −16.4 | −0.9 | −4.1 |
| | SEQ. ID. NO:1291 | | | | | | |
| 1116 | TTATATGAATCCATAATAAA | −5.5 | −13.8 | 46.8 | −7.2 | −1 | −3.9 |
| | SEQ. ID. NO:1292 | | | | | | |
| 1315 | AGCTTCAACCGCAGACCCTT | −5.5 | −28.5 | 76 | −22.3 | −0.5 | −4.3 |
| | SEQ. ID. NO:1293 | | | | | | |
| 1422 | GTTATATATTCATCAGAGAT | −5.5 | −17.9 | 57.8 | −12.4 | 0 | −3.9 |
| | SEQ. ID. NO:1294 | | | | | | |
| 1748 | GCACGTCCCAGATTTCACAG | −5.5 | −26.6 | 74.1 | −21.1 | 0 | −4.6 |
| | SEQ. ID. NO:1295 | | | | | | |
| 1970 | AATGCAGGATTCCCTGGAGC | −5.5 | −26.6 | 74.2 | −18.1 | −3 | −8.7 |
| | SEQ. ID. NO:1296 | | | | | | |
| 2090 | CAACTCTTTAATAAAATATA | −5.5 | −12.6 | 44.7 | −7.1 | 0 | −3.7 |
| | SEQ. ID. NO:1297 | | | | | | |
| 276 | GTGTCTGAAGTTTCATCTTG | −5.4 | −21.5 | 67.1 | −16.1 | 0 | −4.5 |
| | SEQ. ID. NO:1298 | | | | | | |
| 341 | ATCCCATCCAAATTTTTCAA | −5.4 | −21.6 | 62.1 | −16.2 | 0 | −4.6 |
| | SEQ. ID. NO:1299 | | | | | | |
| 356 | TGAGATTCATTTTTGATCCC | −5.4 | −21.8 | 65.1 | −15.5 | −0.8 | −4.5 |
| | SEQ. ID. NO:1300 | | | | | | |
| 468 | GGTTCCACTTCCAGGTTCTG | −5.4 | −27.7 | 80.3 | −22.3 | 0 | −3.6 |
| | SEQ. ID. NO:1301 | | | | | | |
| 791 | TCCTGAAGAAACCTTTACAC | −5.4 | −20.5 | 60.2 | −15.1 | 0 | −2.8 |
| | SEQ. ID. NO:1302 | | | | | | |
| 1237 | GAATTCTACAAGAACCTGTA | −5.4 | −19.1 | 58.1 | −12.7 | −0.9 | −6.8 |
| | SEQ. ID. NO:1303 | | | | | | |
| 1436 | AACTAAACATAGGTGTTATA | −5.4 | −16.4 | 52.9 | −9.7 | −1.2 | −5.3 |
| | SEQ. ID. NO:1304 | | | | | | |
| 1568 | GAAGTGGCTCCTGAAGCTTC | −5.4 | −25.4 | 73.5 | −17.9 | −2.1 | −9.8 |
| | SEQ. ID. NO:1305 | | | | | | |
| 1740 | CAGATTTCACAGAGAAGTGG | −5.4 | −20.4 | 62.3 | −14.1 | −0.7 | −4.6 |
| | SEQ. ID. NO:1306 | | | | | | |
| 1749 | TGCACGTCCCAGATTTCACA | −5.4 | −26.6 | 73.6 | −21.2 | 0 | −4.7 |
| | SEQ. ID. NO:1307 | | | | | | |
| 1760 | ATCCCCATCACTGCACGTCC | −5.4 | −30.4 | 80.5 | −25 | 0 | −4.8 |
| | SEQ. ID. NO:1308 | | | | | | |
| 1865 | TATTCATCAAGATTTCTTGA | −5.4 | −18.1 | 57.7 | −10.5 | −2.2 | −10.9 |
| | SEQ. ID. NO:1309 | | | | | | |
| 2112 | GCTTTATTGCCAAGATTGAA | −5.4 | −21.1 | 62.2 | −15.7 | 0 | −3.7 |
| | SEQ. ID. NO:1310 | | | | | | |
| 230 | AACTAAGAGAAGCAGTGTTC | −5.3 | −19.3 | 60 | −14 | 0 | −5.5 |
| | SEQ. ID. NO:1311 | | | | | | |
| 305 | TTATGGTGGTCTTCAAAAAA | −5.3 | −17.6 | 55 | −12.3 | 0 | −3.3 |
| | SEQ. ID. NO:1312 | | | | | | |
| 715 | ACACAGCTCATCCCCTTTGA | −5.3 | −27.7 | 76.7 | −22.4 | 0 | −4.4 |
| | SEQ. ID. NO:1313 | | | | | | |
| 823 | ACACTTGTACACAGCGTTTT | −5.3 | −22.9 | 67.3 | −17.6 | 0 | −6.3 |
| | SEQ. ID. NO:1314 | | | | | | |
| 1084 | CTGTTGATCTGGGGTGAGTT | −5.3 | −24.8 | 74.3 | −19.5 | 0 | −4.2 |
| | SEQ. ID. NO:1315 | | | | | | |
| 1097 | AATGTAGAAGAGTCTGTTGA | −5.3 | −19.1 | 60.2 | −13.8 | 0.1 | −5.8 |
| | SEQ. ID. NO:1316 | | | | | | |
| 1611 | TTTTCAGGCTGGTGAATCTT | −5.3 | −23 | 69 | −17 | −0.5 | −5.7 |
| | SEQ. ID. NO:1317 | | | | | | |
| 1729 | GAGAAGTGGGGTAAACTTGT | −5.3 | −21.2 | 63.6 | −14.9 | −0.9 | −4.1 |
| | SEQ. ID. NO:1318 | | | | | | |
| 137 | TTTTGGGTCAGAGATGGACT | −5.2 | −23.1 | 69.3 | −16.7 | −1.1 | −5.3 |
| | SEQ. ID. NO:1319 | | | | | | |
| 208 | TTTGAGCTATGTTTCTAAGT | −5.2 | −20.1 | 63.1 | −14.9 | 0 | −5.1 |
| | SEQ. ID. NO:1320 | | | | | | |
| 433 | CACTTGTTCTGTTAAAACAC | −5.2 | −18.5 | 57.5 | −12.4 | −0.7 | −5.5 |
| | SEQ. ID. NO:1321 | | | | | | |
| 587 | TTCCAGGAGAGTACCACTCT | −5.2 | −25.8 | 74.9 | −18.1 | −2.5 | −9.1 |
| | SEQ. ID. NO:1322 | | | | | | |
| 872 | GACACTTTCTTCGCATGTAC | −5.2 | −23 | 68.1 | −17.8 | 0 | −4.8 |
| | SEQ. ID. NO:1323 | | | | | | |
| 955 | TCGCTTAGATTTACACTGAA | −5.2 | −20.1 | 60.5 | −14.9 | 0 | −3.1 |
| | SEQ. ID. NO:1324 | | | | | | |
| 1081 | TTGATCTGGGGTGAGTTCAG | −5.2 | −23.8 | 72 | −18.6 | 0 | −4.9 |
| | SEQ. ID. NO:1325 | | | | | | |
| 1104 | ATAATAAAATGTAGAAGAGT | −5.2 | −13.2 | 46 | −8 | 0 | −1.2 |
| | SEQ. ID. NO:1326 | | | | | | |
| 1360 | AGACGGAAGTTTCTTATTGA | −5.2 | −19.9 | 60.7 | −13.8 | −0.8 | −5.7 |
| | SEQ. ID. NO:1327 | | | | | | |
| 1607 | CAGGCTGGTGAATCTTACAC | −5.2 | −23.1 | 68.1 | −17.2 | −0.5 | −4.9 |
| | SEQ. ID. NO:1328 | | | | | | |
| 1608 | TCAGGCTGGTCAATCTTACA | −5.2 | −23.3 | 69.1 | −18.1 | 0 | −4.3 |
| | SEQ. ID. NO:1329 | | | | | | |
| 1992 | GCAAACAGGGCTTGCCAATT | −5.2 | −25.1 | 69.2 | −18.1 | −1.8 | −8.5 |
| | SEQ. ID. NO:1330 | | | | | | |
| 2005 | TCAATTTAATTAGGCAAACA | −5.2 | −16.6 | 52.6 | −11.4 | 0 | −4.1 |
| | SEQ. ID. NO:1331 | | | | | | |
| 54 | AATGCTCAGAATCCAATTTC | −5.1 | −20 | 60.2 | −14.9 | 0 | −3.6 |
| | SEQ. ID. NO:1332 | | | | | | |
| 197 | TTTCTAAGTCTTCTTTTCTT | −5.1 | −20.1 | 64.5 | −15 | 0 | −2.7 |
| | SEQ. ID. NO:1333 | | | | | | |
| 238 | ATCCAGGAAACTAAGAGAAG | −5.1 | −18.1 | 55.6 | −12.4 | −0.3 | −5.7 |
| | SEQ. ID. NO:1334 | | | | | | |
| 393 | GAAAATTCATCTGTGGTAGG | −5.1 | −19.5 | 59.9 | −14.4 | 0 | −4.1 |
| | SEQ. ID. NO:1335 | | | | | | |
| 595 | TTCATATATTCCAGGAGAGT | −5.1 | −21.4 | 65.5 | −16.3 | 0 | −5.3 |
| | SEQ. ID. NO:1336 | | | | | | |
| 596 | GTTCATATATTCCAGGAGAG | −5.1 | −21.4 | 65.5 | −16.3 | 0 | −5.3 |
| | SEQ. ID. NO:1337 | | | | | | |
| 831 | CCGTTTTTACACTTGTACAC | −5.1 | −22.2 | 65.2 | −16.4 | −0.4 | −6.6 |
| | SEQ. ID. NO:1338 | | | | | | |
| 950 | TAGATTTACACTGAATTTCA | −5.1 | −17.4 | 55.5 | −12.3 | 0 | −5.7 |
| | SEQ. ID. NO:1339 | | | | | | |
| 1026 | TGTCGCAAGTCACGACCTTC | −5.1 | −25.7 | 71.9 | −17.8 | −2.8 | −7.8 |
| | SEQ. ID. NO:1340 | | | | | | |
| 1027 | TTGTCGCAAGTCACGACCTT | −5.1 | −25.4 | 70.7 | −17.5 | −2.8 | −7.8 |
| | SEQ. ID. NO:1341 | | | | | | |
| 1108 | ATCCATAATAAAATGTAGAA | −5.1 | −14.5 | 48.1 | −9.4 | 0 | −2.8 |
| | SEQ. ID. NO:1342 | | | | | | |
| 1235 | ATTCTACAAGAACCTGTACA | −5.1 | −20.1 | 60.5 | −14 | −0.9 | −7.6 |
| | SEQ. ID. NO:1343 | | | | | | |
| 1323 | AGGAACATAGCTTCAACCGC | −5.1 | −23.7 | 66.7 | −18.1 | −0.2 | −4.6 |
| | SEQ. ID. NO:1344 | | | | | | |
| 1399 | ACTATTTCGAATTCTTTCTT | −5.1 | −19.1 | 59.5 | −13.2 | −0.6 | −6.4 |
| | SEQ. ID. NO:1345 | | | | | | |
| 1478 | ACTCCTCTTGAGTCATTTTC | −5.1 | −23.4 | 71.7 | −16.8 | −1.4 | −5.8 |
| | SEQ. ID. NO:1346 | | | | | | |
| 1490 | TAAGCAGAGCATACTCCTCT | −5.1 | −24 | 70.4 | −17.4 | −1.4 | −6.3 |
| | SEQ. ID. NO:1347 | | | | | | |
| 1570 | AAGAAGTGGCTCCTGAAGCT | −5.1 | −24.2 | 9.4 | −17 | −2.1 | −6.3 |
| | SEQ. ID. NO:1348 | | | | | | |
| 2000 | TTAATTAGGCAAACAGGGCT | −5.1 | −21.2 | 62.3 | −15.4 | −0.5 | −7.1 |
| | SEQ. ID. NO:1349 | | | | | | |
| 2069 | GCAATATGGTAAGATGAGCA | −5.1 | −20.6 | 61.6 | −15.5 | 0 | −4.2 |
| | SEQ. ID. NO:1350 | | | | | | |
| 2111 | CTTTATTGCCAAGATTGAAT | −5.1 | −19.3 | 58.3 | −14.2 | 0 | −3.7 |
| | SEQ. ID. NO:1351 | | | | | | |
| 109 | CCTGGGAGGATTCTGGACTG | −5 | −26 | 73.9 | −20.5 | −0.1 | −3.6 |
| | SEQ. ID. NO:1352 | | | | | | |
| 177 | CTTTCACTCCTTCTACGATG | −5 | −23.3 | 68 | −18.3 | 0 | −3.5 |
| | SEQ. ID. NO:1353 | | | | | | |
| 563 | GCTGCTGGGGGTAGAAACCC | −5 | −28.5 | 77.5 | −20.5 | −3 | −11.2 |
| | SEQ. ID. NO:1354 | | | | | | |
| 582 | GGAGAGTACCACTCTTCAGG | −5 | −25 | 73.9 | −17.3 | −2.7 | −8.6 |
| | SEQ. ID. NO:1355 | | | | | | |
| 586 | TCCAGGAGAGTACCACTCTT | −5 | −25.8 | 74.9 | −18.1 | −2.7 | −8.3 |
| | SEQ. ID. NO:1356 | | | | | | |
| 655 | AGGCATCTCTGCTACCTCAG | −5 | −26.9 | 78.2 | −19.7 | −2.2 | −5.6 |
| | SEQ. ID. NO:1357 | | | | | | |
| 854 | ACATATCCATCACACAGTTG | −5 | −21.9 | 65.1 | −16.9 | 0 | −2.6 |
| | SEQ. ID. NO:1358 | | | | | | |
| 866 | TTCTTCGCATGTACATATCC | −5 | −23.1 | 67.9 | −17.6 | 0 | −8 |
| | SEQ. ID. NO:1359 | | | | | | |
| 1150 | TTTATTTGTTATTTCCTGAG | −5 | −19.2 | 60.5 | −14.2 | 0 | −1.9 |
| | SEQ. ID. NO:1360 | | | | | | |
| 1161 | TCTTTTAAAATTTTATTTGT | −5 | −14.6 | 49.6 | −9.1 | −0.2 | −7.7 |
| | SEQ. ID. NO:1361 | | | | | | |
| 1266 | AAAGTCTGAAATCCTGGTAG | −5 | −19.7 | 59.7 | −14.7 | 0 | −4.6 |
| | SEQ. ID. NO:1362 | | | | | | |
| 1640 | GACCCAGGAGACAGGCAAAG | −5 | −25.1 | 69.5 | −20.1 | 0 | −4 |
| | SEQ. ID. NO:1363 | | | | | | |
| 1819 | GGAAAGTTATACATCAGATT | −5 | −17.8 | 56.2 | −12.8 | 0 | −3.4 |
| | SEQ. ID. NO:1364 | | | | | | |
| 1866 | ATATTCATCAAGATTTCTTG | −5 | −17.5 | 56.3 | −11.4 | −1 | −8.5 |
| | SEQ. ID. NO:1365 | | | | | | |
| 2040 | TTTCCCTAGTTCAACAGATA | −5 | −22.1 | 65.8 | −17.1 | 0 | −3.5 |
| | SEQ. ID. NO:1366 | | | | | | |
| 2096 | TGAATACAACTCTTTAATAA | −5 | −14.4 | 48.4 | −9.4 | 0 | −2.5 |
| | SEQ. ID. NO:1367 | | | | | | |
| 88 | GTCTTCCTCTCCAGATCCCA | −4.9 | −30 | 84.3 | −25.1 | 0 | −4.5 |
| | SEQ. ID. NO:1368 | | | | | | |
| 233 | GGAAACTAAGAGAAGCAGTG | −4.9 | −18.7 | 57.2 | −13.8 | 0 | −4.1 |
| | SEQ. ID. NO:1369 | | | | | | |
| 300 | GTGGTCTTCAAAAAAAACTC | −4.9 | −16.7 | 52.9 | −11.8 | 0 | −2.5 |
| | SEQ. ID. NO:1370 | | | | | | |
| 325 | TCAATTGAAATGCACTTTCT | −4.9 | −18.8 | 57.6 | −12.3 | −1.6 | −9.2 |
| | SEQ. ID. NO:1371 | | | | | | |
| 456 | AGGTTCTGTCCCAGAGGACC | −4.9 | −28.7 | 81.6 | −20.8 | −3 | −9.7 |
| | SEQ. ID. NO:1372 | | | | | | |
| 597 | AGTTCATATATTCCAGGAGA | −4.9 | −21.4 | 65.5 | −16.5 | 0 | −5.3 |
| | SEQ. ID. NO:1373 | | | | | | |
| 625 | GTAGAGAGTCTCAGCTGGCA | −4.9 | −26.1 | 78.9 | −19.8 | −1.1 | −10 |
| | SEQ. ID. NO:1374 | | | | | | |
| 1397 | TATTTCGAATTCTTTCTTCC | −4.9 | −20.4 | 62.2 | −14.7 | −0.6 | −6.7 |
| | SEQ. ID. NO:1375 | | | | | | |
| 1400 | CACTATTTCGAATTCTTTCT | −4.9 | −19.7 | 60.4 | −14 | −0.6 | −6.7 |
| | SEQ. ID. NO:1376 | | | | | | |
| 1487 | GCAGAGCATACTCCTCTTGA | −4.9 | −25.7 | 74.8 | −19.3 | −1.4 | −5.8 |
| | SEQ. ID. NO:1377 | | | | | | |
| 1695 | CATGACATCAGCATCTCAGC | −4.9 | −24 | 70.9 | −19.1 | 0 | −4.1 |
| | SEQ. ID. NO:1378 | | | | | | |
| 1888 | TACAGATGTAATTACAACAT | −4.9 | −16.6 | 52.8 | −10.5 | −0.2 | −10.3 |
| | SEQ. ID. NO:1379 | | | | | | |
| 1934 | TAGAGAAAGTTGTTCTATCT | −4.9 | −18.4 | 59 | −12 | −1.4 | −5.6 |
| | SEQ. ID. NO:1380 | | | | | | |
| 2067 | AATATGGTAAGATGAGCAAA | −4.9 | −16.7 | 52.8 | −11.8 | 0 | −4.1 |
| | SEQ. ID. NO:1381 | | | | | | |
| 2073 | ATATGCAATATGGTAAGATG | −4.9 | −17.2 | 54.3 | −11.8 | −0.2 | −5.6 |
| | SEQ. ID. NO:1382 | | | | | | |
| 2084 | TTTAATAAAATATATGCAAT | −4.9 | −12 | 43.4 | −7.1 | 0 | −5.6 |
| | SEQ. ID. NO:1383 | | | | | | |
| 2114 | TTGCTTTATTGCCAAGATTG | −4.9 | −21.3 | 63.2 | −16.4 | 0 | −3.6 |
| | SEQ. ID. NO:1384 | | | | | | |
| 21 | TCGGGGAGACAATGAGGTGA | −4.8 | −23.8 | 68 | −19 | 0 | −3.1 |
| | SEQ. ID. NO:1385 | | | | | | |
| 135 | TTGGGTCAGAGATGGACTTT | −4.8 | −23.1 | 69.3 | −17.1 | −1.1 | −5.3 |
| | SEQ. ID. NO:1386 | | | | | | |
| 271 | TGAAGTTTCATCTTGAGGAA | −4.8 | −19.5 | 60.4 | −14.7 | 0 | −5.3 |
| | SEQ. ID. NO:1387 | | | | | | |
| 348 | ATTTTTGATCCCATCCAAAT | −4.8 | −21.8 | 62.7 | −16.3 | −0.5 | −4.3 |
| | SEQ. ID. NO:1388 | | | | | | |
| 377 | TAGGTAAATGGGAATGTTCA | −4.8 | −19.1 | 58.6 | −14.3 | 0 | −5.7 |
| | SEQ. ID. NO:1389 | | | | | | |
| 854 | CGCTTAGATTTACACTGAAT | −4.8 | −19.7 | 59.2 | −14.9 | 0 | −3.1 |
| | SEQ. ID. NO:1390 | | | | | | |
| 1092 | AGAAGAGTCTGTTGATCTGG | −4.8 | −21.4 | 66.1 | −16.1 | −0.1 | −5.8 |
| | SEQ. ID. NO:1391 | | | | | | |
| 1402 | ACCACTATTTCGAATTCTTT | −4.8 | −20.6 | 61.4 | −15.8 | 0 | −6.7 |
| | SEQ. ID. NO:1392 | | | | | | |
| 195 | TCTAAGTCTTCTTTTCTTCT | −4.7 | −21.2 | 67.6 | −15.9 | −0.3 | −3 |
| | SEQ. ID. NO:1393 | | | | | | |
| 282 | TCCAAAGTGTCTGAAGTTTC | −4.7 | −21.1 | 64.3 | −16.4 | 0 | −3 |
| | SEQ. ID. NO:1394 | | | | | | |
| 479 | ATTGCGAGTATGGTTCCACT | −4.7 | −24.9 | 71.6 | −20.2 | 0 | −5.6 |
| | SEQ. ID. NO:1395 | | | | | | |
| 1077 | TCTGGGGTGAGTTCAGTTTT | −4.7 | −24.6 | 75.3 | −19.4 | −0.2 | −3.7 |
| | SEQ. ID. NO:1396 | | | | | | |
| 1604 | GCTGGTGAATCTTACACAAC | −4.7 | −21.4 | 63.6 | −15.1 | −1.6 | −5 |
| | SEQ. ID. NO:1397 | | | | | | |
| 1786 | AAAAGGAGCTAGACCCCTCC | −4.7 | −26.2 | 71.1 | −19.9 | −1.6 | −7.2 |
| | SEQ. ID. NO:1398 | | | | | | |
| 1838 | TGGGTACAAGTGAAATAAAG | −4.7 | −16.2 | 51.7 | −11.5 | 0 | −5.2 |
| | SEQ. ID. NO:1399 | | | | | | |
| 2044 | TAACAATCAATTTAATTAGG | −4.7 | −13.8 | 47.1 | −9.1 | 0 | −4.1 |
| | SEQ. ID. NO:1400 | | | | | | |
| 81 | TCTCCAGATCCCAGCGATTT | −4.6 | −27.5 | 75.7 | −22.9 | 0 | −4.5 |
| | SEQ. ID. NO:1401 | | | | | | |
| 264 | TCATCTTGAGGAAATGTCCA | −4.6 | −21.8 | 64.6 | −15.1 | −2.1 | −5.7 |
| | SEQ. ID. NO:1402 | | | | | | |
| 521 | AGGAAATCTGTGGTTGAACT | −4.6 | −20.4 | 61.6 | −15.8 | 0 | −3.4 |
| | SEQ. ID. NO:1403 | | | | | | |
| 1176 | TCTGCACTGAATTCTTCTTT | −4.6 | −21.8 | 66.3 | −16.5 | −0.4 | −6.9 |
| | SEQ. ID. NO:1404 | | | | | | |
| 1177 | TTCTGCACTGAATTCTTCTT | −4.6 | −21.8 | 66.3 | −16.5 | −0.4 | −6.9 |
| | SEQ. ID. NO:1405 | | | | | | |
| 1330 | TGAACGAAGGAACATAGCTT | −4.6 | −19.3 | 57.4 | −14.7 | 0 | −4.6 |
| | SEQ. ID. NO:1406 | | | | | | |
| 1472 | CTTGAGTCATTTTCAGTTCC | −4.6 | −23 | 70.6 | −18.4 | 0 | −5.8 |
| | SEQ. ID. NO:1407 | | | | | | |
| 1916 | CTAGCCCAATATTTACAGTT | −4.6 | −22.2 | 65.1 | −17.6 | 0 | −4.1 |
| | SEQ. ID. NO:1408 | | | | | | |
| 2078 | AAAATATATGCAATATGGTA | −4.6 | −14.9 | 49.1 | −9.8 | −0.2 | −6.5 |
| | SEQ. ID. NO:1409 | | | | | | |
| 2086 | TCTTTAATAAAATATATGCA | −4.6 | −14 | 47.6 | −9.4 | 0 | −5.2 |
| | SEQ. ID. NO:1410 | | | | | | |
| 241 | GAAATCCAGGAAACTAAGAG | −4.5 | −17.4 | 53.7 | −12.3 | −0.3 | −5.7 |
| | SEQ. ID. NO:1411 | | | | | | |
| 340 | TCCCATCCAAATTTTTCAAT | −4.5 | −21.6 | 62.1 | −17.1 | 0 | −4.6 |
| | SEQ. ID. NO:1412 | | | | | | |
| 381 | GTGGTAGGTAAATGGGAATG | −4.5 | −20.3 | 61.1 | −15.8 | 0 | −1.2 |
| | SEQ. ID. NO:1413 | | | | | | |
| 474 | GAGTATGGTTCCACTTCCAG | −4.5 | −25.4 | 74.3 | −20.4 | −0.2 | −5.1 |
| | SEQ. ID. NO:1414 | | | | | | |
| 868 | CTTTCTTCGCATGTACATAT | −4.5 | −21.7 | 64.9 | −16.7 | 0 | −8 |
| | SEQ. ID. NO:1415 | | | | | | |
| 871 | ACACTTTCTTCGCATGTACA | −4.5 | −23.1 | 67.9 | −18.6 | 0 | −6.4 |
| | SEQ. ID. NO:1416 | | | | | | |
| 1087 | AGTCTGTTGATCTGGGGTGA | −4.5 | −25.1 | 75.7 | −20.6 | 0 | −4.9 |
| | SEQ. ID. NO:1417 | | | | | | |
| 1322 | GGAACATAGCTTCAACCGCA | −4.5 | −24.4 | 67.6 | −19.2 | −0.5 | −4.6 |
| | SEQ. ID. NO:1418 | | | | | | |
| 1527 | ATGTATTGTCTATCTGGAGA | −4.5 | −20.9 | 65.2 | −16.4 | 0 | −3.3 |
| | SEQ. ID. NO:1419 | | | | | | |
| 1551 | TTCTCTACTGCCTCTCTATC | −4.5 | −24.9 | 75.4 | −20.4 | 0 | −3 |
| | SEQ. ID. NO:1420 | | | | | | |
| 1750 | CTGCACGTCCCAGATTTCAC | −4.5 | −26.8 | 74.4 | −22.3 | 0 | −6 |
| | SEQ. ID. NO:1421 | | | | | | |
| 2036 | CCTAGTTCAACAGATAGAAT | −4.5 | −19.4 | 59.3 | −14.9 | 0 | −3.7 |
| | SEQ. ID. NO:1422 | | | | | | |
| 2083 | TTAATAAAATATATGCAATA | −4.5 | −11.6 | 42.6 | −7.1 | 0 | −5.6 |
| | SEQ. ID. NO:1423 | | | | | | |
| 31 | TTAGGATAAGTCGGGGAGAC | −4.4 | −22 | 65.2 | −16.5 | −1 | −4.7 |
| | SEQ. ID. NO:1424 | | | | | | |
| 156 | CTTCTACCTCCTTGGATTGT | −4.4 | −25.6 | 74.1 | −20.5 | −0.5 | −4.6 |
| | SEQ. ID. NO:1425 | | | | | | |
| 480 | TATTGCGAGTATGGTTCCAC | −4.4 | −23.7 | 69 | −19.3 | 0 | −5.6 |
| | SEQ. ID. NO:1426 | | | | | | |
| 1028 | CTTGTCGCAAGTCACGACCT | −4.4 | −26.2 | 72.2 | −19 | −2.8 | −8 |
| | SEQ. ID. NO:1427 | | | | | | |
| 1244 | TTTTTGTGAATTCTACAAGA | −4.4 | −17.4 | 55.6 | −11.6 | −0.7 | −10.5 |
| | SEQ. ID. NO:1428 | | | | | | |
| 1318 | CATAGCTTCAACCGCAGACC | −4.4 | −25.9 | 71 | −20.8 | −0.5 | −4.6 |
| | SEQ. ID. NO:1429 | | | | | | |
| 1359 | GACGGAAGTTTCTTATTGAA | −4.4 | −19.2 | 58.6 | −13.9 | −0.8 | −5.7 |
| | SEQ. ID. NO:1430 | | | | | | |
| 1744 | GTCCCAGATTTCACAGAGAA | −4.4 | −23.6 | 68.7 | −18.7 | −0.1 | −4.4 |
| | SEQ. ID. NO:1431 | | | | | | |
| 1820 | AGGAAAGTTATACATCAGAT | −4.4 | −17.7 | 56.1 | −13.3 | 0 | −3.3 |
| | SEQ. ID. NO:1432 | | | | | | |
| 1867 | AATATTCATCAAGATTTCTT | −4.4 | −16.8 | 54.4 | −12.4 | 0 | −4.7 |
| | SEQ. ID. NO:1433 | | | | | | |
| 2079 | TAAAATATATGCAATATGGT | −4.4 | −14.9 | 49.1 | −9.8 | −0.5 | −6.5 |
| | SEQ. ID. NO:1434 | | | | | | |
| 390 | AATTCATCTGTGGTAGGTAA | −4.3 | −20.5 | 63.3 | −16.2 | 0 | −2.8 |
| | SEQ. ID. NO:1435 | | | | | | |
| 769 | CTCACAGGTCAGTGCATTAT | −4.3 | −23.9 | 71.7 | −18.9 | −0.5 | −5.4 |
| | SEQ. ID. NO:1436 | | | | | | |
| 818 | TGTACACAGCGTTTTTGGTA | −4.3 | −23 | 68.2 | −18.7 | 0 | −5.9 |
| | SEQ. ID. NO:1437 | | | | | | |
| 861 | CGCATGTACATATCCATCAC | −4.3 | −23.2 | 66.6 | −18.4 | 0 | −8 |
| | SEQ. ID. NO:1438 | | | | | | |
| 948 | GATTTACACTGAATTTCAGT | −4.3 | −18.9 | 59.1 | −12.3 | −2.3 | −11 |
| | SEQ. ID. NO:1439 | | | | | | |
| 1175 | CTGCACTGAATTCTTCTTTT | −4.3 | −21.5 | 65.1 | −16.5 | −0.4 | −6.9 |
| | SEQ. ID. NO:1440 | | | | | | |
| 1410 | TCAGAGATACCACTATTTCG | −4.3 | −21.1 | 62.9 | −16.1 | −0.5 | −3.6 |
| | SEQ. ID. NO:1441 | | | | | | |
| 1467 | GTCATTTTCAGTTCCCCAAT | −4.3 | −25.4 | 72.9 | −21.1 | 0 | −1.5 |
| | SEQ. ID. NO:1442 | | | | | | |
| 1468 | AGTCATTTTCAGTTCCCCAA | −4.3 | −25.4 | 73.2 | −21.1 | 0 | −0.9 |
| | SEQ. ID. NO:1443 | | | | | | |
| 1501 | AACAATTGCTGTAAGCAGAG | −4.3 | −19.6 | 59.4 | −12.2 | −3.1 | −9.1 |
| | SEQ. ID. NO:1444 | | | | | | |
| 1856 | AGATTTCTTGAGTGAAACTG | −4.3 | −18.3 | 57.6 | −12.8 | −1.1 | −5.5 |
| | SEQ. ID. NO:1445 | | | | | | |
| 1969 | ATGCAGGATTCCCTGGAGCC | −4.3 | −29.3 | 80.2 | −22 | −3 | −9.1 |
| | SEQ. ID. NO:1446 | | | | | | |
| 2037 | CCCTAGTTCAACAGATAGAA | −4.3 | −21.4 | 63 | −17.1 | 0 | −3.7 |
| | SEQ. ID. NO:1447 | | | | | | |
| 2102 | CAAGATTGAATACAACTCTT | −4.3 | −17 | 53.7 | −10.8 | −1.9 | −5.4 |
| | SEQ. ID. NO:1448 | | | | | | |
| 25 | TAAGTCGGGGAGACAATGAG | −4.2 | −21 | 61.9 | −14.7 | −2.1 | −4.9 |
| | SEQ. ID. NO:1449 | | | | | | |
| 181 | TCTTCTTTCACTCCTTCTAC | −4.2 | −23.7 | 72.5 | −19.5 | 0 | −0.2 |
| | SEQ. ID. NO:1450 | | | | | | |
| 368 | GGGAATGTTCAATGAGATTC | −4.2 | −19.7 | 60.5 | −15.5 | 0.2 | −6.4 |
| | SEQ. ID. NO:1451 | | | | | | |
| 465 | TCCACTTCCAGGTTCTGTCC | −4.2 | −28.8 | 82.7 | −24.1 | −0.2 | −3.8 |
| | SEQ. ID. NO:1452 | | | | | | |
| 1411 | ATCAGAGATACCACTATTTC | −4.2 | −20.3 | 62.4 | −16.1 | 0 | −3.3 |
| | SEQ. ID. NO:1453 | | | | | | |
| 1706 | CGTTTACTCTCCATGACATC | −4.2 | −23.3 | 68.1 | −19.1 | 0 | −4.5 |
| | SEQ. ID. NO:1454 | | | | | | |
| 1999 | TAATTAGGCAAACAGGGCTT | −4.2 | −21.2 | 62.3 | −16.3 | −0.5 | −6.1 |
| | SEQ. ID. NO:1455 | | | | | | |
| 2033 | AGTTCAACAGATAGAATTGA | −4.2 | −17.5 | 55.6 | −12.6 | −0.4 | −4.2 |
| | SEQ. ID. NO:1456 | | | | | | |
| 2070 | TGCAATATGGTAAGATGAGC | −4.2 | −19.9 | 60.3 | −15.7 | 0 | −4.7 |
| | SEQ. ID. NO:1457 | | | | | | |
| 134 | TGGGTCAGAGATGGACTTTC | −4.1 | −23.4 | 70.6 | −18.1 | −1.1 | −5 |
| | SEQ. ID. NO:1458 | | | | | | |
| 186 | TCTTTTCTTCTTTCACTCCT | −4.1 | −24 | 73.3 | −19.9 | 0 | 0 |
| | SEQ. ID. NO:1459 | | | | | | |
| 534 | TAATAGGATGACGAGGAAAT | −4.1 | −17.1 | 53 | −13 | 0 | −3.5 |
| | SEQ. ID. NO:1460 | | | | | | |
| 535 | ATAATAGGATGACGAGGAAA | −4.1 | −17.1 | 53 | −13 | 0 | −3.5 |
| | SEQ. ID. NO:1461 | | | | | | |
| 770 | CCTCACAGGTCAGTGCATTA | −4.1 | −25.9 | 75.6 | −21.1 | −0.5 | −5.4 |
| | SEQ. ID. NO:1462 | | | | | | |
| 771 | CCCTCACAGGTCAGTGCATT | −4.1 | −28.2 | 79.9 | −23.4 | −0.5 | −6.2 |
| | SEQ. ID. NO:1463 | | | | | | |
| 820 | CTTGTACACAGCGTTTTTGG | −4.1 | −23.1 | 67.8 | −19 | 0 | −6.2 |
| | SEQ. ID. NO:1464 | | | | | | |
| 1316 | TAGCTTCAACCGCAGACCCT | −4.1 | −28.1 | 75.1 | −23.3 | −0.5 | −4.6 |
| | SEQ. ID. NO:1465 | | | | | | |
| 1629 | CAGGCAAAGTGTTGAGGATT | −4.1 | −22 | 65.3 | −17 | −0.7 | −4 |
| | SEQ. ID. NO:1466 | | | | | | |
| 1632 | AGACAGGCAAAGTGTTGAGG | −4.1 | −22.1 | 65.7 | −17.1 | −0.7 | −4 |
| | SEQ. ID. NO:1467 | | | | | | |
| 1711 | GTGGTCGTTTACTCTCCATG | −4.1 | −25.4 | 74.4 | −20.6 | −0.4 | −3.9 |
| | SEQ. ID. NO:1468 | | | | | | |
| 1752 | CACTGCACGTCCCAGATTTC | −4.1 | −26.8 | 74.4 | −22 | −0.5 | −7.5 |
| | SEQ. ID. NO:1469 | | | | | | |
| 2076 | AATATATGCAATATGGTAAG | −4.1 | −15.6 | 50.8 | −10.8 | −0.5 | −6.5 |
| | SEQ. ID. NO:1470 | | | | | | |
| 2097 | TTGAATACAACTCTTTAATA | −4.1 | −15.2 | 50.3 | −10.5 | −0.5 | −3.1 |
| | SEQ. ID. NO:1471 | | | | | | |
| 105 | GGAGGATTCTGGACTGAGTC | −4 | −24.1 | 72.5 | −19.6 | −0.1 | −5 |
| | SEQ. ID. NO:1472 | | | | | | |
| 355 | GAGATTCATTTTTGATCCCA | −4 | −22.5 | 66.4 | −17.6 | −0.8 | −4.6 |
| | SEQ. ID. NO:1473 | | | | | | |
| 429 | TGTTCTGTTAAAACACCAAA | −4 | −17.9 | 54.9 | −13.2 | −0.5 | −5.3 |
| | SEQ. ID. NO:1474 | | | | | | |
| 457 | CAGGTTCTGTCCCAGAGGAC | −4 | −27.4 | 79 −20.8 | −2.6 | −8.3 | |
| | SEQ. ID. NO:1475 | | | | | | |
| 754 | ATTATAGTGGTATCCAGAGG | −4 | −21.7 | 66.2 | −16.9 | −0.6 | −6.9 |
| | SEQ. ID. NO:1476 | | | | | | |
| 833 | CCCCGTTTTTACACTTGTAC | −4 | −25.3 | 70.7 | −20.6 | −0.4 | −4.5 |
| | SEQ. ID. NO:1477 | | | | | | |
| 867 | TTTCTTCGCATGTACATATC | −4 | −21.2 | 64.5 | −16.7 | 0 | −8 |
| | SEQ. ID. NO:1478 | | | | | | |
| 926 | ACAAGCATTCAGCCAACATT | −4 | −22.7 | 65.2 | −17.7 | −0.9 | −4.1 |
| | SEQ. ID. NO:1479 | | | | | | |
| 1193 | AAATGAGAAAATTTTCTTCT | −4 | −14.7 | 49.1 | −8.8 | −0.4 | −11.9 |
| | SEQ. ID. NO:1480 | | | | | | |
| 1329 | GAACGAAGGAACATAGCTTC | −4 | −19.7 | 58.7 | −14.7 | −0.9 | −4.6 |
| | SEQ. ID. NO:1481 | | | | | | |
| 1502 | TAACAATTGCTGTAAGCAGA | −4 | −19.3 | 58.6 | −12.2 | −3.1 | −9.1 |
| | SEQ. ID. NO:1482 | | | | | | |
| 1561 | CTCCTGAAGCTTCTCTACTG | −4 | −24.3 | 71.5 | −19.2 | 0 | −10.1 |
| | SEQ. ID. NO:1483 | | | | | | |
| 1730 | AGAGAAGTGGGGTAAACTTC | −4 | −20 | 60.7 | −15 | −0.9 | −4.1 |
| | SEQ. ID. NO:1484 | | | | | | |
| 1768 | CCCCTGTAATCCCCATCACT | −4 | −30.4 | 79 | −26.4 | 0 | −1.8 |
| | SEQ. ID. NO:1485 | | | | | | |
| 2023 ATAGAATTGAAGTAACAATC | −4 | −14.4 | 48.5 | −9.7 | −0.4 | −3.9 | |
| | SEQ. ID. NO:1486 | | | | | | |
| 184 | TTTTCTTCTTTCACTCCTTC | −3.9 | −23.2 | 71.6 | −19.3 | 0 | 0 |
| | SEQ. ID. NO:1487 | | | | | | |
| 388 | TTCATCTGTGGTAGGTAAAT | −3.9 | −20.5 | 63.3 | −16.6 | 0 | −2.8 |
| | SEQ. ID. NO:1488 | | | | | | |
| 394 | AGAAAATTCATCTGTGGTAG | −3.9 | −18.3 | 57.5 | −14.4 | 0 | −4.8 |
| | SEQ. ID. NO:1489 | | | | | | |
| 648 | TCTGCTACCTCAGTTTCTCC | −3.9 | −27 | 79.6 | −22.6 | −0.2 | −3.6 |
| | SEQ. ID. NO:1490 | | | | | | |
| 1747 | CACGTCCCAGATTTCACAGA | −3.9 | −25.4 | 71.2 | −21.5 | 0 | −4.6 |
| | SEQ. ID. NO:1491 | | | | | | |
| 1771 | CCTCCCCTGTAATCCCCATC | −3.9 | −31.9 | 82.3 | −28 | 0 | −1.6 |
| | SEQ. ID. NO:1492 | | | | | | |
| 1887 | ACACATGTAATTACAACATA | −3.9 | −16.6 | 52.8 | −11.6 | −0.6 | −9.8 |
| | SEQ. ID. NO:1493 | | | | | | |
| 2038 | TCCCTAGTTCAACAGATAGA | −3.9 | −22.5 | 66.6 | −18.6 | 0 | −3.6 |
| | SEQ. ID. NO:1494 | | | | | | |
| 2055 | TGAGCAAAATGAGATTTTCC | −3.9 | −18.9 | 57.5 | −14.1 | −0.7 | −4.8 |
| | SEQ. ID. NO:1495 | | | | | | |
| 2071 | ATGCAATATGGTAAGATGAG | −3.9 | −18.1 | 56.3 | −14.2 | 0 | −5.6 |
| | SEQ. ID. NO:1496 | | | | | | |
| 251 | ATGTCCAGAAGAAATCCAGG | −3.8 | −21.7 | 63.1 | −17.9 | 0 | −3.3 |
| | SEQ. ID. NO:1497 | | | | | | |
| 267 | GTTTCATCTTGAGGAAATGT | −3.8 | −20.1 | 62 | −15.4 | −0.7 | −7.9 |
| | SEQ. ID. NO:1498 | | | | | | |
| 389 | ATTCATCTGTGGTAGGTAAA | −3.8 | −20.5 | 63.3 | −16.7 | 0 | −2.8 |
| | SEQ. ID. NO:1499 | | | | | | |
| 391 | AAATTCATCTGTGGTAGGTA | −3.8 | −20.5 | 63.3 | −16.7 | 0 | −3.1 |
| | SEQ. ID. NO:1500 | | | | | | |
| 519 | GAAATCTGTGGTTGAACTTG | −3.8 | −19.3 | 59.1 | −15.5 | 0 | −3.4 |
| | SEQ. ID. NO:1501 | | | | | | |
| 594 | TCATATATTCCAGGAGAGTA | −3.8 | −21 | 64.5 | −17.2 | 0 | −5.3 |
| | SEQ. ID. NO:1502 | | | | | | |
| 719 | CAACACACAGCTCATCCCCT | −3.8 | −27.8 | 75.1 | −24 | 0 | −4.4 |
| | SEQ. ID. NO:1503 | | | | | | |
| 830 | CGTTTTTACACTTGTACACA | −3.8 | −20.9 | 62.7 | −16.4 | −0.4 | −6.6 |
| | SEQ. ID. NO:1504 | | | | | | |
| 855 | TACATATCCATCACACAGTT | −3.8 | −21.6 | 64.7 | −17.8 | 0 | −2.6 |
| | SEQ. ID. NO:1505 | | | | | | |
| 949 | AGATTTACACTGAATTTCAG | −3.8 | −17.7 | 56.3 | −12.3 | −1.6 | −9.6 |
| | SEQ. ID. NO:1506 | | | | | | |
| 1201 | TTCCGTCAAAATGAGAAAAT | −3.8 | −16.6 | 51.4 | −12.8 | 0.4 | −3.3 |
| | SEQ. ID. NO:1507 | | | | | | |
| 1504 | GATAACAATTGCTGTAAGCA | −3.8 | −19.3 | 58.4 | −12.6 | −2.9 | −7.7 |
| | SEQ. ID. NO:1508 | | | | | | |
| 1641 | CGACCCAGGAGACAGGCAAA | −3.8 | −25.9 | 69.3 | −22.1 | 0 | −4 |
| | SEQ. ID. NO:1509 | | | | | | |
| 2054 | GAGCAAAATGAGATTTTCCC | −3.8 | −20.9 | 61.2 | −16.1 | −0.9 | −4.8 |
| | SEQ. ID. NO:1510 | | | | | | |
| 285 | AACTCCAAAGTGTCTGAAGT | −3.7 | −20.9 | 62.5 | −16.5 | −0.5 | −5 |
| | SEQ. ID. NO:1511 | | | | | | |
| 538 | GGAATAATAGGATGACGAGG | −3.7 | −19 | 57.1 | −15.3 | 0 | −3.5 |
| | SEQ. ID. NO:1512 | | | | | | |
| 631 | TCCCTGGTAGAGAGTCTCAG | −3.7 | −26.2 | 77.8 | −21.1 | −1.1 | −10 |
| | SEQ. ID. NO:1513 | | | | | | |
| 746 | GGTATCCAGAGGCTCTGTCT | −3.7 | −27.5 | 81.5 | −22.2 | −1.5 | −8 |
| | SEQ. ID. NO:1514 | | | | | | |
| 790 | CCTGAAGAAACCTTTACACC | −3.7 | −22.1 | 62.4 | −18.4 | 0 | −2.8 |
| | SEQ. ID. NO:1515 | | | | | | |
| 1333 | AGCTGAACGAAGGAACATAG | −3.7 | −19.2 | 57.3 | −15.5 | 0 | −4.3 |
| | SEQ. ID. NO:1516 | | | | | | |
| 1635 | AGGAGACAGGCAAAGTGTTG | −3.7 | −22.1 | 65.7 | −17.8 | −0.3 | −4 |
| | SEQ. ID. NO:1517 | | | | | | |
| 1694 | ATGACATCAGCATCTCAGCG | −3.7 | −24.1 | 6.8 | −19.4 | −0.9 | −4.1 |
| | SEQ. ID. NO:1518 | | | | | | |
| 1751 | ACTGCACGTCCCAGATTTCA | −3.7 | −26.8 | 74.4 | −22.4 | −0.5 | −7.5 |
| | SEQ. ID. NO:1519 | | | | | | |
| 1828 | TGAAATAAAGGAAAGTTATA | −3.7 | −12.6 | 44.6 | −8.9 | 0 | −2.8 |
| | SEQ. ID. NO:1520 | | | | | | |
| 2028 | AACAGATAGAATTGAAGTAA | −3.7 | −14.6 | 48.8 | −10.9 | 0 | −3.1 |
| | SEQ. ID. NO:1521 | | | | | | |
| 76 | AGATCCCAGCGATTTTGCTA | −3.6 | −25.6 | 71.8 | −20.4 | −1.6 | −7.7 |
| | SEQ. ID. NO:1522 | | | | | | |
| 304 | TATGGTGGTCTTCAAAAAAA | −3.6 | −16.8 | 52.9 | −13.2 | 0 | −3.3 |
| | SEQ. ID. NO:1523 | | | | | | |
| 326 | TTCAATTGAAATGCACTTTC | −3.6 | −18 | 56.1 | −13.2 | −0.8 | −9.9 |
| | SEQ. ID. NO:1524 | | | | | | |
| 797 | TGCTTCTCCTGAAGAAACCT | −3.6 | −23.6 | 67.1 | −17.8 | −2.2 | −5.7 |
| | SEQ. ID. NO:1525 | | | | | | |
| 821 | ACTTGTACACAGCGTTTTTG | −3.6 | −22.1 | 65.8 | −18.5 | 0 | −6.3 |
| | SEQ. ID. NO:1526 | | | | | | |
| 1731 | CAGAGAAGTGGGGTAAACTT | −3.6 | −20.7 | 62 | −16.6 | −0.1 | −3.4 |
| | SEQ. ID. NO:1527 | | | | | | |
| 1861 | CATCAAGATTTCTTGAGTGA | −3.6 | −19.7 | 61.1 | −13.7 | −2.4 | −11.2 |
| | SEQ. ID. NO:1528 | | | | | | |
| 1915 | TAGCCCAATATTTACAGTTG | −3.6 | −21.3 | 63.1 | −17.7 | 0 | −4.1 |
| | SEQ. ID. NO:1529 | | | | | | |
| 133 | GGGTCAGAGATGGACTTTCA | −3.5 | −24.1 | 72 | −19.4 | −1.1 | −5.3 |
| | SEQ. ID. NO:1530 | | | | | | |
| 138 | GTTTTGGGTCAGAGATGGAC | −3.5 | −23.4 | 70.7 | −19 | −0.7 | −4.7 |
| | SEQ. ID. NO:1531 | | | | | | |
| 242 | AGAAATCCAGGAAACTAAGA | −3.5 | −17.4 | 53.7 | −13.3 | −0.3 | −5.2 |
| | SEQ. ID. NO:1532 | | | | | | |
| 250 | TGTCCAGAAGAAATCCAGGA | −3.5 | −22.3 | 64.4 | −17.9 | −0.7 | −5.3 |
| | SEQ. ID. NO:1533 | | | | | | |
| 392 | AAAATTCATCTGTGGTAGGT | −3.5 | −20.1 | 61.7 | −16.6 | 0 | −3.1 |
| | SEQ. ID. NO:1534 | | | | | | |
| 448 | TCCCAGAGGACCTGCCACTT | −3.5 | −30.3 | 81.1 | −25.7 | −1 | −6.7 |
| | SEQ. ID. NO:1535 | | | | | | |
| 782 | AACCTTTACACCCCTCACAG | −3.5 | −26.3 | 71.6 | −22.8 | 0 | −1.2 |
| | SEQ. ID. NO:1536 | | | | | | |
| 1078 | ATCTGGGGTGAGTTCAGTTT | −3.5 | −24.5 | 74.9 | −20.5 | −0.2 | −3.7 |
| | SEQ. ID. NO:1537 | | | | | | |
| 1115 | TATATGAATCCATAATAAAA | −3.5 | −13 | 45.1 | −8.4 | −1 | −4.2 |
| | SEQ. ID. NO:1538 | | | | | | |
| 1204 | CATTTCCGTCAAAATGAGAA | −3.5 | −18.8 | 56.1 | −14.1 | −1.1 | −5.2 |
| | SEQ. ID. NO:1539 | | | | | | |
| 1319 | ACATAGCTTCAACCGCAGAC | −3.5 | −24.1 | 68.1 | −20.6 | 0.3 | −4.6 |
| | SEQ. ID. NO:1540 | | | | | | |
| 1550 | TCTCTACTGCCTCTCTATCC | −3.5 | −26.8 | 78.9 | −23.3 | 0 | −3 |
| | SEQ. ID. NO:1541 | | | | | | |
| 1769 | TCCCCTGTAATCCCCATCAC | −3.5 | −29.9 | 78.8 | −26.4 | 0 | −1.6 |
| | SEQ. ID. NO:1542 | | | | | | |
| 376 | AGGTAAATGGGAATGTTCAA | −3.4 | −18.7 | 57.3 | −15.3 | 0 | −5.7 |
| | SEQ. ID. NO:1543 | | | | | | |
| 1073 | GGGTGAGTTCAGTTTTCTCC | −3.4 | −25.8 | 78.6 | −21.8 | −0.3 | −3.6 |
| | SEQ. ID. NO:1544 | | | | | | |
| 1353 | AGTTTCTTATTGAAAATCTC | −3.4 | −16.8 | 54.8 | −11.9 | −1.4 | −4.5 |
| | SEQ. ID. NO:1545 | | | | | | |
| 1488 | AGCAGAGCATACTCCTCTTG | −3.4 | −25.1 | 73.7 | −20.2 | −1.4 | −6.3 |
| | SEQ. ID. NO:1546 | | | | | | |
| 1862 | TCATCAAGATTTCTTGAGTG | −3.4 | −19.5 | 61.2 | −13.7 | −2.4 | −11.2 |
| | SEQ. ID. NO:1547 | | | | | | |
| 1883 | ATGTAATTACAACATAAATA | −3.4 | −13.1 | 45.6 | −8.5 | −0.4 | −10.3 |
| | SEQ. ID. NO:1548 | | | | | | |
| 2029 | CAACAGATAGAATTGAAGTA | −3.4 | −16 | 51.7 | −12.6 | 0 | −3.1 |
| | SEQ. ID. NO:1549 | | | | | | |
| 2035 | CTAGTTCAACAGATAGAATT | −3.4 | −17.5 | 55.8 | −14.1 | 0 | −3.7 |
| | SEQ. ID. NO:1550 | | | | | | |
| 2052 | GCAAAATGAGATTTTCCCTA | −3.4 | −20.9 | 61 | −16.5 | −0.9 | −4.3 |
| | SEQ. ID. NO:1551 | | | | | | |
| 209 | CTTTGAGCTATGTTTCTAAG | −3.3 | −19.8 | 61.9 | −16.5 | 0 | −4.5 |
| | SEQ. ID. NO:1552 | | | | | | |
| 1630 | ACAGGCAAAGTGTTGAGGAT | −3.3 | −22.1 | 65.5 | −18.8 | 0 | −4 |
| | SEQ. ID. NO:1553 | | | | | | |
| 1917 | TCTAGCCCAATATTTACAGT | −3.3 | −22.5 | 66.2 | −19.2 | 0 | −4.1 |
| | SEQ. ID. NO:1554 | | | | | | |
| 1919 | TATCTAGCCCAATATTTACA | −3.3 | −21 | 62.3 | −17.7 | 0 | −4.1 |
| | SEQ. ID. NO:1555 | | | | | | |
| 182 | TTCTTCTTTCACTCCTTCTA | −3.2 | −23.6 | 72.3 | −20.4 | 0 | 0 |
| | SEQ. ID. NO:1556 | | | | | | |
| 395 | AAGAAAATTCATCTGTGGTA | −3.2 | −17.6 | 55.4 | −14.4 | 0 | −4.8 |
| | SEQ. ID. NO:1557 | | | | | | |
| 428 | GTTCTGTTAAAACACCAAAT | −3.2 | −17.9 | 54.9 | −14.7 | 0 | −5.5 |
| | SEQ. ID. NO:1558 | | | | | | |
| 621 | AGAGTCTCAGCTGGCATACG | −3.2 | −25.3 | 73.6 | −21.5 | 0 | −8.6 |
| | SEQ. ID. NO:1559 | | | | | | |
| 629 | CCTGGTAGAGAGTCTCAGCT | −3.2 | −26.5 | 78.9 | −21.9 | −1.1 | −10 |
| | SEQ. ID. NO:1560 | | | | | | |
| 858 | ATGTACATATCCATCACACA | −3.2 | −21.5 | 64 | −17.8 | 0 | −7.6 |
| | SEQ. ID. NO:1561 | | | | | | |
| 1178 | CTTCTGCACTGAATTCTTCT | −3.2 | −22.6 | 67.9 | −18.7 | −0.4 | −6.9 |
| | SEQ. ID. NO:1562 | | | | | | |
| 1286 | CAATCTGGTCTTCATGGTCC | −3.2 | −25 | 73.6 | −21.8 | 0 | −4.7 |
| | SEQ. ID. NO:1563 | | | | | | |
| 1437 | AAACTAAACATAGGTGTTAT | −3.2 | −16 | 51.7 | −11.1 | −1.7 | −5.8 |
| | SEQ. ID. NO:1564 | | | | | | |
| 1732 | ACAGAGAAGTGGGGTAAACT | −3.2 | −20.8 | 62.2 | −17.6 | 0 | −2.9 |
| | SEQ. ID. NO:1565 | | | | | | |
| 1918 | ATCTAGCCCAATATTTACAG | −3.2 | −21.3 | 63 | −18.1 | 0 | −4.1 |
| | SEQ. ID. NO:1566 | | | | | | |
| 2080 | ATAAAATATATGCAATATGG | −3.2 | −13.7 | 46.6 | −9.8 | −0.5 | −6 |
| | SEQ. ID. NO:1567 | | | | | | |
| 279 | AAAGTGTCTGAAGTTTCATC | −3.1 | −19.1 | 60.3 | −16 | 0 | −4.7 |
| | SEQ. ID. NO:1568 | | | | | | |
| 731 | TGTCTCCACAAACAACACAC | −3.1 | −21.3 | 61.9 | −18.2 | 0 | −2.8 |
| | SEQ. ID. NO:1569 | | | | | | |
| 1174 | TGCACTGAATTCTTCTTTTA | −3.1 | −20.3 | 62.5 | −16.5 | −0.4 | −6.9 |
| | SEQ. ID. NO:1570 | | | | | | |
| 1741 | CCAGATTTCACAGAGAAGTG | −3.1 | −21.2 | 63.6 | −17.5 | −0.3 | −4.5 |
| | SEQ. ID. NO:1571 | | | | | | |
| 1743 | TCCCAGATTTCACAGAGAAG | −3.1 | −22.4 | 65.7 | −18.7 | −0.3 | −3.7 |
| | SEQ. ID. NO:1572 | | | | | | |
| 1774 | ACCCCTCCCCTGTAATCCCC | −3.1 | −36 | 86.5 | −31.9 | 0 | −1.7 |
| | SEQ. ID. NO:1573 | | | | | | |
| 26 | ATAAGTCGGGGAGACAATGA | −3 | −21 | 61.7 | −15.9 | −2.1 | −5.1 |
| | SEQ. ID. NO:1574 | | | | | | |
| 179 | TTCTTTCACTCCTTCTACGA | −3 | −23.8 | 70.1 | −20.8 | 0 | −3.5 |
| | SEQ. ID. NO:1575 | | | | | | |
| 235 | CAGGAAACTAAGAGAAGCAG | −3 | −18.2 | 55.9 | −14.6 | −0.3 | −4.7 |
| | SEQ. ID. NO:1576 | | | | | | |
| 334 | CCAAATTTTTCAATTGAAAT | −3 | −15.4 | 49.6 | −10.3 | −0.5 | −12.4 |
| | SEQ. ID. NO:1577 | | | | | | |
| 387 | TCATCTGTGGTAGGTAAATG | −3 | −20.4 | 62.8 | −17.4 | 0 | −2.8 |
| | SEQ. ID. NO:1578 | | | | | | |
| 458 | CCAGGTTCTGTCCCAGAGGA | −3 | −29.2 | 82 | −24.8 | −1.3 | −6.8 |
| | SEQ. ID. NO:1579 | | | | | | |
| 460 | TTCCAGGTTCTGTCCCAGAG | −3 | −27.9 | 80.2 | −23.6 | −1.2 | −7 |
| | SEQ. ID. NO:1580 | | | | | | |
| 497 | GAAACTGAACATTGCTGTAT | −3 | −18.8 | 57.3 | −15.1 | −0.5 | −3.9 |
| | SEQ. ID. NO:1581 | | | | | | |
| 768 | TCACAGGTCAGTGCATTATA | −3 | −22.7 | 69 | −19 | −0.5 | −5.4 |
| | SEQ. ID. NO:1582 | | | | | | |
| 956 | GTCGCTTAGATTTACACTGA | −3 | −22 | 65.7 | −19 | 0 | −3.1 |
| | SEQ. ID. NO:1583 | | | | | | |
| 1197 | GTCAAAATGAGAAAATTTTC | −3 | −14 | 47.5 | −9.8 | −0.7 | −10.1 |
| | SEQ. ID. NO:1584 | | | | | | |
| 1205 | CCATTTCCGTCAAAATGAGA | −3 | −21.5 | 61.4 | −16.9 | −1.6 | −6 |
| | SEQ. ID. NO:1585 | | | | | | |
| 1403 | TACCACTATTTCGAATTCTT | −3 | −20.2 | 60.5 | −17.2 | 0 | −6.7 |
| | SEQ. ID. NO:1586 | | | | | | |
| 1508 | ACAGGATAACAATTGCTGTA | −3 | −19.6 | 59.4 | −15.6 | −0.9 | −7.7 |
| | SEQ. ID. NO:1587 | | | | | | |
| 161 | GATGTCTTCTACCTCCTTGG | −2.9 | −25.9 | 75.5 | −22.5 | −0.1 | −3.2 |
| | SEQ. ID. NO:1588 | | | | | | |
| 178 | TCTTTCACTCCTTCTACGAT | −2.9 | −23.7 | 69.7 | −20.8 | 0 | −3.5 |
| | SEQ. ID. NO:1589 | | | | | | |
| 632 | CTCCCTGGTAGAGAGTCTCA | −2.9 | −27.1 | 79.5 | −22.8 | −1.1 | −10 |
| | SEQ. ID. NO:1590 | | | | | | |
| 1103 | TAATAAAATGTAGAAGAGTC | −2.9 | −13.6 | 47 | −10.7 | 0 | −3.5 |
| | SEQ. ID. NO:1591 | | | | | | |
| 1705 | GTTTACTCTCCATGACATCA | −2.9 | −23.2 | 69.2 | −20.3 | 0 | −4.5 |
| | SEQ. ID. NO:1592 | | | | | | |
| 1870 | ATAAATATTCATCAAGATTT | −2.9 | −14.4 | 48.7 | −11.5 | 4 | −4.6 |
| | SEQ. ID. NO:1593 | | | | | | |
| 249 | GTCCAGAAGAAATCCAGGAA | −2.8 | −21.6 | 62.5 | −17.8 | −0.9 | −5.7 |
| | SEQ. ID. NO:1594 | | | | | | |
| 396 | AAAGAAAATTCATCTGTGGT | −2.8 | −17.2 | 54.2 | −14.4 | 0 | −4.8 |
| | SEQ. ID. NO:1595 | | | | | | |
| 628 | CTGGTAGAGAGTCTCAGCTG | −2.8 | −24.5 | 74.7 | −20.3 | −1.1 | −10 |
| | SEQ. ID. NO:1596 | | | | | | |
| 1194 | AAAATGAGAAAATTTTCTTC | −2.8 | −13.1 | 45.8 | −8.1 | −1 | −12.5 |
| | SEQ. ID. NO:1597 | | | | | | |
| 1466 | TCATTTTCAGTTCCCCAATA | −2.8 | −23.9 | 69 −21.1 | 0 | −1.7 | |
| | SEQ. ID. NO:1598 | | | | | | |
| 1708 | GTCGTTTACTCTCCATGACA | −2.8 | −24.5 | 71.5 | −21.1 | −0.3 | −4.6 |
| | SEQ. ID. NO:1599 | | | | | | |
| 20 | CGGGGAGACAATGACCTGAG | −2.7 | −23.4 | 66.8 | −20.7 | 0 | −3.1 |
| | SEQ. ID. NO:1600 | | | | | | |
| 30 | TAGGATAACTCGGGGAGACA | −2.7 | −22.6 | 66.1 | −17.8 | −2.1 | −4.9 |
| | SEQ. ID. NO:1601 | | | | | | |
| 59 | CTACAAATGCTCAGAATCCA | −2.7 | −20.9 | 61.2 | −18.2 | 0 | −3.6 |
| | SEQ. ID. NO:1602 | | | | | | |
| 187 | TTCTTTTCTTCTTTCACTCC | −2.7 | −23.2 | 71.6 | −20.5 | 0 | 0 |
| | SEQ. ID. NO:1603 | | | | | | |
| 383 | CTGTGGTAGGTAAATGGGAA | −2.7 | −21.2 | 63.1 | −18.5 | 0 | −1.2 |
| | SEQ. ID. NO:1604 | | | | | | |
| 452 | TCTGTCCCAGAGGACCTGCC | −2.7 | −30.9 | 84.3 | −25.2 | −3 | −8.6 |
| | SEQ. ID. NO:1605 | | | | | | |
| 475 | CGAGTATGGTTCCACTTCCA | −2.7 | −26.2 | 73.8 | −22.8 | −0.5 | −5.6 |
| | SEQ. ID. NO:1606 | | | | | | |
| 522 | GAGGAAATCTGTGGTTGAAC | −2.7 | −20.1 | 60.9 | −17.4 | 0 | −3 |
| | SEQ. ID. NO:1607 | | | | | | |
| 779 | CTTTACACCCCTCACAGGTC | −2.7 | −27.6 | 77.3 | −24.2 | −0.5 | −4.1 |
| | SEQ. ID. NO:1608 | | | | | | |
| 937 | AATTTCAGTTAACAAGCATT | −2.7 | −17.7 | 55.7 | −15 | 0 | −7.3 |
| | SEQ. ID. NO:1609 | | | | | | |
| 1021 | CAAGTCACGACCTTCACTGT | −2.7 | −24.5 | 69.8 | −21.8 | 0 | −4.7 |
| | SEQ. ID. NO:1610 | | | | | | |
| 1321 | GAACATAGCTTCAACCGCAG | −2.7 | −23.2 | 65.4 | −19.8 | −0.5 | −4.6 |
| | SEQ. ID. NO:1611 | | | | | | |
| 1339 | AATCTCAGCTGAACGAAGGA | −2.7 | −21 | 61.4 | −17.2 | 0 | −10.1 |
| | SEQ. ID. NO:1612 | | | | | | |
| 1484 | GAGCATACTCCTCTTGAGTC | −2.7 | −24.8 | 74.5 | −20.4 | −1.7 | −7.5 |
| | SEQ. ID. NO:1613 | | | | | | |
| 1507 | CAGGATAACAATTGCTGTAA | −2.7 | −18.7 | 57 | −15.3 | −0.4 | −7 |
| | SEQ. ID. NO:1614 | | | | | | |
| 1699 | TCTCCATGACATCAGCATCT | −2.7 | −24.8 | 72.5 | −22.1 | 0 | −4.5 |
| | SEQ. ID. NO:1615 | | | | | | |
| 1998 | AATTAGGCAAACAGGGCTTG | −2.7 | −21.5 | 62.8 | −18.1 | −0.5 | −4 |
| | SEQ. ID. NO:1616 | | | | | | |
| 449 | GTCCCAGAGGACCTGCCACT | −2.6 | −31.4 | 84.3 | −26.5 | −2.3 | −7.6 |
| | SEQ. ID. NO:1617 | | | | | | |
| 714 | CACAGCTCATCCCCTTTGAT | −2.6 | −27.5 | 76.1 | −24.9 | 0 | −4.4 |
| | SEQ. ID. NO:1618 | | | | | | |
| 927 | AACAAGCATTCAGCCAACAT | −2.6 | −21.9 | 62.8 | −18.8 | −0.1 | −3.9 |
| | SEQ. ID. NO:1619 | | | | | | |
| 958 | CAGTCGCTTAGATTTACACT | −2.6 | −22.1 | 66 | −19.5 | 0 | −3.1 |
| | SEQ. ID. NO:1620 | | | | | | |
| 1192 | AATGAGAAAATTTTCTTCTG | −2.6 | −15.4 | 50.7 | −10.6 | −1 | −12.5 |
| | SEQ. ID. NO:1621 | | | | | | |
| 1412 | CATCAGAGATACCACTATTT | −2.6 | −20.6 | 62.2 | −18 0 | −3.5 | |
| | SEQ. ID. NO:1622 | | | | | | |
| 1465 | CATTTTCAGTTCCCCAATAC | −2.6 | −23.7 | 68 | −21.1 | 0 | −2 |
| | SEQ. ID. NO:1623 | | | | | | |
| 1770 | CTCCCCTGTAATCCCCATCA | −2.6 | −30.6 | 80.1 | −28 | 0 | −1.7 |
| | SEQ. ID. NO:1624 | | | | | | |
| 2032 | GTTCAACAGATAGAATTGAA | −2.6 | −16.8 | 53.6 | −12.6 | −1.6 | −5.7 |
| | SEQ. ID. NO:1625 | | | | | | |
| 29 | AGGATAAGTCGGGGAGACAA | −2.5 | −22.2 | 64.5 | −17.6 | −2.1 | −4.9 |
| | SEQ. ID. NO:1626 | | | | | | |
| 248 | TCCAGAAGAAATCCAGGAAA | −2.5 | −19.7 | 57.8 | −16.5 | −0.4 | −5.7 |
| | SEQ. ID. NO:1627 | | | | | | |
| 332 | AAATTTTTCAATTGAAATGC | −2.5 | −14.5 | 48.3 | −10 | −0.5 | −12.1 |
| | SEQ. ID. NO:1628 | | | | | | |
| 374 | GTAAATGGGAATGTTCAATG | −2.5 | −17.5 | 54.6 | −15 | 0 | −5.7 |
| | SEQ. ID. NO:1629 | | | | | | |
| 539 | TGGAATAATAGGATGACGAG | −2.5 | −17.8 | 54.7 | −15.3 | 0 | −3.5 |
| | SEQ. ID. NO:1630 | | | | | | |
| 591 | TATATTCCAGGAGAGTACCA | −2.5 | −22.8 | 67.4 | −19.6 | −0.5 | −5 |
| | SEQ. ID. NO:1631 | | | | | | |
| 624 | TAGAGAGTCTCAGCTGGCAT | −2.5 | −24.9 | 74.9 | −21 | −1.1 | −10 |
| | SEQ. ID. NO:1632 | | | | | | |
| 788 | TGAAGAAACCTTTACACCCC | −2.5 | −23.2 | 64 | −20.7 | 0 | −2.8 |
| | SEQ. ID. NO:1633 | | | | | | |
| 953 | GCTTAGATTTACACTGAATT | −2.5 | −19 | 58.9 | −16.5 | 0 | −3.6 |
| | SEQ. ID. NO:1634 | | | | | | |
| 1083 | TGTTGATCTGGGGTGAGTTC | −2.5 | −24.3 | 74 | −21.8 | 0 | −4.9 |
| | SEQ. ID. NO:1635 | | | | | | |
| 1241 | TTGTGAATTCTACAAGAACC | −2.5 | −18.6 | 57.1 | −14.9 | −0.9 | −9.9 |
| | SEQ. ID. NO:1636 | | | | | | |
| 1421 | TTATATATTCATCAGAGATA | −2.5 | −16.4 | 54.1 | −13.9 | 0 | −3.9 |
| | SEQ. ID. NO:1637 | | | | | | |
| 1505 | GGATAACAATTGCTGTAAGC | −2.5 | −19.8 | 59.7 | −15.5 | −1.8 | −7.1 |
| | SEQ. ID. NO:1638 | | | | | | |
| 1628 | AGGCAAAGTGTTGAGGATTT | −2.5 | −21.4 | 64.4 | −18 | −0.7 | −4 |
| | SEQ. ID. NO:1639 | | | | | | |
| 331 | AATTTTTCAATTGAAATGCA | −2.4 | −15.9 | 51.1 | −11.4 | −0.4 | −12.4 |
| | SEQ. ID. NO:1640 | | | | | | |
| 375 | GGTAAATGGGAATGTTCAAT | −2.4 | −18.7 | 57.1 | −16.3 | 0 | −5.7 |
| | SEQ. ID. NO:1641 | | | | | | |
| 427 | TTCTGTTAAAACACCAAATA | −2.4 | −16.4 | 51.8 | −14 | 0 | −5.5 |
| | SEQ. ID. NO:1642 | | | | | | |
| 459 | TCCAGGTTCTGTCCCAGAGG | −2.4 | −29 | 82.5 | −25.3 | −1.2 | −7 |
| | SEQ. ID. NO:1643 | | | | | | |
| 716 | CACACAGCTCATCCCCTTTG | −2.4 | −27.8 | 76.4 | −25.4 | 0 | −4.2 |
| | SEQ. ID. NO:1644 | | | | | | |
| 934 | TTCAGTTAACAAGCATTCAG | −2.4 | −19.4 | 60.1 | −17 | 0 | −7.3 |
| | SEQ. ID. NO:1645 | | | | | | |
| 1203 | ATTTCCGTCAAAATGAGAAA | −2.4 | −17.4 | 53.3 | −14 | −0.9 | −5.1 |
| | SEQ. ID. NO:1646 | | | | | | |
| 1328 | AACGAAGGAACATAGCTTCA | −2.4 | −19.8 | 58.6 | −15.4 | −2 | −5.6 |
| | SEQ. ID. NO:1647 | | | | | | |
| 1463 | TTTTCAGTTCCCCAATACTT | −2.4 | −24 | 69.2 | −21.6 | 0 | −2.7 |
| | SEQ. ID. NO:1648 | | | | | | |
| 2082 | TAATAAAATATATGCAATAT | −2.4 | −11.5 | 42.4 | −8.5 | −0.3 | −6.2 |
| | SEQ. ID. NO:1649 | | | | | | |
| 2085 | CTTTAATAAAATATATGCAA | −2.4 | −12.9 | 45.1 | −10.5 | 0 | −5.6 |
| | SEQ. ID. NO:1650 | | | | | | |
| 18 | GGGAGACAATGAGGTGAGGA | −2.3 | −23.2 | 67.9 | −20.9 | 0 | −3.1 |
| | SEQ. ID. NO:1651 | | | | | | |
| 384 | TCTGTGGTAGGTAAATGGGA | −2.3 | −22.3 | 66.8 | −20 | 0 | −1.9 |
| | SEQ. ID. NO:1652 | | | | | | |
| 832 | CCCGTTTTTACACTTGTACA | −2.3 | −24 | 68.3 | −21 | −0.4 | −6.4 |
| | SEQ. ID. NO:1653 | | | | | | |
| 929 | TTAACAAGCATTCAGCCAAC | −2.3 | −21 | 61.5 | −17.7 | −0.9 | −4.1 |
| | SEQ. ID. NO:1654 | | | | | | |
| 1076 | CTGGGGTGAGTTCAGTTTTC | −2.3 | −24.6 | 75.3 | −22.3 | 0 | −3.4 |
| | SEQ. ID. NO:1655 | | | | | | |
| 1162 | TTCTTTTAAAATTTTATTTG | −2.3 | −13.5 | 47.2 | −10.6 | −0.2 | −8 |
| | SEQ. ID. NO:1656 | | | | | | |
| 1471 | TTGAGTCATTTTCAGTTCCC | −2.3 | −24.1 | 72.4 | −21.8 | 0 | −5.8 |
| | SEQ. ID. NO:1657 | | | | | | |
| 1625 | CAAAGTGTTGAGGATTTTCA | −2.3 | −19.6 | 60.4 | −17.3 | 0 | −3 |
| | SEQ. ID. NO:1658 | | | | | | |
| 1868 | AAATATTCATCAAGATTTCT | −2.3 | −16 | 52.3 | −13.7 | 4.1 | −4.6 |
| | SEQ. ID. NO:1659 | | | | | | |
| 382 | TGTGGTAGGTAAATGGGAAT | −2.2 | −20.3 | 61.1 | −18.1 | 0 | −1.2 |
| | SEQ. ID. NO:1660 | | | | | | |
| 451 | CTGTCCCAGAGGACCTGCCA | −2.2 | −31.2 | 83.4 | −26 | −3 | −8.6 |
| | SEQ. ID. NO:1661 | | | | | | |
| 585 | CCAGGAGAGTACCACTCTTC | −2.2 | −25.8 | 74.9 | −21.3 | −2.3 | −7.5 |
| | SEQ. ID. NO:1662 | | | | | | |
| 772 | CCCCTCACAGGTCAGTGCAT | −2.2 | −30.1 | 83 | −27.2 | −0.5 | −6.2 |
| | SEQ. ID. NO:1663 | | | | | | |
| 817 | GTACACAGCGTTTTTGGTAA | −2.2 | −22.3 | 66 | −20.1 | 0 | −4.6 |
| | SEQ. ID. NO:1664 | | | | | | |
| 1166 | ATTCTTCTTTTAAAATTTTA | −2.2 | −14.7 | 49.9 | −12 | 0 | −7.7 |
| | SEQ. ID. NO:1665 | | | | | | |
| 1320 | AACATAGCTTCAACCGCAGA | −2.2 | −23.2 | 65.4 | −20.3 | −0.5 | −4.3 |
| | SEQ. ID. NO:1666 | | | | | | |
| 1664 | TGAATGTCCGTAATTCAGTC | −2.2 | −21.3 | 63.7 | −17.6 | −1.4 | −5.9 |
| | SEQ. ID. NO:1667 | | | | | | |
| 1855 | GATTTCTTGAGTGAAACTGG | −2.2 | −19.5 | 60 | −16.1 | −1.1 | −5.5 |
| | SEQ. ID. NO:1668 | | | | | | |
| 185 | CTTTTCTTCTTTCACTCCTT | −2.1 | −23.7 | 71.9 | −21.6 | 0 | 0 |
| | SEQ. ID. NO:1669 | | | | | | |
| 335 | TCCAAATTTTTCAATTGAAA | −2.1 | −15.8 | 50.6 | −11.7 | −0.5 | −12.1 |
| | SEQ. ID. NO:1670 | | | | | | |
| 352 | ATTCATTTTTGATCCCATCC | −2.1 | −23.7 | 68.7 | −20.7 | −0.8 | −4.3 |
| | SEQ. ID. NO:1671 | | | | | | |
| 354 | AGATTCATTTTTGATCCCAT | −2.1 | −21.9 | 65 | −18.9 | −0.8 | −4.5 |
| | SEQ. ID. NO:1672 | | | | | | |
| 545 | CCAGGTTGGAATAATAGGAT | −2.1 | −20.8 | 61.5 | −18.1 | −0.3 | −3.5 |
| | SEQ. ID. NO:1673 | | | | | | |
| 787 | GAAGAAACCTTTACACCCCT | −2.1 | −24.1 | 65.8 | −22 | 0 | −2.8 |
| | SEQ. ID. NO:1674 | | | | | | |
| 856 | GTACATATCCATCACACAGT | −2.1 | −22.7 | 67.6 | −20.6 | 0 | −4.6 |
| | SEQ. ID. NO:1675 | | | | | | |
| 1082 | GTTGATCTGGGGTGAGTTCA | −2.1 | −25 | 75.4 | −22.9 | 0 | −4.9 |
| | SEQ. ID. NO:1676 | | | | | | |
| 1088 | GAGTCTGTTGATCTGGGGTG | −2.1 | −25.1 | 75.7 | −23 | 0 | −4.9 |
| | SEQ. ID. NO:1677 | | | | | | |
| 1522 | TTGTCTATCTGGAGACAGGA | −2.1 | −22.7 | 69.9 | −18.2 | −2.4 | −8.9 |
| | SEQ. ID. NO:1678 | | | | | | |
| 1746 | ACGTCCCAGATTTCACAGAG | −2.1 | −24.7 | 70.3 | −22.6 | 0 | −4.4 |
| | SEQ. ID. NO:1679 | | | | | | |
| 1882 | TGTAATTACAACATAAATAT | −2.1 | −13.1 | 45.6 | −10.2 | 0 | −9.4 |
| | SEQ. ID. NO:1680 | | | | | | |
| 270 | GAAGTTTCATCTTGAGGAAA | −2 | −18.8 | 58.4 | −16.1 | −0.5 | −7.7 |
| | SEQ. ID. NO:1681 | | | | | | |
| 1102 | AATAAAATGTAGAAGAGTCT | −2 | −14.8 | 49.5 | −12.8 | 0 | −5.5 |
| | SEQ. ID. NO:1682 | | | | | | |
| 1107 | TCCATAATAAAATGTAGAAG | −2 | −14.5 | 48.2 | −12.5 | 0 | −2.8 |
| | SEQ. ID. NO:1683 | | | | | | |
| 1243 | TTTTGTGAATTCTACAAGAA | −2 | −16.6 | 53.4 | −13.2 | −0.7 | −10.5 |
| | SEQ. ID. NO:1684 | | | | | | |
| 1438 | AAAACTAAACATAGGTGTTA | −2 | −15.3 | 50.1 | −11.6 | −1.7 | −5.8 |
| | SEQ. ID. NO:1685 | | | | | | |
| 1493 | CTGTAAGCAGAGCATACTCC | −2 | −23.9 | 70 −20.4 | −1.4 | −7.9 | |
| | SEQ. ID. NO:1686 | | | | | | |
| 1511 | GAGACAGGATAACAATTGCT | −2 | −19.9 | 59.8 | −17.9 | 0 | −7 |
| | SEQ. ID. NO:1687 | | | | | | |
| 1521 | TGTCTATCTGGAGACAGGAT | −2 | −22.6 | 68.5 | −18.2 | −2.4 | −8.6 |
| | SEQ. ID. NO:1688 | | | | | | |
| 2077 | AAATATATGCAATATGGTAA | −2 | −14.9 | 49.1 | −12.2 | −0.5 | −6.5 |
| | SEQ. ID. NO:1689 | | | | | | |
| 196 | TTCTAAGTCTTCTTTTCTTC | −2 | −20.4 | 65.8 | −17.9 | −0.3 | −3 |
| | SEQ. ID. NO:1690 | | | | | | |
| 373 | TAAATGGGAATGTTCAATGA | −1.9 | −16.9 | 53.1 | −15 | 0 | −5.7 |
| | SEQ. ID. NO:1691 | | | | | | |
| 386 | CATCTGTGGTAGGTAAATGG | −1.9 | −21.2 | 64 | −19.3 | 0 | −2.5 |
| | SEQ. ID. NO:1692 | | | | | | |
| 750 | TAGTGGTATCCAGAGGCTCT | −1.9 | −25.9 | 77.1 | −23.2 | −0.6 | −4.8 |
| | SEQ. ID. NO:1693 | | | | | | |
| 957 | AGTCGCTTAGATTTACACTG | −1.9 | −21.4 | 64.6 | −19.5 | 0 | −3.1 |
| | SEQ. ID. NO:1694 | | | | | | |
| 1498 | AATTGCTGTAAGCAGAGCAT | −1.9 | −21.9 | 65 | −16.9 | −3.1 | −10.7 |
| | SEQ. ID. NO:1695 | | | | | | |
| 1767 | CCCTGTAATCCCCATCACTG | −1.9 | −28.4 | 75.6 | −26.5 | 0 | −2.3 |
| | SEQ. ID. NO:1696 | | | | | | |
| 58 | TACAAATGCTCAGAATCCAA | −1.8 | −19.3 | 57.6 | −17.5 | 0 | −3.6 |
| | SEQ. ID. NO:1697 | | | | | | |
| 755 | CATTATAGTGGTATCCAGAG | −1.8 | −21.2 | 64.8 | −18.6 | −0.6 | −6.9 |
| | SEQ. ID. NO:1698 | | | | | | |
| 800 | TAATGCTTCTCCTGAAGAAA | −1.8 | −19.5 | 58.6 | −16.1 | −1.5 | −6.7 |
| | SEQ. ID. NO:1699 | | | | | | |
| 1196 | TCAAAATGAGAAAATTTTCT | −1.8 | −13.7 | 46.7 | −9.8 | −0.8 | −12.3 |
| | SEQ. ID. NO:1700 | | | | | | |
| 1202 | TTTCCGTCAAAATGAGAAAA | −1.8 | −16.7 | 51.7 | −14.1 | −0.6 | −4.5 |
| | SEQ. ID. NO:1701 | | | | | | |
| 1358 | ACGGAAGTTTCTTATTGAAA | −1.8 | −17.9 | 55.5 | −14.8 | −1.2 | −6.6 |
| | SEQ. ID. NO:1702 | | | | | | |
| 1742 | CCCAGATTTCACAGAGAAGT | −1.8 | −23.2 | 67.4 | −20.8 | −0.3 | −3.7 |
| | SEQ. ID. NO:1703 | | | | | | |
| 1886 | CACATGTAATTACAACATAA | −1.8 | −15.7 | 50.6 | −12.6 | −0.6 | −10.3 |
| | SEQ. ID. NO:1704 | | | | | | |
| 2002 | ATTTAATTAGGCAAACAGGG | −1.8 | −18.6 | 56.9 | −16.8 | 0 | −4.1 |
| | SEQ. ID. NO:1705 | | | | | | |
| 71 | CCAGCGATTTTGCTACAAAT | −1.7 | −22.1 | 62.8 | −18.8 | −1.6 | −7.2 |
| | SEQ. ID. NO:1706 | | | | | | |
| 108 | CTGGGAGGATTCTGGACTGA | −1.7 | −24.6 | 71.6 | −22.9 | 0 | −72.7 |
| | SEQ. ID. NO:1707 | | | | | | |
| 339 | CCCATCCAAATTTTTCAATT | −1.7 | −21.3 | 61.1 | −19 | −0.3 | −4.6 |
| | SEQ. ID. NO:1708 | | | | | | |
| 369 | TGGGAATGTTCAATGAGATT | −1.7 | −19.3 | 59 | −17.6 | 0 | −5.7 |
| | SEQ. ID. NO:1709 | | | | | | |
| 583 | AGGAGAGTACCACTCTTCAG | −1.7 | −23.8 | 71.4 | −18.7 | −3.4 | −8.6 |
| | SEQ. ID. NO:1710 | | | | | | |
| 592 | ATATATTCCAGGAGAGTACC | −1.7 | −22.1 | 66.2 | −20.4 | 0 | −5.3 |
| | SEQ. ID. NO:1711 | | | | | | |
| 717 | ACACACAGCTCATCCCCTTT | −1.7 | −28 | 77.2 | −26.3 | 0 | −4.4 |
| | SEQ. ID. NO:1712 | | | | | | |
| 730 | GTCTCCACAAACAACACACA | −1.7 | −22 | 63.2 | −20.3 | 0 | −2.2 |
| | SEQ. ID. NO:1713 | | | | | | |
| 799 | AATGCTTCTCCTGAAGAAAC | −1.7 | −20 | 59.7 | −16.1 | −2.2 | −6.7 |
| | SEQ. ID. NO:1714 | | | | | | |
| 816 | TACACAGCGTTTTTGGTAAT | −1.7 | −21.1 | 62.9 | −19.4 | 0 | −4.1 |
| | SEQ. ID. NO:1715 | | | | | | |
| 1163 | CTTCTTTTAAAATTTTATTT | −1.7 | −14.4 | 49.1 | −12.2 | 0 | −8 |
| | SEQ. ID. NO:1716 | | | | | | |
| 1624 | AAAGTGTTGAGGATTTTCAG | −1.7 | −18.9 | 59.3 | −17.2 | 0 | −3.2 |
| | SEQ. ID. NO:1717 | | | | | | |
| 1775 | GACCCCTCCCCTGTAATCCC | −1.7 | −33.6 | 84.7 | −31.9 | 0 | −2 |
| | SEQ. ID. NO:1718 | | | | | | |
| 1906 | ATTTACAGTTGTGGAAGTTA | −1.7 | −19.4 | 61 | −17.7 | 0 | −3.4 |
| | SEQ. ID. NO:1719 | | | | | | |
| 2068 | CAATATGGTAAGATGAGCAA | −1.7 | −19.1 | 55.8 | −16.4 | 0 | −4.1 |
| | SEQ. ID. NO:1720 | | | | | | |
| 268 | AGTTTCATCTTGAGGAAATG | −1.6 | −18.9 | 59.1 | −16.4 | −0.7 | −7.9 |
| | SEQ. ID. NO:1721 | | | | | | |
| 353 | GATTCATTTTTGATCCCATC | −1.6 | −22.3 | 66.3 | −19.8 | −0.8 | −4.3 |
| | SEQ. ID. NO:1722 | | | | | | |
| 536 | AATAATAGGATGACGAGGAA | −1.6 | −17.1 | 53 | −15.5 | 0 | −3.5 |
| | SEQ. ID. NO:1723 | | | | | | |
| 546 | CCCAGGTTGGAATAATAGGA | −1.6 | −22.8 | 65.1 | −20.3 | −0.8 | −4.3 |
| | SEQ. ID. NO:1724 | | | | | | |
| 815 | ACACAGCGTTTTTGGTAATG | −1.6 | −21.4 | 63.3 | −19.8 | 0 | −3.7 |
| | SEQ. ID. NO:1725 | | | | | | |
| 1707 | TCGTTTACTCTCCATGACAT | −1.6 | −23.3 | 68.1 | −21.7 | 0 | −4.5 |
| | SEQ. ID. NO:1726 | | | | | | |
| 1824 | ATAAAGGAAAGTTATACATC | −1.6 | −14.7 | 49.2 | −13.1 | 0 | −2.7 |
| | SEQ. ID. NO:1727 | | | | | | |
| 2031 | TTCAACAGATAGAATTGAAG | −1.6 | −15.6 | 51 | −12.6 | −1.3 | −5.1 |
| | SEQ. ID. NO:1728 | | | | | | |
| 146 | CTTGGATTGTTTTGGGTCAG | −1.5 | −23.1 | 69.7 | −21.6 | 0 | −3.4 |
| | SEQ. ID. NO:1729 | | | | | | |
| 333 | CAAATTTTTCAATTGAAATG | −1.5 | −13.4 | 46 | −9.8 | −0.5 | −12.4 |
| | SEQ. ID. NO:1730 | | | | | | |
| 523 | CGAGGAAATCTGTGGTTGAA | −1.5 | −20.7 | 60.9 | −19.2 | 0 | −2.6 |
| | SEQ. ID. NO:1731 | | | | | | |
| 747 | TGGTATCCAGAGGCTCTGTC | −1.5 | −26.6 | 79.1 | −23.5 | −1.5 | −8 |
| | SEQ. ID. NO:1732 | | | | | | |
| 1340 | AAATCTCAGCTGAACGAAGG | −1.5 | −19.7 | 58.3 | −17.2 | 0 | −9.9 |
| | SEQ. ID. NO:1733 | | | | | | |
| 1413 | TCATCAGAGATACCACTATT | −1.5 | −20.9 | 63.3 | −19.4 | 0 | −3.5 |
| | SEQ. ID. NO:1734 | | | | | | |
| 1523 | ATTGTCTATCTGGAGACAGG | −1.5 | −22.1 | 67.5 | −18.2 | −2.4 | −8.2 |
| | SEQ. ID. NO:1735 | | | | | | |
| 72 | CCCAGCGATTTTGCTACAAA | −1.4 | −24.1 | 66.2 | −21.1 | −1.6 | −7.1 |
| | SEQ. ID. NO:1736 | | | | | | |
| 106 | GGGAGGATTCTGGACTGAGT | −1.4 | −24.9 | 73.5 | −23.5 | 0 | −3.1 |
| | SEQ. ID. NO:1737 | | | | | | |
| 254 | GAAATGTCCAGAAGAAATCC | −1.4 | −19 | 56.8 | −17.6 | 0 | −2.2 |
| | SEQ. ID. NO:1738 | | | | | | |
| 1324 | AAGGAACATAGCTTCAACCG | −1.4 | −21.2 | 60.9 | −19.3 | −0.2 | −4.6 |
| | SEQ. ID. NO:1739 | | | | | | |
| 1470 | TGAGTCATTTTCAGTTCCCC | −1.4 | −26 | 75.9 | −24.6 | 0 | −5.4 |
| | SEQ. ID. NO:1740 | | | | | | |
| 1491 | GTAAGCAGAGCATACTCCTC | −1.4 | −24.3 | 71.9 | −21.4 | −1.4 | −6.3 |
| | SEQ. ID. NO:1741 | | | | | | |
| 1627 | GGCAAAGTGTTGAGGATTTT | −1.4 | −21.5 | 64.5 | −19.2 | −0.7 | −4 |
| | SEQ. ID. NO:1742 | | | | | | |
| 1878 | ATTACAACATAAATATTCAT | −1.4 | −14.1 | 47.7 | −12.7 | 0 | −4.6 |
| | SEQ. ID. NO:1743 | | | | | | |
| 70 | CAGCGATTTTGCTACAAATG | −1.3 | −20.1 | 59.2 | −17.2 | −1.6 | −7.2 |
| | SEQ. ID. NO:1744 | | | | | | |
| 155 | TTCTACCTCCTTGGATTGTT | −1.3 | −24.8 | 72.5 | −23.5 | 0.2 | −4.6 |
| | SEQ. ID. NO:1745 | | | | | | |
| 180 | CTTCTTTCACTCCTTCTACG | −1.3 | −24.1 | 70.7 | −22.8 | 0 | −3 |
| | SEQ. ID. NO:1746 | | | | | | |
| 524 | ACGAGGAAATCTGTGGTTGA | −1.3 | −21.6 | 63.4 | −20.3 | 0 | −3.5 |
| | SEQ. ID. NO:1747 | | | | | | |
| 525 | GACGAGGAAATCTGTGGTTG | −1.3 | −21.6 | 63.4 | −20.3 | 0 | −3.5 |
| | SEQ. ID. NO:1748 | | | | | | |
| 562 | CTGCTGGGGGTAGAAACCCA | −1.3 | −27.4 | 74.4 | −22 | −4.1 | 10.8 |
| | SEQ. ID. NO:1749 | | | | | | |
| 1404 | ATACCACTATTTCGAATTCT | −1.3 | −20.1 | 60.2 | −18.8 | 0 | −6.7 |
| | SEQ. ID. NO:1750 | | | | | | |
| 1464 | ATTTTCAGTTCCCCAATACT | −1.3 | −23.9 | 68.8 | −22.6 | 0 | −2.8 |
| | SEQ. ID. NO:1751 | | | | | | |
| 1526 | TGTATTGTCTATCTGGAGAC | −1.3 | −21.1 | 65.9 | −18.7 | −1 | −4.8 |
| | SEQ. ID. NO:1752 | | | | | | |
| 1560 | TCCTGAAGCTTCTCTACTGC | −1.3 | −25.2 | 73.9 | −22.5 | 0 | −10.8 |
| | SEQ. ID. NO:1753 | | | | | | |
| 1920 | CTATCTAGCCCAATATTTAC | −1.3 | −21.2 | 63 | −19.9 | 0 | −4.1 |
| | SEQ. ID. NO:1754 | | | | | | |
| 2034 | TAGTTCAACAGATAGAATTG | −1.3 | −16.6 | 53.8 | −15.3 | 0 | −3.7 |
| | SEQ. ID. NO:1755 | | | | | | |
| 338 | CCATCCAAATTTTTCAATTG | −1.2 | −19.3 | 57.6 | −17.4 | −0.5 | −6.1 |
| | SEQ. ID. NO:1756 | | | | | | |
| 453 | TTCTGTCCCAGAGGACCTGC | −1.2 | −29 | 81.2 | −24.8 | −3 | −8.2 |
| | SEQ. ID. NO:1757 | | | | | | |
| 559 | CTGGGGGTAGAAACCCAGGT | −1.2 | −27.1 | 74.6 | −21.8 | −4.1 | −9.8 |
| | SEQ. ID. NO:1758 | | | | | | |
| 589 | TATTCCAGGAGAGTACCACT | −1.2 | −24.2 | 70.6 | −22.1 | −0.5 | −8.9 |
| | SEQ. ID. NO:1759 | | | | | | |
| 623 | AGAGAGTCTCAGCTGGCATA | −1.2 | −24.9 | 74.9 | −22.3 | −1.1 | −10 |
| | SEQ. ID. NO:1760 | | | | | | |
| 748 | GTGGTATCCAGAGGCTCTGT | −1.2 | −27.4 | 81 | −24.6 | −1.5 | −8 |
| | SEQ. ID. NO:1761 | | | | | | |
| 1191 | ATGAGAAAATTTTCTTCTGC | −1.2 | −17.9 | 56.4 | −14.5 | −1 | −12.5 |
| | SEQ. ID. NO:1762 | | | | | | |
| 1242 | TTTGTGAATTCTACAAGAAC | −1.2 | −16.7 | 53.6 | −14.1 | −0.9 | −10.5 |
| | SEQ. ID. NO:1763 | | | | | | |
| 1469 | GAGTCATTTTCAGTTCCCCA | −1.2 | −26.7 | 77.2 | −25.5 | 0 | −4.1 |
| | SEQ. ID. NO:1764 | | | | | | |
| 2024 | GATAGAATTGAAGTAACAAT | −1.1 | −14.6 | 48.7 | −12.6 | −0.7 | −4.2 |
| | SEQ. ID. NO:1765 | | | | | | |
| 28 | GGATAAGTCGGGGAGACAAT | −1 | −22.2 | 64.3 | −19.1 | −2.1 | −5.5 |
| | SEQ. ID. NO:1766 | | | | | | |
| 263 | CATCTTGAGGAAATGTCCAG | −1 | −21.4 | 63.4 | −18.3 | −2.1 | −5.7 |
| | SEQ. ID. NO:1767 | | | | | | |
| 289 | AAAAAACTCCAAAGTGTCTG | −1 | −17 | 52.8 | −16 | 0 | −3 |
| | SEQ. ID. NO:1768 | | | | | | |
| 290 | AAAAAAACTCCAAAGTGTCT | −1 | −16.3 | 51.2 | −14.6 | −0.5 | −3 |
| | SEQ. ID. NO:1769 | | | | | | |
| 472 | GTATGGTTCCACTTCCAGGT | −1 | −27.2 | 79 | −25.3 | −0.7 | −5.6 |
| | SEQ. ID. NO:1770 | | | | | | |
| 518 | AAATCTGTGGTTCAACTTGG | −1 | −19.9 | 60.3 | −18.9 | 0 | −3.4 |
| | SEQ. ID. NO:1771 | | | | | | |
| 798 | ATGCTTCTCCTGAAGAAACC | −1 | −22.7 | 65.2 | −19.5 | −2.2 | −5.7 |
| | SEQ. ID. NO:1772 | | | | | | |
| 1075 | TGGGGTGAGTTCAGTTTTCT | −1 | −24.6 | 75.3 | −23.6 | 0 | −2.9 |
| | SEQ. ID. NO:1773 | | | | | | |
| 1165 | TTCTTCTTTTAAAATTTTAT | −1 | −14.7 | 49.9 | −13.2 | 0 | −8 |
| | SEQ. ID. NO:1774 | | | | | | |
| 1167 | AATTCTTCTTTTAAAATTTT | −1 | −14.3 | 48.8 | −13.3 | 0 | −6.5 |
| | SEQ. ID. NO:1775 | | | | | | |
| 1499 | CAATTGCTGTAAGCAGAGCA | −1 | −22.6 | 66.2 | −18.5 | −3.1 | −10.6 |
| | SEQ. ID. NO:1776 | | | | | | |
| 1500 | ACAATTGCTGTAAGCAGAGC | −1 | −22.1 | 65.5 | −18.3 | −2.8 | −9 |
| | SEQ. ID. NO:1777 | | | | | | |
| 1644 | AGGCGACCCAGGAGACAGGC | −1 | −29.6 | 79.5 | −27.6 | −0.9 | −5.4 |
| | SEQ. ID. NO:1778 | | | | | | |
| 2025 | AGATAGAATTGAAGTAACAA | −1 | −14.6 | 48.8 | −13.6 | 0 | −3.3 |
| | SEQ. ID. NO:1779 | | | | | | |
| 2030 | TCAACAGATAGAATTGAAGT | −1 | −16.7 | 53.5 | −15.1 | −0.3 | −4.1 |
| | SEQ. ID. NO:1780 | | | | | | |
| 191 | AGTCTTCTTTTCTTCTTTCA | −0.9 | −22.2 | 70.9 | −21.3 | 0 | −1.5 |
| | SEQ. ID. NO:1781 | | | | | | |
| 192 | AAGTCTTCTTTTCTTCTTTC | −0.9 | −20.8 | 66.9 | −19.9 | 0 | −2.4 |
| | SEQ. ID. NO:1782 | | | | | | |
| 246 | CAGAAGAAATCCAGGAAACT | −0.9 | −18.4 | 55.4 | −17 | −0.2 | −5.7 |
| | SEQ. ID. NO:1783 | | | | | | |
| 397 | AAAAGAAAATTCATCTGTGG | −0.9 | −15.3 | 49.8 | −14.4 | 0 | −4.8 |
| | SEQ. ID. NO:1784 | | | | | | |
| 498 | GGAAACTGAACATTGCTGTA | −0.9 | −20 | 59.7 | −18.4 | −0.5 | −3.9 |
| | SEQ. ID. NO:1785 | | | | | | |
| 590 | ATATTCCAGGAGAGTACCAC | −0.9 | −23.3 | 68.6 | −21.7 | −0.5 | −5.3 |
| | SEQ. ID. NO:1786 | | | | | | |
| 636 | GTTTCTCCCTGGTAGAGAGT | −0.9 | −26.5 | 79 | −24.5 | −1 | −7 |
| | SEQ. ID. NO:1787 | | | | | | |
| 1327 | ACGAAGGAACATAGCTTCAA | −0.9 | −19.8 | 58.6 | −16.9 | −2 | −5.6 |
| | SEQ. ID. NO:1788 | | | | | | |
| 1341 | AAAATCTCAGCTGAACGAAG | −0.9 | −17.8 | 54.3 | −15.8 | 0 | −10.1 |
| | SEQ. ID. NO:1789 | | | | | | |
| 1512 | GGAGACAGGATAACAATTGC | −0.9 | −20.2 | 60.5 | −19.3 | 0 | −7 |
| | SEQ. ID. NO:1790 | | | | | | |
| 1825 | AATAAAGGAAAGTTATACAT | −0.9 | −13.6 | 46.6 | −12.7 | 0 | −2.8 |
| | SEQ. ID. NO:1791 | | | | | | |
| 286 | AAACTCCAAAGTGTCTGAAG | −0.8 | −19 | 57.6 | −17.5 | −0.5 | −5 |
| | SEQ. ID. NO:1792 | | | | | | |
| 533 | AATAGGATGACGAGGAAATC | −0.8 | −17.8 | 54.7 | −17 | 0 | −3.5 |
| | SEQ. ID. NO:1793 | | | | | | |
| 638 | CAGTTTCTCCCTGGTAGAGA | −0.8 | −26 | 76.4 | −24.5 | −0.5 | −6.3 |
| | SEQ. ID. NO:1794 | | | | | | |
| 1195 | CAAAATGAGAAAATTTTCTT | −0.8 | −13.4 | 46 | −10.4 | −1 | −12.5 |
| | SEQ. ID. NO:1795 | | | | | | |
| 1881 | GTAATTACAACATAAATATT | −0.8 | −13.2 | 45.9 | −11.9 | 0 | −8.1 |
| | SEQ. ID. NO:1796 | | | | | | |
| 69 | AGCGATTTTGCTACAAATGC | −0.7 | −21.2 | 61.9 | −18.9 | −1.5 | −8 |
| | SEQ. ID. NO:1797 | | | | | | |
| 337 | CATCCAAATTTTTCAATTGA | −0.7 | −17.9 | 55.2 | −16.5 | −0.5 | −8.1 |
| | SEQ. ID. NO:1798 | | | | | | |
| 633 | TCTCCCTGGTAGAGAGTCTC | −0.7 | −26.8 | 80.4 | −25.2 | −0.7 | −8.7 |
| | SEQ. ID. NO:1799 | | | | | | |
| 951 | TTAGATTTACACTGAATTTC | −0.7 | −16.8 | 54.5 | −16.1 | 0 | −3.8 |
| | SEQ. ID. NO:1800 | | | | | | |
| 1497 | ATTGCTGTAAGCAGAGCATA | −0.7 | −22.3 | 66.6 | −18.5 | −3.1 | −10.7 |
| | SEQ. ID. NO:1801 | | | | | | |
| 1556 | GAAGCTTCTCTACTGCCTCT | −0.7 | −26.1 | 76.2 | −24.4 | 0 | −10 |
| | SEQ. ID. NO:1802 | | | | | | |
| 154 | TCTACCTCCTTGGATTGTTT | −0.6 | −24.8 | 72.5 | −23.5 | −0.5 | −4.6 |
| | SEQ. ID. NO:1803 | | | | | | |
| 593 | CATATATTCCAGGAGAGTAC | −0.6 | −20.8 | 63.5 | −20.2 | 0 | −5.3 |
| | SEQ. ID. NO:1804 | | | | | | |
| 728 | CTCCACAAACAACACACAGC | −0.6 | −22.2 | 63 | −21.6 | 0 | −2.8 |
| | SEQ. ID. NO:1805 | | | | | | |
| 1414 | TTCATCAGAGATACCACTAT | −0.6 | −20.9 | 63.3 | −20.3 | 0 | −3.5 |
| | SEQ. ID. NO:1806 | | | | | | |
| 1439 | AAAAACTAAACATAGGTGTT | −0.6 | −14.9 | 49 | −12.7 | −1.5 | −5.5 |
| | SEQ. ID. NO:1807 | | | | | | |
| 1626 | GCAAAGTGTTGAGGATTTTC | −0.6 | −20.7 | 63.4 | −19.2 | −0.7 | −3.4 |
| | SEQ. ID. NO:1808 | | | | | | |
| 1879 | AATTACAACATAAATATTCA | −0.6 | −13.4 | 46.2 | −12.8 | 0 | −4.6 |
| | SEQ. ID. NO:1809 | | | | | | |
| 252 | AATGTCCAGAAGAAATCCAG | −0.5 | −19.8 | 58.8 | −19.3 | 0 | −2.2 |
| | SEQ. ID. NO:1810 | | | | | | |
| 532 | ATAGGATGACGAGGAAATCT | −0.5 | −19.4 | 58.3 | −18.4 | −0.1 | −3.5 |
| | SEQ. ID. NO:1811 | | | | | | |
| 859 | CATGTACATATCCATCACAC | −0.5 | −21.5 | 64 | −20.5 | 0 | −8 |
| | SEQ. ID. NO:1812 | | | | | | |
| 1074 | GGGGTGAGTTCAGTTTTCTC | −0.5 | −25 | 77.5 | −24.5 | 0 | −3.4 |
| | SEQ. ID. NO:1813 | | | | | | |
| 1168 | GAATTCTTCTTTTAAAATTT | −0.5 | −14.8 | 49.7 | −14.3 | 0 | −6.3 |
| | SEQ. ID. NO:1814 | | | | | | |
| 1520 | GTCTATCTGGAGACAGGATA | −0.5 | −22.3 | 68 | −19.4 | −2.4 | −9.5 |
| | SEQ. ID. NO:1815 | | | | | | |
| 1993 | GGCAAACAGGGCTTGCCAAT | −0.5 | −26.2 | 71.2 | −22 | −3.7 | −10.4 |
| | SEQ. ID. NO:1816 | | | | | | |
| 721 | AACAACACACAGCTCATCCC | −0.4 | −24.4 | 68.2 | −24 | 0 | −4.4 |
| | SEQ. ID. NO:1817 | | | | | | |
| 749 | AGTGGTATCCAGAGGCTCTG | −0.4 | −26.2 | 77.5 | −24.5 | −1.2 | −7.6 |
| | SEQ. ID. NO:1818 | | | | | | |
| 828 | TTTTTACACTTGTACACAGC | −0.4 | −20.7 | 63.5 | −20.3 | 0 | −6.3 |
| | SEQ. ID. NO:1819 | | | | | | |
| 938 | GAATTTCAGTTAACAAGCAT | −0.4 | −18.2 | 56.6 | −17.8 | 0 | −7.3 |
| | SEQ. ID. NO:1820 | | | | | | |
| 952 | CTTAGATTTACACTGAATTT | −0.4 | −17.3 | 55.2 | −16.9 | 0 | −3.8 |
| | SEQ. ID. NO:1821 | | | | | | |
| 1506 | AGGATAACAATTGCTGTAAG | −0.4 | −18 | 56 | −16.9 | −0.4 | −7 |
| | SEQ. ID. NO:1822 | | | | | | |
| 1517 | TATCTGGAGACAGGATAACA | −0.4 | −20 | 60.8 | −17.2 | −2.4 | −9.5 |
| | SEQ. ID. NO:1823 | | | | | | |
| 78 | CCAGATCCCAGCGATTTTGC | −0.3 | −27.7 | 74.9 | −26.5 | −0.7 | −5.9 |
| | SEQ. ID. NO:1824 | | | | | | |
| 193 | TAAGTCTTCTTTTCTTCTTT | −0.3 | −20.1 | 64.5 | 1−9.2 | −0.3 | −3 |
| | SEQ. ID. NO:1825 | | | | | | |
| 370 | ATGGGAATGTTCAATGAGAT | −0.3 | −19.2 | 58.7 | −18.9 | 0 | −5.7 |
| | SEQ. ID. NO:1826 | | | | | | |
| 634 | TTCTCCCTGGTAGAGAGTCT | −0.3 | −26.5 | 78.8 | −25.1 | −1 | −7 |
| | SEQ. ID. NO:1827 | | | | | | |
| 773 | ACCCCTCACAGGTCAGTGCA | −0.3 | −30.3 | 83.7 | −29.3 | −0.5 | −6 |
| | SEQ. ID. NO:1828 | | | | | | |
| 789 | CRFAAFAAACCRRRACACCC | −0.3 | −22.1 | 62.4 | −21.8 | 0 | −2.8 |
| | SEQ. ID. NO:1829 | | | | | | |
| 1735 | TTCACAGAGAAGTGGGGTAA | −0.3 | −21.6 | 64.9 | −20.4 | −0.7 | −4.6 |
| | SEQ. ID. NO:1830 | | | | | | |
| 2081 | AATAAAATATATGCAATATG | −0.3 | −11.8 | 42.9 | −10.8 | −0.5 | −6.5 |
| | SEQ. ID. NO:1831 | | | | | | |
| 77 | CAGATCCCAGCGATTTTGCT | −0.2 | −26.6 | 73.4 | −24.8 | −1.5 | −7.4 |
| | SEQ. ID. NO:1832 | | | | | | |
| 635 | TTTCTCCCTGGTAGAGAGTC | −0.2 | −25.7 | 77.1 | −24.4 | −1 | −7 |
| | SEQ. ID. NO:1833 | | | | | | |
| 720 | ACAACACACAGCTCATCCCC | −0.2 | −27.1 | 73.8 | −26.9 | 0 | −4.4 |
| | SEQ. ID. NO:1834 | | | | | | |
| 778 | TTTACACCCCTCACAGGTCA | −0.2 | −27.4 | 76.4 | −26.5 | −0.5 | −3.9 |
| | SEQ. ID. NO:1835 | | | | | | |
| 801 | GTAATGCTTCTCCTGAAGAA | −0.2 | −21.4 | 63.5 | −19 | −2.2 | −6.7 |
| | SEQ. ID. NO:1836 | | | | | | |
| 1407 | GAGATACCACTATTTCGAAT | −0.2 | −19.9 | 59.4 | −19.7 | 0 | −6.7 |
| | SEQ. ID. NO:1837 | | | | | | |
| 1633 | GAGACAGGCAAAGTGTTGAG | −0.2 | −21.5 | 64.5 | −20.4 | −0.7 | −4 |
| | SEQ. ID. NO:1838 | | | | | | |
| 247 | CCAGAAGAAATCCAGGAAAC | −0.1 | −19.5 | 57.1 | −19.4 | 0 | −5.7 |
| | SEQ. ID. NO:1839 | | | | | | |
| 426 | TCTGTTAAAACACCAAATAA | −0.1 | −15.6 | 49.9 | −15.5 | 0 | −5.5 |
| | SEQ. ID. NO:1840 | | | | | | |
| 829 | GTTTTTACACTTGTACACAG | −0.1 | −20.1 | 62.5 | −20 | 0 | −6.2 |
| | SEQ. ID. NO:1841 | | | | | | |
| 1462 | TTTCAGTTCCCCAATACTTT | −0.1 | −24 | 69.2 | −23.9 | 0 | −2.9 |
| | SEQ. ID. NO:1842 | | | | | | |
| 1494 | GCTGTAAGCAGAGCATACTC | −0.1 | −23.7 | 70.7 | −20.4 | −3.2 | −8.2 |
| | SEQ. ID. NO:1843 | | | | | | |
| 1524 | TATTGTCTATCTGGAGACAG | −0.1 | −20.6 | 64.1 | −18.2 −2.3 | −7.8 | |
| | SEQ. ID. NO:1844 | | | | | | |
| 15 | AGACAATGAGGTGAGGAGGA | 0 | −22 | 65.5 | −22 | 0 | −3.1 |
| | SEQ. ID. NO:1845 | | | | | | |
| 1515 | TCTGGAGACAGGATAACAAT | 0 | −19.6 | 59.4 | −17.2 | −2.4 | −9.5 |
| | SEQ. ID. NO:1846 | | | | | | |
| 1516 | ATCTGGAGACAGGATAACAA | 0 | −19.6 | 59.4 | −17.2 | −2.4 | −9.5 |
| | SEQ. ID. NO:1847 | | | | | | |
| 1559 | CCTGAAGCTTCTCTACTGCC | 0 | −26.8 | 75.9 | −25.4 | 0 | −10.8 |
| | SEQ. ID. NO:1848 | | | | | | |
| 1877 | TTACAACATAAATATTCATC | 0 | −14.5 | 48.8 | −14.5 | 0 | −4.6 |
| | SEQ. ID. NO:1849 | | | | | | |
| 27 | GATAAGTCGGGGAGACAATG | 0.1 | −21 | 61.7 | −19.7 | −1.3 | −4.5 |
| | SEQ. ID. NO:1850 | | | | | | |
| 188 | CTTCTTTTCTTCTTTCACTC | 0.1 | −22.1 | 69.7 | −22.2 | 0 | 0 |
| | SEQ. ID. NO:1851 | | | | | | |
| 939 | TGAATTTCAGTTAACAAGCA | 0.1 | −18.2 | 56.6 | −18.3 | 0 | −7.3 |
| | SEQ. ID. NO:1852 | | | | | | |
| 1186 | AAAATTTTCTTCTGCACTGA | 0.1 | −19.1 | 58.6 | −19.2 | 0 | −6.3 |
| | SEQ. ID. NO:1853 | | | | | | |
| 1871 | CATAAATATTCATCAAGATT | 0.1 | −15 | 49.7 | −15.1 | 0 | −4.6 |
| | SEQ. ID. NO:1854 | | | | | | |
| 19 | GGGGAGACAATGAGGTGAGG | 0.2 | −23.8 | 69.1 | −24 | 0 | −3.1 |
| | SEQ. ID. NO:1855 | | | | | | |
| 245 | AGAAGAAATCCAGGAAACTA | 0.2 | −17.4 | 53.7 | −17 | −0.3 | −5.7 |
| | SEQ. ID. NO:1856 | | | | | | |
| 541 | GTTGGAATAATAGGATGACG | 0.2 | −18.5 | 56.3 | −18.7 | 0 | −3 |
| | SEQ. ID. NO:1857 | | | | | | |
| 544 | CAGGTTGGAATAATAGGATG | 0.2 | −18.8 | 57.7 | −19 | 0 | −1.6 |
| | SEQ. ID. NO:1858 | | | | | | |
| 1099 | AAAATGTAGAAGAGTCTGTT | 0.2 | −17.1 | 54.9 | −16.8 | −0.2 | −5.8 |
| | SEQ. ID. NO:1859 | | | | | | |
| 1190 | TGAGAAAATTTTCTTCTGCA | 0.2 | −18.6 | 57.7 | −16.6 | −1 | −12.5 |
| | SEQ. ID. NO:1860 | | | | | | |
| 1503 | ATAACAATTGCTGTAAGCAG | 0.2 | −18.7 | 57.4 | −15.8 | −3.1 | −7.9 |
| | SEQ. ID. NO:1861 | | | | | | |
| 1513 | TGGAGACAGGATAACAATTG | 0.2 | −18.4 | 56.5 | −17.9 | −0.4 | −7.4 |
| | SEQ. ID. NO:1862 | | | | | | |
| 1736 | TTTCACAGAGAAGTGGGGTA | 0.2 | −22.4 | 67.6 | −21.7 | −0.7 | −4.8 |
| | SEQ. ID. NO:1863 | | | | | | |
| 463 | CACTTCCAGGTTCTGTCCCA | 0.3 | −29.1 | 81.8 | −28.9 | −0.2 | −3.7 |
| | SEQ. ID. NO:1864 | | | | | | |
| 756 | GCATTATAGTGGTATCCAGA | 0.3 | −23 | 68.9 | −22.5 | −0.6 | −6.9 |
| | SEQ. ID. NO:1865 | | | | | | |
| 1357 | CGGAAGTTTCTTATTGAAAA | 0.3 | −17 | 53.2 | −15.8 | −1.4 | −6.6 |
| | SEQ. ID. NO:1866 | | | | | | |
| 1406 | AGATACCACTATTTCGAATT | 0.3 | −19.4 | 58.5 | −19.7 0 | −6.7 | |
| | SEQ. ID. NO:1867 | | | | | | |
| 1409 | CAGAGATACCACTATTTCGA | 0.3 | −21.3 | 62.7 | −20.9 −0.5 | −5.5 | |
| | SEQ. ID. NO:1868 | | | | | | |
| 1440 | TAAAAACTAAACATAGGTGT | 0.3 | −14.5 | 48.2 | −14.1 −0.5 | −3.5 | |
| | SEQ. ID. NO:1869 | | | | | | |
| 1557 | TGAAGCTTCTCTACTGCCTC | 0.3 | −25.2 | 73.9 | −24.1 | 0 | −10.8 |
| | SEQ. ID. NO:1870 | | | | | | |
| 1823 | TAAAGGAAAGTTATACATCA | 0.3 | −15.4 | 50.5 | −15.7 | 0 | −2.6 |
| | SEQ. ID. NO:1871 | | | | | | |
| 257 | GAGGAAATGTCCAGAAGAAA | 0.4 | −18.4 | 55.8 | −16.7 | −2.1 | −4.9 |
| | SEQ. ID. NO:1872 | | | | | | |
| 336 | ATCCAAATTTTTCAATTGAA | 0.4 | −16.5 | 52.3 | −15.8 | 0 | −10.1 |
| | SEQ. ID. NO:1873 | | | | | | |
| 399 | GAAAAAGAAAATTCATCTGT | 0.4 | −14 | 47.2 | −14.4 | 0 | −4.8 |
| | SEQ. ID. NO:1874 | | | | | | |
| 461 | CTTCCAGGTTCTGTCCCAGA | 0.4 | −28.8 | 81.9 | −−28.1 | −1 | −5.3 |
| | SEQ. ID. NO:1875 | | | | | | |
| 517 | AATCTGTGGTTGAACTTGGG | 0.4 | −21.8 | 65 | −22.2 | 0 | −3.4 |
| | SEQ. ID. NO:1876 | | | | | | |
| 537 | GAATAATAGGATGACGAFFA | 0.4 | −18.4 | 55.9 | −18.8 | 0 | −3.5 |
| | SEQ. ID. NO:1877 | | | | | | |
| 588 | ARRCCAFFAFAFRACCACRC | 0.4 | −24.9 | 72.9 | −23.8 | −1.4 | −8.5 |
| | SEQ. ID. NO:1878 | | | | | | |
| 639 | RCAGTTTCTCCCTGGTAGAG | 0.4 | −25.8 | 76.8 | −25.7 | −0.2 | −4.6 |
| | SEQ. ID. NO:1879 | | | | | | |
| 777 | TTACACCCCTCACAGGTCAG | 0.4 | −27.3 | 76.4 | −27 | −0.5 | −4.1 |
| | SEQ. ID. NO:1880 | | | | | | |
| 860 | GCATGTACATATCCATCACA | 0.4 | −23.1 | 67.6 | −23 | 0 | −8 |
| | SEQ. ID. NO:1881 | | | | | | |
| 1492 | TGTAAGCAGAGCATACTCCT | 0.4 | −23.9 | 70 | −22.8 | −1.4 | −6.4 |
| | SEQ. ID. NO:1882 | | | | | | |
| 1869 | TAAATATTCATCAAGATTTC | 0.4 | −14.8 | 49.8 | −15.2 | 3.8 | −4.6 |
| | SEQ. ID. NO:1883 | | | | | | |
| 385 | ATCTGTGGTAGGTAAATGGG | 0.5 | −21.7 | 65.4 | −22.2 | 0 | −1.9 |
| | SEQ. ID. NO:1884 | | | | | | |
| 718 | AACACACAGCTCATCCCCTT | 0.5 | −27.2 | 74.4 | −27.7 | 0 | −4.4 |
| | SEQ. ID. NO:1885 | | | | | | |
| 946 | TTTACACTGAATTTCAGTTA | 0.5 | −18.1 | 57.5 | −16.3 | −2.3 | −11.1 |
| | SEQ. ID. NO:1886 | | | | | | |
| 1408 | AGAGATACCACTATTTCGAA | 0.5 | −19.9 | 59.6 | −19.7 | −0.5 | −6.5 |
| | SEQ. ID. NO:1887 | | | | | | |
| 1733 | CACAGAGAAGTGGGGTAAAC | 0.5 | −20.6 | 61.5 | −20.6 | −0.1 | −4.2 |
| | SEQ. ID. NO:1888 | | | | | | |
| 555 | GGGTAGAAACCCAGGTTGGA | 0.6 | −25.7 | 71.8 | −23 | −3.3 | −8.9 |
| | SEQ. ID. NO:1889 | | | | | | |
| 1183 | ATTTTCTTCTGCACTGAATT | 0.6 | −20.6 | 63.1 | −21.2 | 0 | −4.9 |
| | SEQ. ID. NO:1890 | | | | | | |
| 1452 | CCAATACTTTTATAAAAACT | 0.6 | −14.8 | 48.5 | −14.9 | 0 | −7.8 |
| | SEQ. ID. NO:1891 | | | | | | |
| 2004 | CAATTTAATTAGGCAAACAG | 0.6 | −16.2 | 51.6 | −16.8 | 0 | −4 |
| | SEQ. ID. NO:1892 | | | | | | |
| 298 | GGTCTTCAAAAAAAACTCCA | 0.7 | −18.2 | 55 | −18.9 | 0 | −2.8 |
| | SEQ. ID. NO:1893 | | | | | | |
| 464 | CCACTTCCAGGTTCTGTCCC | 0.7 | −30.4 | 84.3 | −30.6 | −0.2 | −3.7 |
| | SEQ. ID. NO:1894 | | | | | | |
| 553 | GTAGAAACCCAGGTTGGAAT | 0.7 | −22.6 | 64.7 | −22.4 | −0.8 | −6.5 |
| | SEQ. ID. NO:1895 | | | | | | |
| 1444 | TTTATAAAAACTAAACATAG | 0.7 | −10.8 | 41.2 | −11.5 | 0 | −5.5 |
| | SEQ. ID. NO:1896 | | | | | | |
| 1696 | CCATGACATCAGCATCTCAG | 0.7 | −24.2 | 70.3 | −24.9 | 0 | −4.5 |
| | SEQ. ID. NO:1897 | | | | | | |
| 1737 | ATTTCACAGAGAAGTGGGGT | 0.7 | −22.7 | 68.1 | −22.5 | −0.7 | −4.8 |
| | SEQ. ID. NO:1898 | | | | | | |
| 1826 | AAATAAAGGAAAGTTATACA | 0.7 | −12.9 | 45.1 | −13.6 | 0 | −2.8 |
| | SEQ. ID. NO:1899 | | | | | | |
| 4 | TGAGGAGGAGGAGAGAGTCT | 0.8 | −23.7 | 71.9 | −24.5 | 0 | −5.7 |
| | SEQ. ID. NO:1900 | | | | | | |
| 189 | TCTTCTTTTCTTCTTTCACT | 0.8 | −22.1 | 69.7 | −22.9 | 0 | 0 |
| | SEQ. ID. NO:1901 | | | | | | |
| 255 | GGAAATGTCCAGAAGAAATC | 0.8 | −18.2 | 55.6 | −17.6 | −1.3 | −4.4 |
| | SEQ. ID. NO:1902 | | | | | | |
| 288 | AAAAACTCCAAAGTGTCTGA | 0.8 | −18.3 | 55.7 | −18.4 | −0.5 | −3.6 |
| | SEQ. ID. NO:1903 | | | | | | |
| 947 | ATTTACACTGAATTTCAGTT | 0.8 | −18.4 | 58.1 | −16.7 | −2.5 | −11.3 |
| | SEQ. ID. NO:1904 | | | | | | |
| 1022 | GCAAGTCACGACCTTCACTG | 0.8 | −25.1 | 70.7 | −25.9 | 0 | −4.7 |
| | SEQ. ID. NO:1905 | | | | | | |
| 1098 | AAATGTAGAAGAGTCTGTTG | 0.8 | −17.8 | 56.8 | −18.1 | −0.2 | −5.8 |
| | SEQ. ID. NO:1906 | | | | | | |
| 1326 | CGAAGGAACATAGCTTCAAC | 0.8 | −19.8 | 58.6 | −18.6 | −2 | −5.6 |
| | SEQ. ID. NO:1907 | | | | | | |
| 1420 | TATATATTCATCAGAGATAC | 0.8 | −16.5 | 54.3 | −17.3 | 0 | −3.9 |
| | SEQ. ID. NO:1908 | | | | | | |
| 1461 | TTCAGTTCCCCAATACTTTT | 0.8 | −24 | 69.2 | −24.8 | 0 | −2.9 |
| | SEQ. ID. NO:1909 | | | | | | |
| 1885 | ACATGTAATTACAACATAAA | 0.8 | −14.3 | 47.8 | −13.8 | −0.6 | −10.3 |
| | SEQ. ID. NO:1910 | | | | | | |
| 281 | CCAAAGTGTCTGAAGTTTCA | 0.9 | −21.4 | 64 | −22.3 | 0 | −4.5 |
| | SEQ. ID. NO:1911 | | | | | | |
| 502 | TTGGGGAAACTGAACATTGC | 0.9 | −20.7 | 60.7 | −21.1 | −0.2 | −2.9 |
| | SEQ. ID. NO:1912 | | | | | | |
| 1089 | AGAGTCTGTTGATCTGGGGT | 0.9 | −25.1 | 76.3 | −26 | 0 | −5 |
| | SEQ. ID. NO:1913 | | | | | | |
| 398 | AAAAAGAAAATTCATCTGTG | 1 | −13.4 | 46 | −14.4 | 0 | −4.6 |
| | SEQ. ID. NO:1914 | | | | | | |
| 473 | AGTATGGTTCCACTTCCAGG | 1 | −26 | 75.6 | −26.1 | −0.7 | −5.6 |
| | SEQ. ID. NO:1915 | | | | | | |
| 499 | GGGAAACTGAACATTGCTGT | 1 | −21.5 | 62.7 | −21.8 | −0.5 | −4 |
| | SEQ. ID. NO:1916 | | | | | | |
| 729 | TCTCCACAAACAACACACAG | 1 | −20.8 | 60.5 | −21.8 | 0 | −1.3 |
| | SEQ. ID. NO:1917 | | | | | | |
| 1405 | GATACCACTATTTCGAATTC | 1 | −19.8 | 59.6 | −20.8 | 0 | −6.7 |
| | SEQ. ID. NO:1918 | | | | | | |
| 1872 | ACATAAATATTCATCAAGAT | 1 | −15.1 | 49.9 | −16.1 | 0 | −4.1 |
| | SEQ. ID. NO:1919 | | | | | | |
| 450 | TGTCCCAGAGGACCTGCCAC | 1.1 | −30.5 | 82.1 | −28.6 | −3 | −8.6 |
| | SEQ. ID. NO:1920 | | | | | | |
| 552 | TAGAAACCCAGGTTGGAATA | 1.1 | −21.1 | 61.3 | −21.3 | −0.8 | −7 |
| | SEQ. ID. NO:1921 | | | | | | |
| 727 | TCCACAAACAACACACAGCT | 1.1 | −22.2 | 63 | −23.3 | 0 | −4.3 |
| | SEQ. ID. NO:1922 | | | | | | |
| 1200 | TCCGTCAAAATGAGAAAATT | 1.1 | −16.6 | 51.4 | −17.2 | −0.1 | −3.2 |
| | SEQ. ID. NO:1923 | | | | | | |
| 1445 | TTTTATAAAAACTAAACATA | 1.1 | −10.9 | 41.4 | −11.5 | 0 | −7.5 |
| | SEQ. ID. NO:1924 | | | | | | |
| 1525 | GTATTGTCTATCTGGAGACA | 1.1 | −21.8 | 67.3 | −20.8 | −2.1 | −9.3 |
| | SEQ. ID. NO:1925 | | | | | | |
| 1697 | TCCATGACATCAGCATCTCA | 1.1 | −24.6 | 71.7 | −25.7 | 0 | −4.5 |
| | SEQ. ID. NO:1926 | | | | | | |
| 415 | ACCAAATAAATTTTCAGAAA | 1.2 | −14.4 | 47.6 | −15.6 | 0 | −5.3 |
| | SEQ. ID. NO:1927 | | | | | | |
| 1704 | TTTACTCTCCATGACATCAG | 1.2 | −22 | 66.1 | −23.2 | 0 | −4.5 |
| | SEQ. ID. NO:1928 | | | | | | |
| 2003 | AATTTAATTAGGCAAACAGG | 1.2 | −16.7 | 52.7 | −17.9 | 0 | −4.1 |
| | SEQ. ID. NO:1929 | | | | | | |
| 253 | AAATGTCCAGAAGAAATCCA | 1.3 | −19.1 | 56.8 | −20.4 | 0 | −2.2 |
| | SEQ. ID. NO:1930 | | | | | | |
| 371 | AATGGGAATGTTCAATGAGA | 1.3 | −18.5 | 56.8 | −19.8 | 0 | −4.9 |
| | SEQ. ID. NO:1931 | | | | | | |
| 503 | CTTGGGGAAACTGAACATTG | 1.3 | −19.8 | 58.7 | −1.1 | 0.6 | −2.3 |
| | SEQ. ID. NO:1932 | | | | | | |
| 641 | CCTCAGTTTCTCCCTGGTAG | 1.3 | −28.1 | 80.9 | −28.9 | −0.2 | −4.2 |
| | SEQ. ID. NO:1933 | | | | | | |
| 1091 | GAAGAGTCTGTTGATCTGGG | 1.3 | −22.6 | 68.6 | −23.4 | −0.1 | −5.8 |
| | SEQ. ID. NO:1934 | | | | | | |
| 1419 | ATATATTCATCAGAGATACC | 1.3 | −18.8 | 58.9 | −20.1 | 0 | −3.6 |
| | SEQ. ID. NO:1935 | | | | | | |
| 1700 | CTCTCCATGACATCAGCATC | 1.3 | −24.8 | 72.5 | −26.1 | 0 | −4.1 |
| | SEQ. ID. NO:1936 | | | | | | |
| 1 | GGAGGAGGAGAGAGTCTCGT | 1.4 | −25.5 | 75.7 | −24.5 | −2.4 | −10 |
| | SEQ. ID. NO:1937 | | | | | | |
| 107 | TGGGAGGATTCTGGACTGAG | 1.4 | −23.7 | 69.9 | −25.1 | 0 | −2.9 |
| | SEQ. ID. NO:1938 | | | | | | |
| 291 | AAAAAAAACTCCAAAGTGTC | 1.4 | −14.7 | 48.1 | −15.4 | −0.5 | −3 |
| | SEQ. ID. NO:1939 | | | | | | |
| 299 | TGGTCTTCAAAAAAAACTCC | 1.4 | −17.5 | 53.8 | −18.9 | 0 | −2.5 |
| | SEQ. ID. NO:1940 | | | | | | |
| 414 | CCAAATAAATTTTCAGAAAA | 1.4 | −13.5 | 45.8 | −14.4 | −0.1 | −7.7 |
| | SEQ. ID. NO:1941 | | | | | | |
| 713 | ACAGCTCATCCCCTTTGATC | 1.4 | −27.2 | 76.7 | −28.6 | 0 | −4.4 |
| | SEQ. ID. NO:1942 | | | | | | |
| 1199 | CCGTCAAAATGAGAAAATTT | 1.4 | −16.3 | 50.7 | −17.2 | −0.1 | −5 |
| | SEQ. ID. NO:1943 | | | | | | |
| 1354 | AAGTTTCTTATTGAAAATCT | 1.4 | −15.7 | 51.7 | −15.6 | −1.4 | −4.5 |
| | SEQ. ID. NO:1944 | | | | | | |
| 280 | CAAAGTGTCTGAAGTTTCAT | 1.5 | −19.4 | 60.2 | −20.9 | 0 | −4.7 |
| | SEQ. ID. NO:1945 | | | | | | |
| 526 | TGACGAGGAAATCTGTGGTT | 1.5 | −21.6 | 63.4 | −23.1 | 0 | −3.5 |
| | SEQ. ID. NO:1946 | | | | | | |
| 551 | AGAAACCCAGGTTGGAATAA | 1.5 | −20.7 | 59.9 | −21.3 | −0.8 | −7 |
| | SEQ. ID. NO:1947 | | | | | | |
| 857 | TGTACATATCCATCACACAG | 1.5 | −21.5 | 64.2 | −23 | 0 | −5.9 |
| | SEQ. ID. NO:1948 | | | | | | |
| 1182 | TTTTCTTCTGCACTGAATTC | 1.5 | −21 | 64.6 | −22.5 | 0 | −5.9 |
| | SEQ. ID. NO:1949 | | | | | | |
| 1184 | AATTTTCTTCTGCACTGAAT | 1.5 | −19.8 | 60.6 | −21.3 | 0 | −4.9 |
| | SEQ. ID. NO:1950 | | | | | | |
| 1835 | GTACAAGTGAAATAAAGGAA | 1.5 | −14.9 | 49 | −16.4 | 0 | −4.6 |
| | SEQ. ID. NO:1951 | | | | | | |
| 1876 | TACAACATAAATATTCATCA | 1.5 | −15.1 | 49.8 | −16.6 | 0 | −4.6 |
| | SEQ. ID. NO:1952 | | | | | | |
| 14 | GACAATGAGGTGAGGAGGAG | 1.6 | −22 65.5 | −23.6 | 0 | −3.1 | |
| | SEQ. ID. NO:1953 | | | | | | |
| 262 | ATCTTGAGGAAATGTCCAGA | 1.6 | −21.3 | 63.5 | −20.8 | −2.1 | −6.6 |
| | SEQ. ID. NO:1954 | | | | | | |
| 404 | TTTCAGAAAAAGAAAATTCA | 1.6 | −12.8 | 44.9 | −13.8 | −0.3 | −5.1 |
| | SEQ. ID. NO:1955 | | | | | | |
| 416 | CACCAAATAAATTTTCAGAA | 1.6 | −15.8 | 50.3 | −17.4 | 0 | −4.7 |
| | SEQ. ID. NO:1956 | | | | | | |
| 766 | ACAGGTCAGTGCATTATAGT | 1.6 | −22.8 | 69.9 | −24.4 | 0 | −5.4 |
| | SEQ. ID. NO:1957 | | | | | | |
| 259 | TTGAGGAAATGTCCAGAAGA | 1.7 | −19.9 | 59.7 | −19.5 | −2.1 | −5.2 |
| | SEQ. ID. NO:1958 | | | | | | |
| 767 | CACAGGTCAGTGCATTATAG | 1.7 | −22.3 | 67.6 | −24 | 0 | −5.4 |
| | SEQ. ID. NO:1959 | | | | | | |
| 1451 | CAATACTTTTATAAAAACTA | 1.7 | −12.5 | 44.4 | −13.7 | 0 | −7.8 |
| | SEQ. ID. NO:1960 | | | | | | |
| 1822 | AAAGGAAAGTTATACATCAG | 1.7 | −15.7 | 51.2 | −17.4 | 0 | −2.9 |
| | SEQ. ID. NO:1961 | | | | | | |
| 287 | AAAACTCCAAAGTGTCTGAA | 1.8 | −18.3 | 55.7 | −19.4 | −0.5 | −5 |
| | SEQ. ID. NO:1962 | | | | | | |
| 640 | CTCAGTTTCTCCCTGGTAGA | 1.8 | −26.7 | 78.5 | −28 | −0.2 | −4.2 |
| | SEQ. ID. NO:1963 | | | | | | |
| 943 | ACACTGAATTTCAGTTAACA | 1.8 | −18.4 | 57.3 | −17.7 | −2.5 | −11.3 |
| | SEQ. ID. NO:1964 | | | | | | |
| 16 | GAGACAATGAGGTGAGGAGG | 1.9 | −22 | 65.5 | −23.9 | 0 | −3.1 |
| | SEQ. ID. NO:1965 | | | | | | |
| 405 | TTTTCAGAAAAAGAAAATTC | 1.9 | −12.2 | 43.9 | −12.7 | −1.3 | −7.1 |
| | SEQ. ID. NO:1966 | | | | | | |
| 406 | ATTTTCAGAAAAAGAAAATT | 1.9 | −11.8 | 43 | −11.5 | −2.2 | −8.1 |
| | SEQ. ID. NO:1967 | | | | | | |
| 516 | ATCTGTGGTTGAACTTGGGG | 1.9 | −23.7 | 69.9 | −25.6 | 0 | −3.4 |
| | SEQ. ID. NO:1968 | | | | | | |
| 542 | GGTTGGAATAATAGGATGAC | 1.9 | −18.9 | 58.1 | −20.8 | 0 | −2 |
| | SEQ. ID. NO:1969 | | | | | | |
| 722 | AAACAACACACAGCTCATCC | 1.9 | −21.7 | 62.7 | −23.6 | 0 | −4.4 |
| | SEQ. ID. NO:1970 | | | | | | |
| 786 | AAGAAACCTTTACACCCCTC | 1.9 | −23.9 | 66 | −28.8 | 0 | −2.4 |
| | SEQ. ID. NO:1971 | | | | | | |
| 1100 | TAAAATGTAGAAGAGTCTGT | 1.9 | −16.7 | 54 | −18.1 | −0.2 | −5.8 |
| | SEQ. ID. NO:1972 | | | | | | |
| 1170 | CTGAATTCTTCTTTTAAAAT | 1.9 | −15.5 | 51 | −16.7 | −0.4 | −6.9 |
| | SEQ. ID. NO:1973 | | | | | | |
| 1180 | TTCTTCTGCACTGAATTCTT | 1.9 | −21.8 | 66.3 | −23.7 | 0 | −6.9 |
| | SEQ. ID. NO:1974 | | | | | | |
| 1181 | TTTCTTCTGCACTGAATTCT | 1.9 | −21.8 | 66.3 | −23.7 | 0 | −6.9 |
| | SEQ. ID. NO:1975 | | | | | | |
| 1325 | GAAGGAACATAGCTTCAACC | 1.9 | −21 | 61.7 | −21.3 | −1.5 | −5.4 |
| | SEQ. ID. NO:1976 | | | | | | |
| 1441 | ATAAAAACTAAACATAGGTG | 1.9 | −13.3 | 45.8 | −15.2 | 0 | −3.5 |
| | SEQ. ID. NO:1977 | | | | | | |
| 190 | GTCTTCTTTTCTTCTTTCAC | 2 | −22.4 | 71.2 | −24.4 | 0 | −0.8 |
| | SEQ. ID. NO:1978 | | | | | | |
| 194 | CTAAGTCTTCTTTTCTTCTT | 2 | −20.9 | 66.3 | −22.3 | −0.3 | −3 |
| | SEQ. ID. NO:1979 | | | | | | |
| 540 | TTGGAATAATAGGATGACGA | 2 | −17.9 | 54.8 | −19.9 | 0 | −3.5 |
| | SEQ. ID. NO:1980 | | | | | | |
| 550 | GAAACCCAGGTTGGAATAAT | 2 | −20.7 | 59.7 | −22.1 | −0.3 | −7 |
| | SEQ. ID. NO:1981 | | | | | | |
| 726 | CCACAAACAACACACAGCTC | 2 | −22.2 | 63 | −24.2 | 0 | −4.4 |
| | SEQ. ID. NO:1982 | | | | | | |
| 776 | TACACCCCTCACAGGTCAGT | 2 | −28.4 | 79.5 | −29.7 | −0.5 | −4.1 |
| | SEQ. ID. NO:1983 | | | | | | |
| 1169 | TGAATTCTTCTTTTAAAATT | 2 | −14.7 | 49.4 | −16 | −0.4 | −6.9 |
| | SEQ. ID. NO:1984 | | | | | | |
| 1496 | TTGCTGTAAGCAGAGCATAC | 2 | −22.5 | 67.2 | −21.4 | −3.1 −10.7 | |
| | SEQ. ID. NO:1985 | | | | | | |
| 1698 | CTCCATGACATCAGCATCTC | 2 | −24.8 | 72.5 | −26.8 | 0 | −4.5 |
| | SEQ. ID. NO:1986 | | | | | | |
| 1734 | TCACAGAGAAGTCCCCTAAA | 2 | −20.8 | 62.4 | −21.9 | −0.7 | −4.6 |
| | SEQ. ID. NO:1987 | | | | | | |
| 1836 | GGTACAAGTGAAATAAAGGA | 2 | −16.8 | 52.9 | −18.8 | 0 | −5.2 |
| | SEQ. ID. NO:1988 | | | | | | |
| 527 | ATGACGAGGAAATCTGTGGT | 2.1 | −21.5 | 63.1 | −23.6 | 0 | −3.5 |
| | SEQ. ID. NO:1989 | | | | | | |
| 557 | GGGGGTAGAAACCCAGGTTG | 2.1 | −26.3 | 73.1 | −24.3 | −4.1 | −9.1 |
| | SEQ. ID. NO:1990 | | | | | | |
| 783 | AAACCTTTACACCCCTCACA | 2.1 | −25.6 | 69.2 | −27.7 | 0 | −1.4 |
| | SEQ. ID. NO:1991 | | | | | | |
| 1090 | AAGAGTCTGTTGATCTGGGG | 2.1 | −23.2 | 69.9 | −24.8 | −0.1 | −5.8 |
| | SEQ. ID. NO:1992 | | | | | | |
| 1198 | CGTCAAAATGAGAAAATTTT | 2.1 | −14.4 | 47.5 | −15.8 | −0.5 | −7.2 |
| | SEQ. ID. NO:1993 | | | | | | |
| 1418 | TATATTCATCAGAGATACCA | 2.1 | −19.5 | 60.2 | −21.6 | 0 | −3.5 |
| | SEQ. ID. NO:1994 | | | | | | |
| 1884 | CATCTAATTACAACATAAAT | 2.1 | −14.1 | 47.4 | −14.9 | −0.6 | −10.3 |
| | SEQ. ID. NO:1995 | | | | | | |
| 261 | TCTTGAGGAAATGTCCAGAA | 2.3 | −20.6 | 61.5 | −20.8 | −2.1 | −6.3 |
| | SEQ. ID. NO:1996 | | | | | | |
| 548 | AACCCAGGTTGGAATAATAG | 2.3 | −20.5 | 60 | −21.9 | −0.8 | −6.1 |
| | SEQ. ID. NO:1997 | | | | | | |
| 549 | AAACCCAGGTTGGAATAATA | 2.3 | −19.8 | 58.1 | −21.2 | −0.8 | −7 |
| | SEQ. ID. NO:1998 | | | | | | |
| 854 | CAGGAGAGTACCACTCTTCA | 2.3 | −24.5 | 72.3 | −23.4 | −3.4 | −8.6 |
| | SEQ. ID. NO:1999 | | | | | | |
| 785 | AGAAACCTTTACACCCCTCA | 2.3 | −25.3 | 69.1 | −27.6 | 0 | −2.5 |
| | SEQ. ID. NO:2000 | | | | | | |
| 1189 | GAGAAAATTTTCTTCTGCAC | 2.3 | −18.8 | 58.3 | −18.9 | −1 | −12.5 |
| | SEQ. ID. NO:2001 | | | | | | |
| 6 | GGTGAGGAGGAGGAGAGAGT | 2.4 | −24.8 | 74.5 | −27.2 | 0 | 0 |
| | SEQ. ID. NO:2002 | | | | | | |
| 269 | AAGTTTCATCTTGAGGAAAT | 2.4 | −18.2 | 57.1 | −19.7 | −0.7 | −7.9 |
| | SEQ. ID. NO:2003 | | | | | | |
| 297 | GTCTTCAAAAAAAACTCCAA | 2.4 | −16.3 | 51.2 | −18.7 | 0 | −1.9 |
| | SEQ. ID. NO:2004 | | | | | | |
| 530 | AGGATGACGAGGAAATCTGT | 2.4 | −20.9 | 61.6 | −22.8 | −0.1 | −3.5 |
| | SEQ. ID. NO:2005 | | | | | | |
| 637 | AGTTTCTCCCTGGTAGAGAG | 2.4 | −25.3 | 75.5 | −26.6 | −1 | −7 |
| | SEQ. ID. NO:2006 | | | | | | |
| 1449 | ATACTTTTATAAAAACTAAA | 2.4 | −11.1 | 41.8 | −13 | 0 | −7.8 |
| | SEQ. ID. NO:2007 | | | | | | |
| 400 | AGAAAAAGAAAATTCATCTG | 2.5 | −12.8 | 44.9 | −14.4 | −0.7 | −4.8 |
| | SEQ. ID. NO:2008 | | | | | | |
| 514 | CTGTGGTTGAACTTGGGGAA | 2.5 | −23.2 | 67.3 | −25.7 | 0 | −3.1 |
| | SEQ. ID. NO:2009 | | | | | | |
| 531 | TAGGATGACGAGGAAATCTG | 2.5 | −19.4 | 58.2 | −21.4 | −0.1 | −3.5 |
| | SEQ. ID. NO:2010 | | | | | | |
| 558 | TGGGGGTAGAAACCCAGGTT | 2.5 | −26.3 | 73.1 | −24.7 | −4.1 | −9 |
| | SEQ. ID. NO:2011 | | | | | | |
| 1703 | TTACTCTCCATGACATCAGC | 2.5 | −23.7 | 70.1 | −26.2 | 0 | −4.5 |
| | SEQ. ID. NO:2012 | | | | | | |
| 1518 | CTATCTGGAGACAGGATAAC | 2.6 | −20.2 | 61.5 | −20.4 | −2.4 | −9.5 |
| | SEQ. ID. NO:2013 | | | | | | |
| 1701 | ACTCTCCATGACATCAGCAT | 2.6 | −24.6 | 71.4 | −27.2 | 0 | −4.5 |
| | SEQ. ID. NO:2014 | | | | | | |
| 505 | AACTTGGGGAAACTGAACAT | 2.7 | −19.2 | 57.2 | −21.4 | −0.2 | −2.5 |
| | SEQ. ID. NO:2015 | | | | | | |
| 1495 | TGCTGTAAGCAGAGCATACT | 2.7 | −23.3 | 68.9 | −23.1 | −2.9 | −9 |
| | SEQ. ID. NO:2016 | | | | | | |
| 506 | GAACTTGGGGAAACTGAACA | 2.8 | −19.8 | 58.3 | −22.1 | −0.2 | −2.5 |
| | SEQ. ID. NO:2017 | | | | | | |
| 543 | AGGTTGGAATAATAGGATGA | 2.8 | −18.7 | 57.8 | −21.5 | 0 | −1.3 |
| | SEQ. ID. NO:2018 | | | | | | |
| 547 | ACCCAGGTTGGAATAATAGG | 2.8 | −22.4 | 64.4 | −24.3 | −0.8 | −4.3 |
| | SEQ. ID. NO:2019 | | | | | | |
| 556 | GGGGTAGAAACCCAGGTTGG | 2.8 | −26.3 | 73.1 | −25 | −4.1 | −9.1 |
| | SEQ. ID. NO:2020 | | | | | | |
| 944 | TACACTGAATTTCAGTTAAC | 2.8 | −17.4 | 55.5 | −17.7 | −2.5 | −11.3 |
| | SEQ. ID. NO:2021 | | | | | | |
| 1355 | GAAGTTTCTTATTGAAAATC | 2.8 | −15.4 | 51.1 | −16.7 | −1.4 | −5.8 |
| | SEQ. ID. NO:2022 | | | | | | |
| 1448 | TACTTTTATAAAAACTAAAC | 2.8 | −11.3 | 42.2 | −13.6 | 0 | −7.8 |
| | SEQ. ID. NO:2023 | | | | | | |
| 1450 | AATACTTTTATAAAAACTAA | 2.8 | −11.1 | 41.8 | −13.4 | 0 | −7.6 |
| | SEQ. ID. NO:2024 | | | | | | |
| 1837 | GGGTACAAGTGAAATAAAGG | 2.8 | −17.4 | 54.1 | −20.2 | 0 | −5.2 |
| | SEQ. ID. NO:2025 | | | | | | |
| 8 | GAGGTGAGGAGGAGGAGAGA | 2.9 | −24.2 | 72.3 | −27.1 | 0 | −0 |
| | SEQ. ID. NO:2026 | | | | | | |
| 417 | ACACCAAATAAATTTTCAGA | 2.9 | −16.7 | 52.4 | −19.6 | 0 | −4.7 |
| | SEQ. ID. NO:2027 | | | | | | |
| 554 | GGTAGAAACCCAGGTTGGAA | 2.9 | −23.8 | 67.2 | −25.8 | −0.8 | −7 |
| | SEQ. ID. NO:2028 | | | | | | |
| 561 | TGCTGGGGGTAGAAACCCAG | 2.9 | −26.5 | 72.8 | −25.3 | −4.1 | −10.8 |
| | SEQ. ID. NO:2029 | | | | | | |
| 1172 | CACTGAATTCTTCTTTTAAA | 2.9 | −17.1 | 54.5 | −19.3 | −0.4 | −6.9 |
| | SEQ. ID. NO:2030 | | | | | | |
| 1447 | ACTTTTATAAAAACTAAACA | 2.9 | −12.3 | 44 | −14.7 | 0 | −7.8 |
| | SEQ. ID. NO:2031 | | | | | | |
| 1453 | CCCAATACTTTTATAAAAAC | 2.9 | −15.9 | 50.3 | −18.3 | 0 | −7.8 |
| | SEQ. ID. NO:2032 | | | | | | |
| 1457 | GTTCCCCAATACTTTTATAA | 2.9 | −21.5 | 62.8 | −24.4 | 0 | −3.7 |
| | SEQ. ID. NO:2033 | | | | | | |
| 1875 | ACAACATAAATATTCATCAA | 2.9 | −14.7 | 48.7 | −17.6 | 0 | −4.6 |
| | SEQ. ID. NO:2034 | | | | | | |
| 17 | GGAGACAATGAGGTGAGGAG | 3 | −22 | 65.5 | −25 | 0 | −2.7 |
| | SEQ. ID. NO:2035 | | | | | | |
| 407 | AATTTTCAGAAAAAGAAAAT | 3 −11 | 41.4 | −11.5 | −2.5 | −8.1 | |
| | SEQ. ID. NO:2036 | | | | | | |
| 945 | TTACACTGAATTTCAGTTAA | 3 −17.3 | 55.3 | −17.8 | −2.5 | −11.3 | |
| | SEQ. ID. NO:2037 | | | | | | |
| 1185 | AAATTTTCTTCTGCACTGAA | 3 −19.1 | 58.6 | −22.1 | 0 | −4.8 | |
| | SEQ. ID. NO:2038 | | | | | | |
| 2 | AGGAGGAGGAGAGAGTCTCG | 3.1 | −24.3 | 72.4 | −25 | −2.4 | −10 |
| | SEQ. ID. NO:2039 | | | | | | |
| 504 | ACTTGGGGAAACTGAACATT | 3.1 | −20 | 59.3 | −22.6 | −0.2 | −2.5 |
| | SEQ. ID. NO:2040 | | | | | | |
| 1179 | TCTTCTGCACTGAATTCTTC | 3.1 | −22.1 | 67.5 | −25.2 | 0 | −6.9 |
| | SEQ. ID. NO:2041 | | | | | | |
| 1442 | TATAAAAACTAAACATAGGT | 3.1 | −13 | 45.3 | −16.1 | 0 | −3.2 |
| | SEQ. ID. NO:2042 | | | | | | |
| 1558 | CTGAAGCTTCTCTACTGCCT | 3.1 | −25.7 | 74.2 | −27.4 | 0 | −10.8 |
| | SEQ. ID. NO:2043 | | | | | | |
| 1702 | TACTCTCCATGACATCAGCA | 3.1 | −24.3 | 70.9 | −27.4 | 0 | −4.5 |
| | SEQ. ID. NO:2044 | | | | | | |
| 1873 | AACATAAATATTCATCAAGA | 3.1 | −14.4 | 48.3 | −17.5 | 0 | −4.6 |
| | SEQ. ID. NO:2045 | | | | | | |
| 1880 | TAATTACAACATAAATATTC | 3.1 | −12.4 | 44.4 | −15.5 | 0 | −4.6 |
| | SEQ. ID. NO:2046 | | | | | | |
| 1171 | ACTGAATTCTTCTTTTAAAA | 3.2 | −15.7 | 51.5 | −18.2 | −0.4 | −6.9 |
| | SEQ. ID. NO:2047 | | | | | | |
| 1173 | GCACTGAATTCTTCTTTTAA | 3.2 | −19.6 | 60.5 | −22.8 | 0.3 | −6.2 |
| | SEQ. ID. NO:2048 | | | | | | |
| 403 | TTCAGAAAAAGAAAATTCAT | 3.3 | −12.7 | 44.6 | −15.1 | −0.7 | −4.8 |
| | SEQ. ID. NO:2049 | | | | | | |
| 1827 | GAAATAAAGGAAAGTTATAC | 3.3 | −12.8 | 45 | −16.1 | 0 | −2.8 |
| | SEQ. ID. NO:2050 | | | | | | |
| 258 | TGAGGAAATGTCCAGAAGAA | 3.4 | −19.1 | 57.5 | −20.4 | −2.1 | −4.9 |
| | SEQ. ID. NO:2051 | | | | | | |
| 292 | CAAAAAAAACTCCAAAGTGT | 3.4 | −15 | 48.3 | −17.7 | −0.5 | −3 |
| | SEQ. ID. NO:2052 | | | | | | |
| 372 | AAATGGGAATGTTCAATGAG | 3.5 | −17.2 | 53.8 | −20.7 | 0 | −5.7 |
| | SEQ. ID. NO:2053 | | | | | | |
| 1188 | AGAAAATTTTCTTCTGCACT | 3.5 | −19.1 | 58.9 | −20.9 | −0.5 | −11.6 |
| | SEQ. ID. NO:2054 | | | | | | |
| 1634 | GGAGACAGGCAAAGTGTTGA | 3.5 | −22.7 | 66.8 | −25.3 | −0.7 | −4 |
| | SEQ. ID. NO:2055 | | | | | | |
| 7 | AGGTGAGGAGGAGGAGAGAG | 3.6 | −23.6 | 71.2 | −27.2 | 0 | 0 |
| | SEQ. ID. NO:2056 | | | | | | |
| 500 | GGGGAAACTGAACATTGCTG | 3.6 | −21.5 | 62.2 | −24.6 | −0.2 | −3.8 |
| | SEQ. ID. NO:2057 | | | | | | |
| 784 | GAAACCTTTACACCCCTCAC | 3.6 | −25.5 | 69.4 | −29.1 | 0 | −2 |
| | SEQ. ID. NO:2058 | | | | | | |
| 1514 | CTGGAGACAGGATAACAATT | 3.6 | −19.3 | 58.4 | −21.1 | −1.8 | −5.9 |
| | SEQ. ID. NO:2059 | | | | | | |
| 256 | AGGAAATGTCCAGAAGAAAT | 3.7 | −17.8 | 54.6 | −19.4 | −2.1 | −4.9 |
| | SEQ. ID. NO:2060 | | | | | | |
| 515 | TCTGTGGTTGAACTTGGGGA | 3.7 | −24.3 | 71.2 | −28 | 0 | −3.4 |
| | SEQ. ID. NO:2061 | | | | | | |
| 775 | ACACCCCTCACAGGTCAGTG | 3.8 | −28.7 | 79.9 | −31.4 | −1 | −5.4 |
| | SEQ. ID. NO:2062 | | | | | | |
| 401 | CAGAAAAAGAAAATTCATCT | 3.9 | −13.5 | 46.1 | −16.5 | −0.7 | −4.8 |
| | SEQ. ID. NO:2063 | | | | | | |
| 260 | CTTGAGGAAATGTCCAGAAG | 4 −20.2 | 60.3 | −22.8 | −1.3 | −5.5 | |
| | SEQ. ID. NO:2064 | | | | | | |
| 408 | AAATTTTCAGAAAAAGAAAA | 4 −10.3 | 40.1 | −12.7 | −1.6 | −8.1 | |
| | SEQ. ID. NO:2065 | | | | | | |
| 409 | TAAATTTTCAGAAAAAGAAA | 4 −10.7 | 40.9 | −13.8 | −0.8 | −8.1 | |
| | SEQ. ID. NO:2066 | | | | | | |
| 723 | CAAACAACACACAGCTCATC | 4 −20.4 | 60.2 | −24.4 | 0 | −4.4 | |
| | SEQ. ID. NO:2067 | | | | | | |
| 1459 | CAGTTCCCCAATACTTTTAT | 4 −23.2 | 66.7 | −27.2 | 0 | −2.9 | |
| | SEQ. ID. NO:2068 | | | | | | |
| 13 | ACAATGAGGTGAGGAGGAGG | 4.1 | −22.6 | 66.8 | −26.7 | 0 | −3.1 |
| | SEQ. ID. NO:2069 | | | | | | |
| 295 | CTTCAAAAAAAACTCCAAAG | 4.1 | −14 46.5 | −18.1 | 0 | −2 | |
| | SEQ. ID. NO:2070 | | | | | | |
| 462 | ACTTCCAGGTTCTGTCCCAG | 4.1 | −28.4 | 81.2 | −32 | −0.1 | −3.7 |
| | SEQ. ID. NO:2071 | | | | | | |
| 402 | TCAGAAAAAGAAAATTCATC | 4.2 | −13 45.3 | −16.3 | −0.7 | −4.8 | |
| | SEQ. ID. NO:2072 | | | | | | |
| 940 | CTGAATTTCAGTTAACAAGC | 4.2 | −18.4 | 57.2 | −21.5 | −1 | −8.4 |
| | SEQ. ID. NO:2073 | | | | | | |
| 1356 | GGAAGTTTCTTATTGAAAAT | 4.2 | −16.2 | 52.4 | −19.4 | −0.9 | −6.6 |
| | SEQ. ID. NO:2074 | | | | | | |
| 1446 | CTTTTATAAAAACTAAACAT | 4.2 | −12.1 | 43.5 | −15.8 | 0 | −7.8 |
| | SEQ. ID. NO:2075 | | | | | | |
| 410 | ATAAATTTTCAGAAAAAGAA | 4.3 | −11.4 | 42.2 | −15.1 | −0.3 | −7.6 |
| | SEQ. ID. NO:2076 | | | | | | |
| 1458 | AGTTCCCCAATACTTTTATA | 4.3 | −22.2 | 65 | −26.5 | 0 | −2.8 |
| | SEQ. ID. NO:2077 | | | | | | |
| 413 | CAAATAAATTTTCAGAAAAA | 4.4 | −10.8 | 41 | −14.4 | −0.6 | −8.1 |
| | SEQ. ID. NO:2078 | | | | | | |
| 420 | AAAACACCAAATAAATTTTC | 4.4 | −13.3 | 45.4 | −17.7 | 0 | −4.7 |
| | SEQ. ID. NO:2079 | | | | | | |
| 622 | GAGAGTCTCAGCTGGCATAC | 4.4 | −25.1 | 75.3 | −28.6 | −0.3 | −9.3 |
| | SEQ. ID. NO:2080 | | | | | | |
| 501 | TGGGGAAACTGAACATTGCT | 4.5 | −21.5 | 62.2 | −25.5 | −0.2 | −3.8 |
| | SEQ. ID. NO:2081 | | | | | | |
| 2039 | TTCCCTAGTTCAACAGATAG | 4.5 | −22 | 65.7 | −26.5 | 0 | −3.6 |
| | SEQ. ID. NO:2082 | | | | | | |
| 725 | CACAAACAACACACAGCTCA | 4.6 | −20.9 | 60.6 | −25.5 | 0 | −4.4 |
| | SEQ. ID. NO:2083 | | | | | | |
| 942 | CACTGAATTTCAGTTAACAA | 4.6 | −17.5 | 54.9 | −19.6 | −2.5 | −11.3 |
| | SEQ. ID. NO:2084 | | | | | | |
| 1456 | TTCCCCAATACTTTTATAAA | 4.6 | −19.6 | 58 | −24.2 | 0 | −5.7 |
| | SEQ. ID. NO:2085 | | | | | | |
| 296 | TCTTCAAAAAAAACTCCAAA | 4.8 | −14.4 | 47.3 | −19.2 | 0 | −1 |
| | SEQ. ID. NO:2086 | | | | | | |
| 423 | GTTAAAACACCAAATAAATT | 4.8 | −13.7 | 46.1 | −18.5 | 0 | −4.1 |
| | SEQ. ID. NO:2087 | | | | | | |
| 763 | GGTCAGTGCATTATAGTGGT | 4.8 | −24.3 | 74.1 | −29.1 | 0 | −5.4 |
| | SEQ. ID. NO:2088 | | | | | | |
| 9 | TGAGGTGAGGAGGAGGAGAG | 4.9 | −23.5 | 70.7 | −28.5 | 0 | 0 |
| | SEQ. ID. NO:2089 | | | | | | |
| 560 | GCTGGGGGTAGAAACCCAGG | 4.9 | −27.7 | 75.4 | −28.3 | −4.3 | −10.9 |
| | SEQ. ID. NO:2090 | | | | | | |
| 1460 | TCAGTTCCCCAATACTTTTA | 4.9 | −23.6 | 68.3 | −28.5 | 0 | −2.9 |
| | SEQ. ID. NO:2091 | | | | | | |
| 244 | GAAGAAATCCAGGAAACTAA | 5 | −16.7 | 51.9 | −21.1 | −0.3 | −5.7 |
| | SEQ. ID. NO:2092 | | | | | | |
| 418 | AACACCAAATAAATTTTCAG | 5.1 | −15.4 | 49.6 | −20.5 | 0 | −4.7 |
| | SEQ. ID. NO:2093 | | | | | | |
| 528 | GATGACGAGGAAATCTGTGG | 5.1 | −20.9 | 61.4 | −26 | 0 | −3.3 |
| | SEQ. ID. NO:2094 | | | | | | |
| 1187 | GAAAATTTTCTTCTGCACTG | 5.1 | −19.1 | 58.6 | −23.1 | 0 | −10.1 |
| | SEQ. ID. NO:2095 | | | | | | |
| 765 | CAGGTCAGTGCATTATAGTG | 5.2 | −22.6 | 69.1 | −27.8 | 0 | −5.4 |
| | SEQ. ID. NO:2096 | | | | | | |
| 774 | CACCCCTCACAGGTCAGTGC | 5.2 | −30.3 | 83.7 | −34.8 | −0.5 | −5.9 |
| | SEQ. ID. NO:2097 | | | | | | |
| 1443 | TTATAAAAACTAAACATAGG | 5.2 | −11.9 | 43.1 | −17.1 | 0 | −3.5 |
| | SEQ. ID. NO:2098 | | | | | | |
| 3 | GAGGAGGAGGAGAGAGTCTC | 5.3 | −24.1 | 74 | −28 | −1.3 | −8.7 |
| | SEQ. ID. NO:2099 | | | | | | |
| 724 | ACAAACAACACACAGCTCAT | 5.4 | −20.2 | 59.5 | −25.6 | 0 | −4.4 |
| | SEQ. ID. NO:2100 | | | | | | |
| 529 | GGATGACGAGGAAATCTGTG | 5.5 | −20.9 | 61.4 | −25.9 | −0.1 | −3.7 |
| | SEQ. ID. NO:2101 | | | | | | |
| 762 | GTCAGTGCATTATAGTGGTA | 5.6 | −22.8 | 70.5 | −28.4 | 0 | −5 |
| | SEQ. ID. NO:2102 | | | | | | |
| 422 | TTAAAACACCAAATAAATTT | 5.7 | −12.6 | 44.1 | −18.3 | 0 | −4.5 |
| | SEQ. ID. NO:2103 | | | | | | |
| 411 | AATAAATTTTCAGAAAAAGA | 5.8 | −11.4 | 52.2 | −16.3 | −0.8 | −8.1 |
| | SEQ. ID. NO:2104 | | | | | | |
| 762 | AGGTCAGTGCATTATAGTGG | 5.8 | −23.1 | 70.7 | −28.9 | 0 | −5.4 |
| | SEQ. ID. NO:2105 | | | | | | |
| 243 | AAGAAATCCAGGAAACTAAG | 5.9 | −16.1 | 50.9 | −21.4 | −0.3 | −5.7 |
| | SEQ. ID. NO:2106 | | | | | | |
| 1101 | ATAAAATGTAGAAGAGTCTG | 5.9 | −15.5 | 51.1 | −20.9 | −0.2 | −5.8 |
| | SEQ. ID. NO:2107 | | | | | | |
| 5 | GTGAGGAGGAGGAGAGAGTC | 6 | −24 | 73.5 | −30 | 0 | −3.5 |
| | SEQ. ID. NO:2108 | | | | | | |
| 1874 | CAACATAAATATTCATCAAG | 6 | −14.5 | 48.3 | −20.5 | 0 | −4.6 |
| | SEQ. ID. NO:2109 | | | | | | |
| 425 | CTGTTAAAACACCAAATAAA | 6.2 | −14.5 | 47.5 | −20.7 | 0 | −5.5 |
| | SEQ. ID. NO:2110 | | | | | | |
| 941 | ACTGAATTTCAGTTAACAAG | 6.3 | −16.8 | 53.8 | −20.8 | −2.3 | −11 |
| | SEQ. ID. NO:2111 | | | | | | |
| 512 | GTGGTTGAACTTGGGGAAAC | 6.4 | −21.8 | 64 | −28.2 | 0 | −3.4 |
| | SEQ. ID. NO:2112 | | | | | | |
| 10 | ATGAGGTGAGGAGGAGGAGA | 6.5 | −23.6 | 70.4 | −30.1 | 0 | −0.3 |
| | SEQ. ID. NO:2113 | | | | | | |
| 424 | TGTTAAAACACCAAATAAAT | 6.6 | −13.6 | 45.8 | −20.2 | 0 | −5.4 |
| | SEQ. ID. NO:2114 | | | | | | |
| 1519 | TCTATCTGGAGACAGGATAA | 6.6 | −20.4 | 62.4 | −25.2 | −1.8 | −9.5 |
| | SEQ. ID. NO:2115 | | | | | | |
| 421 | TAAAACACCAAATAAATTTT | 6.7 | −12.6 | 44.1 | −19.3 | 0 | −4.7 |
| | SEQ. ID. NO:2116 | | | | | | |
| 419 | AAACACCAAATAAATTTTCA | 6.8 | −14.7 | 48 | −21.5 | 0 | −4.7 |
| | SEQ. ID. NO:2117 | | | | | | |
| 507 | TGAACTTGGGGAAACTGAAC | 6.9 | −19.1 | 57.1 | −25.5 | −0.2 | −1.8 |
| | SEQ. ID. NO:2118 | | | | | | |
| 513 | TGTGGTTGAACTTGGGGAAA | 7 −21.6 | 63.3 | −28.6 | 0 | −3.4 | |
| | SEQ. ID. NO:2119 | | | | | | |
| 510 | GGTTGAACTTGGGGAAACTG | 7.1 | −21.5 | 62.8 | −28.1 | −0.2 | −3.6 |
| | SEQ. ID. NO:2120 | | | | | | |
| 412 | AAATAAATTTTCAGAAAAAG | 7.3 | −10.1 | 39.8 | −16.5 | −0.8 | −8.1 |
| | SEQ. ID. NO:2121 | | | | | | |
| 294 | TTCAAAAAAAACTCCAAAGT | 7.5 | −14.3 | 47.2 | −21.2 | −0.3 | −2.9 |
| | SEQ. ID. NO:2122 | | | | | | |
| 511 | TGGTTGAACTTGGGGAAACT | 7.5 | −21.5 | 62.8 | −28.5 | −0.2 | −3.6 |
| | SEQ. ID. NO:2123 | | | | | | |
| 758 | GTGCATTATAGTGGTATCCA | 7.6 | −23.6 | 70.6 | −30.5 | −0.4 | −6.2 |
| | SEQ. ID. NO:2124 | | | | | | |
| 1417 | ATATTCATCAGAGATACCAC | 7.6 | −20 | 61.3 | −27.6 | 0 | −3.5 |
| | SEQ. ID. NO:2125 | | | | | | |
| 1416 | TATTCATCAGAGATACCACT | 7.7 | −20.9 | 63.3 | −28.6 | 0 | −3.5 |
| | SEQ. ID. NO:2126 | | | | | | |
| 11 | AATGAGGTGAGGAGGAGGAG | 7.8 | −22.3 | 66.6 | −30.1 | 0 | −1.2 |
| | SEQ. ID. NO:2127 | | | | | | |
| 508 | TTGAACTTGGGGAAACTGAA | 7.9 | −19 | 57 | −26.4 | −0.2 | −1.8 |
| | SEQ. ID. NO:2128 | | | | | | |
| 757 | TGCATTATAGTGGTATCCAG | 7.9 | −22.4 | 67.4 | −29.5 | −0.6 | −5.8 |
| | SEQ. ID. NO:2129 | | | | | | |
| 1415 | ATTCATCAGAGATACCACTA | 8 | −20.9 | 63.3 | −28.9 | 0 | −3.5 |
| | SEQ. ID. NO:2130 | | | | | | |
| 12 | CAATGAGGTGAGGAGGAGGA | 8.1 | −23 | 67.6 | −31.1 | 0 | −1.6 |
| | SEQ. ID. NO:2131 | | | | | | |
| 761 | TCAGTGCATTATAGTGGTAT | 8.5 | −21.6 | 66.9 | −30.1 | 0 | −6.3 |
| | SEQ. ID. NO:2132 | | | | | | |
| 509 | GTTGAACTTGGGGAAACTGA | 8.6 | −20.9 | 61.6 | −29 | −0.2 | −3.2 |
| | SEQ. ID. NO:2133 | | | | | | |
| 1455 | TCCCCAATACTTTTATAAAA | 8.7 | −18.8 | 56 | −27 | 0 | −7.5 |
| | SEQ. ID. NO:2134 | | | | | | |
| 1454 | CCCCAATACTTTTATAAAAA | 8.8 | −17.7 | 53.3 | −26 | 0 | −7.8 |
| | SEQ. ID. NO:2135 | | | | | | |
| 293 | TCAAAAAAAACTCCAAAGTG | 8.9 | −14.2 | 46.9 | −22.4 | −0.5 | −3 |
| | SEQ. ID. NO:2136 | | | | | | |
| 759 | AGTGCATTATAGTGGTATCC | 9.6 | −22.9 | 69.6 | −32.5 | 0 | −6.3 |
| | SEQ. ID. NO:2137 | | | | | | |
| 760 | CAGTGCATTATAGTGGTATC | 14.3 | −21.6 | 66.9 | −35.9 | 0 | −6.3 |
| | SEQ. ID. NO:2138 | | | | | | |

### Example 15

Western Blot Analysis of FXR Protein Levels

Western blot analysis (immunoblot analysis) is carried out using standard methods. Cells are harvested 16-20 h after oligonucleotide treatment, washed once with PBS, suspended in Laemmli buffer (100 ul/well), boiled for 5 minutes and loaded on a 16% SDS-PAGE gel. Gels are run for 1.5 hours at 150 V, and transferred to membrane for western blotting. Appropriate primary antibody directed to FXR is used, with a radiolabeled or fluorescently labeled secondary antibody directed against the primary antibody species. Bands are visualized using a PHOSPHORIMAGER™ (Molecular Dynamics, Sunnyvale Calif.).

What is claimed is:

1. An antisense compound 8 to 30 nucleobases in length targeted to a nucleic acid molecule encoding FXR, wherein said antisense compound specifically hybridizes with and inhibits the expression of FXR.

2. The antisense compound of claim 1 which is an antisense oligonucleotide.

3. The antisense compound of claim 2 wherein said antisense oligonucleotide comprises at least 8 contiguous nucleic acids of a nucleic acid sequence of SEQ ID NO.1-SEQ ID NO:2138.

4. The antisense compound of claim 2 wherein said antisense oligonucleotide comprises a nucleic acid sequence of SEQ ID NO.1-SEQ ID NO:2138.

5. The antisense compound of claim 2 wherein said antisense oligonucleotide consists of at least 8 contiguous nucleic acids of a nucleic acid sequence of SEQ ID NO.1-SEQ ID NO:2138.

6. The antisense compound of claim 2 wherein said antisense oligonucleotide consists of a nucleic acid sequence of SEQ ID NO.1-SEQ ID NO:2138.

7. The antisense compound of claim 2 wherein the antisense oligonucleotide comprises at least one modified internucleoside linkage.

8. The antisense compound of claim 7 wherein the modified internucleoside linkage is a phosphorothioate linkage.

9. The antisense compound of claim 2 or 7 wherein the antisense oligonucleotide comprises at least one modified sugar moiety.

10. The antisense compound of claim 9 wherein the modified sugar moiety is a 2′-O-methoxyethyl sugar moiety.

11. The antisense compound of claim 2 wherein the antisense oligonucleotide comprises at least one modified nucleobase.

12. The antisense compound of claim 11 wherein the modified nucleobase is a 5-methylcytosine.

13. The antisense compound of claim 9 wherein the antisense oligonucleotide comprises at least one modified nucleobase.

14. The antisense compound of claim 13 wherein the modified nucleobase is a 5-methylcytosine.

15. The antisense compound of claim 2 wherein the antisense oligonucleotide is a chimeric oligonucleotide.

16. A composition comprising the antisense compound of claim 2 and a pharmaceutically acceptable carrier or diluent.

17. The composition of claim 16 further comprising a colloidal dispersion system.

18. A method of inhibiting the expression of FXR in cells or tissues comprising contacting said cells or tissues with the antisense compound of claim 2 so that expression of FXR is inhibited.

19. A method of treating a human having a disease or condition associated with FXR comprising administering to said animal a therapeutically or prophylactically effective amount of the antisense compound of claim 2 so that expression of FXR is inhibited.

20. The method of claim 19 wherein the disease or condition is diabetes.

21. The method of claim 19 wherein the disease or condition is an immunological disorder.

22. The method of claim 19 wherein the disease or condition is a cardiovascular disorder such as dyslipidemia and the symptoms thereof, atherosclerosis, low HDL, elevated LDL, hypercholesterolemia, gall stones, hypertriglyceridemia, and obesity.

23. The method of claim 19 wherein the disease or condition is a neurologic disorder.

24. The method of claim 19 wherein the disease or condition is ischemia/reperfusion injury.
